(19) United States
(12) Patent Application Publication
(10) Pub. No.: US 2026/0098075 A1
(43) Pub. Date: Apr. 9, 2026

(54) CHIMERIC ANTIGEN RECEPTOR SPACERS

(71) Applicant: Lyell Immunopharma, Inc., South San Francisco, CA (US)

(72) Inventors: Marc Joseph LAJOIE, Seattle, WA (US); Brian Douglas WEITZNER, Seattle, WA (US); Scott Edward BOYKEN, Seattle, WA (US); Spencer PARK, Seattle, WA (US); Yun SONG, Seattle, WA (US)

(73) Assignee: Lyell Immunopharma, Inc., South San Francisco, CA (US)

(21) Appl. No.: 19/322,325

(22) Filed: Sep. 8, 2025

Related U.S. Application Data
(63) Continuation of application No. 17/318,996, filed on May 12, 2021, now abandoned.
(60) Provisional application No. 63/023,751, filed on May 12, 2020.

(51) Int. Cl. C07K 14/725; A61K 38/00; A61K 39/00; A61K 40/11; A61K 40/31; A61K 40/42; A61P 35/00; C07K 7/06; C07K 16/30; C12N 5/0783

(52) U.S. Cl. CPC C07K 14/7051; A61K 40/11; A61K 40/31; A61K 40/4205; A61K 40/421; A61K 40/4211; A61P 35/00; C07K 7/06; C07K 16/30; C12N 5/0636; A61K 38/00; A61K 2039/505; C07K 2317/53; C07K 2319/03

(57) ABSTRACT

The present disclosure related to chimeric antigen receptors (CARs) comprising immunoglobulin (Ig) derived spacers, e.g., hinge or loop regions, fragments thereof, or combinations thereof. Ig derived spacer confers improved properties to the CARs, e.g., increased cytokine release with respect the CARs with spacers not derived from hinge regions and fragments thereof, loop regions from constant domains and fragments thereof, and combinations thereof. Also provided are cells expressing CARs comprising Ig derived spacers regions and methods to use the CARs to treat diseases or disorders, e.g., cancer. Some of the disclosed Ig derived spacers are fragments from, e.g., IgAQ1, IgA2, IgD, IgE, IgG1, IgG2, IgG3, IgG4, or IgM. In some aspects, the disclosed Ig derived spacers are derived from non-human immunoglobulins, e.g., mouse immunoglobulins such IgG2A. Other Ig derived spacer disclosed are modular constructs comprising several concatenated Ig hinges or fragments thereof.

### CROSS-REFERENCE TO RELATED APPLICATIONS

This application is a continuation of U.S. Nonprovisional application Ser. No. 17/318,996, filed on May 12, 2021, which claims the priority benefit of U.S. Provisional Application No. 63/023,751, filed on May 12, 2020, which is herein incorporated by reference in its entirety.

### REFERENCE TO SEQUENCE LISTING SUBMITTED ELECTRONICALLY

The content of the electronically submitted sequence listing in ASCII text file (Name: 4385_0160003_Seqlisting_ST26; Size: 6,501,713 bytes; and Date of Creation: Aug. 29, 2025) filed with the application is herein incorporated by reference in its entirety.

### TECHNICAL FIELD

The present disclosure provides chimeric antigen receptors (CAR) comprising a spacer derived from immunoglobulin hinge or constant regions.

### BACKGROUND ART

Adoptive immunotherapy using chimeric antigen receptor (CAR) expressing T cells is a promising cancer treatment, because these cells can directly recognize and kill antigen-expressing tumor cells in a human leukocyte antigen (HLA)-independent manner. However, besides a careful choice of the target tumor associated antigen, this therapeutic approach is highly dependent on the optimal molecular design of the CAR.

Accordingly, there is a need for methodologies that allow the systematic optimization of the extracellular spacer in CARs to allow for maximum CAR T cell efficacy.

### BRIEF SUMMARY

The present disclosure provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein (a) the spacer is between about 150 amino acids and about 125 amino acids in length; and, the distance between the epitope and the surface of the target cell membrane is less than about 10 Å; (b) the spacer is between about 125 amino acids and about 100 amino acids in length; and, the distance between the epitope and the surface of the target cell membrane is less than about 10 Å; (c) the spacer is between about 100 amino acids and about 75 amino acids in length; and, the distance between the epitope and the surface of the target cell membrane is less than about 10 Å; (d) the spacer is between about 75 amino acids and about 36 amino acids in length; and, the distance between the epitope and the surface of the target cell membrane is less than about 15 Å; (e) the spacer is between about 35 amino acids and about 21 amino acids in length; and, the distance between the epitope and the surface of the target cell membrane is between about 15 Å and about 25 Å; (f) the spacer is between about 20 amino acids and about 16 amino acids in length; and, the distance between the epitope and the surface of the target cell membrane is between about 25 Å and about 35 Å; (g) the spacer is between about 15 amino acids and about 11 amino acids in length; and, the distance between the epitope and the surface of the target cell membrane is between about 35 Å and about 45 Å; or, (h) the spacer is between about 10 amino acids and about 5 amino acids in length; and, the distance between the epitope and the surface of the target cell membrane is more than about 45 Å.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein (a) the spacer is between about 450 Å and about 375 Å in length; and, the distance between the epitope and the surface of the target cell membrane is less than about 10 Å; (b) the spacer is between about 375 Å and about 300 Å in length; and, the distance between the epitope and the surface of the target cell membrane is less than about 10 Å; (c) the spacer is between about 300 Å and about 225 Å in length; and, the distance between the epitope and the surface of the target cell membrane is less than about 10 Å; (d) the spacer is between about 225 Å and about 100 Å in length; and, the distance between the epitope and the surface of the target cell membrane is less than about 15 Å; (e) the spacer is between about 100 Å and about 60 Å in length; and, the distance between the epitope and the surface of the target cell membrane is between about 15 Å and about 25 Å; (f) the spacer is between about 60 Å and about 45 Å in length; and, the distance between the epitope and the surface of the target cell membrane is between about 25 Å and about 35 Å; (g) the spacer is between about 45 Å and about 30 Å in length; and, the distance between the epitope and the surface of the target cell membrane is between about 35 Å and about 45 Å; or, (h) the spacer is between about 30 Å and about 15 Å in length; and, the distance between the epitope and the surface of the target cell membrane is more than about 45 Å.

In some aspects, (a) the distance between the epitope and the surface of the target cell membrane is less than about 10 Å and the spacer is 125, 126, 127, 128, 129, 130, 131, 132, 133, 134, 135, 136, 137, 138, 139, 140, 141, 142, 143, 144, 145, 146, 147, 148, 149 or 150 amino acids in length; (b) the distance between the epitope and the surface of the target cell membrane is less than about 10 Å and the spacer is 100, 101, 102, 103, 104, 105, 106, 107, 108, 109, 110, 111, 112, 113, 114, 115, 116, 117, 118, 119, 120, 121, 122, 123, 124 or 125 amino acids amino acids in length; (c) the distance between the epitope and the surface of the target cell membrane is less than about 10 Å and the spacer is 75, 76, 77, 78, 79, 80, 81, 82, 83, 84, 85, 86, 87, 88, 89, 90, 91, 92, 93, 94, 95, 96, 97, 98, 99 or 100 amino acids in length; (d) the distance between the epitope and the surface of the target cell membrane is less than about 15 Å and the spacer is 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 amino acids in length; (e) the distance between the epitope and the surface of the target cell membrane is between about 15 Å and about 25 Å and the spacer is 21, 22, 23, 24, 25, 26, 27, 28, 29, 30, 31, 32, 33, 34 or 35 amino acids in length; (f) the distance between the epitope and the surface of the target cell membrane is between about 25 Å and about 35 Å and the spacer is 16, 17, 18, 19 or 20 amino acids in length; (g) the distance between the epitope and the surface of the target cell membrane is between about 35 Å and about 45 Å and the spacer is 11, 12, 13, 14, or 15 amino acids in length; or, (h) the distance between the epitope and the surface of the target cell membrane is more than about 45 Å and the spacer is 5, 6, 7, 8, 9 or 10 amino acids in length.

In some aspects, (a) the distance between the epitope and the surface of the target cell membrane is less than about 10 Å and the spacer is about 450 Å, about 440 Å, about 430 Å, about 420 Å, about 410 Å, about 400 Å, about 390 Å, about 380 Å or about 375 Å in length; (b) the distance between the epitope and the surface of the target cell membrane is less than about 10 Å and the spacer is about 375 Å, about 370 Å, about 360 Å, about 350 Å, about 340 Å, about 330 Å, about 320 Å, about 310 Å, or about 300 Å in length; (c) the distance between the epitope and the surface of the target cell membrane is less than about 10 Å and the spacer is about 300 Å, about 290 Å, about 280 Å, about 270 Å, about 260 Å, about 250 Å, about 240 Å, about 230 Å, or about 225 Å in length; (d) the distance between the epitope and the surface of the target cell membrane is less than about 15 Å the spacer is about 225 Å, about 220 Å, about 210 Å, about 200 Å, about 190 Å, about 180 Å, about 170 Å, about 160 Å, about 150 Å, about 140 Å, about 130 Å, about 120 Å, about 110 Å, or about 100 Å in length; (e) the distance between the epitope and the surface of the target cell membrane is between about 15 Å and about 25 Å and the spacer is about 100 Å, about 95 Å, about 90 Å, about 85 Å, about 80 Å, about 75 Å, about 70 Å, about 65 Å, or about 60 Å in length; (f) the distance between the epitope and the surface of the target cell membrane is between about 25 Å and about 35 Å and the spacer is about 60 Å, about 55 Å, about 50 Å, and about 45 Å in length; (g) the distance between the epitope and the surface of the target cell membrane is between about 35 Å and about 45 Å and the spacer is about 45 Å, about 40 Å, about 35 Å, or about 30 Å in length; or, (h) the distance between the epitope and the surface of the target cell membrane is more than about 45 Å and the spacer is about 30 Å, about 25 Å, about 20 Å, or about 15 Å in length.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence of formula CN—(X₁PRX₂P)ₘ-[L-(X₁PRX₂P)]ₙ—C_{C} wherein: (a) the spacer is located between the ligand-binding domain and the transmembrane domain of the CAR; (b) the spacer has a length of at least 15 amino acids; (c) m is an integer selected from 0 or 1; (d) n is an integer between 1 to 20; (e) L is a linker polypeptide sequence; (f) CN is an optional N-terminal capping sequence; (g) Cc is an optional C-terminal capping sequence; and, (h) X₁ and X₂ are independently selected from cysteine, glycine, alanine, or serine.

In some aspects, the spacer comprises two, three, four, five, or six X₁PRX₂P motifs. In some aspects, X₁PRX₂P comprises at least one cysteine. In some aspects, X₁PRX₂P is SEQ ID NO:4749. In some aspects, the L comprises a polypeptide of SEQ ID NO: 4223 or a fragment or variant thereof. In some aspects, when n>1, all L are identical. In some aspects, when n>1, at least one L is different from the other L. In some aspects, the CN comprises a polypeptide of SEQ ID NO: 4088 or a fragment or variant thereof.

In some aspects, the Cc comprises a polypeptide of SEQ ID NO: 4533 or a fragment or variant thereof. In some aspects, the CAR spacer comprises the sequence of formula (CPRCP)ₒ(EPKSCDTPPPCPRCP)ₚ, wherein o is an integer which is 0 or 1, and p is an integer which is 1, 2 or 3, wherein CPRCP is the sequence set forth in SEQ ID NO:4740, and wherein EPKSCDTPPPCPRCP is the sequence set forth in SEQ ID NO: 4477.

In some aspects, the CAR spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected the group consisting of SEQ ID NOS: 2813, 2903, 2983, 3358, 3728, and 4477.

In some aspects, the CAR spacer comprises a Cc capping sequence comprising a subsequence of the polypeptide of SEQ ID NO: 4533 or a fragment or variant thereof. In some aspects, the subsequence of the polypeptide of SEQ ID NO: 5 is the N-terminal AP. In some aspects, the CAR spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS 4833, 4834, 4835, 4836, 4837, and 4838.

In some aspects, the CAR spacer comprises a C_{N} capping sequence comprising a subsequence of the polypeptide of SEQ ID NO:4088 or a fragment or variant thereof. In some aspects, the CAR spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to the amino acid sequence of SEQ ID NO: 4833.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge region or a functional fragment thereof, wherein the spacer comprises an amino acid sequence of SEQ ID NO: 4841.

The disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge region or a functional fragment thereof, wherein the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 4839.

Also provided is a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge region or a functional fragment thereof, wherein the spacer comprises an amino acid sequence of formula (EX₃KX₄X₅X₆X₇DTX₈X₉X₁₀TCPRCP)q, wherein q is an integer between 1 and 10, and wherein: X₃ is L or P; X₄ is T or S; X₅ is P or C; X₆ is L, or none; X₇ is G, or none; X₈ is T or P; X₉ is H or P; and, X₁₀ is T or P.

Also provided is a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge region or a functional fragment thereof, wherein the spacer comprises at least one amino sequence A of SEQ ID NO: 4466 and/or at least one amino acid sequence B of SEQ ID NO: 4477, wherein the amino acid sequence of the CAR spacer corresponds to the formula A, B, AB, ABB, ABBB, ABBBB, ABBBBB, AA, AAA, AAAA, AAAAA, BB, BBB, BBBB, or BBBBB.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence selected from the sequences in TABLE 11 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 11.

In some aspects, the spacer further comprises an N-terminal subsequence comprising 1, 2, 3, 4, or 5 IgG3 CH1 C-terminal domain amino acids and/or a C-terminal subsequence comprising 1, 2, 3, 4, or 5 IgG3 CH2 N-terminal domain amino acids. In some aspects, the N-terminal subsequence comprising 1, 2, 3, 4, or 5 IgG3 CH1 C-terminal domain amino acids and/or C-terminal subsequence comprising 1, 2, 3, 4, or 5 IgG3 CH2 N-terminal domain amino acids comprises an amino acid sequence selected from the sequences in TABLE 12.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids SEQ ID NO: 1.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS: 2015, 1889, 1768, 4852, and any combination thereof.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence selected from the sequences in TABLE 1 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 1.

In some aspects, the CAR spacer further comprises (i) an N-terminal subsequence comprising 1, 2, 3, 4, or 5 IgD CH1 C-terminal domain amino acids and/or (ii) a C-terminal subsequence comprising 1, 2, 3, 4, or 5 IgD CH2 N-terminal domain amino acids. In some aspects, the IgD CH1 and CH2 sequence C-terminal domain amino acids and/or IgD CH1 and CH2 sequence N-terminal domain amino acids comprise an amino acid sequence selected from the sequences in TABLE 2.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 2560.

In some aspects, the spacer comprises an amino acid sequence selected from the sequences in TABLE 3 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 3. In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS: 4847, 4845, 4846, 2560, 4844, and any combination thereof.

In some aspects, the spacer further comprises an N-terminal subsequence comprising 1, 2, 3, 4, or 5 IgAQ1 CH1 C-terminal domain amino acids and/or a C-terminal subsequence comprising 1, 2, 3, 4, or 5 IgAQ1 CH2 N-terminal domain amino acids. In some aspects, the N-terminal subsequence comprising 1, 2, 3, 4, or 5 IgAQ1 CH1 C-terminal domain amino acids and/or C-terminal subsequence comprising 1, 2, 3, 4, or 5 IgAQ1 CH2 N-terminal domain amino acids comprises an amino acid sequence selected from the sequences in TABLE 4.

The present disclosure provides polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 4848.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS: 4523, 4850, 2713, 4524, 4525, and any combination thereof.

In some aspects, the spacer comprises an amino acid sequence selected from the sequences in TABLE 5 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 5. In some aspects, the spacer further comprises an N-terminal subsequence comprising 1, 2, 3, 4, or 5 IgA2 CH1 C-terminal domain amino acids and/or a C-terminal subsequence comprising 1, 2, 3, 4, or 5 IgA2 CH2 N-terminal domain amino acids. In some aspects, the N-terminal subsequence comprising 1, 2, 3, 4, or 5 IgA2 CH1 C-terminal domain amino acids and/or C-terminal subsequence comprising 1, 2, 3, 4, or 5 IgA2 CH2 N-terminal domain amino acids comprises an amino acid sequence selected from the sequences in TABLE 6.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 2723 or SEQ ID NO: 4843.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS: 4839, 4840, 4843, and any combination thereof. In some aspects, the spacer comprises an amino acid sequence selected from the sequences in TABLE 7 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 7. In some aspects, the spacer further comprises an N-terminal subsequence comprising 1, 2, 3, 4, or 5 IgG1 CH1 C-terminal domain amino acids and/or a C-terminal subsequence comprising 1, 2, 3, 4, or 5 IgG1 CH2 N-terminal domain amino acids. In some aspects, the N-terminal subsequence comprising 1, 2, 3, 4, or 5 IgG1 CH1 C-terminal domain amino acids and/or C-terminal subsequence comprising 1, 2, 3, 4, or 5 IgG1 CH2 N-terminal domain amino acids comprises an amino acid sequence selected from the sequences in TABLE 8.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 2768 or SEQ ID NO: 4842.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 4842. In some aspects, the spacer comprises an amino acid sequence selected from the sequences in TABLE 9A or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 9A. In some aspects, the spacer further comprises an N-terminal subsequence comprising 1, 2, 3, 4, or 5 IgG2 CH1 C-terminal domain amino acids and/or a C-terminal subsequence comprising 1, 2, 3, 4, or 5 IgG2 CH2 N-terminal domain amino acids. In some aspects, the N-terminal subsequence comprising 1, 2, 3, 4, or 5 IgG2 CH1 C-terminal domain amino acids and/or C-terminal subsequence comprising 1, 2, 3, 4, or 5 IgG2 CH2 N-terminal domain amino acids comprises an amino acid sequence selected from the sequences in TABLE 10A.

In some aspects, the present disclosure provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 4926.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS: 4830, 4831, 4832, and any combination thereof.

In some aspects, the spacer comprises an amino acid sequence selected from the sequences in TABLE 9B or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 9B. In some aspects, the spacer further comprises an N-terminal subsequence comprising 1, 2, 3, 4, or 5 IgG2 CH1 C-terminal domain amino acids and/or a C-terminal subsequence comprising 1, 2, 3, 4, or 5 IgG2 CH2 N-terminal domain amino acids. In some aspects, the N-terminal subsequence comprising 1, 2, 3, 4, or 5 IgG2 CH1 C-terminal domain amino acids and/or C-terminal subsequence comprising 1, 2, 3, 4, or 5 IgG2 CH2 N-terminal domain amino acids comprises an amino acid sequence selected from the sequences in TABLE 10B.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 4374.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 4856. In some aspects, the spacer comprises an amino acid sequence selected from the sequences in TABLE 15 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 15. In some aspects, the spacer further comprises an N-terminal subsequence comprising 1, 2, 3, 4, or 5 IgE CH1 C-terminal domain amino acids and/or a C-terminal subsequence comprising 1, 2, 3, 4, or 5 IgE CH2 N-terminal domain amino acids. In some aspects, the N-terminal subsequence comprising 1, 2, 3, 4, or 5 IgE CH1 C-terminal domain amino acids and/or C-terminal subsequence comprising 1, 2, 3, 4, or 5 IgE CH2 N-terminal domain amino acids comprises an amino acid sequence selected from the sequences in TABLE 16.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer located between the antigen-binding domain and the transmembrane domain comprising an amino acid sequence derived from a human immunoglobulin hinge and/or constant region or a functional fragment thereof, wherein the spacer comprises an amino acid sequence comprising at least five, six, or seven consecutive amino acids of SEQ ID NO: 4857.

In some aspects, the spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to amino acid sequence of SEQ ID NOS 4858, 4959, 4857, and any combination thereof.

In some aspects, the spacer comprises an amino acid sequence selected from the sequences in TABLE 17 or at least about 70%, at least about 75%, at least about 80%, at least about 85%, at least about 90%, at least about 95%, at least about 96%, at least about 97%, at least about 98%, at least about 99%, or about 100% sequence identity to a sequence in TABLE 17. In some aspects, the spacer further comprises an N-terminal subsequence comprising 1, 2, 3, 4, or 5 IgM CH1 C-terminal domain amino acids and/or a C-terminal subsequence comprising 1, 2, 3, 4, or 5 IgM CH2 N-terminal domain amino acids. In some aspects, the N-terminal subsequence comprising 1, 2, 3, 4, or 5 IgM CH1 C-terminal domain amino acids and/or C-terminal subsequence comprising 1, 2, 3, 4, or 5 IgM CH2 N-terminal domain amino acids comprises an amino acid sequence selected from the sequences in TABLE 18. In some aspects, the CAR spacer comprises an optional L1 linker and/or an optional L2 linker. In some aspects, an optional L1 linker and/or an optional L2 linker is a flexible linker. In some aspects, the optional L1 linker and/or an optional L2 linker is between 1 and 100 amino acids in length. In some aspects, the L1 linker is between 1 and 10 amino acids in length. In some aspects, the L1 linker comprises a Gly-Ser linker. In some aspects, the L1 linker comprises the sequence set forth in SEQ ID NO: 4818. In some aspects, the L2 linker is between 1 and 10 amino acids in length. In some aspects, the L2 comprises the sequence PGG. In some aspects, the human immunoglobulin hinge region is from IgAQ1, IgA2, IgD, IgE, IgG1, IgG2, IgG3, or IgM. In some aspects, the functional fragment of a human immunoglobulin hinge and/or constant region comprises (a) an internal subsequence of a hinge and/or constant region; (b) a C-terminal subsequence of a hinge and/or constant region; (c) an N-terminal subsequence of a hinge and/or constant region; (d) a hinge region extended 1 to 10 amino acids towards the N-terminal CH1 domain and/or C-terminal CH2 domain; (e) a subsequence of a hinge region extended 1 to 10 amino acids towards the N-terminal CH1 domain; (f) a subsequence of a hinge region extended 1 to 10 amino acids towards the C-terminal CH2 domain; (g) a sequence comprising 2 or more repeats of (a)-(f); (h) a combination of (a)-(g) corresponding to the same hinge and/or constant region; (i) a combination of (a)-(g) corresponding to different hinges and/or constant regions; or, (j) any combination thereof. In some aspects, the distance between the epitope and the surface of the target cell membrane is estimated using x-ray crystallography, NMR, or cryo-EM structure. In some aspects, the distance between the epitope and the surface of the target cell membrane is estimated using Fluorescence Resonance Energy Transfer (FRET). In some aspects, the CAR induces an increased IFNγ and/or IL-2 expression compared to a corresponding CAR comprising a reference spacer. In some aspects, the CAR induces an increased IFNγ expression by at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 20 fold, compared to a corresponding CAR comprising a reference spacer. In some aspects, the CAR induces an increased IL-2 expression by at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 20 fold, compared to a corresponding CAR comprising a reference spacer. In some aspects, the antigen-binding domain comprises an antibody or an antigen-binding fragment thereof that specifically binds to an epitope on a tumor antigen. In some aspects, the tumor antigen comprises ROR1, HER2, AFP, TRAC, TCRP, BCMA, CLL-1, CS1, CD38, CD19, TSHR, CD123, CD22, CD30, CD70, CD171, CD33, EGFRvIII, GD2, GD3, Tn Ag, PSMA, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, ber-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LTLRA2, CD300LF, CLECi2A, BST2, EMR2, LY75, GPC3, FCRL5, IGLL1, CD2, CD3F, CD4, CD5, CD7, the extracellular portion of the APRTL protein, and any combinations thereof.

In some aspects, the tumor antigen comprises ROR1, CD19 or Her2. In some aspects, the antigen-binding domain comprises an anti-ROR1 antibody or an antigen-binding fragment thereof. In some aspects, the antigen-binding domain cross-competes with the R11 antibody, R12 antibody, or 2A2 antibody. In some aspects, the antigen-binding domain binds to the same epitope as the R11 antibody, R12 antibody, or 2A2 antibody. In some aspects, the antigen-binding domain comprises heavy chain variable region (VH) CDR3 of the R11 antibody, R12 antibody, or 2A2 antibody. In some aspects, the antigen-binding domain further comprises VH CDR1 and VH CDR2. In some aspects, the VH CDR1 comprises the VH CDR1 of the R11 antibody, R12 antibody, or 2A2 antibody and/or the VH CDR2 comprises the VH CDR2 of the R11 antibody, R12 antibody, or 2A2 antibody. In some aspects, the antigen-binding domain further comprises light chain variable region (VL) CDR1, VL CDR2, and/or VL CDR3. In some aspects, the VL CDR1 comprises the VL CDR1 of the R11 antibody, R12 antibody, or 2A2 antibody, the VL CDR2 comprises the VL CDR2 of the R11 antibody, R12 antibody, or 2A2 antibody, and/or the VL CDR3 comprises the VL CDR3 of the R11 antibody, R12 antibody, or 2A2 antibody. In some aspects, the antigen-binding domain comprises: (i) VH CDR1 of SEQ ID NO: 4888; VH CDR2 of SEQ ID NO: 4889; and VH CDR3 of SEQ ID NO: 4890; and/or VL CDR1 of SEQ ID NO: 4892; VL CDR2 of SEQ ID NO: 4893; and VL CDR3 of SEQ ID NO: 4894; (ii) VH CDR1 of SEQ ID NO: 4896; VH CDR2 of SEQ ID NO: 4897; and VH CDR23 of SEQ ID NO: 4898; and/or VL CDR1 of SEQ ID NO: 4900; VL CDR2 of SEQ ID NO: 4901; and VL CDR3 of SEQ ID NO: 4902; or (iii) VH CDR1 of SEQ ID NO: 4904; VH CDR2 of SEQ ID NO: 4905; and VH CDR23 of SEQ ID NO: 4906; and/or VL CDR1 of SEQ ID NO: 4908; VL CDR2 of SEQ ID NO: 4909; and VL CDR3 of SEQ ID NO: 4910.

In some aspects, the antigen-binding domain comprises a heavy chain variable region (VH) and a light chain variable region (VL), and wherein: (i) the VH comprises SEQ ID NO: 4887; or the VL comprises SEQ ID NO: 4891; (ii) the VH comprises SEQ ID NO: 4895; or the VL comprises SEQ ID NO: 4899; or (iii) the VH comprises SEQ ID NO: 4903; or the VL comprises SEQ ID NO: 4907. In some aspects, the antigen-binding domain comprises a VH comprising SEQ ID NO: 4895 and a VL comprising SEQ ID NO: 4899; and wherein the spacer consists of SEQ ID NO: 4830. In some aspects, the CAR is designed as a standard CAR, a split CAR, an off-switch CAR, an on-switch CAR, a first-generation CAR, a second-generation CAR, a third-generation CAR, or a fourth-generation CAR. In some aspects, the antigen-binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, an affibody, a DARPin, a monobody, an adnectin, an alphabody, or a designed binder. In some aspects, the intracellular domain of the CAR is a signaling domain derived from CD3zeta, FcR gamma, FcR beta, CD3 gamma, CD3 delta, CD3 epsilon, CD5, CD22, CD79a, CD79b, and CD66d. In some aspects, the CAR further comprises a co-stimulatory domain derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, or B7-H3.

In some aspects, the polynucleotide is a DNA molecule, or a RNA molecule. In some aspects, the transmembrane domain is linked to the intracellular domain by a linker. In some aspects, the CAR is a bispecific CAR. In some aspects, the CAR is an inducible CAR.

The present disclosure also provides a vector comprising a polynucleotide disclosed herein operably linked to a regulatory element. In some aspects, the vector is a viral vector, a mammalian vector, or bacterial vector. In some aspects, the vector is a retroviral vector. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector. In some aspects, the vector is a lentivirus.

The present disclosure also provides a composition comprising a polynucleotide disclosed herein or a vector disclosed herein. Also provided is a kit comprising a polynucleotide, a vector, or a composition disclosed herein. Also provided is a CAR encoded by one or more polynucleotide sequences or vector disclosed herein. Also provided is a cell genetically modified to express a CAR, comprising a polynucleotide or a vector disclosed herein. In some aspects, the cell is a T cell, a natural killer (NK) cell, a natural killer T (NKT) cell, an ILC cell, a macrophage, or an antigen presenting cell.

The present disclosure provides a composition comprising a CAR disclosed herein or a cell disclosed herein. The present disclosure also provides a composition disclosed herein for treating a subject in need of a CAR therapy. Also provided is a pharmaceutical composition comprising a cell or a composition disclosed herein for treating cancer in a subject in need thereof. Also provided is a kit comprising a CAR, a cell, a composition or a pharmaceutical composition disclosed herein.

The present disclosure also provides the use of a polynucleotide, a vector, a composition, a kit, a CAR, a cell, a pharmaceutical composition, or a kit disclosed herein for the manufacture of a medicament for treating cancer in a subject in need thereof. Also provided is a method of stimulating a T cell-mediated immune response to a target cell population or tissue in a subject, comprising administering an effective amount of a cell disclosed herein to the subject. Also provided is a method of providing an anti-tumor immunity in a subject in need thereof, the method comprising administering to the subject an effective amount of a cell disclosed herein. Also provided is a method of treating cancer in a subject in need thereof comprising administering to the subject an effective amount of a cell disclosed herein. Also provided is a method of preparing a population of cells for a therapy comprising transducing a population of cells isolated from a subject with a polynucleotide or a vector disclosed herein. In some aspects, the transduction comprises culturing the cell under suitable condition.

The present disclosure also provides a method of generating a persisting population of genetically engineered cells in a subject diagnosed with cancer, the method comprising administering to the subject a cell genetically engineered to express a CAR disclosed herein. Also provided is a method of expanding a population of genetically engineered cells in a subject diagnosed with cancer, the method comprising administering to the subject a cell genetically engineered to express a CAR disclosed herein. In some aspects, the cell is a T cell. In some aspects, the cell is an autologous T cell. In some aspects, the subject is a human subject.

The present disclosure also provides a method to improve one or more properties of a CAR therapy comprising inserting a CAR spacer between an antigen-binding domain and a transmembrane domain of a CAR, wherein the CAR spacer is the spacer recited in a polynucleotide disclosed herein. Also provided is a method to improve one or more properties of a CAR therapy comprising inserting a CAR spacer between an antigen-binding domain and a transmembrane domain of a CAR, wherein the CAR spacer is the spacer recited in a polynucleotide disclosed herein, wherein the spacer is located between the ligand-binding domain and the transmembrane domain. In some aspects, the CAR spacer is the spacer recited in a polynucleotide disclosed herein. In some aspects, the one or more improved properties of the CAR therapy is increased secretion of one or more cytokines. In some aspects, the cytokine secretion induced by the CAR is increased with respect to the secretion observed after administration of a corresponding CAR comprising a reference spacer. In some aspects, the cytokine is an interleukin. In some aspects, the interleukin is interleukin-2. In some aspects, the cytokine is an interferon. In some aspects, the interferon is interferon-gamma.

The present disclosure also provides a method to design a CAR spacer comprising measuring the distance between the target epitope and the target cell surface, wherein the sequence of the spacer is an Ig hinge sequence, a subsequence thereof, or a combination thereof.

### BRIEF DESCRIPTION OF THE DRAWINGS/FIGURES

FIG. 1. Schematic representation of a CAR showing its domain organization. The present disclosure relates to the engineering of the extracellular spacer component of CARs. The binding domain can be, e.g., a single chain variable fragment (scFv) in the VH-VL or VL-VH orientation, or any antigen-binding portion of an antibody or combination thereof. The ectodomain comprises an antigen-binding domain and a CAR spacer. The endodomain can also be referred to as an intracellular or intracytoplasmic domain and comprises an ITAM and optional costimulatory modules.

FIG. 2. Schematic representation showing the differences in structure between different generations of CARs. First generation CARs comprise an antigen-binding domain (e.g., an scFv) connected to ITAM directly via a transmembrane region. Second generation CARs comprise a costimulatory molecule (CM1) interposed between ITAM and the transmembrane domain. Third generation CARs include an additional costimulatory molecule (CM2). In fourth generation CARs a costimulatory molecule has been replaced with an IL-12 inducer. Fifth generation CARs are based on second generation CARs, but they contain a truncated cytoplasmic IL-2 receptor j-chain domain.

FIG. 3. Schematic representation of the architecture of human immunoglobulins.

FIG. 4. Schematic representation showing the domain structures of IgAQ1 and IgA2.

FIG. 5. Schematic representation of IgG1, IgG2, IgG3 and IgG4.

FIG. 6. Sequence repeats (a or b) present (+) or absent (−) in different IgG3 allotypes. The only naturally occurring sequences are ab, abb, and abbb.

FIG. 7. Schematic representation of the relationship between spacer length, distance between the target epitope and target cell membrane surface, and the width of the signaling synapse.

FIG. 8. Schematic representation of the modular structure of the IgG3 hinge, and a library of modular spacers with varying lengths and compositions derived from the IgG3 hinge.

FIG. 9. List of immunoglobulin-derived spacers used in the examples (Spacers 1-11 and Spacers 13-31). Each spacer can comprise an optional Gly-Ser flexible linker sequence (an exemplary GLy-Ser linker sequence of SEQ ID NO: 4818 is shown; in some aspects the linker can be SEQ ID NO:5088). The tables show the length of each spacer in amino acids and theoretical extended conformation length in angstrom (only spacer, or spacer plus linker). The tables also indicate whether cysteines are present.

FIG. 10. Bar graph quantitating the levels of IFN-γ secretion after 24 hours by T cells expressing ROR1 CAR with the indicated spacers and Mock (untransduced) T cells in the absence of the target antigen. R12 CART cells were transduced with R12CAR-P2 Å-EGFRt. R12CAR: a CAR directed against human ROR1. P2 Å: a self-cleaving peptide. EGFRt: a truncated human EGFR containing EGFR extracellular Domains III and IV and an EGFR transmembrane domain while lacking EGFR Domains I and II and EGFR intracellular sequence. “Short” reference spacer means an IgG4 spacer of SEQ ID NO: 4911.

FIG. 11A. Graph summarizing the effects of the indicated spacers on IL-2 and IFN-γ secretion by R12 CART cells during a 24 hr co-culture with H1975 cells expressing human ROR1. R12 CART cells were transduced with R12CAR-P2 Å-EGFRt.

FIG. 11B. Graphs summarizing the effects of the indicated spacer lengths on IL-2 and IFN-γ secretion by R12 CART cells during a 24 hr co-culture with H1975 cells expressing human ROR1. R12 CART cells were transduced with R12CAR-P2 Å-EGFRt.

FIG. 11C. Graphs summarizing the correlation between target lytic capabilities and cytokine release profiles of T cells expressing R12 CAR with indicated spacers during a co-culture with H1975 cells expressing human ROR1. Target lytic capabilities were measured by integrating the area under the normalized target cell killing curves (i.e., area under the curve, or AUC). R12 CART cells were transduced with R12 CAR-P2 Å-EGFRt.

FIGS. 12A, 12B, 12C and 12D. Graphs summarizing effects of the indicated spacers, separated by antibody families from which the spacers originated, on long-term cytotoxic activity of R12 CART cells following repeated exposure to Jeko1 cells that express human ROR1. Mock (untransduced) T cell activity is included in each plot for comparison. The numbers of remaining Jeko1 cells during 3 rounds of co-culture with R12 CART cells were normalized to the first time point of each round (i.e., 0 hr, 68 hr, and 148 hr). R12 CART cells were transduced with R12CAR-P2 Å-EGFRt.

FIG. 12E. Lengths of the top five spacers (Spacers 1, 11, 13, 14, and Short) selected based on the long-term cytotoxic activity measured in FIGS. 12A, 12B, 12C and 12D.

FIGS. 13A and 13B. Percentage of transduced cells measured by surface expression of transduction marker EGFRt on primary T cells in donors D3868 (FIG. 13A) and D4869 (FIG. 13B).

FIGS. 13C and 13D. Median fluorescence intensity (MFI) for bound anti-FMC63 protein on live, transduced cells (EGFRt+ cells) in donor D3868 (FIG. 13C) and donor D4869 (FIG. 13D). Triangles indicate decreasing spacer length within a given immunoglobulin type.

FIGS. 14A-14E. Effects of indicated spacers on FMC63 CAR-T induced cytotoxicity in the presence of NucLight Red-labeled Raji (Raji-NLR) target cells expressing human CD19 target antigen in donor D3868. FIG. 14A shows the IncuCyte kinetic killing curves for each CAR variant with the indicated spacer. FIG. 14B shows the AUC calculated for each killing curve from FIG. 14A.

FIGS. 14C-14E are bar graphs showing concentrations of IFN-γ (FIG. 14C), IL-2 (FIG. 14D) and TNFα (FIG. 14E) in co-culture media 24 hours post co-culturing. “Short” reference spacer is an IgG4 hinge-derived spacer of SEQ ID NO: 4911. “Intermediate” reference spacer is an IgG4 hinge-derived spacer of SEQ ID NO:4912. “Long” reference spacer is an IgG4 hinge-derived spacer of SEQ ID NO: 4913. All IncuCyte kinetic killing curves represent NucLight Red signal over time. Non-transduced primary T cell are indicated as “NTC” in this and all drawings.

FIGS. 15A-15E. Effects of indicated spacers on FMC63 CAR-T induced cytotoxicity in the presence of Raji-NLR target cells expressing human CD19 target antigen in donor D4869. FIG. 15A shows the IncuCyte kinetic killing curves for each CAR variant with the indicated spacer. FIG. 15B shows the AUC calculated for each killing curve from FIG. 14A. FIGS. 15C-15E are bar graphs showing concentrations of IFN-γ (FIG. 15C), IL-2 (FIG. 15D) and TNFα (FIG. 15E) in co-culture media 24 hours post co-culturing.

FIGS. 16A-16E. Effects of indicated spacers on FMC63 CAR-T induced cytotoxicity in the presence of NucLight Red-labeled Nalm6 (Nalm6-NLR) target cells expressing human CD19 target antigen in donor D3868. FIG. 16A shows the IncuCyte kinetic killing curves for each CAR variant with the indicated spacer. FIG. 16B shows the AUC calculated for each killing curve from FIG. 14A. FIGS. 16C-16E are bar graphs showing concentrations of IFN-γ (FIG. 16C), IL-2 (FIG. 16D) and TNFα (FIG. 16E) in co-culture media 24 hours post co-culturing.

FIGS. 17A-17E. Effects of indicated spacers on FMC63 CAR-T induced cytotoxicity in the presence of Nalm6-NLR target cell expressing human CD19 target antigen in donor D4869. FIG. 17A shows the IncuCyte kinetic killing curves for each CAR variant with the indicated spacer.

FIG. 17B shows the AUC calculated for each killing curve from FIG. 14A. FIGS. 17C-17E are bar graphs showing concentrations of IFN-γ (FIG. 17C), IL-2 (FIG. 17D) and TNFα (FIG. 17E) in co-culture media 24 hours post co-culturing.

FIGS. 18A-18E. Effects of indicated spacers on FMC63 CAR-T induced cytotoxicity in the presence of NucLight Red-labeled Raji target cells with CD19 knocked out (Raji_CD19_KO-NLR) in donor D3868. FIG. 18A shows the IncuCyte kinetic killing curves for each CAR variant with the indicated spacer. FIG. 18B shows the AUC calculated for each killing curve from FIG. 14A. FIG. 18C-18E are bar graphs showing concentrations of IFN-γ (FIG. 18C), IL-2 (FIG. 18D) and TNFα (FIG. 18E) in co-culture media 24 hours post co-culturing.

FIGS. 19A-19E. Effects of indicated spacers on FMC63 CAR-T induced cytotoxicity in the presence of NucLight Red-labeled Raji target cells with CD19 knocked out (Raji_CD19_KO-NLR) in donor D4869. FIG. 19A shows the IncuCyte kinetic killing curves for each CAR variant with the indicated spacer. FIG. 19B shows the AUC calculated for each killing curve from FIG. 14A. FIG. 19C-19E are bar graphs showing concentrations of IFN-γ (FIG. 19C), IL-2 (FIG. 19D) and TNFα (FIG. 19E) in co-culture media 24 hours post co-culturing.

FIGS. 20A-20C. Bar graphs showing cytokine secretion profile on FMC63 CAR-T cells derived from donor D3868 expressing indicated spacers in the absence of target cells. The CAR-Ts with spacers exhibiting elevated cytokine production in the absence of target cells are considered to exhibit tonic signaling.

FIGS. 21A-21C. Bar graphs showing cytokine secretion profile on FMC63 CAR-T cells derived from donor D4869 expressing indicated spacers in the absence of target cells. The CAR-Ts with spacers exhibiting elevated cytokine production in the absence of target cells are considered to exhibit tonic signaling.

FIGS. 22A-22B. Scatter plots summarizing target-dependent cytokine secretion versus killing AUC for the FMC63 CAR-T (spacers indicated by labels) cells when co-cultured with Raji-NLR cells. FIG. 22A is donor D3868. FIG. 22B is donor D4869. The best-performing spacers are at the top left of the plot (high IFN-γ, high IL-2, and low AUC).

FIGS. 23A-23D. Scatter plots summarizing the effects of indicated spacer lengths on IL-2 and IFNγ secretion by FMC63 CAR-T cells after a 24-hour co-culture with human CD19 expressing target cells. The shaded boxes indicate the approximate window of spacer lengths that lead to maximal cytokine production. FIG. 23A shows FMC63 CAR-T derived from donor D3868 that was co-cultured with Raji cells. FIG. 23B shows FMC63 CAR-T derived from donor D3868 that was co-cultured with Nalm6 cells. FIG. 23C shows FMC63 CAR-T derived from donor D4869 that was co-cultured with Raji cells. FIG. 23D shows FMC63 CAR-T derived from donor D4869 was co-cultured with Nalm6 cells.

FIGS. 24A and 24B. Scatter plots summarizing the effects of indicated spacer lengths on killing AUC when Raji cells expressing human CD19 were co-cultured with FMC63 CAR-T cells, derived from donor D3868 (FIG. 24A) and donor D4869 (FIG. 24B), respectively. Spacer names are indicated by the labels. The circles represent the approximate window of spacer lengths that lead to fastest killing kinetics.

FIGS. 25A-25D. Graphs summarizing the effects of the indicated spacers on long-term cytotoxic activity of FMC63 CAR-T cells following repeated exposure to target cells that express human CD19 from donor D3868. FIG. 25A shows killing curves of Nalm6-NLR target cells during four rounds of co-culture with FMC63 CAR-T cells expressing CARs comprising indicated spacers.

FIG. 25B highlights a subset of FMC63 CAR-T cells with most persistent long-term cytotoxicity during repeated exposure to Nalm6 cells. FIG. 25C shows killing curves of Raji-NLR target cells during four rounds of co-culture with FMC63 CAR-T cells expressing CARs comprising indicated spacers. FIG. 25D highlights a subset of FMC63 CAR-T cells with most persistent long-term cytotoxicity during repeated exposure to Raji cells.

FIGS. 26A-26D. Graphs summarizing the effects of the indicated spacers on long-term cytotoxic activity of FMC63 CAR-T cells following repeated exposure to target cells that express human CD19 from donor D4869. FIG. 26A shows killing curves of Nalm6-NLR target cells during four rounds of co-culture with FMC63 CAR-T cells expressing CARs comprising indicated spacers.

FIG. 26B highlights a subset of FMC63 CAR-T cells with most persistent long-term cytotoxicity during repeated exposure to Nalm6 cells. FIG. 26C shows killing curves of Raji-NLR target cells during four rounds of co-culture with FMC63 CAR-T cells expressing CARs comprising indicated spacers. FIG. 26D highlights a subset of FMC63 CAR-T cells with most persistent long-term cytotoxicity during repeated exposure to Raji cells.

FIGS. 27A and 27B. Percentage of transduced cells measured by surface expression of transduction marker EGFRt on live primary T cells in donors D13814 (FIG. 27A) and D15842 (FIG. 27B).

FIGS. 27C and 27D. MFI for bound Protein L on live, transduced cells (EGFRt+ cells) in donor D13814 (FIG. 27C) and donor D15842 (FIG. 27D). Triangles indicate decreasing spacer length within a given immunoglobulin type.

FIGS. 28A-28E. Effects of indicated spacers on anti-Her2 CAR-T induced cytotoxicity in the presence of NucLight Red-labeled A549 target cells (A549-NLR) expressing human Her2 (human epidermal growth factor receptor 2) target antigen (using T cells from donor D13814). FIG. 28A shows the IncuCyte kinetic killing curves for each CAR variant with the indicated spacer. FIG. 28B shows the AUC calculated for each killing curve from FIG. 28A. FIGS. 28C-28E are bar graphs showing concentrations of IFN-γ (FIG. 28C), IL-2 (FIG. 28D) and TNFα (FIG. 28E) in co-culture media 24 hours post co-culturing.

FIGS. 29A-29E. Effects of indicated spacers on anti-Her2 CAR-T induced cytotoxicity in the presence of NucLight Red-labeled A549 target cells (A549-NLR) expressing human Her2 (human epidermal growth factor receptor 2) target antigen (using T cells from donor D15842). FIG. 29A shows the IncuCyte kinetic killing curves for each CAR variant with the indicated spacer. FIG. 29B shows the AUC calculated for each killing curve from FIG. 29A. FIGS. 29C-29E are bar graphs showing concentrations of IFN-γ (FIG. 29C), IL-2 (FIG. 29D) and TNFα (FIG. 29E) in co-culture media 24 hours post co-culturing.

FIGS. 30A-30E. Effects of indicated spacers on anti-Her2 CAR-T induced cytotoxicity in the presence of NucLight Red-labeled T47D target cells (T47D-NLR) expressing human Her2 (human epidermal growth factor receptor 2) target antigen (using T cells from donor D13814). FIG. 30A shows the IncuCyte kinetic killing curves for each CAR variant with the indicated spacer. FIG. 30B shows the AUC calculated for each killing curve from FIG. 30A. FIGS. 30C-30E are bar graphs showing concentrations of IFN-γ (FIG. 30C), IL-2 (FIG. 30D) and TNFα (FIG. 30E) in co-culture media 24 hours post co-culturing.

FIGS. 31A-31E. Effects of indicated spacers on anti-Her2 CAR-T induced cytotoxicity in the presence of NucLight Red-labeled T47D target cells (T47D-NLR) expressing human Her2 (human epidermal growth factor receptor 2) target antigen (using T cells from donor D15842). FIG. 31A shows the IncuCyte kinetic killing curves for each CAR variant with the indicated spacer. FIG. 31B shows the AUC calculated for each killing curve from FIG. 31A. FIGS. 31C-31E: are bar graphs showing concentrations of IFN-γ (FIG. 31C), IL-2 (FIG. 31D) and TNFα (FIG. 31E) in co-culture media 24 hours post co-culturing.

FIGS. 32A-32E. Effects of indicated spacers on anti-Her2 CAR-T induced cytotoxicity in the presence of NucLight Red-labeled T47D target cells with Her2 knocked out (T47D_Her2_KO-NLR) (using T cells from donor D13814). FIG. 32A shows the IncuCyte kinetic killing curves for each CAR variant with the indicated spacer. FIG. 32B shows the AUC calculated for each killing curve from FIG. 32A. FIGS. 32C-32E are bar graphs showing concentrations of IFN-γ (FIG. 32C), IL-2 (FIG. 32D) and TNFα (FIG. 32E) in co-culture media 24 hours post co-culturing.

FIGS. 33A-33E. Effects of indicated spacers on anti-Her2 CAR-T induced cytotoxicity in the presence of NucLight Red-labeled T47D target cells with Her2 knocked out (T47D_Her2_KO-NLR) (using T cells from donor D15842). FIG. 33A shows the IncuCyte kinetic killing curves for each CAR variant with the indicated spacer. FIG. 33B shows the AU) calculated for each killing curve from FIG. 33A. FIGS. 33C-33E are bar graphs showing concentrations of IFN-γ (FIG. 33C), IL-2 (FIG. 33D) and TNFα (FIG. 33E) in co-culture media 24 hours post co-culturing.

FIGS. 34A-34C. Bar graphs showing cytokine secretion profile (IFNg, FIG. 34A; IL-2, FIG. 34B; TNFa, FIG. 34C) on anti-Her2 (human epidermal growth factor receptor 2) CAR-T cells derived from donor D13814 expressing indicated spacers in the absence of target cells. The CAR-Ts with spacers exhibiting elevated cytokine production in the absence of target cells are considered to exhibit tonic signaling.

FIGS. 35A-35C. Bar graphs showing cytokine secretion profile (IFNg, FIG. 35A; IL-2, FIG. 35B; TNFa, FIG. 35C) on anti-Her2 CAR-T cells derived from donor D15842 expressing indicated spacers in the absence of target cells. The CAR-Ts with spacers exhibiting elevated cytokine production in the absence of target cells are considered to exhibit tonic signaling.

FIGS. 36A-36D. Scatter plots summarizing target-dependent cytokine secretion versus killing AUC for the anti-Her2 CAR-T (spacers indicated by labels) cells when co-cultured with human Her2 expressing target cells. FIG. 36A: anti-Her2 CAR-T derived from donor D13814 was co-cultured with A549 cells. FIG. 36B: anti-Her2 CAR-T derived from donor D13814 was co-cultured with T47D cells. FIG. 36C: anti-Her2 CAR-T derived from donor D15842 was co-cultured with A549 cells. FIG. 36D: anti-Her2 CAR-T derived from donor D15842 was co-cultured with T47D cells. The best-performing spacers are at the top left of the plot (high IFN-γ, high IL-2, and low AUC).

FIGS. 37A-37D. Scatter plots summarizing the effects of indicated spacer lengths on IL-2 and IFNg secretion by anti-Her2 CAR-T cells after a 24-hour co-culture with human Her2 expressing target cells. The shaded boxes indicate the approximate window of spacer lengths that lead to maximal cytokine production. FIG. 37A: anti-Her2 CAR-T derived from donor D13814 was co-cultured with A549 cells. FIG. 37B: anti-Her2 CAR-T derived from donor D13814 was co-cultured with T47D cells. FIG. 37C: anti-Her2 CAR-T derived from donor D15842 was co-cultured with A549 cells. FIG. 37D: anti-Her2 CAR-T derived from donor D15842 was co-cultured with T47D cells.

FIGS. 38A-38D. Scatter plots summarizing the effects of indicated spacer lengths on killing AUC when target cells expressing human Her2 were co-cultured with anti-Her2 CAR-T cells. FIG. 38A: anti-Her2 CAR-T derived from donor D13814 was co-cultured with A549 cells. FIG. 38B: anti-Her2 CAR-T derived from donor D13814 was co-cultured with T47D cells. FIG. 38C: anti-Her2 CAR-T derived from donor D15842 was co-cultured with A549 cells. FIG. 38D: anti-Her2 CAR-T derived from donor D15842 was co-cultured with T47D cells. Spacer names are indicated by the labels. The circles represent the approximate window of spacer lengths that lead to fastest killing kinetics.

FIGS. 39A-39D. Graphs summarizing the effects of the indicated spacers on long-term cytotoxic activity of anti-Her2 CAR-T cells from donor D13814 following repeated exposure to target cells that express human Her2. FIG. 39A shows killing curves of A549-NLR target cells during four rounds of co-culture with anti-Her2 CAR-T cells expressing CARs comprising indicated spacers. FIG. 39B highlights a subset of anti-Her2 CAR-T cells with most persistent long-term cytotoxicity during repeated exposure to A549 cells. FIG. 39C shows killing curves of T47D-NLR target cells during four rounds of co-culture with anti-Her2 CAR-T cells expressing CARs comprising indicated spacers.

FIG. 39D highlights a subset of anti-Her2 CAR-T cells with most persistent long-term cytotoxicity during repeated exposure to T47D cells.

FIGS. 40A-40D. Graphs summarizing the effects of the indicated spacers on long-term cytotoxic activity of anti-Her2 CAR-T cells from donor D15842 following repeated exposure to target cells that express human Her2. FIG. 40A shows killing curves of A549-NLR target cells during four rounds of co-culture with anti-Her2 CAR-T cells expressing CARs comprising indicated spacers. FIG. 40B highlights a subset of anti-Her2 CAR-T cells with most persistent long-term cytotoxicity during repeated exposure to A549 cells. FIG. 40C shows killing curves of T47D-NLR target cells during four rounds of co-culture with anti-Her2 CAR-T cells expressing CARs comprising indicated spacers.

FIG. 40D highlights a subset of anti-Her2 CAR-T cells with most persistent long-term cytotoxicity during repeated exposure to T47D cells.

FIG. 41. Three-dimensional structure of the Ig fold (A), and the topology of the structural elements in the fold (B). The location of the β-strand secondary structure elements and the location of the loop regions connecting the β-strands is shown. As disclosed below, each loop region or fragment thereof (optionally comprising one or more amino acids from an adjacent β-strand) from an immunoglobulin constant domain (e.g., a CH1, CH2, CH3, or CL constant domain) from a human or murine immunoglobulin can be used as a CAR spacer of the present disclosure.

FIG. 42. Multiple sequence alignment of immunoglobulin heavy chains showing the secondary structure (Conformation) overlaid onto the sequence alignment. As disclosed in the specification, subsequences having a coil secondary structure or enriched in amino acid residues having a coil secondary structure can be used as CAR spacers of the present disclosure. Regions having high concentrations, of proline, cysteine, and glycine, are highlighted.

FIG. 43. Multiple sequence alignment of immunoglobulin light chains showing the secondary structure (Conformation) overlaid onto the sequence alignment. As disclosed in the specification, subsequences having a coil secondary structure or enriched in amino acid residues having a coil secondary structure can be used as CAR spacers of the present disclosure.

FIGS. 44A and 44B. Percentage of transduced cells measured by surface expression of transduction marker EGFRt on live primary T cells in donor D7811 (FIG. 44A) and donor D5018 (FIG. 44B). FIGS. 44C and 44D show the MFI for bound hROR1-Fc on live, transduced cells (EGFRt+ cells) in donor D7811 (FIG. 44C) and donor D5018 (FIG. 44D).

FIGS. 45A-45C. Effects of indicated spacers on R11-CAR-induced cytotoxicity in the presence of NucLight Red-labeled A549 target cells (A549-NLR) expressing human ROR1 target antigen in donor D7811. FIG. 45A shows the AUC calculated for IncuCyte kinetic killing curves for each CAR variant with the indicated spacer. Spacers for which no in vitro functional data was generated are indicated as “nd” (not determined) in this figure and figures below. FIG. 45B-C are bar graphs showing concentrations of IFN-g (B) and IL-2 (C) in co-culture media 24 hours post co-culturing. Open bars indicate measurements outside of assay linear dynamic range, therefore should not be interpreted quantitatively.

FIGS. 46A-46C. Effects of indicated spacers on R11-CAR-induced cytotoxicity in the presence of NucLight Red-labeled A549 target cells (A549-NLR) expressing human ROR1 target antigen in donor D5018. FIG. 46A shows the AUC calculated for IncuCyte kinetic killing curves for each CAR variant with the indicated spacer. FIG. 46B-C are bar graphs showing concentrations of IFN-g (B) and IL-2 (C) in co-culture media 24 hours post co-culturing. Open bars indicate measurements outside of assay linear dynamic range, therefore should not be interpreted quantitatively.

FIGS. 47A-47C. Effects of indicated spacers on R11-CAR-induced cytotoxicity in the presence of NucLight Red-labeled H1975 target cells (H1975-NLR) expressing human ROR1 target antigen in donor D7811. FIG. 47A shows the AUC calculated for IncuCyte kinetic killing curves for each CAR variant with the indicated spacer. FIG. 47B-C are bar graphs showing concentrations of IFN-g (B) and IL-2 (C) in co-culture media 24 hours post co-culturing. Open bars indicate measurements outside of assay linear dynamic range, therefore should not be interpreted quantitatively.

FIGS. 48A-48B. Effects of indicated spacers on R11-CAR-induced cytotoxicity in the presence of NucLight Red-labeled H1975 target cells (H1975-NLR) expressing human ROR1 target antigen in donor D5018. FIG. 48A shows the AUC calculated for IncuCyte kinetic killing curves for each CAR variant with the indicated spacer. FIG. 48B is a bar graph showing concentrations of IFN-g in co-culture media 24 hours post co-culturing.

FIGS. 49A-49B. Effects of indicated spacers on R11-CAR-induced cytotoxicity in the presence of NucLight Red-labeled A549 target cells with ROR1 knocked out (A549_ROR1_KO-NLR) in donor D7811. FIG. 49A shows the AUC calculated for IncuCyte kinetic killing curves for each CAR variant with the indicated spacer. FIG. 49B is a bar graph showing concentrations of IFN-g in co-culture media 24 hours post co-culturing. A small population of ROR1+ cells is present in A549_ROR1_KO-NLR cell line, which accounts for the apparent killing (A) and cytokine production (B) in this assay.

FIGS. 50A-50B. Effects of indicated spacers on R11-CAR-induced cytotoxicity in the presence of NucLight Red-labeled A549 target cells with ROR1 knocked out (A549_ROR1_KO-NLR) in donor D5018. FIG. 50A shows the AUC calculated for IncuCyte kinetic killing curves for each CAR variant with the indicated spacer. FIG. 50B is a bar graph showing concentrations of IFN-g in co-culture media 24 hours post co-culturing. A small population of ROR1+ cells is present in A549_ROR1_KO-NLR cell line, which accounts for the apparent killing (A) and cytokine production (B) in this assay.

FIG. 51. Bar graph showing cytokine secretion profile on R11 CAR-T cells derived from donor D7811 expressing CARs having the indicated spacers in the absence of target cells. No spacer resulted in elevated cytokine production in the absence of target cells to be considered as tonic signaling.

FIG. 52. Bar graph showing cytokine secretion profile on R11 CAR-T cells derived from donor D5018 expressing CARs having the indicated spacers in the absence of target cells. No spacer resulted in elevated cytokine production in the absence of target cells to be considered as tonic signaling.

FIG. 53A-53B. Scatter plots summarizing the effects of indicated spacer lengths on target-dependent cytokine secretion (dot size IFN-g), and killing AUC for the R11 CAR-T cells when co-cultured with human ROR1 expressing target cells. FIG. 53A: R11 CAR-T derived from donor D7811 was co-cultured with A549 cells. FIG. 53B: R11 CAR-T derived from donor D7811 was co-cultured with H1975 cells.

FIG. 54A-54B. Scatter plots summarizing the effects of indicated spacer lengths on target-dependent cytokine secretion (dot size IFN-g), and killing AUC for the R11 CAR-T cells when co-cultured with target cells. FIG. 54A: R11 CAR-T derived from donor D5018 was co-cultured with A549 cells. FIG. 54B: R11 CAR-T derived from donor D5018 was co-cultured with H1975 cells.

FIGS. 55A and 55B. Percentage of transduced cells measured by surface expression of transduction marker EGFRt on live primary T cells in donor D2735 (FIG. 55A) and donor D5018 (FIG. 55B).

FIGS. 55C and 55D. MFI for bound hROR1-Fc on live, transduced cells (EGFRt+ cells) in donor D2735 (FIG. 55C) and donor D5018 (FIG. 55D).

FIGS. 56A-56C. Effects of indicated spacers on R12-CAR-induced cytotoxicity in the presence of NucLight Red-labeled A549 target cells (A549-NLR) expressing human ROR1 target antigen in donor D2735. FIG. 56A shows the AUC calculated for IncuCyte kinetic killing curves for each CAR variant with the indicated spacer. FIGS. 56B and 56C are bar graphs showing concentrations of IFN-g (B) and IL-2 (C) in co-culture media 24 hours post co-culturing. Open bars indicate measurements outside of assay linear dynamic range, therefore should not be interpreted quantitatively.

FIGS. 57A-57C. Effects of indicated spacers on R12-CAR-induced cytotoxicity in the presence of NucLight Red-labeled A549 target cells (A549-NLR) expressing human ROR1 target antigen in donor D5018. FIG. 57A shows the AUC calculated for IncuCyte kinetic killing curves for each CAR variant with the indicated spacer. FIGS. 57B and 57C are bar graphs showing concentrations of IFN-g (B) and IL-2 (C) in co-culture media 24 hours post co-culturing.

FIGS. 58A-58C. Effects of indicated spacers on R12-CAR-induced cytotoxicity in the presence of NucLight Red-labeled H1975 target cells (H1975-NLR) expressing human ROR1 target antigen in donor D2735. FIG. 58A shows the AUC calculated for IncuCyte kinetic killing curves for each CAR variant with the indicated spacer. FIGS. 58B and 58C are bar graphs showing concentrations of IFN-g (B) and IL-2 (C) in co-culture media 24 hours post co-culturing. Open bars indicate measurements outside of assay linear dynamic range, therefore should not be interpreted quantitatively.

FIGS. 59A-59C. Effects of indicated spacers on R12-CAR-induced cytotoxicity in the presence of NucLight Red-labeled H1975 target cells (H1975-NLR) expressing human ROR1 target antigen in donor D5018. FIG. 59A shows the AUC calculated for IncuCyte kinetic killing curves for each CAR variant with the indicated spacer. FIGS. 59B and 59C are bar graphs showing concentrations of IFN-g (B) and IL-2 (C) in co-culture media 24 hours post co-culturing.

FIGS. 60A-60B. Effects of indicated spacers on R12-CAR-induced cytotoxicity in the presence of NucLight Red-labeled A549 target cells with ROR1 knocked out (A549_ROR1_KO-NLR) in donor D2735. FIG. 60A shows the AUC calculated for IncuCyte kinetic killing curves for each CAR variant with the indicated spacer. FIG. 60B is a bar graph showing concentrations of IFN-g in co-culture media 24 hours post co-culturing. A small population of ROR1+ cells is present in A549_ROR1_KO-NLR cell line, which accounts for the apparent killing (A) and cytokine production (B) in this assay.

FIGS. 61A-61B. Effects of indicated spacers on R12-CAR-induced cytotoxicity in the presence of NucLight Red-labeled A549 target cells with ROR1 knocked out (A549_ROR1_KO-NLR) in donor D5018. FIG. 61A shows the AUC calculated for IncuCyte kinetic killing curves for each CAR variant with the indicated spacer. FIG. 61B is a bar graph showing concentrations of IFN-g in co-culture media 24 hours post co-culturing. A small population of ROR1+ cells is present in A549_ROR1_KO-NLR cell line, which accounts for the apparent killing (A) and cytokine production (B) in this assay.

FIG. 62. Bar graph showing cytokine secretion profile on R12 CAR-T cells derived from donor D2735 expressing CARs having the indicated spacers in the absence of target cells. No spacer resulted in elevated cytokine production in the absence of target cells to be considered as tonic signaling.

FIG. 63. Bar graph showing cytokine secretion profile on R12 CAR-T cells derived from donor D5018 expressing CARs having the indicated spacers in the absence of target cells. No spacer resulted in elevated cytokine production in the absence of target cells to be considered as tonic signaling.

FIG. 64A-64B. Scatter plots summarizing the effects of indicated spacer lengths on target-dependent cytokine secretion (dot size IFN-g), and killing AUC for the R12 CAR-T cells when co-cultured with human ROR1 expressing target cells. FIG. 64A: R12 CAR-T derived from donor D2735 was co-cultured with A549 cells. FIG. 64B: R12 CAR-T derived from donor D2735 was co-cultured with H1975 cells.

FIG. 65A-65B. Scatter plots summarizing the effects of indicated spacer lengths on target-dependent cytokine secretion (dot size IFN-g), and killing AUC for the R12 CAR-T cells when co-cultured with target cells. FIG. 65A: R12 CAR-T derived from donor D5018 was co-cultured with A549 cells. FIG. 65B: R12 CAR-T derived from donor D5018 was co-cultured with H1975 cells.

FIGS. 66A and 66B. Percentage of transduced cells measured by surface expression of transduction marker EGFRt on live primary T cells in donor D2089 (FIG. 66A) and donor D5018 (FIG. 66B).

FIGS. 66C and 66D show the MFI for bound hROR1-Fc on live, transduced cells (EGFRt+ cells) in donor D2089 (FIG. 66C) and donor D5018 (FIG. 66D).

FIGS. 67A-67B. Effects of indicated spacers on 2A2-CAR-induced cytotoxicity in the presence of NucLight Red-labeled A549 target cells (A549-NLR) expressing human ROR1 target antigen in donor D2089. FIG. 67A shows the AUC calculated for IncuCyte kinetic killing curves for each CAR variant with the indicated spacer. FIG. 67B is a bar graph showing concentrations of IFN-g in co-culture media 24 hours post co-culturing.

FIGS. 68A-68B summarize the effects of indicated spacers on 2A2-CAR-induced cytotoxicity in the presence of NucLight Red-labeled A549 target cells (A549-NLR) expressing human ROR1 target antigen in donor D5018. FIG. 68A shows the AUC calculated for IncuCyte kinetic killing curves for each CAR variant with the indicated spacer. FIG. 68B is a bar graph showing concentrations of IFN-g in co-culture media 24 hours post co-culturing.

FIGS. 69A-69B summarize the effects of indicated spacers on 2A2-CAR-induced cytotoxicity in the presence of NucLight Red-labeled H1975 target cells (H1975-NLR) expressing human ROR1 target antigen in donor D2089. FIG. 69A shows the AUC calculated for IncuCyte kinetic killing curves for each CAR variant with the indicated spacer. FIG. 69B is a bar graph showing concentrations of IFN-g in co-culture media 24 hours post co-culturing. Open bars indicate measurements outside of assay linear dynamic range, therefore should not be interpreted quantitatively.

FIGS. 70A-70B summarize the effects of indicated spacers on 2A2-CAR-induced cytotoxicity in the presence of NucLight Red-labeled H1975 target cells (H1975-NLR) expressing human ROR1 target antigen in donor D5018. FIG. 70A shows the AUC calculated for IncuCyte kinetic killing curves for each CAR variant with the indicated spacer. FIG. 70B is a bar graph showing concentrations of IFN-g in co-culture media 24 hours post co-culturing. Open bars indicate measurements outside of assay linear dynamic range, therefore should not be interpreted quantitatively.

FIGS. 71A-71B. Effects of indicated spacers on 2A2-CAR-induced cytotoxicity in the presence of NucLight Red-labeled A549 target cells with ROR1 knocked out (A549_ROR1_KO-NLR) in donor D2089. FIG. 71A shows the AUC calculated for IncuCyte kinetic killing curves for each CAR variant with the indicated spacer. FIG. 71B is a bar graph showing concentrations of IFN-g in co-culture media 24 hours post co-culturing. A small population of ROR1+ cells is present in A549_ROR1_KO-NLR cell line, which accounts for the apparent killing (A) and cytokine production (B) in this assay.

FIGS. 72A-72B summarize the effects of indicated spacers on 2A2-CAR-induced cytotoxicity in the presence of NucLight Red-labeled A549 target cells with ROR1 knocked out (A549_ROR1_KO-NLR) in donor D5018. FIG. 72A shows the AUC calculated for IncuCyte kinetic killing curves for each CAR variant with the indicated spacer. FIG. 72B is a bar graph showing concentrations of IFN-g in co-culture media 24 hours post co-culturing. A small population of ROR1+ cells is present in A549_ROR1_KO-NLR cell line, which accounts for the apparent killing (A) and cytokine production (B) in this assay.

FIG. 73. Bar graph showing cytokine secretion profile on 2A2 CAR-T cells derived from donor D2089 expressing CARs having the indicated spacers in the absence of target cells. No spacer resulted in elevated cytokine production in the absence of target cells to be considered as tonic signaling.

FIG. 74. Bar graph showing cytokine secretion profile on 2A2 CAR-T cells derived from donor D5018 expressing CARs having the indicated spacers in the absence of target cells. No spacer resulted in elevated cytokine production in the absence of target cells to be considered as tonic signaling.

FIG. 75A-75B. Scatter plots summarizing the effects of indicated spacer lengths on target-dependent cytokine secretion (dot size IFN-g), and killing AUC for the 2A2 CAR-T cells when co-cultured with human ROR1 expressing target cells. FIG. 75A: 2A2 CAR-T derived from donor D2089 was co-cultured with A549 cells. FIG. 75B: 2A2 CAR-T derived from donor D2089 was co-cultured with H1975 cells.

FIG. 76A-76B. Scatter plots summarizing the effects of indicated spacer lengths on target-dependent cytokine secretion (dot size IFN-g), and killing AUC for the 2A2 CAR-T cells when co-cultured with target cells. FIG. 76A: 2A2 CAR-T derived from donor D5018 was co-cultured with A549 cells. FIG. 76B: 2A2 CAR-T derived from donor D5018 was co-cultured with H1975 cells.

FIGS. 77A-77E. Scatter plots summarizing the differential effects of indicated spacer lengths on target-dependent cytokine secretion (dot size IFN-g), and killing AUC for the FMC63, Her, R11, R12 and 2A2 CAR-T cells when co-cultured with target cells expressing human CARs. The scatter plots indicate the optimal spacer length for R12 (FIG. 77A), 2A2 (FIG. 77B), FMC63 (FIG. 77C), Her (FIG. 77D) and R11 (FIG. 77E) CARs.

FIG. 78A. Tumor growth over time in mice treated with 2,000,000 CAR-positive T cells with the indicated R12-spacer construct.

FIG. 78B. Body weight change over time in mice treated with 2,000,000 CAR-positive T cells with the indicated R12-spacer construct.

FIG. 79A. Tumor growth over time in mice treated with 500,000 CAR-positive T cells with the indicated R12-spacer construct.

FIG. 79B. Body weight change over time in mice treated with 500,000 CAR-positive T cells with the indicated R12-spacer construct.

FIG. 80. Percent survival over time in mice treated with 2,000,000 CAR-positive T cells with the indicated R12-spacer construct.

FIG. 81. Percent survival over time in mice treated with 500,000 CAR-positive T cells with the indicated R12-spacer construct.

FIG. 82. Number of CD3+ cells per mL blood on day 1 post T cell dose. High dose represents 2,000,000 CAR-positive T cells with the indicated R12-spacer construct. Low dose represents 500,000 CAR-positive T cells with the indicated R12-spacer construct.

FIG. 83. Number of CD3+EGFR+ cells (i.e., CAR T cells) per mL blood on day 1 post T cell dose. High dose represents 2,000,000 CAR-positive T cells with the indicated R12-spacer construct. Low dose represents 500,000 CAR-positive T cells with the indicated R12-spacer construct.

FIGS. 84A-84L. Number of T cells per mL blood over time in mice treated with 2,000,000 CAR-positive T cells with the indicated R12-spacer construct. FIG. 84A shows blood PK for CD3+ cells (all T cells), FIG. 84B shows blood PK for CD3+EGFR+ cells (all CAR T cells), FIG. 84C shows blood PK for CD3+CAR+ cells (all CAR T cells), FIG. 84D shows blood PK for CD8+ cells (all CD8+ T cells), FIG. 84E shows blood PK for CD8+EGFR+ cells (CD8+CAR T cells), FIG. 84F shows blood PK for CD8+CAR+ cells (CD8+CAR T cells), FIG. 84G shows blood PK for CD8+CD4+ cells (all CD8+CD4+ T cells), FIG. 84H shows blood PK for CD8+CD4+EGFR+ cells (CD8+CD4+CAR T cells), FIG. 84I shows blood PK for CD8+CD4+CAR+ cells (CD8+CD4+CAR T cells), FIG. 84J shows blood PK for CD4+ cells (all CD4+ T cells), FIG. 84K shows blood PK for CD4+EGFR+ cells (CD4+CAR T cells), and FIG. 84L shows blood PK for CD4+CAR+ cells (CD4+CAR T cells).

FIGS. 85A-85L. Number of T cells per mL blood over time in mice treated with 500,000 CAR-positive T cells with the indicated R12-spacer construct. FIG. 85A shows blood PK for CD3+ cells (all T cells), FIG. 85B shows blood PK for CD3+EGFR+ cells (all CAR T cells), FIG. 85C shows blood PK for CD3+CAR+ cells (all CAR T cells), FIG. 85D shows blood PK for CD8+ cells (all CD8+ T cells), FIG. 85E shows blood PK for CD8+EGFR+ cells (CD8+CAR T cells), FIG. 85F shows blood PK for CD8+CAR+ cells (CD8+CAR T cells), FIG. 85G shows blood PK for CD8+CD4+ cells (all CD8+CD4+ T cells), FIG. 85H shows blood PK for CD8+CD4+EGFR+ cells (CD8+CD4+CAR T cells), FIG. 85I shows blood PK for CD8+CD4+CAR+ cells (CD8+CD4+CAR T cells), FIG. 85J shows blood PK for CD4+ cells (all CD4+ T cells), FIG. 85K shows blood PK for CD4+EGFR+ cells (CD4+CAR T cells), and FIG. 85L shows blood PK for CD4+CAR+ cells (CD4+CAR T cells).

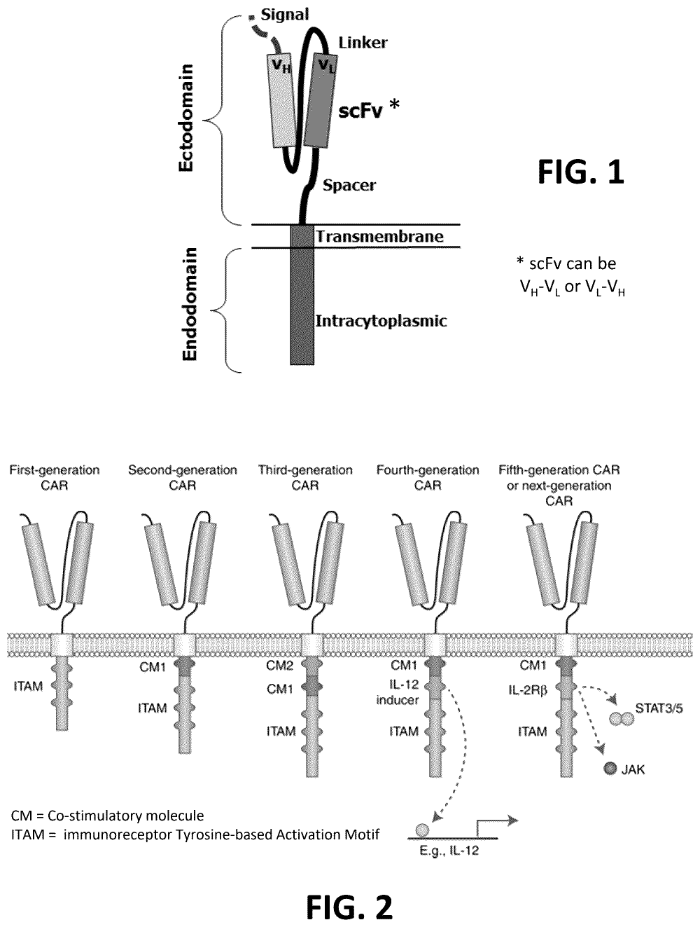

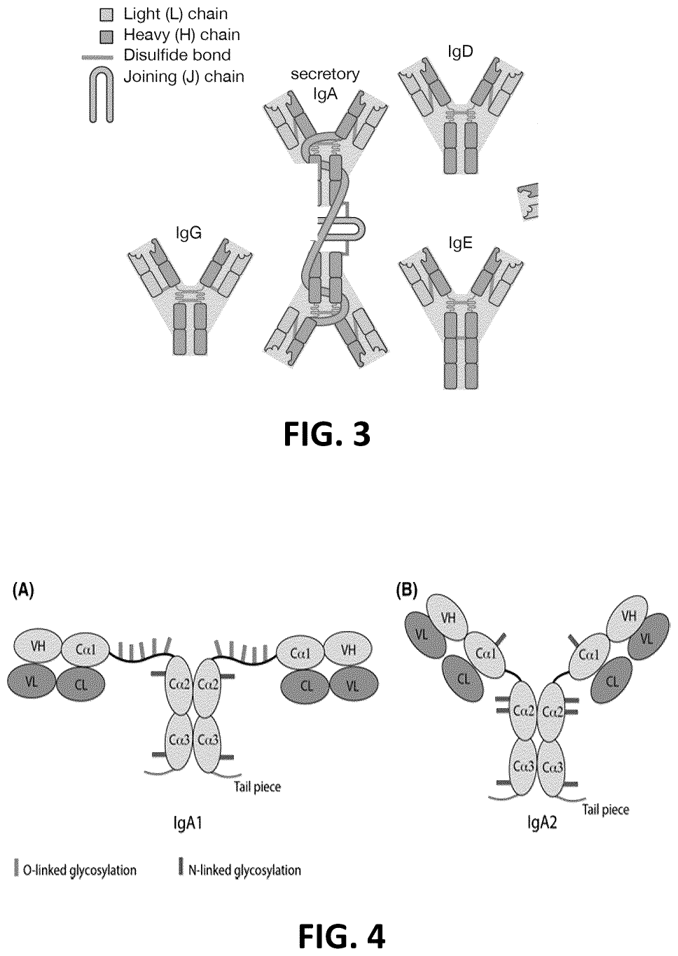

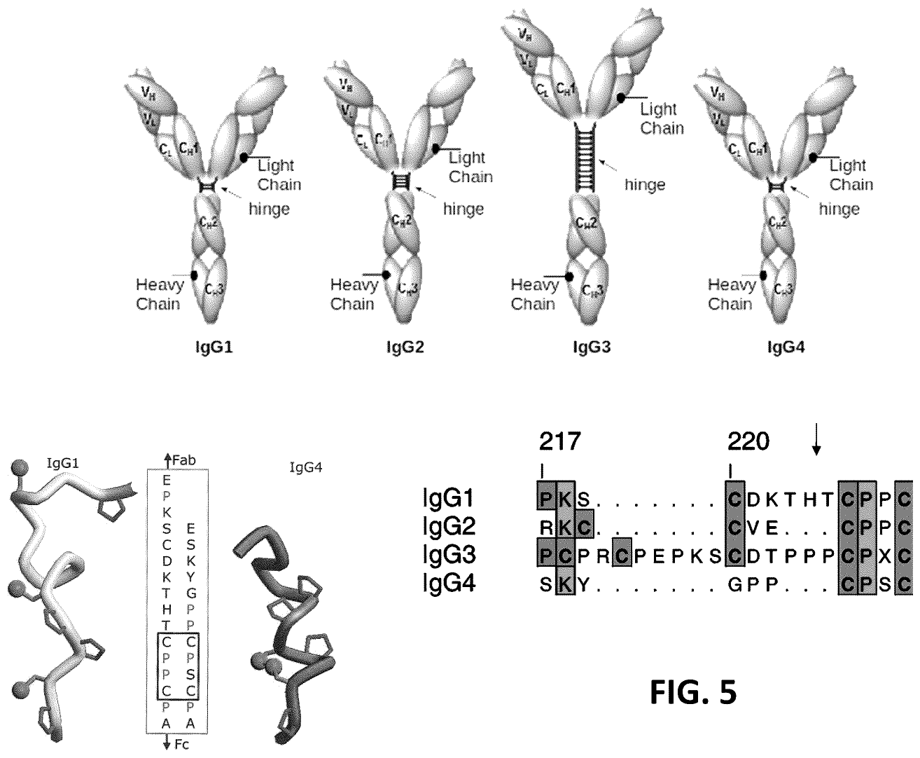

### DETAILED DESCRIPTION

The present disclosure is directed to CARs comprising spacers derived from immunoglobulin (Ig) hinge and/or constant region sequences, polynucleotides encoding such CARs, cells expressing the CARs, and methods for their use. Non-limiting examples of the various aspects are shown in the present disclosure. In some aspects, CAR spacers of the present disclosure are fragments of IgAQ1, IgA2, IgG1, IgG2, IgG3, IgG4, IgD, IgE or IgM hinge and/or constant regions. In some aspects, the CAR spacer comprises a subsequence from an IgM hinge and/or from a constant region, and further contains a short terminal sequence derived from other region(s), e.g., a CAR spacer can comprise a hinge subsequence and a CH1 and/or a CH2 subsequence. In some aspects, a CAR spacer can comprise a sequence connecting two constant regions.

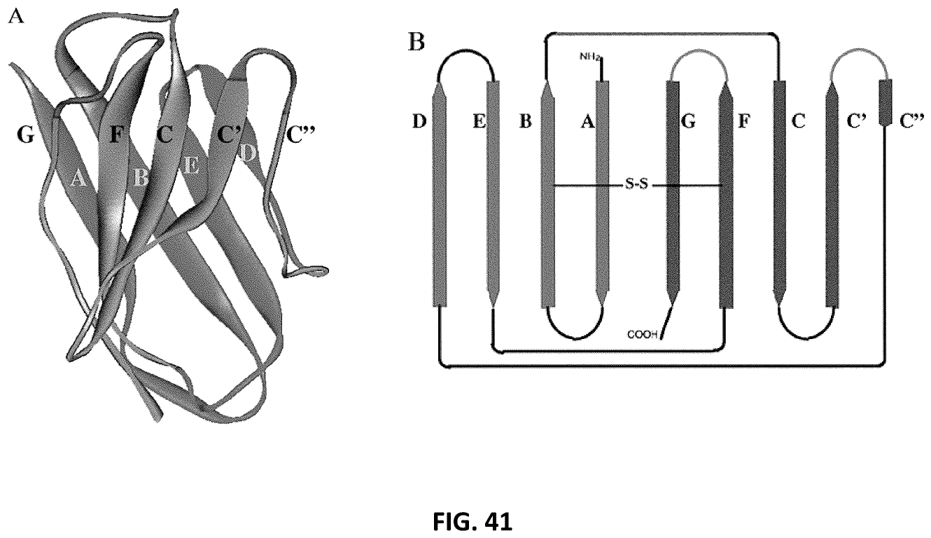

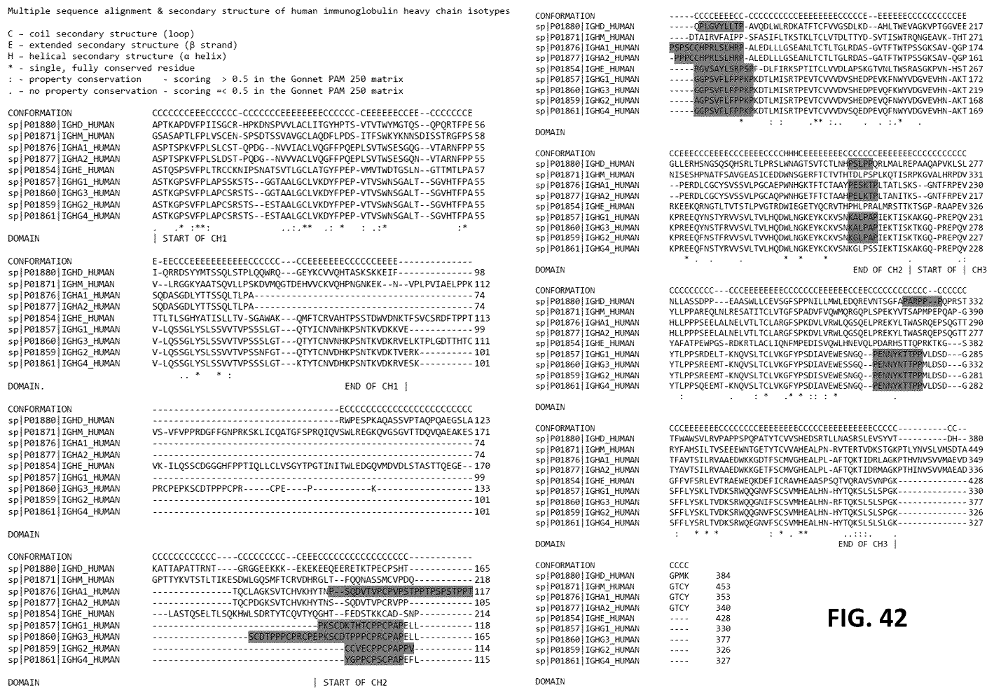

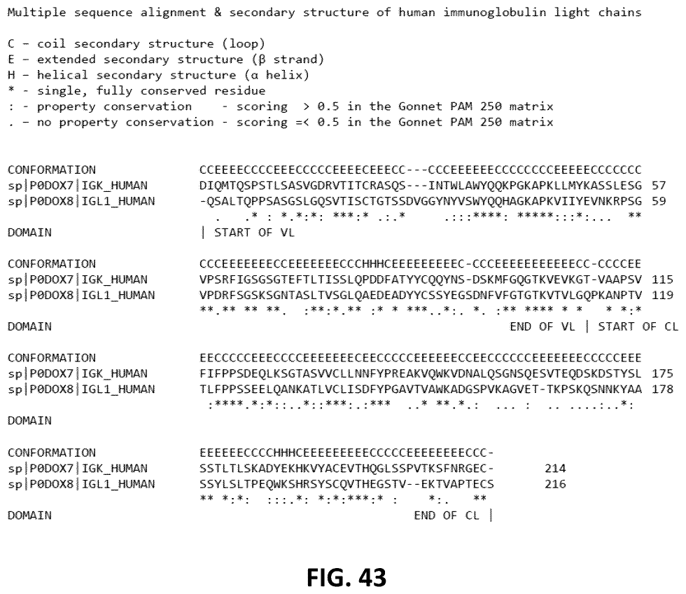

In some aspects, the CAR spacers derived from constant regions (e.g., heavy chain constant regions and/or light chain constant regions) correspond to unstructured sequences with a high content of Proline, Cysteine, Glycine, or combinations thereof, and generally correspond to solvent accessible loops. See, e.g., FIG. 41 and FIG. 42. For example, Spacer 15 corresponds to the N-terminal region of the flexible loop connecting P-strand A and P-strand B of the CH2 region of IgG1, IgG2, IgG3, or IgG4. Accordingly, in some aspects, a CAR spacer of the present disclosure comprises, consists, or consists essentially of an unstructured flexible loop connecting secondary structure elements (e.g., a helices, R strands, or combinations thereof) in an IgAQ1, IgA2, IgG1, IgG2, IgG3, IgG4, IgD, IgE, or IgM human immunoglobulin, or a fragment of such loop. In some aspects, the loop sequence is obtained from a non-human immunoglobulin. The location of loop sequences suitable to be used as CAR spacers of the present disclosure can be identified by a person skilled in the art, e.g., via structural assignment of amino acid residues in a PDB file using a program such as DSSP, P-SEA, DEFINE_S, SSTRUC, STRIDE, PROSS, or PALSSE. In some aspects, the sequence suitable as a CAR spacer of the present disclosure is an immunoglobulin domain loop sequence comprising at least 1, 2, 3, 4, 5 or 6 proline residues. In some aspects, the sequence suitable as a CAR spacer of the present disclosure is an immunoglobulin domain loop sequence wherein at least about 30%, about 40%, about 50%, about 60%, about 70%, about 80%, or about 90% of amino acid residues are Proline, Cysteine, Glycine, or combinations thereof.

In some aspects, the CAR spacers of the present disclosure are rationally designed based, e.g., on measurement of the distance between the epitope targeted by the CAR binding moiety and the surface of plasma membrane of the target cell, and/or the intermembrane distance in the signaling synapse. In some aspects, the CAR spacers of the present disclosure are modular sequences comprising at least one polypeptide sequence derived from an IgAQ1, IgA2, IgG1, IgG2, IgG3, IgG4, IgD, IgE or IgM hinge and/or constant region.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

Headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. Terms

In order that the present description can be more readily understood, certain terms are first defined. Except as otherwise expressly provided herein, each of the following terms shall have the meaning set forth below. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition of the present disclosure (e.g., a polynucleotide encoding a CAR or a cell expressing a CAR), into a subject via a pharmaceutically acceptable route. The introduction of a composition of the present disclosure (e.g., a polynucleotide encoding a CAR or a cell expressing a CAR), into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically.

Administration includes self-administration and the administration by another. A suitable route of administration allows the composition or the agent to perform its intended function. For example, if a suitable route is intravenous, the composition is administered by introducing the composition or agent into a vein of the subject.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. A reference to a “substitution at position X” refers to the substitution of an amino acid present at position X with an alternative amino acid residue. Substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

As used herein, the term “approximately,” as applied to one or more values of interest, refers to a value that is similar to a stated reference value. The term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 75% identical, at least about 80% identical, at least about 85% identical, at least about 90% identical, or at least about 95% identical to one another. In some aspects, two or more sequences are said to be “highly conserved” if they are about 70% identical, about 75% identical, about 80% identical, about 85% identical, about 90% identical, about 95% identical, about 98% identical, or about 99% identical to one another. In some aspects, two or more sequences are said to be “conserved” if they are at least about 30% identical, at least about 35% identical, at least about 40% identical, at least about 45% identical, at least about 50% identical, at least about 55%, at least about 60% identical, at least about 65% identical, at least about 70% identical, at least about 75% identical, at least about 80% identical, at least about 85% identical, at least about 90% identical, or at least about 95% identical to one another. In some aspects, two or more sequences are said to be “conserved” if they are about 30% identical, about 35% identical, about 40% identical, about 45% identical, about 50% identical, about 55% identical, about 60% identical, about 65% identical, about 70% identical, about 75% identical, about 80% identical, about 85% identical, about 90% identical, about 95% identical, about 98% identical, or about 99% identical to one another. Conservation of sequence may apply to the entire length of a polynucleotide or polypeptide or may apply to a portion, region or feature thereof.

“Derived from” as that term is used herein, indicates a relationship (e.g., structural similarity) between a first and a second molecule. For example, in the case of a CAR spacer of the present disclosure comprising an amino acid sequence derived from a human immunoglobulin sequence (e.g., a hinge and/or a constant region sequence), the sequence that is derived from the human immunoglobulin sequence (e.g., a hinge and/or a constant region sequence) can comprise or consist of a full hinge, a hinge fragment, a full hinge or a fragment of an hinge plus additional residues adjacent to the hinge in a wild type immunoglobulin (e.g., one or more amino acids from a constant domain such as a CH1 or CH2 domain), or can comprise or consist of the full sequence of a loop region, a loop region fragment, or a loop region fragment plus additional residues adjacent to the loop in a wild type immunoglobulin (e.g., one or more amino acids from a secondary structure element, e.g., a β-sheet, adjacent to a loop region in a CH1, CH2 or CH3 domain). In some aspects, a spacer derived from constant domain can be derived from a light chain constant domain (CL).

The term “loop region” as used herein refers to a primary sequence of amino acid residues which connects two regions comprising secondary structure, such as an α-helix or β-sheet, in the immediate N-terminal and C-terminal directions of primary structure from the loop region. Examples of loop regions include, but are not limited to, CH2 or CH3 loop regions. The immunoglobulin fold comprises a 2-layer sandwich of 7-9 antiparallel P-strands arranged in two P-sheets with a Greek key topology. See FIG. 41. Accordingly, constant domain derived CAR spacers of the present disclosure can comprise, consist, or consist essentially of a loop sequence (or a fragment thereof) connecting β-sheet A and β-sheet B, β-sheet B and β-sheet C, β-sheet C and β-sheet C′, P-sheet C′ and β-sheet C″, β-sheet C″ and β-sheet D, β-sheet D and β-sheet E, β-sheet E and β-sheet F, or β-sheet F and β-sheet G, in an immunoglobulin domain, e.g., a constant immunoglobulin domain (e.g., CH1, CH2, CH3, or CL).

CAR spacers derived from a human Ig immunoglobulin (e.g., a hinge and/or a constant region sequence), disclosed herein also encompass sequences generated by covalently linking via peptidic bonds a hinge region derived sequence as described above, i.e., the spacer can be a polymer comprising multiple repeats of a full hinge, fragments thereof, or combinations thereof.

In some aspects, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, e.g., naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide.

In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription. The term “upstream” refers to a nucleotide sequence that is located 5′ to a reference nucleotide sequence.

As used herein, the terms “antigen-binding domain” and “antibody” encompass an immunoglobulin whether natural or partly or wholly synthetically produced, and antigen-binding portions thereof. The term also covers any protein having a binding domain that is homologous to an immunoglobulin binding domain. “Antigen-binding domain” and “antibody” further include a polypeptide comprising a framework region from an immunoglobulin gene or portions thereof that specifically binds and recognizes an antigen, and comprises at least one CDR. Use of the terms “antigen-binding domain” and “antibody” is meant to include whole antibodies, polyclonal, monoclonal and recombinant antibodies, portions thereof, and further includes single-chain antibodies, humanized antibodies, murine antibodies, chimeric, mouse-human, mouse-primate, primate-human monoclonal antibodies, anti-idiotype antibodies, antibody constructs, such as, e.g., scFv, (scFv)₂, Fab, Fab′, and F(ab′)₂, F(ab1)₂, Fv, dAb, and Fd, disulfide-linked Fvs (dsFcs), and antibody-related polypeptides.

In some aspects, an “antigen-binding portion” refers to a polypeptide sequence that makes contacts with the antigen, including but not limited to CDRs derived from an antibody.

An antigen-binding portion can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen-binding portions can also be grafted into scaffolds based on polypeptides such as a fibronectin type III (Fn3)(see U.S. Pat. No. 6,703,199, which describes fibronectin polypeptide minibodies). Thus, the terms “antigen-binding domain” and “antibody” include also antibody mimics based on the scaffold of the fibronectin type III domain (monobodies), other scaffolding systems (e.g., tenascin) in which one or more CDRs are grafted, aptamers, etc.

The terms “antigen-binding domain” and “antibody” also include other suitable antigen-binding domains that can be used according to the present disclosure, e.g., nanobody, VHH antibody, DARPin (designed ankyrin repeat proteins), affibody, monobody, adnectin, alphabody, Albumin-binding domain, Adhiron, Affilin and other gamma-B crystallin-derived artificial proteins, Affimer, Affitin (NANOFITIN™), Anticalin, Armadillo repeat proteins (ARM-repeat protein such as, e.g., P-catenin, a-importing, plakoglobin, adenomatous polyposis coli, ARMC4, ARMCX3, etc.), Atrimer (e.g., tetranectin and derived proteins), Avimer/Maxibody, Centyrin, Fynomer and other Fyn SH3 domain-derived proteins, Kunitz domain, Obody/OB-fold, Pronectin, Repebody, or any synthetic and/or computationally designed binding-protein or scaffold.

The modular architecture of antibodies has been exploited to create more than 60 different bispecific or multispecific antibody formats. Accordingly, in some aspects, the antibody can be in a format selected, e.g., from crossMab, DAF (Dual Action Fab) (two-in-one), DAF (four-in-one), DutaMab, DT-IgG, Knobs-in-holes common LC, Knobs-in-holes assembly, Charge pair, Fab-arm exchange, SEEDbody, Triomab, LUZ-Y (bispecific antibody with a leucize zipper inducing heterodimerization of two HCs), Fcab, KX-body, Orthogonal Fab, DVD-IgG (dual variable domain IgG), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG (four-in-one), Nanobody, Nanobody-HSA, BiTE (bispecific T cell engager), Diabody, DART (dual-affinity-retargeting), TandAb (tandem antibody), scDiabody, scDiabody-CH3, Triple Body, Miniantibody, Minibody, TriBi minibody, scFv-CH3 KIH, Fab-scFv, scFv-CH-CL-scFv, F(ab′)2, F(ab′)2-ScFv2, scFv-KIH, Fab-scFv-Fc, Tetravalent HC Ab, scDiabody-Fc, Diabody-Fc, Tandem scFv-Fc, Intrabody, Dock and Locck, ImmTAC, HSAbody, scDiabody-HSA, Tandem scFv-Toxin, IgG-IgG, Cov-X-Body, and scFvl-PEG-scFV2.

“Antigen-binding domain” and “antibody” also include bispecific and multispecific antibodies so long as they exhibit the desired biological activity or function. In some aspects, the CAR of the present disclosure comprising an extracellular antigen-binding domain, e.g., an scFv.

The term “scFv” refers to a fusion protein comprising at least one antibody portion comprising a variable region of a light chain and at least one antibody portion comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The term “antigen” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA.

As used herein, the term “epitope” refers to the moieties of an antigen that specifically interact with an antibody molecule. Such moieties, referred to herein as epitopic determinants, typically comprise, or are part of, elements such as amino acid side chains or sugar side chains. An epitopic determinate can be defined, e.g., by methods known in the art, e.g., by crystallography or by hydrogen-deuterium exchange. At least one or some of the moieties on the antibody molecule that specifically interact with an epitopic determinant are typically located in a CDR(s). Typically an epitope has a specific three dimensional structural characteristics. Typically an epitope has specific charge characteristics. Some epitopes are linear epitopes while others are conformational epitopes.

The term “autologous” refers to any material derived from the same individual to whom it is later to be re-introduced into the individual.

The term “Chimeric Antigen Receptor” or alternatively a “CAR” refers to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (CD3 zeta). In some aspects, the cytoplasmic signaling domain comprises a primary signaling domain (e.g., a primary signaling domain of CD3 zeta). In some aspects, the cytoplasmic signaling domain further comprises one or more functional signaling domains derived from at least one costimulatory molecule defined below. In some aspects, the costimulatory molecule is chosen from the costimulatory molecules described herein, e.g., 4-1BB, CD27, and/or CD28.

In some aspects, the CAR comprises a chimeric fusion protein comprising an antigen-binding domain, a transmembrane domain and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The terms “cancer associated antigen” or “tumor antigen” or variants thereof interchangeably refer to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some aspects, a tumor antigen is a marker expressed by both normal cells and cancer cells, e.g., a lineage marker, e.g., CD19 on B cells. In certain aspects, the tumor antigen is derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

In some aspects, the tumor antigen is an antigen that is common to a specific proliferative disorder. In some aspects, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some aspects, a cancer-associated antigen is a cell surface molecule that is inappropriately synthesized in the cancer cell, for instance, a molecule that contains deletions, additions or mutations in comparison to the molecule expressed on a normal cell. In some aspects, a cancer-associated antigen will be expressed exclusively on the cell surface of a cancer cell, entirely or as a fragment (e.g., MHC/peptide), and not synthesized or expressed on the surface of a normal cell.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition.

An “anti-cancer effect” can also be manifested by the ability of the CAR, polynucleotides encoding CARs, vectors, and cells described herein in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can also be used. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

In the content of the present disclosure, the terms “mutation” and “amino acid substitution” as defined above (sometimes referred simply as a “substitution”) are considered interchangeable.

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (polynucleotide or polypeptide sequences).

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

As used herein, the terms “isolated,” “purified,” “extracted,” and grammatical variants thereof are used interchangeably and refer to the state of a preparation of desired composition of the present disclosure, e.g., a CAR of the present disclosure, that has undergone one or more processes of purification. In some aspects, isolating or purifying as used herein is the process of removing, partially removing (e.g., a fraction) of a composition of the present disclosure, e.g., a CAR of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.9%, at least about 99.99%, at least about 99.999%, at least about 99.9999%, or greater than 99.99999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA).

A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose) and polyribonucleotides (containing D-ribose), including mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects, a polynucleotide disclosed herein comprises a DNA, e.g., a DNA inserted in a vector. In other aspects, a polynucleotide disclosed herein comprises an mRNA. In some aspects, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding” an amino acid sequence,” e.g., a polynucleotide “encoding” a CAR of the present disclosure, includes all nucleotide sequences that are degenerate versions of each other and that encode the same amino acid sequence.

The term “expression” refers to the transcription and/or translation of a particular nucleotide sequence driven by a promoter.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides disclosed herein can be recombinantly produced using methods known in the art. In some aspects, the CARs of the present disclosure are recombinantly produced. In some aspects, the CARs of the present disclosure are produced by cells, e.g., T cells, following transfection with at least one polynucleotide o vector encoding a CAR of the present disclosure.

As used herein, the term “fragment” of a polypeptide (e.g., a Ig hinge) refers to an amino acid sequence of a polypeptide that is shorter than the naturally-occurring sequence, N- and/or C-terminally deleted or any part of the polypeptide deleted in comparison to the naturally occurring polypeptide. Thus, a fragment does not necessary need to have only N- and/or C-terminal amino acids deleted. A polypeptide in which internal amino acids have been deleted with respect to the naturally occurring sequence is also considered a fragment.

As used herein, the term “functional fragment” refers to a polypeptide fragment that retains polypeptide function. Accordingly, in some aspects, a functional fragment of an Ig hinge, retains the ability to position an antigen-binding domain (e.g., an scFv) in a CAR at a distance from a target epitope (e.g., a tumor antigen) such that the antigen-binding domain (e.g., an scFv) can effectively interact with the target epitope (e.g., a tumor antigen).

Whether a Ig hinge fragment is a functional fragment can be assessed by any art known methods to determine the binding of the CAR comprising the spacer to a target antigen, and T-cell activation, including, e.g., Western Blots, FACS analysis, cytokine secretion analyses, cell survival analyses, etc. In certain aspects, a Ig hinge functional fragment is a fragment that when used as a spacer in a CAR, results in a CAR with, e.g., at least about 20%, at least about 25%, at least about 30%, at least about 35%, at least about 40%, at least about 45%, at least about 50%, at least 55%, at least about 60%, at least about 65%, at least about 70%, at least about 75%, at least about 80%, at least about 85%, at least about 90%, at least about 95%, at least about 99%, or about 100% of the activity of a reference CAR. As used herein, the term “reference CAR” refers to a corresponding CAR comprising the same structural components as the tested CAR, but a different spacer (a “reference spacer”). In some aspects, the reference spacer is, e.g., an IgG1 spacer, i.e., a spacer corresponding to the hinge regions of IgG1.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, variants or derivatives include, e.g., modified polypeptides. In some aspects, variants or derivatives of, e.g., polypeptides, polynucleotides, lipids, glycoproteins, are the result of chemical modification and/or endogenous modification. In some aspects, variants or derivatives are the result of in vivo modification. In some aspects, variants or derivatives are the result of in vitro modification. In yet other aspects, variant or derivatives are the result of intracellular modification in producer cells, e.g., T cells.

Modifications present in variants and derivatives include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination.

The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain can generate a signal that promotes an immune effector function of the CAR containing cell, e.g., a CART cell. Examples of immune effector function, e.g., in a CART cell, include cytolytic activity and helper activity, including the secretion of cytokines. In some aspects, the intracellular signal domain is the portion of the protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR T, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (ICOS), FcεRI, CD66d, CD32, DAP10 and DAP12.

The terms “covalently linked,” “fused,” and grammatical variants thereof are used interchangeably and refer to a first moiety, e.g., a first amino acid sequence or nucleotide sequence, covalently or non-covalently joined to a second moiety, e.g., a second amino acid sequence or nucleotide sequence, respectively. The first moiety can be directly joined or juxtaposed to the second moiety or alternatively an intervening moiety can covalently join the first moiety to the second moiety. The term “linked” means not only a fusion of a first moiety to a second moiety at the C-terminus or the N-terminus, but also includes insertion of the whole first moiety (or the second moiety) into any two points, e.g., amino acids, in the second moiety (or the first moiety, respectively). In some aspects, the first moiety is linked to a second moiety by a peptide bond or a linker. The first moiety can be linked to a second moiety by a phosphodiester bond or a linker. The linker can be a peptide or a polypeptide (for polypeptide chains) or a nucleotide or a nucleotide chain (for nucleotide chains) or any chemical moiety (for polypeptide or polynucleotide chains or any chemical molecules).

As used herein, the term “pharmaceutical composition” refers to one or more of the compounds described herein, such as, e.g., a CAR of the present disclosure or a cell expressing a CAR of the present disclosure, mixed or intermingled with, or suspended in one or more other chemical components, such as pharmaceutically-acceptable carriers and excipients. One purpose of a pharmaceutical composition is to facilitate administration of preparations of, e.g., cell expressing a CAR of the present disclosure to a subject.

The terms “excipient” and “carrier” are used interchangeably and refer to an inert substance added to a pharmaceutical composition to further facilitate administration of a compound, e.g., a CAR of the present disclosure.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a CAR of the present disclosure, e.g., to improve hemostasis.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In some aspects, the term “treating” or “treatment” means inducing an immune response in a subject against an antigen.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein the term “therapeutically effective amount” is the amount of reagent or pharmaceutical compound comprising a CAR of the present disclosure that is sufficient to a produce a desired therapeutic effect, pharmacologic and/or physiologic effect on a subject in need thereof.

A therapeutically effective amount can be a “prophylactically effective amount” as prophylaxis can be considered therapy. As used herein, “prophylactic” refers to a therapeutic or course of action used to prevent the onset of a disease or condition, or to prevent or delay a symptom associated with a disease or condition. As used herein, a “prophylaxis” refers to a measure taken to maintain health and prevent the onset of a disease or condition, or to prevent or delay a symptom associated with a disease or condition.

### II. CARs with Ig Derived Spacers

The present disclosure provides Immunoglobulin (Ig) derived CAR spacers (e.g., derived from hinge regions or loop regions) that are useful for cells expressing one or more chimeric antigen receptors. These CAR spacers comprise, e.g., IgAQ1, IgA2, IgG1, IgG2, IgG3, IgG4, IgD, IgE, or IgM hinge regions, fragments thereof (alone or capped by additional sequences, e.g., CH1 or CH2 regions sequences), or combinations of fragments from IgAQ1, IgA2, IgG1, IgG2, IgG3, IgG4, IgD, IgE, or IgM hinge regions. In some aspects, the CAR spacers comprise, e.g., IgAQ1, IgA2, IgG1, IgG2, IgG3, IgG4, IgD, IgE, or IgM constant domain loop regions, fragments thereof (alone or capped by additional sequences, e.g., from adjacent P-strands), or combinations of fragments from IgAQ1, IgA2, IgG1, IgG2, IgG3, IgG4, IgD, IgE, or IgM loop regions. In some aspects, the CAR spacer of the present disclosure comprise hinge region derived sequences, loop region derived sequences, or combinations thereof.

Accordingly, the present disclosures provide polynucleotides encoding a CAR comprising, e.g., (i) an antigen-binding domain, (ii) a transmembrane domain, (iii) an intracellular domain, and (iv) a CAR spacer comprising an amino acid sequence derived from a human immunoglobulin (Ig) hinge region and/or loop region (i.e., a CAR spacer of the present disclosure), and optionally a linker (e.g, a gly-ser rich linker) wherein the spacer is located between the antigen-binding domain and the transmembrane domain. In some aspects, the present disclosure provides a recombinant nucleic acid construct comprising a transgene encoding a CAR of the present disclosure. The present disclosure also provides a CAR encoded by one or more of the polynucleotide sequences or the vectors disclosed herein. In some aspects, the CAR of present disclosure is designed as a standard CAR, a split CAR, an off-switch CAR, an on-switch CAR, a first-generation CAR, a second-generation CAR, a third-generation CAR, a fourth-generation CAR, or a fifth generation CAR.

The term “CAR spacer” as used herein refers to a polypeptide sequence which is capable of covalently linking together two spaced moieties: an antigen-binding domain, and the transmembrane domain of the CAR.

The terms “CAR spacer of the present disclosure” and “Ig derived CAR spacer” are used interchangeably to refers to (i) a “hinge region derived CAR spacer,” i.e., a CAR spacer comprising an amino acid sequence derived from a hinge region located between the CH1 and CH2 constant domains of a human immunoglobulin, e.g., IgAQ1, IgA2, IgG1, IgG2, IgG3, IgG4, IgD, IgE, or IgM, and optionally one or more amino acids from an adjacent CH1 and/or CH2 domain, or a combination thereof (e.g., several concatenated hinge region derived CAR spacer); (ii) a “loop region derived CAR spacer,” i.e., a CAR spacer comprising an amino acid sequence derived from a loop region of a constant domain of a human immunoglobulin, e.g., IgAQ1, IgA2, IgG1, IgG2, IgG3, IgG4, IgD, IgE, or IgM, and optionally one or more amino acids from an adjacent P-strand, or a combination thereof (e.g., several concatenated loop region derived CAR spacers); or, (iii) a combination thereof (e.g., two or more concatenated hinge region derived CAR spacers and loop region derived CAR spacers).

In some aspects, the term CAR spacer of the present disclosure refers to a subsequence of an immunoglobulin heavy chain selected the group consisting of human IgAQ1 (Uniprot: P01876, IGHA1_HUMAN, immunoglobulin heavy constant alpha 1; SEQ ID NO: 4994), human IgA2 (Uniprot P01877, IGHA2_HUMAN, immunoglobulin heavy constant alpha 2; SEQ ID NO: 4995), murine IgG2A (Uniprot P01665, GCAM_MOUSE, immunoglobulin gamma 2 Å chain C region; SEQ ID NO: 4993), human IgG1 (Uniprot P01857, IGHG1_HUMAN, immunoglobulin heavy constant gamma 1; SEQ ID NO: 4998), human IgG2 (Uniprot P01859, IGHG2_HUMAN, immunoglobulin heavy constant gamma 2; SEQ ID NO: 4999), human IgG3 (Uniprot P01860, IGHG3_HUMAN, immunoglobulin heavy constant gamma 3; SEQ ID NO: 5000), human IgG4 (Uniprot P01861, IGHG4, immunoglobulin heavy constant gamma 4; SEQ ID NO: 5001), human IgD (Uniprot P01880, IGHD_HUMAN, immunoglobulin heavy constant delta; SEQ ID NO: 4996), human IgE (Uniprot P01854, IGHE_HUMAN, immunoglobulin heavy constant chain epsilon; SEQ ID NO: 4997), or IgM (Uniprot P01871, IGHM_HUMAN, immunoglobulin heavy constant mu; SEQ ID NO: 5002), wherein the subsequence comprises the CH1-CH2 hinge region or a portion thereof. In some aspects, the subsequence further comprises an adjacent portion of a CH1 and/or CH2 constant domain.

In some aspects, the term CAR spacer of the present disclosure refers to a subsequence of an immunoglobulin heavy chain selected the group consisting of human IgAQ1 (Uniprot: P01876, IGHA1_HUMAN, immunoglobulin heavy constant alpha 1; SEQ ID NO: 4994), human IgA2 (Uniprot P01877, IGHA2_HUMAN, immunoglobulin heavy constant alpha 2; SEQ ID NO: 4995), murine IgG2A (Uniprot P01665, GCAM_MOUSE, immunoglobulin gamma 2 Å chain C region; SEQ ID NO: 4993), human IgG1 (Uniprot P01857, IGHG1_HUMAN, immunoglobulin heavy constant gamma 1; SEQ ID NO: 4998), human IgG2 (Uniprot P01859, IGHG2_HUMAN, immunoglobulin heavy constant gamma 2; SEQ ID NO: 4999), human IgG3 (Uniprot P01860, IGHG3_HUMAN, immunoglobulin heavy constant gamma 3; SEQ ID NO: 5000), human IgG4 (Uniprot P01861, IGHG4, immunoglobulin heavy constant gamma 4; SEQ ID NO: 5001), human IgD (Uniprot P01880, IGHD_HUMAN, immunoglobulin heavy constant delta; SEQ ID NO: 4996), human IgE (Uniprot P01854, IGHE_HUMAN, immunoglobulin heavy constant chain epsilon; SEQ ID NO: 4997), or IgM (Uniprot P01871, IGHM_HUMAN, immunoglobulin heavy constant mu; SEQ ID NO: 5002), wherein the subsequence comprises a loop region from a constant domain or a portion thereof. In some aspects, the subsequence further comprises an adjacent portion of a P-strand.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a hinge region of an immunoglobulin of SEQ ID NO: 4993-5002; or a combination thereof.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a hinge region of an immunoglobulin of SEQ ID NO: 4993-5002, wherein the subsequence further comprises 1, 2, 3, 4, 5, 6, 7, 8, 9, or 10 contiguous amino acids from the CH1 region adjacent to the hinge region subsequence; or a combination thereof.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a hinge region of an immunoglobulin of SEQ ID NO: 4993-5002, wherein the subsequence further comprises 1, 2, 3, 4, 5, 6, 7, 8, 9, or 10 contiguous amino acids from the CH2 region adjacent to the hinge region subsequence; or a combination thereof.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a hinge region of an immunoglobulin of SEQ ID NO: 4993-5002, wherein the subsequence further comprises 1, 2, 3, 4, 5, 6, 7, 8, 9, or 10 contiguous amino acids from the CH1 region adjacent to the hinge region subsequence and 1, 2, 3, 4, 5, 6, 7, 8, 9, or 10 contiguous amino acids from the CH2 region adjacent to the hinge region subsequence; or a combination thereof.

In some aspects, the CAR spacer comprises a hinge region derived spacer and further comprises a glycine-serine flexible linker, e.g., a Gly-Ser of SEQ ID NO: 4818 or 5088.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a loop region of a constant domain (e.g., CH1, CH2 or CH3) of an immunoglobulin of SEQ ID NO: 4993-5002; or a combination thereof.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a loop region of a constant domain (e.g., CH1, CH2 or CH3) of an immunoglobulin of SEQ ID NO: 4993-5002, wherein the subsequence further comprises 1, 2, 3, 4, 5, 6, 7, 8, 9, or 10 contiguous amino acids from a P-strand adjacent to the C-terminus of the loop region subsequence; or a combination thereof.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a loop region of a constant domain (e.g., CH1, CH2 or CH3) of an immunoglobulin of SEQ ID NO: 4993-5002, wherein the subsequence further comprises 1, 2, 3, 4, 5, 6, 7, 8, 9, or 10 contiguous amino acids from a P-strand adjacent to the N-terminus of the loop region subsequence; or a combination thereof.

In some aspects, the CAR spacer comprises a subsequence consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 amino acids from a loop region of a constant domain (e.g., CH1, CH2 or CH3) of an immunoglobulin of SEQ ID NO: 4993-5002, wherein the subsequence further comprises 1, 2, 3, 4, 5, 6, 7, 8, 9, or 10 contiguous amino acids from a P-strand adjacent to the C-terminus of the loop region subsequence; and 1, 2, 3, 4, 5, 6, 7, 8, 9, or 10 contiguous amino acids from the a P-strand adjacent to N-terminus of the loop region subsequence.

In some aspects, the CAR spacer comprises a loop region derived spacer and further comprises a glycine-serine flexible linker, e.g., a Gly-Ser of SEQ ID NO: 4818 or 5088.

In some aspects, a CAR spacer derived from a hinge region disclosed herein comprises, consists, or consist essentially of an amino acid sequence of having at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NOS: 4830-4842, and 4844-4859.

In some aspects, a CAR spacer derived from a loop region disclosed herein comprises, consists, or consist essentially of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4843 (Spacer 15).

### II.A. IgD Derived CAR Spacer

Immunoglobulin D (IgD) is an antibody isotype that makes up about 1% of proteins in the plasma membranes of immature B-lymphocytes where it is usually co-expressed with another cell surface antibody called IgM. Structurally, IgD are similar to IgG.

As used herein, the term “IgD hinge” refers to an amino sequence located between the CH1 region and CH2 regions of the heavy chain of a IgD. IgD heavy chains contain a long hinge region encoded by two exons located between amino acid positions 99 and 162. One exon encodes hinge subregion 1 (SEQ ID NO: 861) and the second exon encodes hinge subregion 2 (SEQ ID NO: 1287). In some aspects, of the present disclosure, the “IgD hinge” is an extended IgD comprising, i.e., a sequence comprising the 64 amino acids IgD hinge core (subregions 1 and 2) plus a 13 amino acids long C-terminal CH2 fragment of SEQ ID NO:2143. Thus, a “full length IgD hinge” as defined in the present application comprises the 77 amino acids long sequence of SEQ ID NO: 1.

As used herein, the term “IgD hinge derived CAR spacer” comprises, e.g., spacer comprising IgD hinge subregion 1 and fragments thereof, IgD hinge subregion 2 and fragments thereof, fragments of a full length IgD hinge of SEQ ID NO: 1, and variants thereof comprising 1, 2, 3, 4, 5 or more CH1 region and/or CH2 region amino acids.

In some aspects, an IgD hinge derived CAR spacer of the present disclosure comprises at least five, six, or seven consecutive amino acids of SEQ ID NO: 1. In some aspects, the IgD hinge derived spacer comprises at least five, six, or seven consecutive amino acids of SEQ ID NO: 861, SEQ ID NO: 1287, and/or SEQ ID ON: 2143.

In some aspects, a CAR comprising an IgD hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgD hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911). As used herein, the term “reference spacer” refers to a spacer present in a CAR prior to being optimized by the replacement of the original spacer with a spacer disclosed herein. In some aspects, the reference spacer is an IgG4 spacer (e.g., a spacer of SEQ ID NO: 4911). In some aspects, the reference spacer can be any spacer known in the art, e.g., a spacer disclosed in any of the documents incorporated by reference in the present disclosure.

In some aspects, a CAR comprising an IgD hinge derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 10000, at least about 120%, at least about 150%, compared to a CAR comprising a reference spacer, e.g., a reference IgG4 spacer (e.g., a spacer of SEQ ID NO: 4911).

In some aspects, an IgD hinge derived CAR spacer of the present disclosure comprises an amino acid sequence having at least about 700%, at least about 7500 at least about 800%, at least about 85%, at least about 90%, at least about 95%, at least about 96%, at least about 97%, at least about 98%, at least about 99%, or about 100% sequence identity to the sequence set forth in SEQ ID NO: 1. In some aspects, the IgD hinge derived spacer comprises an amino acid sequence having at least about 70%, at least about 75%, at least about 80%, at least about 85%, at least about 90%, at least about 95% at least about 96%, at least about 97% at least about 98%, at least about 99% or about 100% sequence identity to SEQ ID NO: 861, SEQ ID NO: 1287, and/or SEQ ID ON: 2143.

In some aspects, an IgD hinge derived CAR spacer of the present disclosure comprises, consists, or consist essentially of a sequence disclosed in TABLE 1.

TABLE 1
IgD derived spacers. SEQ ID NO: 1 is full length IgD hinge.

| Length | SEQ ID | Length | SEQ ID | Length | SEQ ID |
|---|---|---|---|---|---|
| 77 (full length) | 1 | 76 | 2-3 | 75 | 4-6 |
| 74 | 7-10 | 73 | 11-15 | 72 | 16-21 |
| 71 | 22-28 | 70 | 29-36 | 69 | 37-45 |
| 68 | 46-55 | 67 | 56-66 | 66 | 67-78 |
| 65 | 79-91 | 64 | 92-105 | 63 | 106-120 |
| 62 | 121-136 | 61 | 137-153 | 60 | 154-171 |
| 59 | 172-190 | 58 | 191-210 | 57 | 211-231 |
| 56 | 232-253 | 55 | 254-276 | 54 | 277-300 |
| 53 | 301-325 | 52 | 326-351 | 51 | 352-378 |
| 50 | 379-406 | 49 | 407-435 | 48 | 436-465 |
| 47 | 466-496 | 46 | 497-528 | 45 | 529-561 |
| 44 | 562-595 | 43 | 596-630 | 42 | 631-666 |
| 41 | 667-703 | 40 | 704-741 | 39 | 742-780 |
| 38 | 781-820 | 37 | 821-861 | 36 | 862-903 |
| 35 | 904-946 | 34 | 947-989 | 33 | 990-1034 |
| 32 | 1035-1080 | 31 | 1081-1126 | 30 | 1127-1174 |
| 29 | 1175-1223 | 28 | 1124-1273 | 27 | 1274-1324 |
| 26 | 1325-1376 | 25 | 1377-1429 | 24 | 1430-1483 |
| 23 | 1484-1538 | 22 | 1539-1594 | 21 | 1595-1651 |
| 20 | 1652-1709 | 19 | 1710-1768 | 18 | 1769-1828 |
| 17 | 1829-1889 | 16 | 1980-1951 | 15 | 1952-2014 |
| 14 | 2015-2078 | 13 | 2079-2043 | 12 | 2144-2209 |
| 11 | 2210-2276 | 10 | 2277-2344 | 9 | 2345-2413 |
| 8 | 2414-2473 | 7 | 2484-2559 | | |

In some aspects, the spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NO: 2015; SEQ ID NO: 1889; SEQ ID NO: 1768; SEQ ID NO: 1; and any combination thereof.

In some aspects, the spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2015.

In some aspects, the spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 1889.

In some aspects, the spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 1.

In some aspects, the spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 1768.

In some aspects, the IgD hinge derived CAR spacer of the present disclosure comprises a sequence disclosed in TABLE 1 or a sequence shown anywhere in the present disclosure, further comprising an N-terminal subsequence comprising 1, 2, 3, 4, or 5 IgD CH1 C-terminal domain amino acids from TABLE 2 covalently bound to the N-terminus of the IgD sequence from TABLE 1 or a sequence shown anywhere in the present disclosure.

In some aspects, the IgD hinge derived CAR spacer of the present disclosure comprises a sequence disclosed in TABLE 1 or a sequence shown anywhere in the present disclosure, further comprising a C-terminal subsequence comprising 1, 2, 3, 4, or 5 IgD CH2 N-terminal domain amino acids from TABLE 2 covalently bound to the C-terminus of the IgD sequence from TABLE 1 or a sequence shown anywhere in the present disclosure.

In some aspects, the IgD hinge derived CAR spacer of the present disclosure comprises a sequence disclosed in TABLE 1 or a sequence shown anywhere in the present disclosure, further comprising (i) an N-terminal subsequence comprising 1, 2, 3, 4, or 5 IgD CH1 C-terminal domain amino acids from TABLE 2 covalently bound to the N-terminus of the IgD sequence from TABLE 1 or a sequence shown anywhere in the present disclosure, and (ii) a C-terminal subsequence comprising 1, 2, 3, 4, or 5 IgD CH2 N-terminal domain amino acids from TABLE 2 covalently bound to the C-terminus of the IgD sequence from TABLE 1 or a sequence shown anywhere in the present disclosure.

IgD CH1 and CH2 sequences that can be appended to the N- and/or C-termini of the IgD hinge derived CAR spacer sequences of TABLE 1 or a sequence shown anywhere in the present disclosure are presented in TABLE 2.

TABLE 2
IgD. CH1 and CH2 optional N-terminal
CH1 sequences and C-terminal CH2 sequences
(sequences from Uniprot P01880-1 comprising
1 to 5 amino acids flanking the full IgD hinge
of SEQ ID NO: 1)

| CH1 | | CH2 | |
|---|---|---|---|
| Length | Sequence | Length | Sequence |
| 1 | F | 1 | A |
| 2 | IF | 2 | AV |
| 3 | EIF | 3 | AVQ |
| 4 | KEIF (SEQ | 4 | AVQD (SEQ |
| | ID NO: 4519) | | ID NO: 4521) |
| 5 | KKEIF (SEQ | 5 | AVQDL (SEQ |
| | ID NO: 4520) | | ID NO: 4522) |

### I.B. IgA Hinge Derived CAR Spacer

IgA is an antibody that pays a crucial role in the immune function of mucous membranes, and represents up to 15% of total immunoglobulins produced throughout the body. IgA has to subclasses: IgAQ1 and IgA2. In IgA2, the heavy and light chains are not linked with disulfide, but with noncovalent bonds.

The hinge region differs significantly between the two IgA isoforms. The hinge region of IgAQ1 is comprised of 23 residues (SEQ ID NO: 2560) and 5 O-glycosylation sites, while IgA2's hinge region is comprised of 10 residues (SEQ ID NO: 2713) and no sites of glycosylation. Both hinge regions are located at Cys220 on the Ch1 chain and end at Ch2's Pro244.

As used herein, the “IgAQ1 hinge” refers to the hinge region located between the CH1 and CH2 regions of IgAQ1 having the sequence set forth in SEQ ID NO: 2560.

As used herein, the term “IgAQ1 hinge derived CAR spacer” comprises, e.g., spacers comprising the full IgAQ1 hinge of SEQ ID NO: 2560 and fragments thereof, and variants thereof comprising 1, 2, 3, 4, 5 or more CH1 region and/or CH2 region amino acids.

Therefore, in some aspects, an IgAQ1 hinge derived CAR spacer comprises at least five, six, or seven consecutive amino acids of SEQ ID NO: 2560. Therefore, in some aspects, an IgAQ1 hinge derived CAR spacer comprises at least five, six, or seven consecutive amino acids of SEQ ID NO: 2560. In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2560.

In some aspects, a CAR comprising an IgAQ1 hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgAQ1 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgAQ1 derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

As used herein, the term “IgA2 hinge” refers to the IgA2 sequence located between the Cys220 at the C-terminus of the CH1 constant domain of the heavy chain of IgA2 and Pro244 at the N-terminus of the CH2 constant domain of the heavy chain of IgA2. In some aspects, the full IgA2 hinge consists of the sequence set forth in SEQ ID NO: 2713. As used herein, the term “IgA2 hinge” encompasses the hinges of different IgA2 allotypes, e.g., IgA2m, IgA2(ml), IgA2(m2) or IgA2(n). The first Pro in the canonical IgA2 hinge of SEQ ID NO: 2713 disclosed above is an Arg in the IgA2(m2) allotype hinge (i.e., it starts with RV instead of PV). Accordingly, the IgA2 CAR spacers of the present disclosure encompasses also forms in which Proline amino acids corresponding to position 1 of SEQ ID NO: 2713 (e.g., corresponding amino acids present in IgA2 fragments or any IgA2 derived sequences) are replaced with Arginines.

As used herein, the term “IgA2 derived CAR spacer” comprises, e.g., spacers comprising the full IgA2 hinge of SEQ ID NO: 2713 and fragments thereof, and variants thereof comprising 1, 2, 3, 4, 5 or more CH1 region and/or CH2 region amino acids. In some aspects, an IgA2 derived CAR spacer comprises at least five, six, or seven consecutive amino acids of SEQ ID NO: 4848. In some aspects, an IgA2 derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4848.

In some aspects, a CAR comprising an IgA2 hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a pacer of SEQ ID NO: 4911).

In some aspects, a CAR comprising an IgA2 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a pacer of SEQ ID NO: 4911).

In some aspects, a CAR comprising an IgA2 hinge derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, an IgA2 derived CAR spacer of the present disclosure can, e.g., have the sequence set forth in SEQ ID NO: 4523 (i.e., an IgA2 hinge sequence with an N-terminal VPC sequence from CH1), SEQ ID NO: 4850, SEQ ID NO: 4524 (i.e, an IgA2 spacer sequence with an N-terminal VPC sequence from CH1 and a C-terminal PR sequence from CH2), or SEQ ID NO: 4525 (i.e, an IgA2 spacer sequence with an N-terminal PC sequence from CH1 and a C-terminal P sequence from CH2), wherein the VCP and PC N-terminal subsequences from SEQ ID NOS:3523, 4524, and 4525 are from the CH1 region and the C-terminal PR and P subsequences from SEQ ID NOS: 4524 and 4525 are from the CH2 region of the IgA2 immunoglobulin.

In some aspects, an IgAQ1 hinge derived CAR spacer of the present disclosure comprises, consists, or consist essentially of a sequence disclosed in TABLE 3.

TABLE 3
IgA1 derived spacers

| Length | SEQ ID | Length | SEQ ID | Length | SEQ ID |
|---|---|---|---|---|---|
| 23 | 2560 | 22 | 2561-2562 | 21 | 2563-2665 |
| 20 | 2566-2569 | 19 | 2570-2574 | 18 | 2575-2580 |
| 17 | 2581-2587 | 16 | 2588-2595 | 15 | 2596-2604 |
| 14 | 2605-2614 | 13 | 2615-2625 | 12 | 2626-2637 |
| 11 | 2638-2650 | 10 | 2651-2644 | 9 | 2665-2679 |
| 8 | 2680-2695 | 7 | 2696-2712 | | |

In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NO: 4845; SEQ ID NO: 4846; SEQ ID NO: 4847; SEQ ID NO: 4848; SEQ ID NO: 4849; SEQ ID NO: 4850; SEQ ID NO: 4851; SEQ ID NO: 2560; SEQ ID NO: 4844; and any combination thereof.

In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4847.

In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4845.

In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4846.

In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2560.

In some aspects, the IgAQ1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4844.

In some aspects, the IgAQ1 hinge derived CAR spacer of the present disclosure comprises a sequence disclosed in TABLE 3 or any IgAQ1 hinge derived CAR spacer disclosed herein, further comprising an N-terminal subsequence comprising 1, 2, 3, 4, or 5 IgAQ1 CH1 C-terminal domain amino acids from TABLE 4 covalently bound to the N-terminus of the IgAQ1 sequence from TABLE 3 or any IgAQ1 hinge derived CAR spacer disclosed herein.

In some aspects, the IgAQ1 hinge derived CAR spacer of the present disclosure comprises a sequence disclosed in TABLE 3 or any IgAQ1 hinge derived CAR spacer disclosed herein, further comprising a C-terminal subsequence comprising 1, 2, 3, 4, or 5 IgA1 CH2 N-terminal domain amino acids from TABLE 4 covalently bound to the C-terminus of the IgA2 sequence from TABLE 3 or any IgAQ1 hinge derived CAR spacer disclosed herein.

In some aspects, the IgAQ1 hinge derived CAR spacer of the present disclosure comprises a sequence disclosed in TABLE 3 or any IgAQ1 hinge derived CAR spacer disclosed herein, further comprising (i) an N-terminal subsequence comprising 1, 2, 3, 4, or 5 IgAQ1 CH1 C-terminal domain amino acids from TABLE 4 covalently bound to the N-terminus of the IgAQ1 sequence from TABLE 3 or any IgAQ1 hinge derived CAR spacer disclosed herein, and (ii) a C-terminal subsequence comprising 1, 2, 3, 4, or 5 IgAQ1 CH2 N-terminal domain amino acids from TABLE 4 covalently bound to the C-terminus of the IgD sequence from TABLE 3 or any IgAQ1 hinge derived CAR spacer disclosed herein.

IgAQ1 CH1 and CH2 sequences that can be appended to the N- and/or C-termini of the IgAQ1 derived CAR spacer sequences of TABLE 3 or any IgAQ1 hinge derived CAR spacer disclosed herein are presented in TABLE 4.

TABLE 4
IgA1. CH1 and CH2 optional N-terminal
CH1 sequences and C-terminal CH2
(sequences from Uniprot P01876 comprising
1 to 5 amino acids flanking the full
IgAl hinge of sequences SEQ ID NO: 2560)

| CH1 | | CH2 | |
|---|---|---|---|
| Length | Sequence | Length | Sequence |
| 1 | C | 1 | P |
| 2 | PC | 2 | PR |
| 3 | VPC | 3 | PRL |
| 4 | TVPC | 4 | PRLS |
| | (SEQ ID | | (SEQ ID |
| | NO: 4526) | | NO: 4528) |
| 5 | VTVPC | 5 | PRLSL |
| | (SEQ ID | | (SEQ ID |
| | NO: 4527) | | NO: 4529) |

In some aspects, an IgA2 hinge derived CAR spacer of the present disclosure comprises at least five, six, or seven consecutive amino acids of the sequence set forth in SEQ ID NO: 2713. In some aspects, an IgA2 hinge derived CAR spacer of the present disclosure comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2713.

In some aspects, an “IgA2 hinge derived CAR spacer” of the present disclosure comprises, consists, or consist essentially of a sequence disclosed in TABLE 5

TABLE 5
IgA2 derived spacers

| Length | SEQ ID | Length | SEQ ID |
|---|---|---|---|
| 10 | 2713 | 9 | 2714-2715 |
| 8 | 2716-2718 | 7 | 2719-2722 |

In some aspects, the IgA2 hinge derived CAR spacer of the present disclosure comprises a sequence disclosed in TABLE 5 or anywhere in the disclosure, further comprising an N-terminal subsequence comprising 1, 2, 3, 4, or 5 IgA2 CH1 C-terminal domain amino acids from TABLE 6 covalently bound to the N-terminus of the IgA2 sequence from TABLE 5 or anywhere in the disclosure.

In some aspects, the IgA2 hinge derived CAR spacer of the present disclosure comprises a sequence disclosed in TABLE 5 or anywhere in the disclosure, further comprising a C-terminal subsequence comprising 1, 2, 3, 4, or 5 IgA2 CH2 N-terminal domain amino acids from TABLE 6 covalently bound to the C-terminus of the IgA2 sequence from TABLE 5 or anywhere in the disclosure.

In some aspects, the IgA2 hinge derived CAR spacer of the present disclosure comprises a sequence disclosed in TABLE 5 or anywhere in the disclosure, further comprising (i) an N-terminal subsequence comprising 1, 2, 3, 4, or 5 IgA2 CH1 C-terminal domain amino acids from TABLE 6 covalently bound to the N-terminus of the IgA2 sequence from TABLE 5 or anywhere in the disclosure, and (ii) a C-terminal subsequence comprising 1, 2, 3, 4, or 5 IgA2 CH2 N-terminal domain amino acids from TABLE 6 covalently bound to the C-terminus of the IgA2 sequence from TABLE 5 or anywhere in the disclosure.

IgA2 CH1 and CH2 sequences that can be appended to the N- and/or C-termini of the IgA2 derived CAR spacer sequences of TABLE 5 or anywhere in the disclosure are presented in TABLE 6.

In some aspects, the N-terminal proline of a full length IgA2 hinge of SEQ ID NO:2713 can be replaced with an arginine (see, e.g., Uniprot P01877). Accordingly, in some aspects, a full length IgA2 hinge disclosed herein has the sequence set forth in SEQ ID NO: 4530.

TABLE 6
IgA2. CH1 and CH2 optional N-terminal
CH1 sequences and C-terminal CH2
sequences (sequences from Uniprot P01877
comprising 1 to 5 amino acids flanking
the full IgA2 hinge of SEQ ID NO: 2713)

| CH1 | | CH2 | |
|---|---|---|---|
| Length | Sequences | Length | Sequences |
| 1 | C | 1 | P |
| 2 | PC | 2 | PR |
| 3 | VPC | 3 | PRL |
| 4 | TVPC (SEQ | 4 | PRLS (SEQ |
| | ID NO: 4526) | | ID NO: 2528) |
| 5 | VTVPC (SEQ | 5 | PRLSL (SEQ |
| | ID NO: 4527) | | ID NO: 4529) |

In some aspects, the IgA2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4848.

In some aspects, the IgA2 hinge derived CAR spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4849.

In some aspects, the IgA2 hinge derived CAR spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4850.

In some aspects, the IgA2 hinge derived CAR spacer comprises an amino acid sequence at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4851.

### II.C. IgG Hinge Derived CAR Spacer

Of the five immunoglobulin isotypes, immunoglobulin G (IgG) is most abundant in human serum. The four subclasses, IgG1, IgG2, IgG3, and IgG4, which are highly conserved, differ in their constant region, particularly in their hinges and upper CH2 domains. These regions are involved in binding to both IgG-Fc receptors (FcγR) and C1q. As a result, the different subclasses have different effector functions, both in terms of triggering FcγR-expressing cells, resulting in phagocytosis or antibody-dependent cell-mediated cytotoxicity, and activating complement.

The hinge exon of IgG1 encompasses a very flexible hinge (SEQ ID NO:2723). IgG2 has a shorter hinge than IgG1 (SEQ ID NO:2768). The lower hinge region of IgG2 (actually encoded by the CH2 region) also has a one amino acid deletion (lacking one of the double Glycines found at position 235-6 of the IgG1 lower hinge), resulting in IgG2 having the shortest hinge of all the IgG subclasses. In addition, the hinges of IgG2 are even more rigid due to a poly-proline helix, stabilized by up to four (with some exceptions discussed below) extra inter-heavy chain disulfide bridges. These properties restrict the flexibility of the IgG2 molecule. Similarly, the hinge region of IgG4 (SEQ ID NO: 4453) is also shorter than the IgG1 hinge region. The flexibility of the hinge region of IgG4 is intermediate between that of IgG1 and IgG2. Unlike the IgG2 hinge, the IgG4 hinge contains the double Glycines found at position 235-6 of the IgG1 lower hinge.

IgG3 has a much longer hinge region than any other IgG subclasses or Ig human isotype, i.e., about four times as long as the IgG1 hinge, containing up to 62 amino acids (including 21 prolines and 11 cysteines), forming a poly-proline helix with limited flexibility (SEQ ID NO:2813). The exact length of the hinge varies between allotypes of IgG3, which apparently has undergone much more evolutionary radiation than the other subclasses. In IgG3, the Fab fragments are relatively far away from the Fc fragment, giving the molecule a greater flexibility. This long hinge of IgG3 is a result of duplications of a hinge exon, encoded by one exon in IgG1, IgG2, and IgG4, but up to four exons in IgG3. One of those exons is common to all IgG3 allotypes, but it also has 1-3 copies of a homologous second type of IgG3-hinge exon. The elongated hinge in IgG3 is also responsible for its higher molecular weight compared to the other subclasses. The difference in hinge flexibility influences the relative orientation and movement of the Fab arms and Fc tail of the IgG antibody.

In IgG2, structural hinge isomers have been observed as a result of alternative formation of disulfide bonds between the cysteines in the hinge region of the heavy chains and those involved in the formation of disulfide bonds between the light and heavy chain. These isomers were found particularly in IgG2 antibodies with kappa-light chains, but much less for lambda light chains. The major forms are the classical A form, with four disulfide bridges between the two IgG2 heavy chains, and the B form in which one hinge cysteine forms a disulfide bond with the light chain. However, other configurations exist, as these isoforms apparently form independent of each other, giving rise to A/A, B/B, but also A/B isoforms

Two isomers of IgG4 differing in the disulfide bonding of hinge cysteines coexist. The core hinge of IgG is formed by a CXXC motif, also found in redox-reactive proteins such as thioredoxins. Compared to IgG1, with a relatively rigid CPPC (SEQ ID NO: 4531) motif, intra-chain disulfide bonds are more easily formed between these cysteines found at positions 226 and 229 in IgG4, which possesses a CPSC (SEQ ID NO:4532) core hinge. The result is an observable amount of non-covalently linked half-molecules (consisting of one heavy and one light chain, HL, as opposed to the classical configuration of H₂L₂) in addition to covalently linked inter-chain isomers. An S228P mutant of IgG4, thus with an IgG1-core hinge, does not form half-molecules, which is in agreement with the finding that this species does not occur in IgG1.

As used herein, the term “IgG1 hinge” refers to the core plus upper hinge of a IgG1 hinge, i.e., the sequence set forth in SEQ ID NO:2723. In some aspects, the IgG1 hinge also comprises the lower hinge (SEQ ID NO:4533) or a subsequence thereof. In some aspects, the subsequence of the IgG1 lower hinge appended to the C-terminus of the IgG1 hinge of SEQ ID NO:2723 or an N-terminal truncation thereof (i.e., a fragment of SEQ ID NO:2723 in which one or more amino acid residues have been removed from its N-terminal region) is selected from the group consisting of A, AP, APE, APEL (SEQ ID NO: 4534), APELL (SEQ ID NO:4535), APELLG (SEQ ID NO:4536), APELLGG (SEQ ID NO:4537), and APELLGGP (SEQ ID NO:4533).

In some aspects, an IgG1 hinge derived CAR spacer comprises at least five, six, or seven consecutive amino acids of the sequence set forth in SEQ ID NO: 2723 or SEQ ID NO: 4839. In some aspects, the IgG1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2723.

In some aspects, a CAR comprising an IgG1 hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgG1 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgG1 derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, the IgG1 hinge derived spacer is not the sequence set forth in SEQ ID NO: 2723. In some aspects, an IgG1 hinge derived CAR spacer comprises at least five, six, or seven consecutive amino acids of SEQ ID NO: 2723, but is not SEQ ID NO: 2723.

As used herein, the term “IgG1 hinge derived CAR spacer” comprises, e.g., a spacer comprising an IgG1 hinge of SEQ ID NO: 2723 (upper plus core hinge), an IgG1 hinge of SEQ ID NO: 4538 (upper hinge, core hinge, and lower hinge), fragments thereof, and variants thereof comprising 1, 2, 3, 4, 5 or more additional CH1 region and/or CH2 region amino acids. In some aspects, the term IgG1 derived CAR spacer refers to a subsequence of an IgG1 hinge of SEQ ID NO: 4538 (upper hinge, core hinge, and lower hinge), wherein the subsequence comprises 7, 8, 9, 10, 11, 12, 13, 14, 15, 16, 17, 18, 19, 20, 21, or 22 consecutive amino acids from the polypeptide sequence set forth in SEQ ID NO: 4538.

In some aspects, an “IgG1 hinge derived CAR spacer” of the present disclosure comprises, consists, or consist essentially of a sequence disclosed in TABLE 7.

TABLE 7
IgG1 derived spacers

| Length | SEQ ID | Length | SEQ ID | Length | SEQ ID |
|---|---|---|---|---|---|
| 15 | 2723 | 14 | 2724-2725 | 13 | 2726-2728 |
| 12 | 2729-2732 | 11 | 2733-2737 | 10 | 2738-2743 |
| 9 | 2744-2750 | 8 | 2751-2758 | 7 | 2759-2767 |

In some aspects, the IgG1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence sert forth in SEQ ID NO: 4840.

In some aspects, the IgG1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4839.

In some aspects, the IgG1 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4843.

In some aspects, the IgG1 hinge derived CAR spacer of the present disclosure comprises a sequence disclosed in TABLE 7 or anywhere in the disclosure, further comprising an N-terminal subsequence comprising 1, 2, 3, 4, or 5 IgG1 CH1 C-terminal domain amino acids from TABLE 8 covalently bound to the N-terminus of the IgG1 sequence from TABLE 7 or anywhere in the disclosure.

In some aspects, the IgG1 hinge derived CAR spacer of the present disclosure comprises a sequence disclosed in TABLE 7 or anywhere in the disclosure, further comprising a C-terminal subsequence comprising 1, 2, 3, 4, or 5 IgG1 CH2 N-terminal domain amino acids from TABLE 8 covalently bound to the C-terminus of the IgG1 sequence from TABLE 7 or anywhere in the disclosure.

In some aspects, the IgG1 hinge derived CAR spacer of the present disclosure comprises a sequence disclosed in TABLE 7 or anywhere in the disclosure, further comprising (i) an N-terminal subsequence comprising 1, 2, 3, 4, or 5 IgG1 CH1 C-terminal domain amino acids from TABLE 8 covalently bound to the N-terminus of the IgG1 sequence from TABLE 7 or anywhere in the disclosure, and (ii) a C-terminal subsequence comprising 1, 2, 3, 4, or 5 IgG1 CH2 N-terminal domain amino acids from TABLE 8 covalently bound to the C-terminus of the IgG1 sequence from TABLE 7 or anywhere in the disclosure.

IgG1 CH1 and CH2 sequences that can be appended to the N- and/or C-termini of the IgG1 hinge derived CAR spacer sequences of TABLE 7 or anywhere in the disclosure are presented in TABLE 8.

TABLE 8
IgG1. CH1 and CH2 optional N-terminal
CH1 sequences and C-terminal CH2
sequences (sequences from Uniprot P01857
comprising 1 to 5 amino acids flanking
the full IgG1 hinge of SEQ ID NO: 2723)

| CH1 | | CH2 | |
|---|---|---|---|
| Length | Sequences | Length | Sequences |
| 1 | V | 1 | A |
| 2 | KV | 2 | AP |
| 3 | KKV | 3 | APE |
| 4 | DKKV | 4 | APEL |
| | (SEQ ID | | (SEQ ID |
| | NO: 4539) | | NO: 4534) |
| 5 | VDKKV | 5 | APELL |
| | (SEQ ID | | (SEQ ID |
| | NO: 4540) | | NO: 4535) |

As used herein, the term “IgG2 hinge” refers to the core plus upper hinge of a IgG2 hinge, i.e., the sequence set forth in SEQ ID NO:2768. In some aspects, the IgG2 hinge comprises also the lower hinge, i.e., the sequence set forth in SEQ ID NO:4453 or a subsequence thereof. In some aspects, the subsequence of the IgG2 lower hinge appended to the C-terminus of the IgG2 hinge of SEQ ID NO:2768 or an N-terminal truncation thereof (i.e., a fragment of SEQ ID NO:2768 in which one or more amino acid residues have been removed from its N-terminal region) is selected from the group consisting of A, AP, APE, APEL (SEQ ID NO: 4534), APELL (SEQ ID NO:4535), APELLG (SEQ ID NO:4536), APELLGG (SEQ ID NO:4537), and APELLGGP (SEQ ID NO:4533).

In some aspects, an IgG2 hinge derived CAR spacer comprises at least five, six, or seven consecutive amino acids of the sequence set forth in SEQ ID NO: 2768. In some aspects, the IgG2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2768.

In some aspects, a CAR comprising an IgG2 hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgG2 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising an IgG2 derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, an IgG2 hinge derived CAR spacer comprises at least five, six, or seven consecutive amino acids of the sequence set forth in SEQ ID NO: 2768, but is not SEQ ID NO:4985.

In some aspects, the term “IgG2 hinge derived CAR spacer” refers, e.g., to a spacer comprising a human IgG2 hinge of SEQ ID NO: 2768 (upper plus core hinge), a human IgG2 hinge of SEQ ID NO: 4541 (upper hinge, core hinge, and lower hinge), fragments thereof, and variants thereof comprising 1, 2, 3, 4, 5 or more additional CH1 region and/or CH2 region amino acids. In some aspects, the term IgG2 derived CAR spacer refers to a subsequence of a human IgG2 hinge of SEQ ID NO: 4541 (upper hinge, core hinge, and lower hinge), wherein the subsequence comprises 7, 8, 9, 10, 11, 12, 13, 14, 15, 16, 17, 18, 19, 20, 21, or 22 consecutive amino acids from the polypeptide sequence set forth in SEQ ID NO: 4541.

In some aspects, an “IgG2 hinge derived CAR spacer” of the present disclosure comprises, consists, or consist essentially of a sequence disclosed in TABLE 9A.

TABLE 9A
Human IgG2 derived spacers

| Length | SEQ ID | Length | SEQ ID | Length | SEQ ID |
|---|---|---|---|---|---|
| 11 | 2768 | 10 | 2769-2770 | 9 | 2771-2773 |
| 8 | 2774-2777 | 7 | 2778-2782 | | |

In some aspects, the IgG2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4842.

In some aspects, the IgG2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2768.

In some aspects, a CAR comprising a human IgG2 hinge derived spacer disclosed herein is capable of inducing an increased Interferon-y level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising the IgG2 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911). In some aspects, the IgG2 hinge derived CAR spacer is not the sequence set forth in SEQ ID NO: 4986.

In some aspects, a CAR comprising the IgG2 derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, the human IgG2 hinge derived CAR spacer of the present disclosure comprises a sequence disclosed in TABLE 9A or anywhere in the disclosure, further comprising an N-terminal subsequence comprising 1, 2, 3, 4, or 5 IgG2 CH1 C-terminal domain amino acids from TABLE 10A covalently bound to the N-terminus of the IgG2 sequence from TABLE 9A or anywhere in the disclosure.

In some aspects, the human IgG2 hinge derived CAR spacer of the present disclosure comprises a sequence disclosed in TABLE 9A or anywhere in the disclosure, further comprising a C-terminal subsequence comprising 1, 2, 3, 4, or 5 IgG2 CH2 N-terminal domain amino acids from TABLE 10A covalently bound to the C-terminus of the IgG2 sequence from TABLE 9A or anywhere in the disclosure.

In some aspects, the human IgG2 hinge derived CAR spacer of the present disclosure comprises a sequence disclosed in TABLE 9A or anywhere in the disclosure, further comprising (i) an N-terminal subsequence comprising 1, 2, 3, 4, or 5 IgG2 CH1 C-terminal domain amino acids from TABLE 10A covalently bound to the N-terminus of the IgG2 sequence from TABLE 9A or anywhere in the disclosure, and (ii) a C-terminal subsequence comprising 1, 2, 3, 4, or 5 IgG2 CH2 N-terminal domain amino acids from TABLE 10A covalently bound to the C-terminus of the IgG2 sequence from TABLE 9A or anywhere in the disclosure.

IgG2 CH1 and CH2 sequences that can be appended to the N- and/or C-termini of the IgG2 hinge derived CAR spacer sequences of TABLE 9A are presented in TABLE 10A.

TABLE 10A
Human IgG2. CH1 and CH2 optional
N-terminal CH1 sequences and C-terminal CH2
sequences (sequences from Uniprot P01859
comprising 1 to 5 amino acids flanking
the full IgG2 hinge of SEQ ID NO: 2768)

| CH1 | | CH2 | |
|---|---|---|---|
| Length | Sequence | Length | Sequence |
| 1 | V | 1 | P |
| 2 | TV | 2 | PA |
| 3 | KTV | 3 | PAP |
| 4 | DKTV | 4 | PAPP |
| | (SEQ ID | | (SEQ ID |
| | NO: 4542) | | NO: 4544) |
| 5 | KDKTV | 5 | PAPPV |
| | (SEQ ID | | (SEQ ID |
| | NO: 4543) | | NO: 4545) |

In some aspects, the term “IgG2 hinge derived CAR spacer” refers, e.g., to a spacer comprising a murine IgG2 hinge of SEQ ID NO: 4926 (upper plus core hinge), a murine IgG2 hinge of SEQ TD NO: 4992 (upper hinge, core hinge, and lower hinge), fragments thereof, and variants thereof comprising 1, 2, 3, 4, 5 or more additional CH1 region and/or CH2 region amino acids. In some aspects, the term IgG2 derived CAR spacer refers to a subsequence of a murine IgG2 hinge of SEQ TD NO: 4992 (upper hinge, core hinge, and lower hinge), wherein the subsequence comprises 7, 8, 9, 10, 11, 12, 13, 14, 15, 16, 17, 18, 19, 20, 21, or 22 consecutive amino acids from the polypeptide sequence set forth in SEQ TD NO: 4992.

In some aspects, an “IgG2 hinge derived CAR spacer” of the present disclosure is a “murine IgG2 hinge derived CAR spacer” comprising, consisting, or consisting essentially of a sequence disclosed in TABLE 9B.

TABLE 9B
Murine IgG2A hinge derived CAR spacers

| Length | SEQ ID | Length | SEQ ID | Length | SEQ ID |
|---|---|---|---|---|---|
| 16 | 4926 | 15 | 4927-4928 | 14 | 4929-4931 |
| 13 | 4932-4935 | 12 | 4936-4940 | 11 | 4941-4946 |
| 10 | 4947-4953 | 9 | 4954-4961 | 8 | 4962-4970 |
| 7 | 4971-4980 | | | | |

In some aspects, the murine IgG2 hinge derived CAR spacer comprises at least five, six, or seven consecutive amino acids of the sequence set forth in SEQ ID NO: 4830. In some aspects, the murine IgG2 hinge derived CAR spacer comprises at least five, six, or seven consecutive amino acids of the sequence set forth in SEQ ID NO: 4831. In some aspects, the murine IgG2 hinge derived CAR spacer comprises at least five, six, or seven consecutive amino acids of the sequence set forth in SEQ ID NO: 4832.

In some aspects, the murine IgG2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4830. In some aspects, the murine IgG2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4831. In some aspects, the murine IgG2 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4832.

In some aspects, a CAR comprising a murine IgG2 hinge derived CAR spacer disclosed herein is capable of inducing an increased Interferon-y level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising a murine IgG2 hinge derived CAR spacer disclosed herein is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911). In some aspects, the murine IgG2 hinge derived CAR spacer is not the sequence set forth in SEQ ID NO: 4986.

In some aspects, a CAR comprising the murine IgG2 derived CAR spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, the murine IgG2 hinge derived CAR spacer of the present disclosure comprises a sequence disclosed in TABLE 9B or anywhere in the disclosure, further comprising an N-terminal subsequence comprising 1, 2, 3, 4, or 5 IgG2 CH1 C-terminal domain amino acids from TABLE 10B covalently bound to the N-terminus of the IgG2 sequence from TABLE 9B or anywhere in the disclosure.

In some aspects, the murine IgG2 hinge derived CAR spacer of the present disclosure comprises a sequence disclosed in TABLE 9B or anywhere in the disclosure, further comprising a C-terminal subsequence comprising 1, 2, 3, 4, or 5 IgG2 CH2 N-terminal domain amino acids from TABLE 10B covalently bound to the C-terminus of the IgG2 sequence from TABLE 9B or anywhere in the disclosure.

In some aspects, the murine IgG2 hinge derived CAR spacer of the present disclosure comprises a sequence disclosed in TABLE 9B or anywhere in the disclosure, further comprising (i) an N-terminal subsequence comprising 1, 2, 3, 4, or 5 IgG2 CH1 C-terminal domain amino acids from TABLE 10B covalently bound to the N-terminus of the IgG2 sequence from TABLE 9B or anywhere in the disclosure, and (ii) a C-terminal subsequence comprising 1, 2, 3, 4, or 5 IgG2 CH2 N-terminal domain amino acids from TABLE 10B covalently bound to the C-terminus of the IgG2 sequence from TABLE 9B or anywhere in the disclosure.

IgG2 CH1 and CH2 sequences that can be appended to the N- and/or C-termini of the IgG2 hinge derived CAR spacer sequences of TABLE 9B are presented in TABLE 10B.

TABLE 10B
Murine IgG2A.
CH1 and CH2 optional N-terminal CH1
sequences and C-terminal CH2 sequences
(sequences from Uniprot P01865 comprising
1 to 5 amino acids flanking the full
murine IgG2A hinge of SEQ ID NO: 4926)

| CH1 | | CH2 | |
|---|---|---|---|
| Length | Sequence | Length | Sequence |
| 1 | I | 1 | A |
| 2 | KI | 2 | AP |
| 3 | KKI | 3 | APN |
| 4 | DKKI | 4 | APNL |
| | (SEQ ID | | (SEQ ID |
| | NO: 4981) | | NO: 4983) |
| 5 | VDKKI | 5 | APNLL |
| | (SEQ ID | | (SEQ ID |
| | NO: 4982) | | NO: 4984) |

As used herein, the term “IgG3 hinge” refers to the core plus upper hinge of a IgG3 hinge, i.e., the sequence set forth in SEQ ID NO:2813. In some aspects, the IgG3 hinge comprises also the lower hinge (APELLGGP; SEQ ID NO:4533).

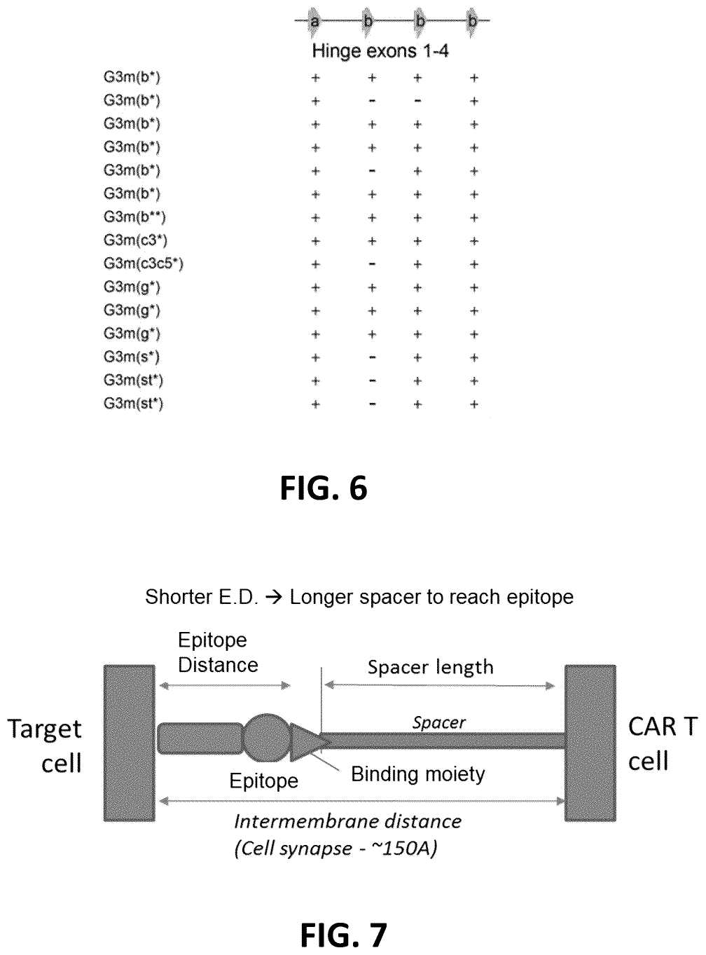

The long hinge of IgG3 is a result of duplications of a hinge exon, encoded by one exon in IgG1, IgG2, and IgG4, but up to four exons in IgG3. One of those exons is common to all IgG3 allotypes, but it also has 1-3 copies of a homologous second type of IgG3-hinge exon. Depending on the number of sequence repeats, the hinge region can vary from 27 to 83 amino acid residues between different IgG3 allotypes (see FIG. 6). As shown in FIG. 6, the different IgG3 allotypes identified include a hinge with a pattern corresponding to a, ab, abb, or abbb, wherein “a” is the sequence set forth in SEQ ID NO: 3848 and “b” is the sequence set forth in SEQ ID NO: 3958. In some aspects, the “b” sequence is a sequence set forth in SEQ ID NO: 4546 (human variant), SEQ ID NO:4547 (Pan troglodytes), SEQ ID NO: 4548 (Pan troglodytes), SEQ ID NO:4549 (Pongo pygmaeus), SEQ ID NO:4550 (Gorilla gorilla), or SEQ ID NO: 4551 (Pongo abelii).

In some aspects, an IgG3 hinge derived CAR spacer comprises at least five, six, or seven consecutive amino acids of the sequence set forth in SEQ ID NO: 2813.

In some aspects, a CAR comprising a IgG3 hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising a IgG3 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising the IgG3 derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, an IgG3 hinge derived CAR spacer is not SEQ ID NO:2813.

In some aspects, the IgG3 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 2813.

As used herein, the term “IgG3 hinge derived CAR spacer” comprises, e.g., a spacer comprising an IgG3 hinge of SEQ ID NO: 2813 (upper plus core hinge), an IgG3 hinge of SEQ ID NO: 4552 (upper hinge, core hinge, and lower hinge), fragments thereof, and variants thereof comprising 1, 2, 3, 4, 5 or more additional CH1 region and/or CH2 region amino acids.

In some aspects, an IgG3 hinge derived CAR spacer disclosed comprises one of more mutations replacing the Threonine (T) in the subsequence set forth in SEQ ID NO:4553. These threonines are susceptible to glycosylation and also are substrates for trypsin and proteinase K. Accordingly, their replacement can reduce the immunogenicity caused by the CAR spacer and increase resistance to proteases, thus improving the stability of the CAR in vivo.

In some aspects, an IgG3 hinge derived CAR spacer of the present disclosure comprises, consists, or consist essentially of a sequence disclosed in TABLE 11.

TABLE 11
IgG3 derived spacers

| Length | SEQ ID | Length | SEQ ID | Length | SEQ ID |
|---|---|---|---|---|---|
| 62 | 2813 | 61 | 2814-2815 | 60 | 2816-2818 |
| 59 | 2819-2822 | 58 | 2823-2827 | 57 | 2828-2833 |
| 56 | 2834-2840 | 55 | 2841-2848 | 54 | 2849-2857 |
| 53 | 2858-2867 | 52 | 2868-2878 | 51 | 2879-2890 |
| 50 | 2891-2903 | 49 | 2904-2917 | 48 | 2918-2932 |
| 47 | 2933-2948 | 46 | 2949-2965 | 45 | 2966-2983 |
| 44 | 2984-3002 | 43 | 3003-3022 | 42 | 3023-3043 |
| 41 | 3044-3065 | 40 | 3066-3088 | 39 | 3089-3112 |
| 38 | 3113-3137 | 37 | 3138-3163 | 36 | 3164-3190 |
| 35 | 3191-3218 | 34 | 3219-3247 | 33 | 3248-3277 |
| 32 | 3278-3308 | 31 | 3309-3340 | 30 | 3341-3373 |
| 29 | 3374-3407 | 28 | 3408-3442 | 27 | 3443-3478 |
| 26 | 3479-3515 | 25 | 3516-3553 | 24 | 3554-3592 |
| 23 | 3593-3632 | 22 | 3633-3673 | 21 | 3674-3715 |
| 20 | 3716-3758 | 19 | 3759-3802 | 18 | 3803-3847 |
| 17 | 3848-3893 | 16 | 3894-3940 | 15 | 3941-3988 |
| 14 | 3989-4037 | 13 | 4038-4087 | 12 | 4088-4138 |
| 11 | 4139-4190 | 10 | 4191-4243 | 9 | 4244-4297 |
| 8 | 4298-4352 | 7 | 4755-4810 | | |

In some aspects, the IgG3 hinge derived CAR spacer comprises an amino acid sequence having at least about 70%, at least about 75%, at least about 80%, at least about 85%, at least about 90%, at least about 9500 at least about 96%, at least about 9700 at least about 98%, at least about 9900, or about 10000 sequence identity to a sequence selected from the group consisting of SEQ ID NO: 4833; SEQ ID NO: 4834; SEQ ID NO: 4835; SEQ ID NO: 4836; SEQ ID NO: 4837; SEQ ID NO: 4838, SEQ ID NO: 4841; and any combination thereof.

In some aspects, the IgG3 hinge derived CAR spacer comprises an amino acid sequence having at least about 70%, at least about 75%, at least about 80%, at least about 85%, at least about 90%, at least about 9500 at least about 96%, at least about 9700 at least about 98%, at least about 99, or about 100% sequence identity to the sequence set forth in SEQ TD NO: 4833.

In some aspects, the IgG3 hinge derived CAR spacer comprises an amino acid sequence having at least about 70%, at least about 75%, at least about 80%, at least about 85%, at least about 9000, at least about 9500, at least about 9600, at least about 9700, at least about 9800, at least about 99, or about 1000% sequence identity to the sequence set forth in SEQ TD NO: 4834.

In some aspects, the IgG3 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4835.

In some aspects, the IgG3 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4836.

In some aspects, the IgG3 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4837.

In some aspects, the IgG3 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4838.

In some aspects, the IgG3 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4841.

In some aspects, the IgG3 hinge derived CAR spacer of the present disclosure comprises a sequence disclosed in TABLE 11 or anywhere in the disclosure, further comprising an N-terminal subsequence comprising 1, 2, 3, 4, or 5 IgG3 CH1 C-terminal domain amino acids from TABLE 12 covalently bound to the N-terminus of the IgG3 sequence from TABLE 11 or anywhere in the disclosure.

In some aspects, the IgG3 hinge derived CAR spacer of the present disclosure comprises a sequence disclosed in TABLE 11 or anywhere in the disclosure, further comprising a C-terminal subsequence comprising 1, 2, 3, 4, or 5 IgG3 CH2 N-terminal domain amino acids from TABLE 12 covalently bound to the C-terminus of the IgG3 sequence from TABLE 11 or anywhere in the disclosure.

In some aspects, the IgG3 hinge derived CAR spacer of the present disclosure comprises a sequence disclosed in TABLE 11 or anywhere in the disclosure, further comprising (i) an N-terminal subsequence comprising 1, 2, 3, 4, or 5 IgG3 CH1 C-terminal domain amino acids from TABLE 12 covalently bound to the N-terminus of the IgG3 sequence from TABLE 11 or anywhere in the disclosure, and (ii) a C-terminal subsequence comprising 1, 2, 3, 4, or 5 IgG3 CH2 N-terminal domain amino acids from TABLE 12 covalently bound to the C-terminus of the IgG3 sequence from TABLE 11 or anywhere in the disclosure.

IgG3 CH1 and CH2 sequences that can be appended to the N- and/or C-termini of the IgG3 hinge derived CAR spacer sequences of TABLE 11 or anywhere in the disclosure are presented in TABLE 12.

TABLE 12
IgG3. CH1 and CH2 optional N-terminal
CH1 sequences and C-terminal CH2 sequences
(sequences from Uniprot P01860 comprising
1 to 5 amino acids flanking the full
IgG3 hinge of SEQ ID NO: 2813)

| CH1 | | CH2 | |
|---|---|---|---|
| Length | Sequences | Length | Sequences |
| 1 | V | 1 | A |
| 2 | RV | 2 | AP |
| 3 | KRV | 3 | APE |
| 4 | DKRV | 4 | APEL |
| | (SEQ ID | | (SEQ ID |
| | NO: 4554) | | NO: 4534) |
| 5 | VDKRV | 5 | APELL |
| | (SEQ ID | | (SEQ ID |
| | NO: 4555) | | NO: 4535) |

As used herein, the term “IgG4 hinge” refers to the core plus upper hinge of a IgG4 hinge, i.e., the sequence set forth in SEQ ID NO:4353. In some aspects, the IgG4 hinge comprises also the lower hinge (APEFLGGP; SEQ ID NO:4556) or a subsequence thereof. In some aspects, the subsequence of the IgG4 lower hinge appended to the C-terminus of the IgG4 hinge of SEQ ID NO:4353 or an N-terminal truncation thereof (i.e., a fragment of SEQ ID NO:4353 in which one or more amino acid residues have been removed from its N-terminal region) is selected from the group consisting of A, AP, APE, APEF (SEQ ID NO: 4557), APEFL (SEQ ID NO:4558), APEFLG (SEQ ID NO:4559), APEFLGG (SEQ ID NO:4560), and APEFLGGP (SEQ ID NO:4556).

As used herein, the term “IgG4 hinge derived CAR spacer” comprises, e.g., a spacer comprising an IgG4 hinge of SEQ ID NO: 4353 (upper plus core hinge), an IgG4 hinge of SEQ ID NO: 4561 (upper hinge, core hinge, and lower hinge), fragments thereof, and variants thereof comprising 1, 2, 3, 4, 5 or more additional CH1 region and/or CH2 region amino acids. In some aspects, the term IgG4 derived CAR spacer refers to a subsequence of an IgG4 hinge of SEQ ID NO: 4561 (upper hinge, core hinge, and lower hinge), wherein the subsequence comprises 7, 8, 9, 10, 11, 12, 13, 14, 15, 16, 17, 18, 19, 20, 21, or 22 consecutive amino acids from the polypeptide sequence set forth in SEQ ID NO: 4561.

In some aspects, an IgG4 hinge derived CAR spacer comprises at least five, six, or seven consecutive amino acids of the sequence set forth in SEQ ID NO: 4353. In some aspects, the IgG4 hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4353.

In some aspects, a CAR comprising the IgG4 hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising the IgG4 hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, the IgG4 hinge derived CAR spacer is not SEQ ID NO: 4987. In some aspects, the IgG4 hinge derived CAR spacer is not SEQ ID NO: 4988. In some aspects, the IgG4 hinge derived CAR spacer is not SEQ ID NO: 4989. In some aspects, the IgG4 hinge derived CAR spacer is not SEQ ID NO: 4990. In some aspects, the IgG4 hinge derived CAR spacer is not SEQ ID NO: 4991.

In some aspects, an “IgG4 hinge derived CAR spacer” of the present disclosure comprises, consists, or consist essentially of a sequence disclosed in TABLE 13.

TABLE 13
Ig G4 derived spacers

| Length | SEQ ID | Length | SEQ ID | Length | SEQ ID |
|---|---|---|---|---|---|
| 9 | 4353 | 11 | 4354-4355 | 10 | 4356-4358 |
| 8 | 4359-4362 | 8 | 4363-4367 | 7 | 4368-4373 |

In some aspects, the IgG4 hinge derived CAR spacer of the present disclosure comprises a sequence disclosed in TABLE 13 or anywhere in the disclosure, further comprising an N-terminal subsequence comprising 1, 2, 3, 4, or 5 IgG4 CH1 C-terminal domain amino acids from TABLE 14 covalently bound to the N-terminus of the IgG4 sequence from TABLE 13 or anywhere in the disclosure. In some aspects, the IgG4 hinge derived CAR spacer of the present disclosure comprises a sequence disclosed in TABLE 13 or anywhere in the disclosure, further comprising a C-terminal subsequence comprising 1, 2, 3, 4, or 5 IgG4 CH2 N-terminal domain amino acids from TABLE 14 covalently bound to the C-terminus of the IgG4 sequence from TABLE 13 or anywhere in the disclosure.

In some aspects, the IgG4 hinge derived CAR spacer of the present disclosure comprises a sequence disclosed in TABLE 13 or anywhere in the disclosure, further comprising (i) an N-terminal subsequence comprising 1, 2, 3, 4, or 5 IgG4 CH1 C-terminal domain amino acids from TABLE 14 covalently bound to the N-terminus of the IgG4 sequence from TABLE 13 or anywhere in the disclosure, and (ii) a C-terminal subsequence comprising 1, 2, 3, 4, or 5 IgG4 CH2 N-terminal domain amino acids from TABLE 14 covalently bound to the C-terminus of the IgG4 sequence from TABLE 13 or anywhere in the disclosure.

IgG4 CH1 and CH2 sequences that can be appended to the N- and/or C-termini of the IgG4 hinge derived CAR spacer sequences of TABLE 13 or anywhere in the disclosure are presented in TABLE 14.

TABLE 14
IgG4. CH1 and CH2 optional N-terminal
CH1 sequences and C-terminal CH2 sequences
(sequences from Uniprot P01861 comprising
1 to 5 amino acids flanking the full
IgG4 hinge of SEQ ID NO: 4353).

| CH1 | | CH2 | |
|---|---|---|---|
| Length | Sequence | Length | Sequence |
| 1 | V | 1 | A |
| 2 | RV | 2 | AP |
| 3 | KRV | 3 | APE |
| 4 | DKRV | 4 | APEF |
| | (SEQ ID | | (SEQ ID |
| | NO: 4554) | | NO: 4557) |
| 5 | VDKRV | 5 | APEFL |
| | (SEQ ID | | (SEQ ID |
| | NO: 4555) | | NO: 4558) |

### II.D. IgE Hinge Derived CAR Spacer

Immunoglobulin E (IgE) is a type of immunoglobulin (Ig) isotype comprising two heavy chains (F chain) and two light chains, with the F chain containing 4 Ig-like constant domains (Cε1-Cε4). IgE is unique in that it lacks an actual hinge region and gets replaced by the Cε2 domain.

As used herein, the term “IgE hinge” refers to the sequence located between the C-terminus of Cε1 and the N-terminus of C2, i.e., the sequence located between amino acids 241 and 260. Accordingly, as defined herein, a full length “IgE hinge” would correspond to the sequence set forth in SEQ ID NO: 4374.

As used herein, the term “IgE hinge derived CAR spacer” comprises, e.g., a spacer comprising the sequence set forth in SEQ ID NO: 4374, fragments thereof, and variants thereof comprising 1, 2, 3, 4, 5 or more additional Cε1 region and/or Cε2 region amino acids.

In some aspects, an IgE hinge derived CAR spacer comprises at least five, six, or seven consecutive amino acids of the sequence set forth in SEQ ID NO: 4374. In some aspects, the IgE hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4374.

In some aspects, a CAR comprising the IgE hinge derived spacer is capable of inducing an increased Interferon-7 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising the IgE hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising a IgE hinge derived spacer disclosed herein is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, an IgE hinge derived CAR spacer of the present disclosure comprises, consists, or consist essentially of a sequence disclosed in TABLE 15.

TABLE 15
IgE derived spacers

| Length | SEQ ID | Length | SEQ ID | Length | SEQ ID |
|---|---|---|---|---|---|
| 19 | 4374 | 18 | 4375-4376 | 17 | 4377-4379 |
| 16 | 4380-4383 | 15 | 4384-4388 | 14 | 4389-4394 |
| 13 | 4395-4401 | 12 | 4402-4409 | 11 | 4410-4418 |
| 10 | 4419-4428 | 9 | 4429-4439 | 8 | 4440-4451 |
| 7 | 4452-4464 | | | | |

In some aspects, the IgE hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4856.

In some aspects, the IgE hinge derived CAR spacer of the present disclosure comprises a sequence disclosed in TABLE 15 or anywhere in the disclosure, further comprising an N-terminal subsequence comprising 1, 2, 3, 4, or 5 IgE Cε1 C-terminal domain amino acids from TABLE 16 covalently bound to the N-terminus of the IgE sequence from TABLE 15 or anywhere in the disclosure.

In some aspects, the IgE hinge derived CAR spacer of the present disclosure comprises a sequence disclosed in TABLE 15 or anywhere in the disclosure, further comprising a C-terminal subsequence comprising 1, 2, 3, 4, or 5 IgE Cε2 N-terminal domain amino acids from TABLE 16 covalently bound to the C-terminus of the IgE sequence from TABLE 15 or anywhere in the disclosure.

In some aspects, the IgE hinge derived CAR spacer of the present disclosure comprises a sequence disclosed in TABLE 15 or anywhere in the disclosure, further comprising (i) an N-terminal subsequence comprising 1, 2, 3, 4, or 5 IgE Cel C-terminal domain amino acids from TABLE 16 covalently bound to the N-terminus of the IgE sequence from TABLE 15 or anywhere in the disclosure, and (ii) a C-terminal subsequence comprising 1, 2, 3, 4, or 5 IgE Cε2 N-terminal domain amino acids from TABLE 16 covalently bound to the C-terminus of the IgE sequence from TABLE 15 or anywhere in the disclosure.

IgE Cel and Cε2 sequences that can be appended to the N- and/or C-termini of the IgE hinge derived CAR spacer sequences of TABLE 15 or anywhere in the disclosure are presented in TABLE 16.

TABLE 16
IgE. Cε1 and Cε2 optional N-terminal
Cε1 sequences and C-terminal Cε2 sequences
(sequences from Uniprot P01854 comprising 1
to 5 amino acids flanking the full IgGE
hinge of SEQ ID NO: 4374).

| Cε1 | | Cε2 | |
|---|---|---|---|
| Length | Sequences | Length | Sequences |
| 1 | D | 1 | K |
| 2 | TD | 2 | KI |
| 3 | STD | 3 | KIL |
| 4 | SSTD | 4 | KILQ |
| | (SEQ ID | | (SEQ ID |
| | NO: 4562) | | NO: 4564) |
| 5 | PSSTD | 5 | KILQS |
| | (SEQ ID | | (SEQ ID |
| | NO: 4563) | | NO: 4565) |

For simplicity, the IgE Cel region is considered a CH1 region, and the IgE Cε2 region is considered a CH2 region. Accordingly, references to a CH1 of IgM throughout the specification refer to IgE Cel. Similarly, references to a CH2 of IgE throughout the specification refer to IgE Cε2.

### II.E. IgM Hinge Derived CAR Spacer

Immunoglobulin M (IgM) is the largest antibody, and it is the first antibody to appear in the response to initial exposure to an antigen. IgM includes light chains and heavy chains. The light chain (k or x) is a protein of ˜220 amino acids, composed of a variable domain, VL (a segment of approximately 110 amino acids), and a constant domain, CL (also approximately 110 amino acids long). The p heavy chain of IgM is a protein of ˜576 amino acids, and includes a variable domain (VH-110 amino acids), four distinct constant region domains (Cμ1, Cμ2, Cμ3, Cμ4, each ˜110 amino acids) and a “tailpiece” of ˜20 amino acids. Cμ1 and Cμ2 are connected by the connector sequence set forth in SEQ ID NO:4727.

For the purposes of the present the disclosure, the term “IgM hinge” is defined as an extended sequence surrounding the Cμ1-Cμ2 connector. Accordingly, as defined herein, a full length “IgM hinge” of the present disclosure would correspond to the sequence set forth in SEQ ID NO: 4465.

As used herein, the term “IgM hinge derived CAR spacer” comprises, e.g., a spacer comprising the sequence set forth in SEQ ID NO: 4465, fragments thereof, and variants thereof comprising 1, 2, 3, 4, 5 or more additional Cp region and/or Cp region amino acids. In some aspects, the IgM hinge derived CAR spacer is a Cμ1-Cμ2 connector having the sequence set forth in SEQ ID NO:4727.

In some aspects, an IgM hinge derived CAR spacer comprises at least five, six, or seven consecutive amino acids of the sequence set forth in SEQ ID NO: 4465. In some aspects, the IgM hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4465.

In some aspects, a CAR comprising the IgM hinge derived spacer is capable of inducing an increased Interferon-γ level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising the IgM hinge derived spacer is capable of inducing an increased Interleukin-2 level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, a CAR comprising the IgM hinge derived spacer is capable of inducing an increased TNF-α level, e.g., at least 10%, at least about 20%, at least about 30%, at least about 40%, at least about 50%, at least about 60%, at least about 70%, at least about 80%, at least about 90%, at least about 100%, at least about 120%, at least about 150%, compared to a CAR comprising a reference spacer, e.g., a reference IgG4 spacer (e.g., a spacer of SEQ ID NO: 4911).

In some aspects, an IgM hinge derived CAR spacer of the present disclosure comprises, consists, or consist essentially of a sequence disclosed in TABLE 17.

TABLE 17
IgM derived spacers

| Length | SEQ ID | Length | SEQ ID | Length | SEQ ID |
|---|---|---|---|---|---|
| 26 | 4465 | 25 | 2783-2784 | 24 | 2785-2787 |
| 23 | 2788-2791 | 22 | 2792-2796 | 21 | 2797-2802 |
| 20 | 2803-2809 | 19 | 2810-2812, | 18 | 4571-4579 |
| | | | 4566-4570 | | |
| 17 | 4580-4589 | 16 | 4590-4600 | 15 | 4601-4612 |
| 14 | 4613-4625 | 13 | 4626-4639 | 12 | 4640-4654 |
| 11 | 4655-4670 | 10 | 4671-4687 | 9 | 4688-4705 |
| 8 | 4706-4724 | 7 | 4725-4744 | | |

In some aspects, the IgM hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to a sequence selected from the group consisting of SEQ ID NO: 4857; SEQ ID NO: 4858; SEQ ID NO: 4859; and any combination thereof.

In some aspects, the IgM hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4857.

In some aspects, the IgM hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4858.

In some aspects, the IgM hinge derived CAR spacer comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 4859.

In some aspects, the IgM hinge derived CAR spacer of the present disclosure comprises a sequence disclosed in TABLE 17 or anywhere in the disclosure, further comprising an N-terminal subsequence comprising 1, 2, 3, 4, or 5 IgM Cμ1 C-terminal domain amino acids from TABLE 18 covalently bound to the N-terminus of the IgM sequence from TABLE 17 or anywhere in the disclosure.

In some aspects, the IgM hinge derived CAR spacer of the present disclosure comprises a sequence disclosed in TABLE 17 or anywhere in the disclosure, further comprising a C-terminal subsequence comprising 1, 2, 3, 4, or 5 IgM Cμ2 N-terminal domain amino acids from TABLE 18 covalently bound to the C-terminus of the IgM sequence from TABLE 17 or anywhere in the disclosure.

In some aspects, the IgM hinge derived CAR spacer of the present disclosure comprises a sequence disclosed in TABLE 17 or anywhere in the disclosure, further comprising (i) an N-terminal subsequence comprising 1, 2, 3, 4, or 5 IgN Cμ1 C-terminal domain amino acids from TABLE 18 covalently bound to the N-terminus of the IgM sequence from TABLE 17 or anywhere in the disclosure, and (ii) a C-terminal subsequence comprising 1, 2, 3, 4, or 5 IgM Cμ2 N-terminal domain amino acids from TABLE 18 covalently bound to the C-terminus of the IgM sequence from TABLE 17 or anywhere in the disclosure.

IgM Cμ1 and Cμ2 sequences that can be appended to the N- and/or C-termini of the IgM hinge derived CAR spacer sequences of TABLE 17 or anywhere in the disclosure are presented in TABLE 18.

TABLE 18
IgM. Cμ1 and Cμ2 optional N-terminal
Cμ1 sequences and C-terminal Cμ2 sequences
(sequences from Uniprot P01871 comprising 1
to 5 amino acids flanking the full IgM hinge
of SEQ ID NO: 4465).

| Cμ1 | | Cμ2 | |
|---|---|---|---|
| Length | Sequences | Length | Sequences |
| 1 | V | 1 | R |
| 2 | NV | 2 | RK |
| 3 | KNV | 3 | RKS |
| 4 | EKNV | 4 | RKSK |
| | (SEQ ID | | (SEQ ID |
| | NO: 4745) | | NO: 4747) |
| 5 | KEKNV | 5 | RKSKL |
| | (SEQ ID | | (SEQ ID |
| | NO: 4746) | | NO: 4748) |

For simplicity, the IgM Cμ1 region is considered a CH1 region, and the IgM Cμ2 region is considered a CH2 region. Accordingly, references to a CH1 of IgM throughout the specification refer to IgM Cμ1. Similarly, references to a CH2 of IgM throughout the specification refer to IgM Cμ2.

In some aspects, a CAR spacer of the present disclosure comprises a sequence set forth in TABLES 1, 3, 5, 7, 9A, 9B, 11, 13, 15, 17, or a combination thereof. In some aspects, the CAR spacer of the present disclosure comprises a sequence set forth in TABLES 1, 3, 5, 7, 9A, 9B, 11, 13, 15, 17, or a combination thereof and further comprises an N-terminal sequence set forth in TABLES 2, 4, 6, 8, 10A, 10B, 12, 14, 16, 18, or a combination thereof. In some aspects, the CAR spacer of the present disclosure comprises a sequence set forth in TABLES 1, 3, 5, 7, 9A, 9B, 11, 13, 15, 17 or a combination thereof and further comprises a C-terminal sequence set form in TABLES 2, 4, 6, 8, 10A, 10B, 12, 14, 16, 18, or a combination thereof. In some aspects, the CAR spacer of the present disclosure comprises a sequence set forth in TABLES 1, 3, 5, 7, 9A, 9B, 11, 13, 15, 17, or a combination thereof and further comprises (i) an N-terminal sequence set forth in TABLES 2, 4, 6, 8, 10A, 10B, 12, 14, 16, 18, or a combination thereof, and (ii) a C-terminal sequence set form in TABLES 2, 4, 6, 8, 10A, 10B, 12, 14, 16, 18, or a combination thereof.

In some aspects, a CAR spacer of the present disclosure is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 amino acids in length.

In some aspects, a CAR spacer of the present disclosure is less that about 400, less than about 390, less than about 385, less than about 380, less than about 375, less than about 370, less than about 365, less than about 360, less than about 355, less than about 350, less than about 345, less than about 340, less than about 335, less than about 330, less than about 325, less than about 320, less than about 315, less than about 310, less than about 305, less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, less than about 220, less than about 215, less than about 210, less than about 205, less than about 200, less than about 195, less than about 190, less than about 185, less than about 180, less than about 175, less than about 170, less than about 165, less than about 160, less than about 155, less than about 150, less than about 145, less than about 140, less than about 135, less than about 130, less than about 125, less than about 120, less than about 115, less than about 110, less than about 105, less than about 100, less than about 95, less than about 90, less than about 85, less than about 80, less than about 75, less than about 70, less than about 65, less than about 60, less than about 55, less than about 50, less than about 45, less than about 40, less than about 35, less than about 30, less than about 25, less than about 20, less than about 15, less than about 10, or less than about 5 amino acids in length.

In some aspects, a CAR spacer of the present disclosure is between 4 and 8, between 4 and 9, between 4 and 10, between 5 and 8, between 5 and 9, between 5 and 10, between 4 and 12, between 4 and 13, between 4 and 14, between 4 and 15, between 4 and 16, between 5 and 14, between 5 and 15, between 5 and 16, between 6 and 12, between 6 and 13, between 6 and 14, between 6 and 15, between 6 and 16, between 7 and 9, between 7 and 10, between 7 and 11, between 7 and 12, between 7 and 13, between 7 and 14, between 7 and 15, between 7 and 16, between 10 and 15, between 15 and 20, between 20 and 25, between 25 and 30, between 30 and 35, between 35 and 40, between 40 and 45, between 45 and 50, between 50 and 55, between 55 and 60, between 60 and 65, between 65 and 70, between 70 and 75, between 75 and 80, between 80 and 85, between 85 and 90, between 90 and 95, between 95 and 100, between 100 and 105, between 105 and 110, between 110 and 115, between 115 and 120, between 120 and 125, between 125 and 130, between 130 and 135, between 135 and 140, between 140 and 145, between 145 and 150, between 150 and 155, between 155 and 160, between 160 and 165, between 165 and 170, between 170 and 175, between 175 and 180, between 180 and 185, between 185 and 190, between 190 and 195, between 195 and 200 amino acids in length.

In some aspects, a CAR spacer of the present disclosure is between about 400 and about 390, between 390 and about 380, between about 380 and about 370, between about 370 and about 360, between about 360 and about 350, between about 350 and about 340, between about 340 and about 330, between about 330 and about 320, between about 320 and about 310, between about 310 and about 300, between about 300 and about 290, between about 290 and about 280, between about 280 and about 270, between about 270 and about 260, between about 260 and about 250, between about 250 and about 240, between about 240 and about 230, between about 230 and about 220, between about 220 and about 210, between about 210 and about 200, between about 200 and about 190, between about 190 and about 180, between about 180 and about 170, between about 170 and about 160, between about 160 and about 150, between about 150 and about 140, between about 140 and about 130, between about 130 and about 120, between about 120 and about 110, between about 110 and about 100, between about 100 and about 90, between about 90 and about 80, between about 80 and about 70, between about 70 and about 60, between about 60 and about 50, between about 50 and about 40, between 40 and about 30, between about 30 and about 20, or between about 20 and about 10 amino acids in length.

In some aspects, a CAR spacer of the present disclosure is between about 400 and about 380, between about 380 and about 360, between about 360 and about 340, between about 340 and about 320, between about 320 and about 300, between about 300 and about 280, between about 280 and about 260, between about 260 and about 240, between about 240 and 220, between about 220 and about 200, between about 200 and about 180, between about 180 and about 160, between about 160 and about 140, between about 120 and about 100, between about 100 and about 80, between about 80 and about 60, between about 60 and about 40, between about 40 and 20, between about 20 and about 10 amino acids in length.

In some aspects, a CAR spacer of the present disclosure is between about 400 and about 350, between about 350 and about 300, between about 300 and about 250, between about 250 and 200, between about 200 and about 150, between about 150 and about 100, between 100 and about 50, between 50 and about 1 amino acids in length.

In some aspects, a CAR spacer of the present disclosure is between about 400 and about 300, between about 300 and about 200, between about 200 and about 100, or between about 100 and about 1 amino acids in length.

In some aspects, a CAR spacer of the present disclosure, e.g., a CAR spacer disclosed in TABLES 1, 3, 5, 7, 9A, 9B, 11, 13, 15, or 17, is about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, or about 250 angstrom in length.

In some aspects, a CAR spacer of the present disclosure, e.g., a CAR spacer disclosed in TABLES 1, 3, 5, 7, 9A, 9B, 11, 13, 15, or 17, is between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, between about 95 and about 100, between about 100 and about 105, between about 105 and about 110, between about 110 and about 115, between about 115 and about 120, between about 120 and about 125, between about 125 and about 130, between about 130 and about 135, between about 135 and about 140, between about 140 and about 145, between about 145 and about 150, between about 150 and about 155, between about 155 and about 160, between about 160 and about 165, between about 165 and 170, between about 170 and about 175, between about 175 and about 180, between about 180 and about 185, between about 185 and about 190, between about 190 and about 195, between about 195 and about 200, between about 200 and about 205, between about 205 and about 210, between about 210 and about 215, between about 215 and about 220, between about 220 and about 225, between about 225 and about 230, between about 230 and about 235, between about 235 and about 240, between about 240 and about 245, or about between about 245 and about 250 angstrom in length.

In some aspects, a CAR spacer of the present disclosure, e.g., a CAR spacer disclosed in TABLES 1, 3, 5, 7, 9A, 9B, 11, 13, 15, or 17, is between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, or about between about 245 and about 250 angstrom in length.

In some aspects, a CAR spacer of the present disclosure, e.g., a CAR spacer disclosed in TABLES 1, 3, 5, 7, 9A, 9B, 11, 13, 15, or 17, is between about 5 and about 25, between about 25 and about 50, between about 50 and about 75, between about 75 and about 100, between about 100 and about 125, between about 125 and about 150, between about 150 and about 175, between about 175 and about 200, between about 200 and about 225, or between about 225 and about 250 angstrom in length.

In some aspects, a CAR spacer of the present disclosure, e.g., a CAR spacer disclosed in TABLES 1, 3, 5, 7, 9A, 9B, 11, 13, 15, or 17, is less than about 250, less than about 240, less that about 230, less than about 220, less than about 210, less than about 200, less that about 190, less than about 180, less than about 170, less than about 160, less than about 150, less than about 140, less than about 130, less than about 120, less than about 110, less than about 100, less than about 90, less than about 80, less than about 70, less than about 60, less than about 50, less than about 40, less than about 30, less than about 20, or less than about 10 angstrom in length.

In some aspects, a CAR spacer of the present disclosure comprises an amino acid sequence having at least about 60%, at least about 65%, at least about 70%, at least about 75%, at least about 80%, at least about 85%, at least about 90%, at least about 95%, at least about 96%, at least about 97%, at least about 99%, or about 100% identity to a sequence set forth in TABLES 1, 3, 5, 7, 9A, 9B, 11, 13, 15 or 17.

In some aspects, a CAR spacer of the present disclosure comprises an amino acid sequence having 1, 2, 3, 4, 5, 6, 7, 8, 9, or 10 amino acid substitutions with respect to a sequence set forth in TABLES 1, 3, 5, 7, 9A, 9B, 11, 13, 15 or 17. In some aspects, cysteine amino acids are replaced with alanine. In some aspects, all the amino acid substitution are conservative amino acid substitutions. In some aspects, at least one amino acid substitution is conservative. In some aspects, at least one amino acid substitution is nonconservative.

In some aspects, a CAR spacer of the present disclosure comprises a sequence set forth in TABLES 1, 3, 5, 7, 9A, 9B, 11, 13, 15, or 17, wherein the N-terminal amino acid is the N-terminal amino acid of the naturally occurring hinge, and wherein the sequence of TABLES 1, 3, 5, 7, 9A, 9B, 11, 13, 15, or 17, is extended one, two, three, four, five, six, seven, eight, nine or ten amino acids into the corresponding CH1 region (i.e., the naturally occurring CH1 region). In other words the resulting CAR spacer of the present disclosure comprises one, two, three, four, five, six, seven, eight, nine or ten amino acids from the C-terminus of the corresponding CH1 region (e.g., the CH1 region of an IgD for a CAR spacer derived from a IgD hinge) appended to the N-terminus of the sequence of TABLES 1, 3, 5, 7, 9A, 9B, 11, 13, 15, or 17.

In some aspects, a CAR spacer of the present disclosure comprises a sequence set forth in TABLES 1, 3, 5, 7, 9A, 9B, 11, 13, 15, or 17, wherein the C-terminal amino acid is the C-terminal amino acid of the naturally occurring hinge, and wherein the sequence of TABLES 1, 3, 5, 7, 9A, 9B, 11, 13, 15, or 17, is extended one, two, three, four, five, six, seven, eight, nine or ten amino acids into the corresponding CH2 region (i.e., the naturally occurring CH2 region). In other words the resulting CAR spacer comprises one, two, three, four, five, six, seven, eight, nine or ten amino acids from the N-terminus of the corresponding CH2 region (e.g., the CH2 region of an IgD for a CAR spacer derived from a IgD hinge) appended to the C-terminus of the sequence of TABLES 1, 3, 5, 7, 9A, 9B, 11, 13, 15, or 17.

In some aspects, a CAR spacer of the present disclosure comprises a sequence set forth in TABLES 1, 3, 5, 7, 9A, 9B, 11, 13, 15, or 17, wherein (i) the N-terminal amino acid is the N-terminal amino acid of the naturally occurring hinge and the C-terminal amino acid is the C-terminal amino acid of the naturally occurring hinge, and (ii) the sequence of TABLES 1, 3, 5, 7, 9A, 9B, 11, 13, 15 or 17, is extended one, two, three, four, five, six, seven, eight, nine or ten amino acids into the corresponding CH1 region (i.e., the naturally occurring CH1 region) and extended one, two, three, four, five, six, seven, eight, nine or ten amino acids into the corresponding CH2 region (i.e., the naturally occurring CH2 region). In other words the resulting CAR spacer comprises one, two, three, four, five, six, seven, eight, nine or ten amino acids from the C-terminus of the corresponding CH1 region (e.g., the CH1 region of an IgD for a CAR spacer derived from a IgD hinge) appended to the N-terminus of the sequence of TABLES 1, 3, 5, 7, 9A, 9B, 11, 13, 15, or 17, and one, two, three, four, five, six, seven, eight, nine or ten amino acids from the N-terminus of the corresponding CH2 region (e.g., the CH2 region of an IgD for a CAR spacer derived from a IgD hinge) appended to the C-terminus of the sequence of TABLES 1, 3, 5, 7, 9A, 9B, 11, 13, 15, or 17.

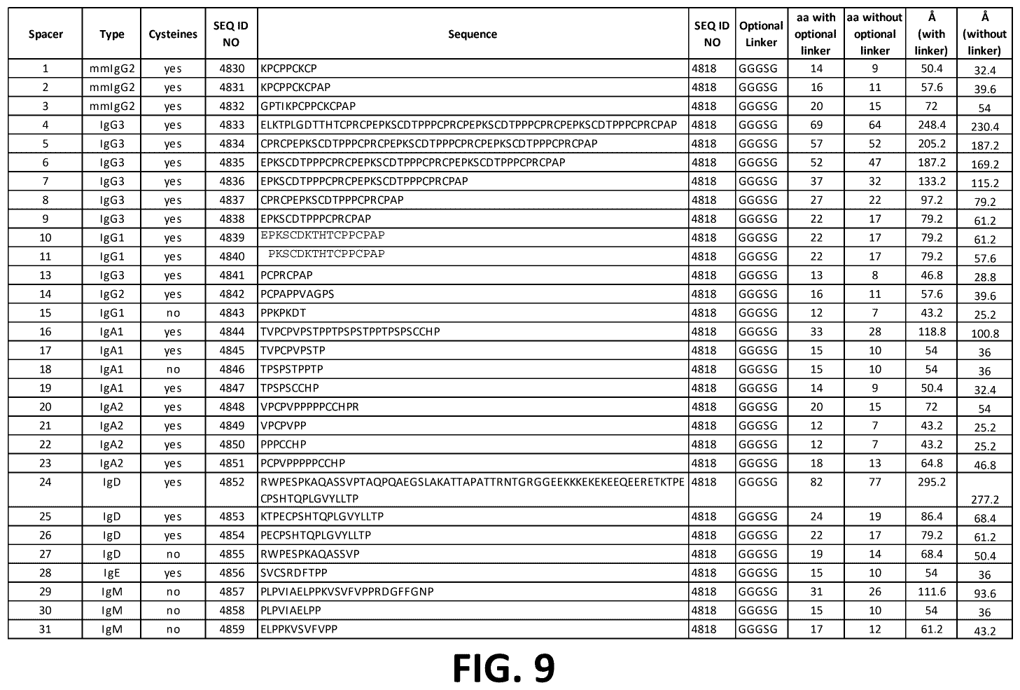

In some specific aspects, a CAR spacer of the present disclosure comprises, consists, or consists essentially of a sequence set forth in FIG. 9. The table includes the sequences of exemplary spacers of the present disclosure, their SEQ ID NOs, length (including an optional flexible linker), Ig hinge source, and also the identifier (“Spacer” column) used for each hinge in the drawings and example section of this specification.

In some specific aspects, a CAR spacer of the present disclosure comprises, consists, or consists essentially of a sequence of an IgAQ1 hinge derived CAR spacer, e.g, Spacer 16 (SEQ ID NO: 4844), Spacer 17 (SEQ ID NO: 4845), Spacer 18 (SEQ ID NO: 4846), or Spacer 19 (SEQ ID NO: 4847), as shown in FIG. 9. In some aspects, the IgAQ1 hinge derived CAR spacer comprises a Gly-Ser linker, e.g., a linker of SEQ ID NO: 4818.

In some specific aspects, a CAR spacer of the present disclosure comprises, consists, or consists essentially of a sequence of an IgA2 hinge derived CAR spacer, e.g, Spacer 20 (SEQ ID NO: 4848), Spacer 21 (SEQ ID NO: 4849), Spacer 22 (SEQ ID NO: 4850), or Spacer 23 (SEQ ID NO:4851) as shown in FIG. 9. In some aspects, the IgA2 hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgD hinge derived CAR spacer, e.g, Spacer 24 (SEQ ID NO: 4852), Spacer 25 (SEQ ID NO: 4853), Spacer 26 (SEQ ID NO: 4854), or Spacer 27 (SEQ ID NO: 4855) as shown in FIG. 9. In some aspects, the IgD hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgE hinge derived CAR spacer, e.g, Spacer 28 (SEQ ID NO:4856) as shown in FIG. 9. In some aspects, the IgE hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgG1 hinge derived CAR spacer, e.g, Spacer 11 (SEQ ID NO: 4840) or Spacer 10 (SEQ ID NO: 4839) as shown in FIG. 9. In some aspects, the IgG1 hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgG2, e.g., a human IgG2 hinge, derived CAR spacer, e.g, Spacer 14 (SEQ ID NO:4842) as shown in FIG. 9. In some aspects, the IgG2 hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgG2 hinge, e.g., a murine IgG2A hinge, derived CAR spacer, e.g, Spacer 1 (SEQ ID NO: 4830), Spacer 2 (SEQ ID NO:4831), Spacer 3 (SEQ ID NO:4832) as shown in FIG. 9. In some aspects, the IgG2 hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgG2 hinge, e.g., a murine IgG2A hinge, derived CAR spacer, e.g, Spacer 1 (SEQ ID NO: 4830), Spacer 2 (SEQ ID NO:4831), Spacer 3 (SEQ ID NO:4832), as shown in FIG. 9, wherein the IgG2 hinge is a hinge region from IgG2A (Uniprot P01865). In some aspects, the CAR spacer comprises a 6-mer, 7-mer, 8-mer, 9-mer, 10-mer, 11-mer, 12-mer or 13-mer amino acid subsequence of a murine IgG2A hinge of SEQ ID NO: 4860 (EPRGPTIKPCPPC). In some aspects, the CAR spacer derived from the murine IgG2A hinge of SEQ ID NO: 4860, further comprises 1, 2, 3, 4, or 5 CH1 amino acids from the murine IgG2A upstream from the hinge sequence (i.e., a VDKKI sequence of SEQ ID NO:4861 or a subsequence thereof), and/or 1, 2, 3, 4, or 5 CH2 amino acids from the murine IgG2A downstream from the hinge sequence (i.e., a KCPAP sequence of SEQ ID NO:4862 or a subsequence thereof). In some aspects, the CAR spacer derived from the murine IgG2A hinge of SEQ ID NO:4860 comprises a KPCPPC (SEQ ID NO:4863) subsequence from the IgG2A hinge and a contiguous KPC subsequence of the CH2 region of murine IgG2 Å, i.e., the CAR spacer has the sequence of Spacer 1. In some aspects, an IgG2 hinge, e.g., a murine IgG2A hinge, derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgG3 hinge derived CAR spacer, e.g, Spacer 4 (SEQ ID NO: 4844), Spacer 5 (SEQ ID NO: 4834), Spacer 6 (SEQ ID NO: 4835), Spacer 7 (SEQ ID NO: 4836), Spacer 8 (SEQ ID NO: 4837), Spacer 9 (SEQ ID NO: 4838) or Spacer 13 (SEQ ID NO: 4841) as shown in FIG. 9., In some aspects, the IgG3 hinge derived CAR spacer comprises a Gly-Ser linker, e.g., a linker of SEQ ID NO: 4818 or 5088.

In some specific aspects, a CAR spacer of the present disclosure comprises, consists, or consists essentially of a sequence of an IgM hinge derived CAR spacer, e.g, Spacer 29 (SEQ ID NO:4857), Spacer 30 (SEQ ID NO:4858) or Spacer 31 (SEQ ID NO:4859) as shown in FIG. 9. In some aspects, the IgM hinge derived CAR spacer comprises a Gly-Ser linker, e.g., a linker of SEQ ID NO: 4818 or 5088.

In some aspects, a CAR spacer of the present disclosure has at least about 70%, at least about 75%, at least about 80%, at least about 85%, at least about 90%, at least about 95%, at least about 96%, at least about 97%, at least about 98%, or at least about 99% sequence identity to a sequence set forth in FIG. 9. In some aspects, a CAR spacer of the present disclosure comprises a sequence identical to any one of the sequences set forth in FIG. 9 except for one, 2, 3, 4, 5, 6, 7, 8, 9, or 10 amino acid substitutions. In some aspects, the amino acid substitutions are conservative amino acid substitutions. In some aspects, the amino acid substitution comprises at least one non-conservative amino acid substitution.

In some aspects, a CAR spacer of the present disclosure comprises a sequence of any of the spacers of SEQ ID NOS: 4830-4859 (Spacer 1 to 31) disclosed in FIG. 9, wherein the spacer sequence further comprises an optional flexible linker (e.g., the linker of SEQ ID NO:4818 or 5088 set forth in FIG. 9 and its legend). Thus, in some aspects, a CAR spacer of the present disclosure comprises a Spacer sequence disclosed in FIG. 9 and an optional C-terminal or N-terminal flexible linker. For example, in some aspects, a CAR spacer of the present disclosure comprises the sequence set forth in SEQ ID NO: 4882, i.e., Spacer 1 plus a C-terminal linker of SEQ ID NO:4818; the sequence set forth in SEQ ID NO:4883, i.e., Spacer 1 plus an N-terminal linker of SEQ ID NO:4818; or the sequence set forth in SEQ ID NO:4884, i.e., Spacer 1 plus both a C-terminal linker and an N-terminal linker of SEQ ID NO:4818. For example, in some aspects, a CAR spacer of the present disclosure comprises the sequence of Spacer 1 plus a C-terminal linker of SEQ ID NO:5088; the sequence of Spacer 1 plus an N-terminal linker of SEQ ID NO:5088; or the sequence of Spacer 1 plus both a C-terminal linker and an N-terminal linker of SEQ ID NO:5088. In some aspects, any optional flexible linkers (e.g., gly/ser rich linker) disclosed herein can be appended to the C-terminus and/or the N-terminus of any spacer sequence disclosed in FIG. 9.

In some aspects, a CAR spacer of the present disclosure comprises a functional fragment of a human immunoglobulin hinge region comprising:
(a) an internal subsequence of a hinge region of an IgAQ1, IgA2, IgD, IgE, IgG1, IgG2 (e.g., human IgG2 or murine IgG2A), IgG3, IgG4, or IgM (e.g., a hinge region from SEQ ID NOS: 4993-5002);
(b) a C-terminal subsequence of a hinge region IgAQ1, IgA2, IgD, IgE, IgG1, IgG2 (e.g., human IgG2 or murine IgG2A), IgG3, IgG4, or IgM (e.g., a hinge region from SEQ ID NOS: 4993-5002);
(c) an N-terminal subsequence of a hinge region IgAQ1, IgA2, IgD, IgE, IgG1, IgG2 (e.g., human IgG2 or murine IgG2A), IgG3, IgG4, or IgM (e.g., a hinge region from SEQ ID NOS: 4993-5002);
(d) a hinge region of an IgAQ1, IgA2, IgD, IgE, IgG1, IgG2 (e.g., human IgG2 or murine IgG2A), IgG3, IgG4, or IgM (e.g., a hinge region from SEQ ID NOS: 4993-5002) extended 1 to 10 amino acids towards the N-terminal CH1 domain and/or C-terminal CH2 domain;
(e) a subsequence of a hinge region of an IgAQ1, IgA2, IgD, IgE, IgG1, IgG2 (e.g., human IgG2 or murine IgG2A), IgG3, IgG4, or IgM (e.g., a hinge region from SEQ ID NOS: 4993-5002) extended 1 to 10 amino acids towards the N-terminal CH1 domain;
(f) a subsequence of a hinge region of an IgAQ1, IgA2, IgD, IgE, IgG1, IgG2 (e.g., human IgG2 or murine IgG2A), IgG3, IgG4, or IgM (e.g., a hinge region from SEQ ID NOS: 4993-5002) extended 1 to 10 amino acids towards the C-terminal CH2 domain;
(g) a sequence comprising 2 or more repeats of (a)-(f);
(h) a combination of (a)-(g) corresponding to the same hinge region;
(h) a combination of (a)-(g) corresponding to different hinge regions; or,
(i) any combination thereof.

In some aspects, the hinge region is a murine hinge region.

In some aspects, ae CAR spacers disclosed herein comprises, consists essentially of, or consists of the sequence set forth in SEQ ID NO: 4831, which is capable of linking an antigen-binding domain that specifically binds to ROR1 with a transmembrane domain.

In some aspects, the present disclosure provides an anti-ROR1 CAR-expressing cell, e.g., CART, e.g., a cell expressing an anti-ROR1 CAR construct or encoded by a ROR1 binding CAR comprising a scFv, CDRs, or VH and VL chains, wherein the anti-ROR1 CAR comprises a CAR spacer of the present disclosure. An anti-ROR1 CAR-expressing cell, e.g., CART can be generated by engineering a ROR1 CAR that comprises a ROR1 binding domain and a CAR spacer of the present disclosure into a cell (e.g., a T cell or NK cell), e.g., for administration to a subject in need thereof.

In some aspects, the ROR1 CAR comprises a sequence set forth in SEQ ID NO: 5070 in which the spacer (subsequence between positions 268 and 281 set forth in SEQ ID NO:4867) has been substituted by a spacer sequence disclosed herein. In some aspects, the spacer sequence replacing the subsequence between positions 268 and 281 in SEQ ID NO:5070 is selected from the group consisting of SEQ ID NOS:4830 to 4859 (Spacer 1 to Spacer 31). In some aspects, the spacer sequence of SEQ ID NO:4830 to 4859 further comprises an optional N-terminal and/or C-terminal linker sequence of SEQ ID NO:4818. In some aspects, the subsequence between position 268 and 281 in in SEQ ID NO:5070 is replaced with Spacer 1 (SEQ ID NO:4830). In some aspects, the ROR1 CAR comprises a CAR sequence disclosed in TABLE 21. In some aspects, the ROR1 CAR-T comprises a CAR sequence disclosed in TABLE 21. In some aspects, the ROR1 CAR comprises a CAR sequence set forth in any of SEQ ID NOS: 5072, 5073, 5074, or 5075. In some aspects, the ROR1 CAR-T comprises a CAR sequence set forth in any of SEQ ID NOS: 5072, 5073, 5074, or 5075.

Non limiting exemplary ROR1-binding domains can be found at TABLE 19, e.g., R12 antibody. Accordingly, in some aspects, the ROR1 CAR comprises a binding moiety derived from antibodies R12, R11, or 2A2 (see TABLE 19). In some aspects, the ROR1 CAR comprises a binding moiety derived from the R12 antibody and a spacer disclosed herein, e.g., Spacer 1 or any of the spacers disclosed in FIG. 9. In some aspects, the ROR1 CAR comprises a binding moiety derived from the R11 antibody and a spacer disclosed herein, e.g., Spacer 1 or any of the spacers disclosed in FIG. 9. In some aspects, the ROR1 CAR comprises a binding moiety derived from the 2A2 antibody and a spacer disclosed herein, e.g., Spacer 1 or any of the spacers disclosed in FIG. 9.

In some aspects, the present disclosure provides an anti-CD19 CAR-expressing cell, e.g., CART, e.g., a cell expressing an anti-CD19 CAR construct or encoded by a CD19 binding CAR comprising a scFv, CDRs, or VH and VL chains, wherein the anti-CD19 CAR comprises a CAR spacer of the present disclosure. An anti-CD19 CAR-expressing cell, e.g., CART can be generated by engineering a CD19 CAR that comprises a CD19 binding domain and a CAR spacer of the present disclosure into a cell (e.g., a T cell or NK cell), e.g., for administration to a subject in need thereof.

In some aspects, the present disclosure provides an anti-CD19 CAR comprising a spacer disclosed herein. In some aspects, the FMC63 CAR comprises a sequence set forth in SEQ ID NO:5076 in which the spacer (subsequence from positions 266 to 277 set forth in SEQ ID NO:4876) has been substituted by a spacer sequence disclosed herein. In some aspects, the spacer sequence replacing the subsequence between positions 266 and 277 in SEQ ID NO:5076 is selected from the group consisting of SEQ ID NOS:4830 to 4859 (Spacer 1 to Spacer 31). In some aspects, the spacer sequence of SEQ ID NO:4830 to 4859 further comprises an optional N-terminal and/or C-terminal linker sequence of SEQ ID NO:4818. In some aspects, the subsequence between position 266 and 277 in SEQ ID NO:5076 is replaced with Spacer 1 (SEQ ID NO:4830). In some aspects, the anti-CD19 CAR comprises a CAR sequence disclosed in TABLE 23. In some aspects, the anti-CD19 CAR-T comprises a CAR sequence disclosed in TABLE 23. In some aspects, the anti-CD19 CAR comprises a CAR sequence set forth in any of SEQ ID NOS: 4918, 4919, 4920, 4921, 4922, 4923, 4924, or 4925.. In some aspects, the anti-CD19 CAR comprises a CAR spacer sequence comprising, consisting, or consisting essentially of a sequence set forth in SEQ ID NOs: 4836, 4837, 4838, 4839, 4840, 4842, 4844, or 4856.

In some aspects, the present disclosure provides an anti-Her2 CAR-expressing cell, e.g., CAR-T, e.g., a cell expressing an anti-Her2 CAR construct or encoded by a Her2 binding CAR comprising a scFv, CDRs, or VH and VL chains, wherein the anti-Her2 CAR comprises a CAR spacer of the present disclosure. An anti-Her2 CAR-expressing cell, e.g., CART can be generated by engineering a Her2 CAR that comprises a Her2 binding domain and a CAR spacer of the present disclosure into a cell (e.g., a T cell or NK cell), e.g., for administration to a subject in need thereof.

In some aspects, the present disclosure provides an anti-Her2 CAR comprising a spacer disclosed herein. In some aspects, the anti-Her2 CAR comprises a CAR sequence disclosed in TABLE 24. In some aspects, the anti-Her2 CAR comprises a sequence set forth in SEQ ID NOS:5005, 5006, 5007 or 5008. In some aspects, the anti-Her2 CAR comprises a sequence set forth in SEQ ID NOS:5005, 5006, 5007 or 5008, in which the scFv linker sequence of SEQ ID NO: 5003 has been replaced with the scFv linker sequence of SEQ ID NO: 5004. In some aspects, the anti-Her2 CAR comprises a CAR spacer sequence comprising, consisting, or consisting essentially of a sequence set forth in SEQ ID NOs: 4835, 4836, 4837, or 4844.

In some aspects, the CAR (e.g., an anti-ROR1 CAR, an anti-CD19 CAR, or an anti-Her2 CAR disclosed herein) comprises an optional leader sequence (e.g., an optional leader sequence described herein), an extracellular antigen-binding domain, a CAR spacer of the present disclosure (e.g., a hinge region derived CAR spacer described herein), a transmembrane domain (e.g., transmembrane domain described herein), and an intracellular stimulatory domain (e.g., intracellular stimulatory domain described herein).

In some aspects, an exemplary CAR construct (e.g., an anti-ROR1 CAR, an anti-CD19 CAR or anti-Her2 CAR disclosed herein) comprises an optional leader sequence (e.g., a leader sequence described herein), an extracellular antigen-binding domain, a CAR spacer of the present disclosure (e.g., a hinge or hinge and constant region derived CAR spacer described herein), a transmembrane domain, an intracellular costimulatory domain (e.g., an intracellular costimulatory domain described herein) and an intracellular stimulatory domain.

In some aspects, the present disclosure encompasses a recombinant nucleic acid construct comprising a nucleic acid molecule encoding a CAR (e.g., an anti-ROR1 CAR, an anti-CD19 CAR, or an anti-Her2 CAR disclosed herein), wherein the nucleic acid molecule comprises the nucleic acid sequence encoding a binding domain (e.g., a ROR1-binding domain in an anti-ROR1 CAR of the present disclosure, or a CD19-binding domain in an anti-CD19 CAR disclosed herein, or a Her2-binding domain of an anti-Her2 CAR disclosed herein), e.g., that is contiguous with and in the same reading frame as a nucleic acid sequence encoding an intracellular signaling domain. An exemplary intracellular signaling domain that can be used in the CAR includes, but is not limited to, one or more intracellular signaling domains of, e.g., CD3-zeta, CD28, 4-1BB, and the like. In some aspects, the CAR (e.g., an anti-ROR1 CAR, an anti-CD19 CAR, or an anti-Her2 CAR disclosed herein) can comprise any combination of CD3-zeta, CD28, 4-1BB, and the like.

In some aspects, the binding domain (e.g., a ROR1-binding domain in an anti-ROR1 CAR of the present disclosure, or a CD19-binding domain in an anti-CD19 CAR disclosed herein, or a Her2-binding domain of an anti-Her2 CAR disclosed herein) is characterized by particular functional features or properties of an antibody or antigen-binding antibody fragment. For example, in some aspects, the portion of a CAR composition of the disclosure that comprises an antigen-binding domain specifically binds a human antigen or a fragment thereof (e.g., a human ROR1-binding domain in an anti-ROR1 CAR of the present disclosure, or a human CD19-binding domain in an anti-CD19 CAR disclosed herein, or a Her2-binding domain of an anti-Her2 CAR disclosed herein). In certain aspects, the scFv is contiguous with and in the same reading frame as a leader sequence.

In some aspects, the binding domain (e.g., a ROR1-binding domain in an anti-ROR1 CAR of the present disclosure, or a CD19-binding domain in an anti-CD19 CAR disclosed herein, or a Her2-binding domain of an anti-Her2 CAR disclosed herein) is a fragment, e.g., a single chain variable fragment (scFv). In some aspects, the binding domain (e.g., a ROR1-binding domain in an anti-ROR1 CAR of the present disclosure, or a CD19-binding domain in an anti-CD19 CAR disclosed herein, or a Her2-binding domain of an anti-Her2 CAR disclosed herein) is a Fv, a Fab, a (Fab′)2, or a bi-functional (e.g. bi-specific) hybrid antibody (e.g., Lanzavecchia et al., Eur. J. Immunol. 17, 105 (1987)). In some aspects, the antibodies and fragments thereof of the disclosure, used to generate a CAR of the present disclosure, binds their target protein (e.g., ROR1, CD19 or Her2) or a fragment thereof with wild-type or enhanced affinity. In some instances, a human scFv used in a CAR of the present disclosure can be derived from a display library.

### Modular Ig Derived Spacers

The present disclosure also provide a polypeptide encoding a “modular CAR spacer,” i.e., a CAR spacer comprising several repetitions of a polypeptide unit. This specific type of CAR spacer of the present disclosure allows tailoring the length of a spacer to optimally position the CAR antigen-binding domain with respect to the epitope of a tumor antigen expressed on a target cell. The modular CAR spacers disclosed herein can be generated by concatenating two or more polypeptides disclosed herein (e.g., hinge region derived CAR spacers, loop region derived CAR spacers, or combinations thereof). In a particular aspect, the modular CAR can be generated by concatenating sequences derived from the IgG3 hinge region and/or polypeptide sequences comprising motifs identified in the IgG3 hinge region.

Accordingly, the present disclosure provides a polynucleotide encoding a CAR comprising (i) an antigen-binding domain that binds to an epitope on a tumor antigen expressed on a target cell; (ii) a transmembrane domain; (iii) an intracellular domain; and (iv) a modular CAR spacer of the present disclosure located between the antigen-binding domain and the transmembrane domain comprising an amino acid sequence derived from a human immunoglobulin hinge region or a functional fragment thereof, wherein the CAR spacer of the present disclosure comprises an amino acid sequence of formula C_{N}—(X₁PRX₂P)ₘ-[L-(X₁PRX₂P)]ₙ-C_{C} wherein (i) the modular CAR spacer is located between the ligand-binding domain and the transmembrane domain of the CAR; (ii) the modular CAR spacer has a length of at least 15 amino acids; (iii) m is an integer selected from 0 or 1; (iv) n is an integer between 1 to 20; (v) L is a linker polypeptide sequence; (vi) CN is an optional N-terminal capping sequence; (vii) Cc is an optional C-terminal capping sequence; and, (viii) X₁ and X₂ are independently selected from cysteine, glycine, alanine, or serine.

In some aspects, the modular CAR spacer comprises two, three, four, five, or six X₁PRX₂P motifs. In some aspects, X₁PRX₂P comprises at least one cysteine. In some aspects X₁PRX₂P is SEQ ID NO: 4749 (CPRCP). In some aspects, L comprises a polypeptide of SEQ ID NO: 4185 or a fragment or variant thereof. In some aspects, when n>1, all L are identical. In some aspects, when n>1, at least one L is different from the other L. In some aspects CN comprises a polypeptide of SEQ ID NO: 4088 or a fragment or variant thereof. In some aspects, Cc comprises a polypeptide of SEQ ID NO: 4453 or a fragment or variant thereof.

In some aspects, the modular CAR spacer comprises the sequence of formula (CPRCP)ₒ(EPKSCDTPPPCPRCP)ₚ, wherein o is an integer which is 0 or 1, and p is an integer which is 1, 2 or 3.

In some aspects, the modular CAR spacer comprises an amino acid sequence selected the group consisting of SEQ ID NOS: 2903; 2983; 3358; 3728; and, 3958.

In some aspects, the modular CAR spacer comprises a Cc capping sequence comprising a subsequence of the polypeptide of SEQ ID NO: 4453 or a fragment or variant thereof. In some aspects, the subsequence of the polypeptide of SEQ ID NO: 4453 is the N-terminal AP. In some aspects, the modular CAR spacer comprises an amino acid sequence selected from the group consisting of SEQ ID NOS: 4750; 4751; 4752; 4753; and, 4754.

In some aspects, the modular CAR spacer comprises a CN capping sequence comprising a subsequence of the polypeptide of SEQ ID NO: 4088 or a fragment or variant thereof. In some aspects, the modular CAR spacer comprises the amino acid sequence of SEQ ID NO: 4811.

In some aspects, the present disclosure provides a polynucleotide encoding a CAR comprising (i) an antigen-binding domain that binds to an epitope on a tumor antigen expressed on a target cell; (ii) a transmembrane domain; (iii) an intracellular domain; and (iv) a modular CAR spacer located between the antigen-binding domain and the transmembrane domain comprising an amino acid sequence derived from a human immunoglobulin hinge region or a functional fragment thereof, wherein the spacer comprises an amino acid sequence of formula (EX₃KX₄X₅X₆X₇DTX₈X₉X₁₀TCPRCP)q (SEQ ID NO: 4812) wherein q is an integer between 1 and 10, and wherein: X₃ is L or P; X₄ is T or S; X₅ is P or C; X₆ is L, or none; X₇ is G, or none; X₅ is T or P; X₉ is H or P; and, X₁₀ is T or P.

The present disclosure also provides a polynucleotide encoding a CAR comprising (i) an antigen-binding domain that binds to an epitope on a tumor antigen expressed on a target cell; (ii) a transmembrane domain; (iii) an intracellular domain; and, (iv) a modular CAR spacer located between the antigen-binding domain and the transmembrane domain comprising an amino acid sequence derived from a human immunoglobulin hinge region or a functional fragment thereof, wherein the modular CAR spacer comprises at least one amino acid sequence A of SEQ ID NO: 4466 and/or at least one amino acid sequence B of SEQ ID NO: 4477, wherein the amino acid sequence of the CAR spacer corresponds, e.g., to the formula AB, ABB, ABBB, ABBBB, A, B, BA, AA, BAB, BAA, AAA, ABA, BBB, AABB, AAAB. In some aspects, the modules (sequence A and sequence B) are concatenated via an amino bond between the C-terminal amino acid in a module and the N-terminal amino acid in the following module.

In some aspects, the modular CAR spacer of the present disclosure corresponds to the formula {[A/B]-(L1)-[A/B]-(L2)]}n wherein A/B is an amino sequence A of SEQ ID NO: 4466 or an amino acid sequence B of SEQ ID NO: 4477; L1 and L2 are optional linkers (e.g., Gly-Ser linkers such as the linker of SEQ ID NO: 4818 or 5088); and, n is an integer between 1 and 100.

Exemplary modular CAR spacers derived from IgG3 comprising combination of sequence A set forth in SEQ ID NO: 4466 and/or sequence B set forth in SEQ ID NO: 4477, are set forth in SEQ ID NOs:4466-4518.

In some aspects, the L1 and/or L2 optional linker is a peptide linker. In some aspects, the L1 and/or L2 optional linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, or at least about 200 amino acids. In some aspects, the L1 and/or L2 optional linker is a glycine/serine (Gly-Ser) linker as described below.

In some aspects, a modular CAR spacer of the present disclosure is about 15 amino acids, about 30 amino acids, about 45 amino acids, about 60 amino acids, about 75 amino acids, about 90 amino acids, about 105 amino acids, about 120 amino acids, about 135 amino acids, about 150 amino acids, about 165 amino acids, about 180 amino acids, about 195 amino acids, about 210 amino acids, about 225 amino acids, about 240 amino acids, about 255 amino acids, about 270 amino acids, about 285 amino acids, about 300 amino acids in length.

In some aspects, a modular CAR spacer of the present disclosure is between about 10 and about 15, between about 15 and about 20, between about 20 and 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 95 and about 100, between about 100 and about 105, between about 105 and about 110, between about 110 and about 115, between about 115 and about 120, between about 120 and about 125, between about 125 and about 130, between about 130 and about 135, between about 135 and about 140, between about 140 and about 145, between about 145 and about 150, between about 150 and about 155, between about 155 and about 160, between about 160 and about 165, between about 165 and about 170, between about 170 and about 175, between about 175 and 180, between about 180 and about 185, between about 185 and about 190, between about 190 and about 196, between about 195 and about 200, between about 200 and about 205, between about 205 and about 210, between about 210 and about 215, between about 215 and about 220, between about 220 and about 225, between about 225 and about 230, between about 230 and about 235, between about 235 and about 240, between about 240 and about 245, between about 245 and about 250, between about 250 and about 255, between about 255 and about 260, between about 260 and about 265, between about 265 and about 270, between about 270 and about 275, between about 275 and about 280, between about 280 and about 285, between about 285 and about 290, between about 290 and about 295, or between about 295 and about 300 amino acids in length.

### Linkers

In some aspects, any CAR spacer of the present disclosure (e.g., any CAR spacer disclosed in TABLES 1, 3, 5, 7, 9A, 9B, 11, 13, 15, or 17, optionally comprising N- and C-terminal sequences disclosed in TABLES 2, 4, 6, 8, 10A, 10B, 12, 14, 16, or 18, or any modular CAR spacer disclosed herein) can comprise an optional N-terminal linker and/or an optional C-terminal linker. Flexible linker sequences known the art can be used as optional linkers. In some aspects, the linker is a glycine/serine linker. As used herein, term “glycine/serine” is used interchangeably with the term “Gly/Ser” and refers to a linker sequence comprising repeats of the amino acids glycine (G, Gly) and/or serine (S, Ser) as exemplified below.

In some aspects, the optional linker is a glycine/serine linker according to the formula [(Gly)n-Ser]m (SEQ ID NO: 4813) where n is any integer from 1 to 100 and m is any integer from 1 to 100. In other aspects, the glycine/serine linker is according to the formula [(Gly)x-Sery]z (SEQ ID NO: 4814) wherein x in an integer from 1 to 4, y is 0 or 1, and z is an integers from 1 to 50. In some aspects, the optional linker comprises the sequence Gn (SEQ ID NO: 4815), where n can be an integer from 1 to 100. In some aspects, the optional linker can comprise the sequence (GlyAla)n (SEQ ID NO: 4816), wherein n is an integer between 1 and 100.

In some aspects, the sequence of the optional linker has the sequence set forth in SEQ ID NO: 4817. In some aspects, the sequence of the optional linker is GGGSG (SEQ ID NO: 4818). In some aspects, the sequence of the optional linker is GGGGSG (SEQ ID NO: 5088).

In other aspects, the optional linker comprises the sequence set forth in SEQ ID NO: 4819. In other aspects, the optional linker comprises the sequence set forth in SEQ ID NO:4820. In other aspects, the optional linker can comprise the sequence set forth in SEQ ID NO: 4821. In other aspects, the optional linker can comprise the sequence set forth in SEQ ID NO: 4822. In these instances, n can be an integer from 1 to 100. In other instances, n can be an integer from one to 20, i.e., 1, 2, 3, 4, 5, 6, 7, 8, 9, 10, 11, 12, 13, 14, 15, 16, 17, 18, 19, or 20. In some aspects n is an integer from 1 to 100.

Examples of the optional linker include, but are not limited to, e.g., the sequences set forth in SEQ ID NOS: 4823, 4824, 4825, 4826, 4827, 4828, or 4829.

In some aspects, the optional linker comprises the sequence PGG. In some aspects, the optional linker comprises additional amino acids in addition to Glycine and Serine. In some aspects, the optional linker comprises 1, 2, 3, 4, or 5 non-gly/non-ser amino acids. In some aspects, the Gly/Ser-linker comprises at least about 60%, at least about 65%, at least about 70%, at least about 80%, at least about 85%, at least about 90%, or at least 95% glycine or serine amino acids.

In some specific aspects, the optional linker is between 1 and 10 amino acids in length. In some aspects, the optional linker as between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, or between about 90 and about 100 amino acids in length.

### Rational Design of Ig Derived CAR Spacers

In some aspects, the present disclosure provides Ig derived CAR spacers selected to position the antigen-binding portion of the CAR at an optimal distanced or in an optimal position to interact with the an epitope located on the target protein. As represented schematically in FIG. 7, it is possible to estimate the optimal length of a CAR spacer, e.g., by having an estimate or measurement of the distance between the epitope at the surface of the target cell, and an estimate or measurement of the signaling synapse, i.e., the distance between the surface of the cell expressing the CAR (e.g., a CART cell) and the surface of the target cell.

In some aspects, the calculation can assume that the width of the signaling synapse is approximately 150 angstroms. In some aspects, the distance between the epitope and the surface of the target cell membrane can be estimated, e.g., using x-ray crystallography, NMR, or cryo-EM structure. In other aspects, the distance between the epitope and the surface of the target cell membrane can be estimated, e.g., using spectroscopic techniques, for example, using Fluorescence Resonance Energy Transfer (FRET).

In some aspect, the estimates can be used to select a subset of candidate CAR spacer sequences for screening. These sequences can be subsequently screened as described in the Examples section of the present application in order to determine, e.g, which spacer (and, therefore, spacer lengths) result in increased cytokine secretion or target cell lysis.

In some aspects, the present disclosure provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer of the present disclosure located between the antigen-binding domain and the transmembrane domain comprising an amino acid sequence derived from a human immunoglobulin hinge region or a functional fragment thereof, wherein
(a) the CAR spacer of the present disclosure is between about 150 amino acids and about 125 amino acids in length; and, the distance between the epitope and the surface of the target cell membrane is less than about 10 Å;
(b) the CAR spacer of the present disclosure is between about 125 amino acids and about 100 amino acids in length; and, the distance between the epitope and the surface of the target cell membrane is less than about 10 Å;
(c) the CAR spacer of the present disclosure is between about 100 amino acids and about 75 amino acids in length; and, the distance between the epitope and the surface of the target cell membrane is less than about 10 Å;
(d) the CAR spacer of the present disclosure is between about 75 amino acids and about 36 amino acids in length; and, the distance between the epitope and the surface of the target cell membrane is less than about 15 Å;
(e) the CAR spacer of the present disclosure is between about 35 amino acids and about 21 amino acids in length; and, the distance between the epitope and the surface of the target cell membrane is between about 15 Å and about 25 Å;
(f) the CAR spacer of the present disclosure is between about 20 amino acids and about 16 amino acids in length; and, the distance between the epitope and the surface of the target cell membrane is between about 25 Å and about 35 Å;
(g) the CAR spacer of the present disclosure is between about 15 amino acids and about 11 amino acids in length; and, the distance between the epitope and the surface of the target cell membrane is between about 35 Å and about 45 Å; or,
(h) the CAR spacer of the present disclosure is between about 10 amino acids and about 5 amino acids in length; and, the distance between the epitope and the surface of the target cell membrane is more than about 45 Å.

In some aspects, the present disclosure provides a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer of the present disclosure located between the antigen-binding domain and the transmembrane domain comprising an amino acid sequence derived from a human immunoglobulin hinge region or a functional fragment thereof, wherein
(a) the CAR spacer of the present disclosure is between about 600 Å and about 500 Å in length; and, the distance between the epitope and the surface of the target cell membrane is less than about 10 Å;
(b) the CAR spacer of the present disclosure is between about 500 Å and about 400 Å in length; and, the distance between the epitope and the surface of the target cell membrane is less than about 10 Å;
(c) the CAR spacer of the present disclosure is between about 400 Å and about 300 Å in length; and, the distance between the epitope and the surface of the target cell membrane is less than about 10 Å;
(d) the CAR spacer of the present disclosure is between about 300 Å and about 150 Å in length; and, the distance between the epitope and the surface of the target cell membrane is less than about 15 Å;
(e) the CAR spacer of the present disclosure is between about 150 Å and about 80 Å in length; and, the distance between the epitope and the surface of the target cell membrane is between about 15 Å and about 25 Å;
(f) the CAR spacer of the present disclosure is between about 80 Å and about 60 Å in length; and, the distance between the epitope and the surface of the target cell membrane is between about 25 Å and about 35 Å;
(g) the CAR spacer of the present disclosure is between about 60 Å and about 40 Å in length; and, the distance between the epitope and the surface of the target cell membrane is between about 35 Å and about 45 Å; or,
(h) the CAR spacer of the present disclosure is between about 40 Å and about 20 Å in length; and, the distance between the epitope and the surface of the target cell membrane is more than about 45 Å.

In some aspects, the distance between the epitope and the surface of the target cell membrane is less than about 10 Å and the spacer is 125, 126, 127, 128, 129, 130, 131, 132, 133, 134, 135, 136, 137, 138, 139, 140, 141, 142, 143, 144, 145, 146, 147, 148, 149 or 150 amino acids in length. In some aspects, the distance between the epitope and the surface of the target cell membrane is less than about 10 Å and the spacer is about 500, about 510, about 520, about 530, about 540, about 550, about 560, about 570, about 580, about 590, or about 600 Å in length.

In some aspects, the distance between the epitope and the surface of the target cell membrane is less than about 10 Å and the spacer is 100, 101, 102, 103, 104, 105, 106, 107, 108, 109, 110, 111, 112, 113, 114, 115, 116, 117, 118, 119, 120, 121, 122, 123, 124 or 125 amino acids in length. In some aspects, the distance between the epitope and the surface of the target cell membrane is less than about 10 Å and the spacer is about 400, about 410, about 420, about 430, about 440, about 450, about 460, about 470, about 480, about 490, or about 500 Å in length.

In some aspects, the distance between the epitope and the surface of the target cell membrane is less than about 10 Å and the spacer is 75, 76, 77, 78, 79, 80, 81, 82, 83, 84, 85, 86, 87, 88, 89, 90, 91, 92, 93, 94, 95, 96, 97, 98, 99 or 100 amino acids in length. In some aspects, the distance between the epitope and the surface of the target cell membrane is less than about 10 Å and the spacer is about 300, about 310, about 320, about 330, about 340, about 350, about 360, about 370, about 380, about 390, or about 400 Å in length.

In some aspects, the distance between the epitope and the surface of the target cell membrane is less than about 15 Å and the spacer is 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 amino acids in length. In some aspects, the distance between the epitope and the surface of the target cell membrane is less than about 15 Å and the spacer is about 150, about 160, about 170, about 180, about 190, about 200, about 210, about 220, about 230, about 240, about 250, about 260, about 270, about 280, about 290 or about 300 Å in length.

In some aspect, the distance between the epitope and the surface of the target cell membrane is between about 15 Å and about 25 Å and the spacer is 21, 22, 23, 24, 25, 26, 27, 28, 29, 30, 31, 32, 33, 34 or 35 amino acids in length. In some aspect, the distance between the epitope and the surface of the target cell membrane is between about 15 Å and about 25 Å and the spacer is about 80, about 90, about 100, about 110, about 120, about 130, about 140 or about 150 Å in length.

In some aspects, the distance between the epitope and the surface of the target cell membrane is between about 25 Å and about 35 Å and the spacer is 16, 17, 18, 19 or 20 amino acids in length. In some aspects, the distance between the epitope and the surface of the target cell membrane is between about 25 Å and about 35 Å and the spacer is about 60, about 65, about 70, about 75 or about 80 Å in length.

In some aspects, the distance between the epitope and the surface of the target cell membrane is between about 35 Å and about 45 Å and the spacer is 11, 12, 13, 14, or 15 amino acids in length. In some aspects, the distance between the epitope and the surface of the target cell membrane is between about 35 Å and about 45 Å and the spacer is about 40, about 45, about 50, about 55 or about 60 Å in length.

In some aspects, the distance between the epitope and the surface of the target cell membrane is more than about 45 Å and the spacer is 5, 6, 7, 8, 9 or 10 amino acids in length. In some aspects, the distance between the epitope and the surface of the target cell membrane is more than about 45 Å and the spacer is about 20, about 25, about 30, about 35, or about 40 Å in length.

Also provided is a polynucleotide encoding a CAR comprising (i) an antigen-binding domain that binds to an epitope on a tumor antigen expressed on a target cell; (ii) a transmembrane domain; (iii) an intracellular domain; and, (iv) a CAR spacer of the present disclosure located between the antigen-binding domain and the transmembrane domain comprising an amino acid sequence derived from a human immunoglobulin hinge region or a functional fragment thereof, wherein
(a) the CAR spacer of the present disclosure is between about 450 Å and about 375 Å in length; and, the distance between the epitope and the surface of the target cell membrane is less than about 10 Å;
(b) the CAR spacer of the present disclosure is between about 375 Å and about 300 Å in length; and, the distance between the epitope and the surface of the target cell membrane is less than about 10 Å;
(c) the CAR spacer of the present disclosure is between about 300 Å and about 225 Å in length; and, the distance between the epitope and the surface of the target cell membrane is less than about 10 Å;
(d) the CAR spacer of the present disclosure is between about 225 Å and about 100 Å in length; and, the distance between the epitope and the surface of the target cell membrane is less than about 15 Å;
(e) the CAR spacer of the present disclosure is between about 100 Å and about 60 Å in length; and, the distance between the epitope and the surface of the target cell membrane is between about 15 Å and about 25 Å;
(f) the CAR spacer of the present disclosure is between about 60 Å and about 45 Å in length; and, the distance between the epitope and the surface of the target cell membrane is between about 25 Å and about 35 Å;
(g) the CAR spacer of the present disclosure is between about 45 Å and about 30 Å in length; and, the distance between the epitope and the surface of the target cell membrane is between about 35 Å and about 45 Å; or,
(h) the CAR spacer of the present disclosure is between about 30 Å and about 15 Å in length; and, the distance between the epitope and the surface of the target cell membrane is more than about 45 Å.

In some aspects, the distance between the epitope and the surface of the target cell membrane is less than about 10 Å and the spacer is about 450 Å, about 440 Å, about 430 Å, about 420 Å, about 410 Å, about 400 Å, about 390 Å, about 380 Å or about 375 Å in length. In some aspects, the distance between the epitope and the surface of the target cell membrane is less than about 10 Å and the spacer is about 375 Å, about 370 Å, about 360 Å, about 350 Å, about 340 Å, about 330 Å, about 320 Å, about 310 Å, or about 300 Å in length. In some aspects, the distance between the epitope and the surface of the target cell membrane is less than about 10 Å and the spacer is about 300 Å, about 290 Å, about 280 Å, about 270 Å, about 260 Å, about 250 Å, about 240 Å, about 230 Å, or about 225 Å in length. In some aspects, the distance between the epitope and the surface of the target cell membrane is less than about 15 Å the spacer is about 225 Å, about 220 Å, about 210 Å, about 200 Å, about 190 Å, about 180 Å, about 170 Å, about 160 Å, about 150 Å, about 140 Å, about 130 Å, about 120 Å, about 110 Å, or about 100 Å in length. In some aspects, the distance between the epitope and the surface of the target cell membrane is between about 15 Å and about 25 Å and the spacer is about 100 Å, about 95 Å, about 90 Å, about 85 Å, about 80 Å, about 75 Å, about 70 Å, about 65 Å, or about 60 Å in length. In some aspects, the distance between the epitope and the surface of the target cell membrane is between about 25 Å and about 35 Å and the spacer is about 60 Å, about 55 Å, about 50 Å, and about 45 Å in length. In some aspects, the distance between the epitope and the surface of the target cell membrane is between about 35 Å and about 45 Å and the spacer is about 45 Å, about 40 Å, about 35 Å, or about 30 Å in length. In some aspects, the distance between the epitope and the surface of the target cell membrane is more than about 45 Å and the spacer is about 30 Å, about 25 Å, about 20 Å, or about 15 Å in length.

### Antigen-Binding Domains

In some aspects, the polypeptide encoding a CAR of the present disclosure, i.e., a CAR comprising a CAR spacer of the present disclosure (i.e., a hinge region derived CAR spacer, a loop region derived CAR spacer, or a combination thereof), comprises an antigen-binding domain comprising an antibody or an antigen-binding fragment thereof (e.g., an ScFv) that specifically binds to an epitope on a tumor antigen, e.g., a protein kinase such as a tyrosine protein kinase.

In some aspects, the tumor antigen is selected from the group consisting of ROR1, HER2, AFP, CD19, TRAC, TCRP, BCMA, CLL-1, CS1, CD38, CD19, TSHR, CD123, CD22, CD30, CD70, CD171, CD33, EGFRvIII, GD2, GD3, Tn Ag, PSMA, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, and any combinations thereof.

In some aspects, the antigen-binding domain of a CAR of the present disclosure is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, or a nanobody. In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of, e.g., at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another aspect, the linker sequence comprises sets of glycine and serine repeats such as (Gly₄Ser)n, where n is a positive integer equal to or greater than 1 (SEQ ID NO:81). In some aspects, the linker can be (Gly₄Ser)₄ (SEQ ID NO:82) or (Gly₄Ser)₃ (SEQ ID NO:83), or any gly-ser rich linker disclosed above.

In some aspects, the linker can be GSTSGSGKPGSGEGSTKG (SEQ ID NO:4885), e.g., in an anti-CD19 scFv such as a FMC63-based scFv. In other aspects, the linker can be GSTSGSGKPGSGEGS (SEQ ID NO:4886), e.g., in an anti-Her2 based.

Variation in the linker length may retain or enhance activity, giving rise to superior efficacy in activity studies.

In some aspects, the amino acid sequence of the antigen-binding domain or other portions or the entire CAR can be modified, e.g., an amino acid sequence described herein can be modified, e.g., by a conservative substitution. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In some specific aspects, the tumor antigen is the tyrosine-protein kinase transmembrane receptor “ROR1,” also known as neurotrophic tyrosinase kinase, receptor-related 1 (NTRKR1). The human amino acid and nucleic acid sequences can be found in a public database, such as GenBank, UniProt and Swiss-Prot. For example, the amino acid sequences of isoforms 1 and 2 precursors of human ROR1 can be found at Accession Nos. NP_005003.2 and NP_001077061.1, respectively, and the mRNA sequences encoding them can be found at Accession Nos. NM_005012.3 and NM_001083592.1, respectively. As used herein, “ROR1” includes proteins comprising mutations, e.g., point mutations, fragments, insertions, deletions and splice variants of full length wild-type ROR1. In some aspects the antigen-binding portion of the CAR recognizes and binds an antigen within the extracellular domain of the ROR1 protein. In some aspects, the ROR1 protein is expressed on a cancer cell.

ROR1 is a member of the receptor tyrosine kinase-like orphan receptor (ROR) family. In humans ROR1 is encoded by the ROR1 gene. The protein encoded by this gene is a receptor tyrosine kinase that modulates growth in the central nervous system and has a role in the metastasis of cancer cells. ROR1 is considered a pseudokinase that lacks significant catalytic activity and interacts with the non-canonical Wnt signaling pathway. Increase expression of ROR1 is associate, e.g., with B-cell chronic lymphocytic leukemia. ROR1 is highly expressed in circulating tumor cells and promotes invasion of pancreatic cancer cells (Xu et al., 2018, Mol. Med. Rep. 18:5087-5094). ROR1 also appears to promote tumor progression in endometrial cancer, similar to its role in ovarian cancer (Henry et al, 2018, Gynecol. Oncol. 148:576-584). ROR1 is expressed in epithelial tumors and is homogenously expressed on a subset of ovarian cancer, triple-negative breast cancer, and lung cancer (Balakrishnan et al., 2017, Clin, Cancer Res. 23:3061-3071). ROR1 expression has also been positively associated with lymph-node metastasis in colorectal cancer patients (Zhou et al., 2017, Oncotarget 8:32864-32872).

In some aspects, anti-ROR1 inhibitors, e.g., CARs of present disclosure or cells expressing those CARs, can be used to treat B-cell malignancies (e.g., leukemias, such as CLL, and B-cell lymphomas, such as mantle cell lymphoma; ALL; small lymphocytic lymphoma; marginal cell B-Cell lymphoma; and Burkett's Lymphoma) or epithelial cancers (e.g., breast cancer, renal cell carcinoma, lung cancer, colorectal cancers, ovarian cancer, and melanoma).

An exemplary anti-ROR1 CAR is described in Hudecek, et al. Clin. Cancer Res. 19.12(2013):3153-64, incorporated herein by reference. In some aspects, a CAR of the present disclosure comprises the anti-ROR1 CART described in Hudecek et al. (for example, generated as described in Hudecek et al. at page 3155, first full paragraph, incorporated herein by reference), wherein the spacer disclosed in Hudecek has been replaced by a CAR spacer of the present disclosure. In other aspects, an anti-ROR1 CAR of the present disclosure includes an antibody or fragment thereof comprising the VH and/or VL sequences of the 2A2, R11, and R12 anti-ROR1 monoclonal antibodies described in Hudecek et al. at paragraph bridging pages 3154-55; Baskar et al. MAbs 4(2012):349-61; and Yang et al. PLoS ONE 6(2011):e21018, incorporated herein by reference.

In some aspects, an antigen-binding domain of the present disclosure is capable of cross-competing with an anti-ROR1 antibody, e.g., R11, R12, or 2A2 antibodies. The R11, R12, and 2A2 antibody sequences are shown in TABLE 19. In some aspects, the antigen-binding domain useful for the present disclosure binds to the same epitope of the R11 antibody, the R12 antibody, or the 2A2 antibody.

TABLE 19
R11, R12 and 2A2 antibody CDRs

| | | |
|---|---|---|
| R11 VH | R12 VH | 2A2 VH |
| (SEQ ID NO: 4887) | (SEQ ID NO: 4895) | (SEQ ID NO: 4903) |
| R11 VH CDR1 | R12 VH CDR1 | 2A2 VH CDR1 |
| (SEQ ID NO: 4888) | (SEQ ID NO: 4896) | (SEQ ID NO: 4904) |
| R11 VH CDR2 | R12 VH CDR2 | 2A2 VH CDR2 |
| (SEQ ID NO: 4889) | (SEQ ID NO: 4897) | (SEQ ID NO: 4905) |
| R11 VH CDR3 | R12 VH CDR3 | 2A2 VH CDR3 |
| (SEQ ID NO: 4890) | (SEQ ID NO: 4898) | (SEQ ID NO: 4906) |
| R11 VL | R12 VL | 2A2 VL |
| (SEQ ID NO: 4891) | (SEQ ID NO: 4899) | (SEQ ID NO: 4907) |
| R11 VL CDR1 | R12 VL CDR1 | 2A2 VL CDR1 |
| (SEQ ID NO: 4892) | (SEQ ID NO: 4900) | (SEQ ID NO: 4908) |
| R11 VL CDR2 | R12 VL CDR2 | 2A2 VL CDR2 |
| (SEQ ID NO: 4893) | (SEQ ID NO: 4901) | (SEQ ID NO: 4909) |
| R11 VL CDR3 | R12 VL CDR3 | 2A2 VL CDR3 |
| (SEQ ID NO: 4894) | (SEQ ID NO: 4902) | (SEQ ID NO: 4910) |

In some aspects, the antigen-binding domain of the present disclosure comprises VH CDR3 of the R11 antibody. In some aspects, the antigen-binding domain of the present disclosure comprises VH CDR1, VH CDR2 and VH CDR3 of the R11 antibody. In some aspects, the antigen-binding domain of the present disclosure comprises VH CDR1, VH CDR2, VH CDR3, VL CDR1, VL CDR2, and VL CDR3 of the R11 antibody. In some aspects, the antigen-binding domain of the present disclosure, e.g., R11 scFv, comprises the VH and the VL of the R11 antibody. In some aspects, the R11 scFv is linked to a transmembrane domain by an IgG2 linker, e.g., Spacer 1 (SEQ ID NO: 4830), and optionally a linker of SEQ ID NO: 4818 or 5088. In some aspects, the R11 scFv is linked to a transmembrane domain by an IgG1 linker, e.g., Spacer 11 (SEQ ID NO: 4840), and optionally a linker of SEQ ID NO: 4818 or 5088. In some aspects, the R11 scFv is linked to a transmembrane domain by an IgG3 linker, e.g., Spacer 13 (SEQ ID NO: 4841), and optionally a linker of SEQ ID NO: 4818. In some aspects, the R11 scFv is linked to a transmembrane domain by an IgG2 linker, e.g., Spacer 14 (SEQ ID NO: 4842), and optionally a linker of SEQ ID NO: 4818 or 5088.

In some aspects, the R11 scFv is linked to a transmembrane domain by an IgG3 linker, e.g., Spacer 4 (SEQ ID NO: 4833), and optionally a linker of SEQ ID NO: 4818 or 5088. In some aspects, the R11 scFv is linked to a transmembrane domain by an IgG3 linker, e.g., Spacer 5 (SEQ ID NO: 4834), and optionally a linker of SEQ ID NO: 4818 or 5088. In some aspects, the R11 scFv is linked to a transmembrane domain by an IgG3 linker, e.g., Spacer 6 (SEQ ID NO: 4835), and optionally a linker of SEQ ID NO: 4818 or 5088.

In some aspects, the antigen-binding domain of the present disclosure comprises VH CDR3 of the R12 antibody. In some aspects, the antigen-binding domain of the present disclosure comprises VH CDR1, VH CDR2 and VH CDR3 of the R12 antibody. In some aspects, the antigen-binding domain of the present disclosure comprises VH CDR1, VH CDR2, VH CDR3, VL CDR1, VL CDR2, and VL CDR3 of the R12 antibody. In some aspects, the antigen-binding domain of the present disclosure, e.g., R12 scFv, comprises the VH and the VL of the R12 antibody. In some aspects, the R12 scFv is linked to a transmembrane domain by an IgG2 linker, e.g., Spacer 1 (SEQ ID NO: 4830), and optionally a linker of SEQ ID NO: 4818 or 5088. In some aspects, the R12 scFv is linked to a transmembrane domain by an IgG1 linker, e.g., Spacer 11 (SEQ ID NO: 4840), and optionally a linker of SEQ ID NO: 4818 or 5088. In some aspects, the R12 scFv is linked to a transmembrane domain by an IgG3 linker, e.g., Spacer 13 (SEQ ID NO: 4841), and optionally a linker of SEQ ID NO: 4818 or 5088. In some aspects, the R12 scFv is linked to a transmembrane domain by an IgG2 linker, e.g., Spacer 14 (SEQ ID NO: 4842), and optionally a linker of SEQ ID NO: 4818 or 5088.

In some aspects, the antigen-binding domain of the present disclosure comprises VH CDR3 of the 2A2 antibody. In some aspects, the antigen-binding domain of the present disclosure comprises VH CDR1, VH CDR2 and VH CDR3 of the 2A2 antibody. In some aspects, the antigen-binding domain of the present disclosure comprises VH CDR1, VH CDR2, VH CDR3, VL CDR1, VL CDR2, and VL CDR3 of the 2A2 antibody. In some aspects, the antigen-binding domain of the present disclosure, e.g., 2A2 scFv, comprises the VH and the VL of the 2A2 antibody. In some aspects, the 2A2 scFv is linked to a transmembrane domain by an IgG2 linker, e.g., Spacer 1 (SEQ ID NO: 4830), and optionally a linker of SEQ ID NO: 4818 or 5088. In some aspects, the 2A2 scFv is linked to a transmembrane domain by an IgG1 linker, e.g., Spacer 11 (SEQ ID NO: 4840), and optionally a linker of SEQ ID NO: 4818 or 5088. In some aspects, the 2A2 scFv is linked to a transmembrane domain by an IgG3 linker, e.g., Spacer 13 (SEQ ID NO: 4841), and optionally a linker of SEQ ID NO: 4818. In some aspects, the 2A2 scFv is linked to a transmembrane domain by an IgG2 linker, e.g., Spacer 14 (SEQ ID NO: 4842), and optionally a linker of SEQ ID NO: 4818 or 5088.

In some aspects, the 2A2 scFv is linked to a transmembrane domain by an IgG3 linker, e.g., Spacer 9 (SEQ ID NO: 4838), and optionally a linker of SEQ ID NO: 4818 or 5088. In some aspects, the 2A2 scFv is linked to a transmembrane domain by an IgG3 linker, e.g., Spacer 10 (SEQ ID NO: 4839), and optionally a linker of SEQ ID NO: 4818 or 5088.

In other aspects, a CAR of the present disclosure targeting ROR1 includes an antibody or fragment thereof (e.g., single chain variable fragment (scFv)) that targets ROR1, including those described in U.S. Patent Nos. U.S. Pat. No. 9,316,646B2, issued Sep. 12, 2017, or U.S. Pat. No. 9,758,586B2, issued Apr. 19, 2016, incorporated herein by reference.

In some aspects, a CAR of the present disclosure comprises an antigen-binding domain, a transmembrane domain, and an intracellular domain, wherein the antigen-binding domain and the transmembrane domain are linked by a CAR spacer of the present disclosure, wherein the spacer comprises the sequence set forth in SEQ ID NO: 4830, and optionally a linker of SEQ ID NO: 4818 or 5088, and wherein the antigen-binding domain comprises VH CDR1, VH CDR2, VH CDR3, VL CDR1, VL CDR2, and VL CDR3 of the R11 antibody, e.g., VH and VL of the R11 antibody. In some aspects, the spacer comprises the sequence set forth in SEQ ID NO: 4840, SEQ ID NO: 4841, or SEQ ID NO: 4842, and optionally a linker of SEQ ID NO: 4818 or 5088. In some aspects, the combined length of the optional linker of SEQ ID NO:4818 or 5088 and the spacer is between about 180 Å and about 250 Å. In some aspects, the combined length of the optional linker of SEQ ID NO:4818 or 5088 and the spacer is about 200 Å. In some aspects, the combined length of the optional linker of SEQ ID NO:4818 or 5088 and the spacer is about 180 Å, about 190 Å, about 200 Å, about 210 Å, about 210 Å, about 230 Å, about 240 Å, or about 250 Å.

In some aspects, a CAR of the present disclosure comprises an antigen-binding domain, a transmembrane domain, and an intracellular domain, wherein the antigen-binding domain and the transmembrane domain are linked by a CAR spacer of the present disclosure, wherein the spacer comprises the sequence set forth in SEQ ID NO: 4830, and optionally a linker of SEQ ID NO: 4818 or 5088, and wherein the antigen-binding domain comprises VH CDR1, VH CDR2, VH CDR3, VL CDR1, VL CDR2, and VL CDR3 of the R12 antibody, e.g., VH and VL of the R12 antibody. In some aspects, the spacer comprises a sequence set forth in SEQ ID NO: 4840, SEQ ID NO: 4841, or SEQ ID NO: 4842, and optionally a linker of SEQ ID NO: 4818 or 5088. In some aspects, the combined length of the optional linker of SEQ ID NO:4818 or 5088 and the spacer is between about 35 Å and about 55 Å. In some aspects, the combined length of the optional linker of SEQ ID NO:4818 or 5088 and the spacer is about 45 Å. In some aspects, the combined length of the optional linker of SEQ ID NO:4818 or 5088 and the spacer is about 35 Å, about 40 Å, about 45 Å, about 50 Å, or about 55 Å.

In some aspects, a CAR of the present disclosure comprises an antigen-binding domain, a transmembrane domain, and an intracellular domain, wherein the antigen-binding domain and the transmembrane domain are linked by a CAR spacer of the present disclosure, wherein the spacer comprises the sequence set forth in SEQ ID NO: 4830, and optionally a linker of SEQ ID NO: 4818 or 5088, and wherein the antigen-binding domain comprises VH CDR1, VH CDR2, VH CDR3, VL CDR1, VL CDR2, and VL CDR3 of the 2A2 antibody, e.g., VH and VL of the 2A2 antibody. In some aspects, the spacer comprises the sequence set forth in SEQ ID NO: 4840, SEQ ID NO: 4841, or SEQ ID NO: 4842, and optionally a linker of SEQ ID NO: 4818 or 5088. In some aspects, the combined length of the optional linker of SEQ ID NO:4818 or 5088 and the spacer is between about 35 Å and about 55 Å. In some aspects, the combined length of the optional linker of SEQ ID NO:4818 or 5088 and the spacer is about 45 Å. In some aspects, the combined length of the optional linker of SEQ ID NO:4818 or 5088 and the spacer is about 35 Å, about 40 Å, about 45 Å, about 50 Å, or about 55 Å.

In some aspects, anti-ROR1 antigen-binding antibody fragments (e.g., scFvs) are conjugated or fused to a biologically active molecule, e.g., to form a CAR of the present disclosure (i.e., a CAR comprising a CAR spacer of the present disclosure, i.e., a hinge derived CAR spacer, a loop derived CAR spacer, or a combination thereof) that directs immune cells, e.g., T cells to respond to ROR1-expressing cells.

In some aspects, a CAR of the present disclosure (i.e., a CAR comprising a CAR spacer of the present disclosure, i.e., a hinge derived CAR spacer, a loop derived CAR spacer, or a combination thereof) inhibiting ROR1 includes an anti-ROR1 monoclonal antibody called UC-961 (Cirmtuzumab) o an antigen-binding portion thereof. See, e.g., Clinical Trial Identifier No. NCT02222688. Cirmtuzumab can be used to treat cancers, such as chronic lymphocytic leukemia (CLL), ovarian cancer, and melanoma. See, e.g., Hojjat-Farsangi et al. PLoS One. 8(4): e61167; and NCT02222688.

In some aspects, a CAR of the present disclosure (i.e., a CAR comprising a CAR spacer of the present disclosure, i.e., a hinge derived CAR spacer, a loop derived CAR spacer, or a combination thereof) inhibiting CD19 includes an anti-CD19 antibody or antigen-binding portion thereof.

In some aspects, a CAR of the present disclosure (i.e., a CAR comprising a CAR spacer of the present disclosure, i.e., a hinge derived CAR spacer, a loop derived CAR spacer, or a combination thereof) inhibiting Her2 includes an anti-Her2 antibody or antigen-binding portion thereof.

### Signaling, Transmembrane, Costimulatory Domains

In some aspects, the intracellular domain of a CAR of the present disclosure (i.e., CAR comprising a CAR spacer of the present disclosure, i.e., a hinge derived CAR spacer, a loop derived CAR spacer, or a combination thereof) is a signaling domain derived from CD3zeta, FcR gamma, FcR beta, CD3 gamma, CD3 delta, CD3 epsilon, CD5, CD22, CD79a, CD79b, and CD66d. In some aspect, the CAR further comprises a co-stimulatory domain derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, or B7-H3. In some aspects, the CAR further comprises a 4-11BBn costimulatory domain. In some aspects, the 4-1BB costimulatory domain comprises SEQ ID NO: 4869.

In some aspects, the CAR of the present disclosure (i.e., CAR comprising a CAR spacer of the present disclosure, i.e., a hinge derived CAR spacer, a loop derived CAR spacer, or a combination thereof) comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. The transmembrane domain may be derived either from a natural or from a recombinant source. Where the source is natural, the domain may be derived from any membrane-bound or transmembrane protein. In some aspects, the transmembrane domain is capable of signaling to the intracellular domain(s) whenever the CAR of the present disclosure has bound to a target.

In some aspects, a transmembrane domain can include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some aspects, the CAR of the present disclosure (i.e., CAR comprising a CAR spacer of the present disclosure, i.e., a hinge derived CAR spacer, a loop derived CAR spacer, or a combination thereof) further comprises a sequence encoding a costimulatory domain, e.g., a costimulatory domain described herein. In some aspects, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137). In some aspects, the costimulatory domain comprises a functional signaling domain of a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In some aspects, the costimulatory domain comprises 4-1BB, CD27, CD28, or ICOS.

In some aspects, the CAR of the present disclosure (i.e., CAR comprising a CAR spacer of the present disclosure, i.e., a hinge derived CAR spacer, a loop derived CAR spacer, or a combination thereof) further comprises a sequence encoding an intracellular signaling domain, e.g., an intracellular signaling domain described herein. In some aspects, the intracellular signaling domain comprises a functional signaling domain of 4-1BB and/or a functional signaling domain of CD3 zeta. In some aspects, the intracellular signaling domain comprises a functional signaling domain of CD27 and/or a functional signaling domain of CD3 zeta.

In some aspects, the CAR of the present disclosure (i.e., CAR comprising a CAR spacer of the present disclosure, i.e., a hinge derived CAR spacer, a loop derived CAR spacer, or a combination thereof) further comprises a leader sequence. In some aspects, the intracellular signaling domain comprises CD3z comprising SEQ ID NO: 4870.

In some aspects, the CAR of the present disclosure (i.e., CAR comprising a CAR spacer of the present disclosure, i.e., a hinge derived CAR spacer, a loop derived CAR spacer, or a combination thereof) comprises an optional leader sequence (e.g., an optional leader sequence described herein), an extracellular antigen-binding domain, a CAR spacer of the present disclosure, a transmembrane domain (e.g., transmembrane domain described herein), and an intracellular stimulatory domain (e.g., intracellular stimulatory domain described herein).

In some aspects, an exemplary CAR construct of the present disclosure comprises an optional leader sequence (e.g., a leader sequence described herein), an extracellular antigen-binding domain, a hinge, a transmembrane domain, an intracellular costimulatory domain (e.g., an intracellular costimulatory domain described herein) and an intracellular stimulatory domain.

In some aspects, the transmembrane domain of a CAR of the present disclosure (e.g., a CAR targeting ROR1, CD19 or Her 2) comprises a transmembrane domain is which linked to the intracellular domain of the CAR by a linker.

In some aspects, the present disclosure provides a CAR comprising: (i) an R12 scFv comprising SEQ ID NO: 4875; (ii) a Gly-Ser linker of SEQ ID NO:4818 or 5088, (iii) Spacer 1 comprising or consisting of SEQ ID NO:4830, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872 or any combination thereof.

In some aspects, the present disclosure provides a CAR comprising: (i) an R12 scFv comprising SEQ ID NO: 4875; (ii) a Gly-Ser linker of SEQ ID NO:4818 or 5088, (iii) Spacer 15 comprising or consisting of SEQ ID NO:4843, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R12 scFv comprising SEQ ID NO: 4875; (ii) a Gly-Ser linker of SEQ ID NO:4818 or 5088, (iii) Spacer 21 comprising or consisting of SEQ ID NO:4849, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R11 scFv comprising SEQ ID NO: 5048; (ii) a Gly-Ser linker of SEQ ID NO:4818 or 5088, (iii) Spacer 4 comprising or consisting of SEQ ID NO:4833, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure comprises provides a CAR comprising: (i) an R11 scFv comprising SEQ ID NO: 5048; (ii) a Gly-Ser linker of SEQ ID NO:4818 or 5088, (iii) Spacer 5 comprising or consisting of SEQ ID NO:4834, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R11 scFv comprising SEQ ID NO: 5048; (ii) a Gly-Ser linker of SEQ ID NO:4818 or 5088, (iii) Spacer 6 comprising or consisting of SEQ ID NO:4835, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) a 2A2 scFv comprising SEQ ID NO: 5047; (ii) a Gly-Ser linker of SEQ ID NO:4818 or 5088, (iii) Spacer 13 comprising or consisting of SEQ ID NO:4841, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) a 2A2 scFv comprising SEQ ID NO: 5047; (ii) a Gly-Ser linker of SEQ ID NO:4818 or 5088, (iii) Spacer 21 comprising or consisting of SEQ ID NO:4889, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) a 2A2 scFv comprising SEQ ID NO: 5047; (ii) a Gly-Ser linker of SEQ ID NO:4818 or 5088, (iii) Spacer 28 comprising or consisting of SEQ ID NO:4856, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R12 scFv comprising SEQ ID NO: 4875; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 35 Å and 55 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R12 scFv comprising SEQ ID NO: 4875; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 45 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R11 scFv comprising SEQ ID NO: 5048; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 180 Å and 250 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) an R11 scFv comprising SEQ ID NO: 5048; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 200 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) a 2A2 scFv comprising SEQ ID NO: 5047; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 35 Å and 55 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising: (i) a 2A2 scFv comprising SEQ ID NO: 5047; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 45 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a CAR comprising, consisting, or consisting essentially of a sequence set forth in SEQ ID NOS: 5049 to 5063.

In some aspects, the present disclosure provides a polynucleotide sequence encoding a CAR comprising: (i) an R12 scFv comprising SEQ ID NO: 4875; (ii) a Gly-Ser linker of SEQ ID NO:4818 or 5088, (iii) Spacer 1 comprising or consisting of SEQ ID NO:4830, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R12 scFv comprising SEQ ID NO: 4875; (ii) a Gly-Ser linker of SEQ ID NO:4818 or 5088, (iii) Spacer 15 comprising or consisting of SEQ ID NO:4843, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R12 scFv comprising SEQ ID NO: 4875; (ii) a Gly-Ser linker of SEQ ID NO:4818 or 5088, (iii) Spacer 21 comprising or consisting of SEQ ID NO:4849, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R11 scFv comprising SEQ ID NO: 5048; (ii) a Gly-Ser linker of SEQ ID NO:4818 or 5088, (iii) Spacer 4 comprising or consisting of SEQ ID NO:4833, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R11 scFv comprising SEQ ID NO: 5048; (ii) a Gly-Ser linker of SEQ ID NO:4818 or 5088, (iii) Spacer 5 comprising or consisting of SEQ ID NO:4834, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R11 scFv comprising SEQ ID NO: 5048; (ii) a Gly-Ser linker of SEQ ID NO:4818 or 5088, (iii) Spacer 6 comprising or consisting of SEQ ID NO:4835, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) a 2A2 scFv comprising SEQ ID NO: 5047; (ii) a Gly-Ser linker of SEQ ID NO:4818 or 5088, (iii) Spacer 13 comprising or consisting of SEQ ID NO:4841, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) a 2A2 scFv comprising SEQ ID NO: 5047; (ii) a Gly-Ser linker of SEQ ID NO:4818 or 5088, (iii) Spacer 21 comprising or consisting of SEQ ID NO:4889, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) 2A2 scFv comprising SEQ ID NO: 5047; (ii) a Gly-Ser linker of SEQ ID NO:4818 or 5088, (iii) Spacer 28 comprising or consisting of SEQ ID NO:4856, (iv) a transmembrane domain comprising SEQ ID NO: 4868 and optionally (v) a 4-1BB costimulatory domain comprising SEQ ID NO: 4869. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R12 scFv comprising SEQ ID NO: 4875; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 35 Å and 55 Å. In some aspects, the CAR comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R12 scFv comprising SEQ ID NO: 4875; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 45 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R11 scFv comprising SEQ ID NO: 5048; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 180 Å and 250 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) an R11 scFv comprising SEQ ID NO: 5048; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 200 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) 2A2 scFv comprising SEQ ID NO: 5047; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 35 Å and 55 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising: (i) 2A2 scFv comprising SEQ ID NO: 5047; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 45 Å. In some aspects, the CAR further comprises (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a polynucleotide sequence encoding a CAR comprising, consisting, or consisting essentially of a protein sequence set forth in SEQ ID NOS: 5049 to 5063.

### Bispecific CARs

In some aspects, the CARs of the present disclosure are bispecific CARs. Accordingly, in some aspects, the polynucleotide encoding a CAR of the present disclosure encodes at least a polypeptide of a bispecific CAR (e.g., a CAR targeting a first antigen and second antigen).

In some aspects, the antigen-binding domain of a CAR of the present disclosure is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some aspects, the first and second epitopes are on the same antigen, e.g., the same protein (or subunit of a multimeric protein). In some aspects, the first and second epitopes overlap. In other aspects, the first and second epitopes do not overlap. In some aspects, the first and second epitopes are on different antigens, e.g., different proteins (or different subunits of a multimeric protein).

In some aspects, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some aspects, a bispecific antibody molecule comprises a half antibody having binding specificity for a first epitope and a half antibody having binding specificity for a second epitope. In some aspects, a bispecific antibody molecule comprises a half antibody, or fragment thereof, having binding specificity for a first epitope and a half antibody, or fragment thereof, having binding specificity for a second epitope. In some aspects, a bispecific antibody molecule comprises a scFv, or fragment thereof, have binding specificity for a first epitope and a scFv, or fragment thereof, have binding specificity for a second epitope.

In certain aspects, the antibody molecule is a multi-specific (e.g., a bispecific or a trispecific) antibody molecule. Protocols for generating bispecific or heterodimeric antibody molecules are known in the art.

Within each antibody or antigen-binding antibody fragment (e.g., scFv) of a bispecific antibody molecule, the VH can be upstream or downstream of the VL. In some aspects, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other aspects, the upstream antibody or antibody fragment (e.g., scFv) is arranged with its VL (VL₁) upstream of its VH (VH₁) and the downstream antibody or antibody fragment (e.g., scFv) is arranged with its VH (V12) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄Ser)n linker, wherein n is 1, 2, 3, 4, 5, or 6, e.g., 4 (SEQ ID NO: 83). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some aspects, a bispecific CAR comprises VLs, VHs, and optionally one or more linkers in an arrangement as described herein.

In some aspects, the antibody molecule is a bispecific antibody molecule having a first epitope located on a first tumor antigen (e.g., ROR1) and a second epitope located on a second antigen, e.g., CD10, CD19, CD20, CD22, CD34, CD123, FLT-3, ROR1, CD79b, CD179b, or CD79a. In some aspects, the bispecific antibody binds to a first epitope, wherein the first epitope is located on CD19, and to a second epitope, wherein the second epitope is located on CD20. In some aspects, the bispecific antibody binds to a first epitope, wherein the first epitope is located on CD19, and to a second epitope, wherein the second epitope is located on CD22. In some aspects, the bispecific antibody binds to a first epitope, wherein the first epitope is located on CD20, and to a second epitope, wherein the second epitope is located on CD22. In certain aspects, the antibody molecule is a bispecific antibody molecule having a first binding specificity for a first B-cell epitope and a second binding specificity for another B-cell antigen. For instance, in some embodiments the bispecific antibody molecule has a first binding specificity for a first B-cell epitope, e.g., for ROR1, and a second binding specificity for one or more of CD10, CD19, CD20, CD22, CD34, CD123, FLT-3, ROR1, CD79b, CD179b, or CD79a B-cell epitopes.

### Inducible CARs

In some aspects, the expression of a CAR of the present disclosure (i.e., CAR comprising a CAR spacer of the present disclosure, i.e., a hinge derived CAR spacer, a loop derived CAR spacer, or a combination thereof) is regulated by a constitutive promoter, e.g., immediate early cytomegalovirus (CMV) promoter, Elongation Growth Factor-1α (EF-la), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. However, the regulation of the expression of a CAR of the present disclosure is not limited to the use of a constitutive promoter.

Thus, in some aspects, the CAR of the present disclosure encoded by a polynucleotide disclosed herein is an inducible CAR. The term “inducible” refers to the presence of an “inducible promoter,” i.e., a nucleotide sequence which, when operably linked with a polynucleotide which encodes or specifies a gene product, e.g., a CAR of the present disclosure, causes the gene product to be produced in a cell substantially only when an inducer which corresponds to the promoter is present in the cell. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In other aspects, a polynucleotide encoding a CAR of the present disclosure comprises a “tissue-specific” promoter, i.e., a nucleotide sequence which, when operably linked with a polynucleotide which encodes or specifies a gene product, e.g., a CAR of the present disclosure, causes the gene product to be produced in a cell substantially only if the cell is a cell of the tissue type corresponding to the promoter.

### Vectors

The present disclosure also provides a vector comprising a polynucleotide encoding a CAR of the present disclosure (i.e., CAR comprising a CAR spacer of the present disclosure, i.e., a hinge derived CAR spacer, a loop derived CAR spacer, or a combination thereof) operably linked to a regulatory element. In some aspects, the polynucleotide encoding a CAR of the present disclosure is a DNA molecule, or a RNA molecule.

In some aspects, the vector is a transfer vector. The term “transfer vector” refers to a composition of matter which comprises an isolated nucleic acid (e.g., a polynucleotide of the present disclosure) and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

In some aspects, the vector is an expression vector. The term “expression vector” refers to a vector comprising a recombinant polynucleotide (e.g., a polypeptide of the present disclosure) comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In some aspects, the vector is a viral vector, a mammalian vector, or bacterial vector. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an AAV vector.

In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ˜33Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In other aspects, the viral vector is a retroviral vector, e.g., a lentiviral vector (e.g., a third or fourth generation lentiviral vector). The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

In some aspects, the present disclosure provides a lentiviral vector comprising a polynucleotide sequence encoding: (i) R12 scFv comprising SEQ ID NO: 4875; (ii) Spacer 1 comprising or consisting of SEQ ID NO: 4830, and optionally a Gly-Ser linker of SEQ ID NO:4818 or 5088; (iii) a transmembrane domain comprising SEQ ID NO: 4868 and optionally (iv) a 4-1BB costimulatory domain comprising SEQ ID NO: 4869. In some aspects, the lentiviral vector further comprises a polynucleotide sequence encoding (v) CD3z comprising SEQ ID NO: 4870; (vi) P2A comprising SEQ ID NO: 4871; (vii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) Spacer 11 comprising or consisting of SEQ ID NO: 4840, and optionally a Gly-Ser linker of SEQ ID NO:4818 or 5088; (iii) a transmembrane domain comprising SEQ ID NO: 4868 and optionally (iv) a 4-1BB costimulatory domain comprising SEQ ID NO: 4869. In some aspects, the lentiviral vector further comprises a polynucleotide sequence encoding (v) CD3z comprising SEQ ID NO: 4870; (vi) P2A comprising SEQ ID NO: 4871; (vii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) Spacer 13 comprising or consisting of SEQ ID NO: 4841, and optionally a Gly-Ser linker of SEQ ID NO:4818 or 5088; (iii) a transmembrane domain comprising SEQ ID NO: 4868 and optionally (iv) a 4-1BB costimulatory domain comprising SEQ ID NO: 4869. In some aspects, the lentiviral vector further comprises a polynucleotide sequence encoding (v) CD3z comprising SEQ ID NO: 4870; (vi) P2A comprising SEQ ID NO: 4871; (vii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) Spacer 14 comprising or consisting of SEQ ID NO:4842, and optionally a Gly-Ser linker of SEQ ID NO:4818 or 5088; (iii) a transmembrane domain comprising SEQ ID NO: 4868 and optionally (iv) a 4-1BB costimulatory domain comprising SEQ ID NO: 4869. In some aspects, the lentiviral vector further comprises a polynucleotide sequence encoding (v) CD3z comprising SEQ ID NO: 4870; (vi) P2A comprising SEQ ID NO: 4871; (vii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) a Gly-Ser linker of SEQ ID NO:4818 or 5088, (iii) Spacer 1 comprising or consisting of SEQ ID NO:4830, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) a Gly-Ser linker of SEQ ID NO:4818 or 5088, (iii) Spacer 15 comprising or consisting of SEQ ID NO:4843, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) a Gly-Ser linker of SEQ ID NO:4818 or 5088, (iii) Spacer 21 comprising or consisting of SEQ ID NO:4849, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R11 scFv comprising SEQ ID NO: 5048; (ii) a Gly-Ser linker of SEQ ID NO:4818 or 5088, (iii) Spacer 4 comprising or consisting of SEQ ID NO:4833, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R11 scFv comprising SEQ ID NO: 5048; (ii) a Gly-Ser linker of SEQ ID NO:4818 or 5088, (iii) Spacer 5 comprising or consisting of SEQ ID NO:4834, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R11 scFv comprising SEQ ID NO: 5048; (ii) a Gly-Ser linker of SEQ ID NO:4818 or 5088, (iii) Spacer 6 comprising or consisting of SEQ ID NO:4835, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) 2A2 scFv comprising SEQ ID NO: 5047; (ii) a Gly-Ser linker of SEQ ID NO:4818 or 5088, (iii) Spacer 13 comprising or consisting of SEQ ID NO:4841, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) 2A2 scFv comprising SEQ ID NO: 5047; (ii) a Gly-Ser linker of SEQ ID NO:4818 or 5088, (iii) Spacer 21 comprising or consisting of SEQ ID NO:4889, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R2A2 scFv comprising SEQ ID NO: 5047; (ii) a Gly-Ser linker of SEQ ID NO:4818 or 5088, (iii) Spacer 28 comprising or consisting of SEQ ID NO:4856, (iv) a transmembrane domain comprising SEQ ID NO: 4868 and optionally (v) a 4-1BB costimulatory domain comprising SEQ ID NO: 4869. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 35 Å and 55 Å. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R12 scFv comprising SEQ ID NO: 4875; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 45 Å. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R11 scFv comprising SEQ ID NO: 5048; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 180 Å and 250 Å. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) R11 scFv comprising SEQ ID NO: 5048; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 200 Å. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) 2A2 scFv comprising SEQ ID NO: 5047; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is between 35 Å and 55 Å. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding: (i) 2A2 scFv comprising SEQ ID NO: 5047; (ii) a Gly-Ser linker of SEQ ID NO:4818 or 5088, (iii) a spacer derived from a human immunoglobulin hinge and/or constant region or a functional fragment thereof, (iv) a transmembrane domain comprising SEQ ID NO: 4868 and optionally (v) a 4-1BB costimulatory domain comprising SEQ ID NO: 4869, wherein the combined length of the linker (ii) and spacer (iii) is about 45 Å. In some aspects, the lentiviral vector further comprises a polynucleotide sequence encoding (vi) CD3z comprising SEQ ID NO: 4870; (vii) P2A comprising SEQ ID NO: 4871; (viii) EGFRt comprising SEQ ID NO: 4872, or any combination thereof.

In some aspects, the present disclosure provides a lentiviral vector comprising a polynucleotide sequence encoding a CAR comprising, consisting, or consisting essentially of a sequence set forth in SEQ ID NOS: 5049 to 5063. In some aspects, the present disclosure provides a lentiviral vector comprising a polynucleotide sequence encoding: (i) FMC63 scFv comprising SEQ ID NO: 4866; (ii) Spacer 7 comprising or consisting of SEQ ID NO: 4836, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 8 comprising or consisting of SEQ ID NO: 4837, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 9 comprising or consisting of SEQ ID NO: 4838, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 10 comprising or consisting of SEQ ID NO: 4939, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 11 comprising or consisting of SEQ ID NO: 4840, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 14 comprising or consisting of SEQ ID NO: 4842, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 16 comprising or consisting of SEQ ID NO: 4844, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 28 comprising or consisting of SEQ ID NO: 4856, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, the present disclosure provides a lentiviral vector comprising a polynucleotide sequence encoding: (i) FMC63 scFv comprising SEQ ID NO: 4866; (ii) Spacer 28 comprising or consisting of SEQ ID NO: 4856, and optionally a Gly-Ser linker of SEQ ID NO:4818 or 5088; (iii) a transmembrane domain comprising SEQ ID NO: 4877 and optionally (iv) a 4-1BB costimulatory domain comprising SEQ ID NO: 4878. In some aspects, the lentiviral vector further comprises a polynucleotide sequence encoding (v) CD3z comprising SEQ ID NO: 4879; (vi) P2A comprising SEQ ID NO: 4880; (vii) EGFRt comprising SEQ ID NO: 4881, or any combination thereof.

In some aspects, non-viral methods can be used to deliver a nucleic acid comprising a polynucleotide encoding a CAR of the present disclosure into a cell or tissue of a subject. In some aspects, the non-viral method includes the use of a transposon. In some aspects, use of a non-viral method of delivery permits reprogramming of cells, e.g., T or NK cells, and direct infusion of the cells into the subject. In some aspects, a nucleic acid sequence comprising a polynucleotide encoding a CAR of the present disclosure can be inserted into the genome of a target cell (e.g., a T cell) or a host cell (e.g., a cell for recombinant expression of the CAR polypeptide) by using CRISPR/Cas systems and genome edition alternatives such as zinc-finger nucleases (ZFNs), transcription activator-like effector nucleases (TALENs), and meganucleases (MNs).

In some, a CAR of the present disclosure (i.e., CAR comprising a CAR spacer of the present disclosure, i.e., a hinge derived CAR spacer, a loop derived CAR spacer, or a combination thereof) can be expressed in a cell using bicistronic or multicistronic expression vectors. In some aspects, bicistronic or multicistronic vectors include, but not limited to, (1) multiple promoters fused to multiple CARs' open reading frames; (2) insertion of splicing signals between units of a CAR; fusion of CARs of which expressions are driven by a single promoter; (3) insertion of proteolytic cleavage sites between units of CAR (self-cleavage peptide); and (iv) insertion of internal ribosomal entry sites (IRESs).

In some aspects, multiple CAR units are expressed in a single open reading frame (ORF), thereby creating a single polypeptide having multiple CAR units, wherein at least one of the CARs is a CAR of the present disclosure. In some aspects, an amino acid sequence or linker containing a high efficiency cleavage site is disposed between each CAR unit. As used herein, high cleavage efficiency is defined as more than 50%, more than 70%, more than 80%, or more than 90% of the translated protein is cleaved. Cleavage efficiency can be measured by Western Blot analysis.

Non-limiting examples of high efficiency cleavage sites include porcine teschovirus-1 2A (P2 Å), FMDV 2 Å (abbreviated herein as F2A); equine rhinitis A virus (ERAV) 2 Å (E2 Å); and Thoseaasigna virus 2 Å (T2A), cytoplasmic polyhedrosis virus 2 Å (BmCPV2A) and flacherie Virus 2 Å (BmIFV2A), or a combination thereof. In some aspects, the high efficiency cleavage site is P2A. High efficiency cleavage sites are described in Kim et al. (2011) High Cleavage Efficiency of a 2A Peptide Derived from Porcine Teschovirus-1 in Human Cell Lines, Zebrafish and Mice. PLoS ONE 6(4): e18556, the contents of which are incorporated herein by reference.

In some aspects, multiple CAR units are expressed in a single open reading frame (ORF), expression is under the control of a strong promoter.

In some aspects, the vector of the present disclosure further comprises an accessory gene. In some aspects, the accessory gene is a non-immunogenic selection tool, a tracking marker, or a suicide gene. In some aspects, the accessory gene is a truncated EGFR gene (EGFRt). An example of a truncated EGFR (EGFRt) gene that can be used in accordance with the embodiments described herein comprises SEQ ID NO: 4872 (includes the GMCSF signal peptide set forth in SEQ ID NO:4865) or SEQ ID NO:5064 (without signal peptide).

### Polynucleotide Modifications

In some aspects, a polynucleotide encoding a CAR of the present disclosure (i.e., CAR comprising a CAR spacer of the present disclosure, i.e., a hinge derived CAR spacer, a loop derived CAR spacer, or a combination thereof) can comprise at least one chemically modified nucleobase, sugar, backbone, or any combination thereof. Thus, a polynucleotide encoding a CAR of the present disclosure can comprise one or more modifications. In some aspects, a polynucleotide encoding a CAR of the present disclosure comprises at least one nucleotide analogue. In some aspects, at least one nucleotide analogue introduced by using IVT (in vitro transcription) or chemical synthesis is selected from the group consisting of a 2′-O-methoxyethyl-RNA (2′-MOE-RNA) monomer, a 2′-fluoro-DNA monomer, a 2′-O-alkyl-RNA monomer, a 2′-amino-DNA monomer, a locked nucleic acid (LNA) monomer, a cEt monomer, a cMOE monomer, a 5′-Me-LNA monomer, a 2′-(3-hydroxy)propyl-RNA monomer, an arabino nucleic acid (ANA) monomer, a 2′-fluoro-ANA monomer, an anhydrohexitol nucleic acid (HNA) monomer, an intercalating nucleic acid (INA) monomer, and a combination of two or more of said nucleotide analogues. In some aspects, the optimized nucleic acid molecule comprises at least one backbone modification, for example, a phosphorothioate internucleotide linkage.

In some aspects, a polynucleotide encoding a CAR of the present disclosure can be chemically modified at terminal locations, for example by introducing M (2′-O-methyl), MS (2′-O-methyl 3′ phosphorothioate), or MSP (2′-O-methy 3′thioPACE, phosphonoacetate) modifications, or combinations thereof at positions 1, 2, 3 respect to the 5′ and/or 3′ termini.

Modified polynucleotides encoding a CAR of the present disclosure (i.e., CAR comprising a CAR spacer of the present disclosure, i.e., a hinge derived CAR spacer, a loop derived CAR spacer, or a combination thereof) need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% modified nucleotides.

In some aspects, a polynucleotide encoding a CAR of the present disclosure (i.e., CAR comprising a CAR spacer of the present disclosure, i.e., a hinge derived CAR spacer, a loop derived CAR spacer, or a combination thereof) can include modifications to prevent rapid degradation by endo- and exo-nucleases. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

Specific examples of synthetic, modified polynucleotides encoding a CAR of the present disclosure (i.e., CAR comprising a CAR spacer of the present disclosure, i.e., a hinge derived CAR spacer, a loop derived CAR spacer, or a combination thereof) useful with the methods described herein include, but are not limited to, polynucleotides encoding a CAR of the present disclosure containing modified or non-natural internucleoside linkages. Synthetic, modified polynucleotides encoding a CAR of the present disclosure having modified internucleoside linkages include, among others, those that do not have a phosphorus atom in the internucleoside linkage. In some aspects, a synthetic, modified polynucleotide encoding a CAR of the present disclosure has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, T-5′ linked analogs of these, and those) having inverted polarity wherein the adjacent pairs of nucleoside units are linked 3′-5′ to 5′-3′ or T-5′ to 5′-T. Various salts, mixed salts and free acid forms are also included.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

In some aspects, a polynucleotide encoding a CAR of the present disclosure (i.e., CAR comprising a CAR spacer of the present disclosure, i.e., a hinge derived CAR spacer, a loop derived CAR spacer, or a combination thereof) can be codon optimized by introducing one or more synonymous codon changes. As used herein, the terms “codon optimization,” “codon optimized,” and grammatical variants thereof refer to the modification of the primary sequence of a nucleic acid by replacing synonymous codons in order to increase its translational efficiency. Accordingly, codon optimization comprises switching the codons used in a polynucleotide encoding a CAR of the present disclosure without changing the amino acid sequence that it encodes for, which typically dramatically increases the abundance of the protein the codon optimized gene encodes because it generally removes “rare” codons and replaces them with abundant codons, or removes codon with a low tRNA recharge rate with codon with high tRNA recharge rates. Such codon optimization can, for example, (i) improve protein yield in recombinant protein expression, or (ii) improve the stability, half life, or other desirable property of an mRNA or a DNA encoding a binding molecule disclosed herein, wherein such mRNA or DNA is administered to a subject in need thereof.

The sequences of polynucleotides encoding a CAR of the present disclosure (i.e., CAR comprising a CAR spacer of the present disclosure, i.e., a hinge derived CAR spacer, a loop derived CAR spacer, or a combination thereof) can be codon optimized using any methods known in the art at the time the present application was filed.

In some aspects, a polynucleotide encoding a CAR of the present disclosure (i.e., CAR comprising a CAR spacer of the present disclosure, i.e., a hinge derived CAR spacer, a loop derived CAR spacer, or a combination thereof) has been sequence optimized. As used herein, the term “sequence optimized” refers to the modification of the sequence of a nucleic acid by to introduce features that increase its translational efficiency, remove features that reduce its translational efficiency, or in general improve properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, or increasing and/or decreasing protein aggregation

The present disclosure contemplates modifications to the entire CAR construct e.g., modifications in one or more amino acid sequences of the various domains of the CAR construct in order to generate functionally equivalent molecules. The CAR construct can be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity of the starting CAR construct. The present disclosure also contemplates modifications of specific regions of a CAR, e.g., modifications in one or more amino acid sequences of one or more CDRs of a CAR construct in order to generate functionally equivalent molecules.

### Cells

The present disclosure also provides a genetically modified cell comprising a polynucleotide encoding a CAR of the present disclosure (i.e., CAR comprising a CAR spacer of the present disclosure, i.e., a hinge derived CAR spacer, a loop derived CAR spacer, or a combination thereof). In some aspects, the CAR is recombinantly expressed by a cell genetically modified to express a CAR, wherein the cell comprises by one or more of the polynucleotide sequences or the vectors encoding a CAR of the present disclosure.

In some aspects, the genetically modified cell disclosed herein has been transfected with a polynucleotide or vector encoding a CAR of the present disclosure. The term “transfected” (or equivalent terms “transformed” and “transduced”) refers to a process by which exogenous nucleic acid, e.g., a polynucleotide or vector encoding a CAR of the present disclosure, is transferred or introduced into the genome of the host cell, e.g., a T cell. A “transfected” cell is one which has been transfected, transformed or transduced with exogenous nucleic acid, e.g., a polynucleotide or vector encoding a CAR of the present disclosure. The cell includes the primary subject cell and its progeny.

In some aspects, the cell (e.g., T cell) is transfected with a vector of the present disclosure, e.g., an AAV vector or a lentiviral vector. In some such aspects, the cell may stably express the CAR of the present disclosure.

In some aspects, the cell (e.g., T cell) is transfected with a nucleic acid, e.g., mRNA, cDNA, DNA, encoding a CAR of the present disclosure. In some such aspects, the cell may transiently express the CAR of the present disclosure. For example, an RNA construct can be directly transfected into a cell. A method for generating mRNA for use in transfection involves in vitro transcription (IVT) of a template with specially designed primers, followed by polyA addition, to produce a construct containing 3′ and 5′ untranslated sequence (UTR), a 5′ cap and/or Internal Ribosome Entry Site (IRES), the nucleic acid to be expressed, and a polyA tail, typically 50-2000 bases in length. RNA so produced can efficiently transfect different kinds of cells. In some aspects, the template includes sequences for the CAR of the present disclosure. In an aspect, an RNA CAR vector is transduced into a T cell by electroporation.

In some aspects, the cell is an immune effector cell. As used herein, term “immune effector cell” refers to a cell that is involved in an immune response, e.g., in the promotion of an immune effector response. “Immune effector function” or “immune effector response,” refer to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines. The intracellular signaling domain of a CAR can generate a signal that promotes an immune effector function of the CAR containing cell, e.g., a CART cell. Examples of immune effector function, e.g., in a CART cell, include cytolytic activity and helper activity, including the secretion of cytokines. In some aspects, the intracellular signal domain is the portion of the CAR which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some aspects, the intracellular signaling domain can comprise a primary intracellular signaling domain. Exemplary primary intracellular signaling domains include those derived from the molecules responsible for primary stimulation, or antigen dependent simulation. In some aspects, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcεRI, CD66d, CD32, DAP10 and DAP12.

Examples of immune effector cells include, e.g., T cells, e.g., alpha/beta T cells and gamma/delta T cells, B cells, natural killer (NK) cells, natural killer T (NKT) cells, mast cells, and myeloid-derived phagocytes. Innate lymphoid cells (ILCs) are a group of innate immune cells that are derived from common lymphoid progenitor (CLP) and belong to the lymphoid lineage. These cells are defined by absence of antigen specific B or T cell receptor because of the lack of recombination activating gene (RAG). ILCs do not express myeloid or dendritic cell markers. TLCs has varying physiological functions; some functions are analogous to helper T cells, while the group also includes cytotoxic NK cells. Accordingly, in some aspects, the cell genetically modified to express a CAR of the present disclosure is, e.g., a T cell, an NK cell, an NKT cell, or an ILC cell.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

### Pharmaceutical Compositions

The present disclosure also provides pharmaceutical compositions comprising compositions disclosed herein, e.g., a polynucleotide encoding a CAR of the present disclosure, a vector comprising a polynucleotide encoding a CAR of the present disclosure, or a genetically modified cell comprising a polynucleotide or a vector encoding a CAR of the present disclosure, which are suitable for administration to a subject.

The pharmaceutical compositions generally comprise polynucleotide, vector, or cell encoding or comprising a CAR of the present disclosure and a pharmaceutically-acceptable excipient or carrier in a form suitable for administration to a subject. Pharmaceutically acceptable excipients or carriers are determined in part by the particular composition being administered, as well as by the particular method used to administer the composition.

There is a wide variety of suitable formulations of pharmaceutical compositions comprising a CAR of the present disclosure (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

In certain aspects, the pharmaceutical composition is co-administered with of one or more additional therapeutic agents, in a pharmaceutically acceptable carrier. In some aspects, the pharmaceutical composition comprising the CAR of the present disclosure is administered prior to administration of the additional therapeutic agent(s). In other aspects, the pharmaceutical composition comprising the CAR of the present disclosure is administered after the administration of the additional therapeutic agent(s). In further aspects, the pharmaceutical composition comprising the CAR of the present disclosure is administered concurrently with the additional therapeutic agent(s).

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the compositions of the present disclosure (e.g., polynucleotides, vectors, or cells), use thereof in the compositions is contemplated.

### VI. Libraries and Methods of Use

The present disclosure also provides libraries comprising Ig hinges, fragment thereof, and combinations thereof (e.g., two or more Ig hinge fragments concatenated). The libraries of the present disclosure can be screened to identify polypeptides with the optimal length and composition to be used as CAR spacer, i.e., spacer resulting in CAR with enhanced activity with respect to a corresponding CAR comprising a reference spacer (e.g., a spacer comprising an IgG1 hinge).

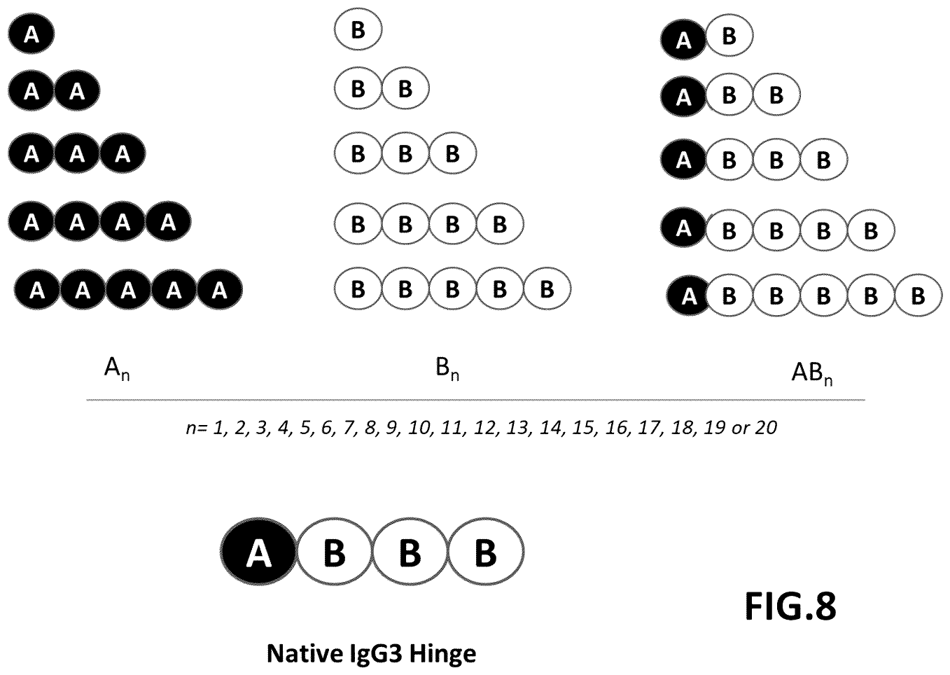

In some aspects, spacer libraries can be generated by concatenating fragments of immunoglobulin hinges disclosed here (see disclosures about modular spacers above). A schematic representation of a small library generated from IgG3 modules is provided in FIG. 8. In some aspects, a spacer library of the present disclosure comprises Spacer 1 to Spacer 31 disclosed in FIG. 9. In some aspects, a spacer library of the present disclosure comprises Spacer 1 to Spacer 31 disclosed in FIG. 9 and/or variants thereof, e.g., spacers corresponding to Spacer 1 to Spacer 31 further comprising a flexible linker, e.g., a flexible linker of SEQ ID NO: 4818 or 5088, linked to the N-terminus and/or C-terminus of the amino sequence of Spacer 1 to Spacer 31 as set forth in FIG. 9.

The present disclosure also provides CAR libraries comprising, e.g., variants of a CAR in which the spacer is replaced by a spacer from a spacer library of the present disclosure (e.g., a library comprising the spacers disclosed in FIG. 9 or variants thereof). Also provided are CAR libraries comprising, e.g., variants of a CAR comprising a spacer disclosed herein (e.g., a spacer disclosed in FIG. 9) in which other CAR components are substituted, e.g., signal peptide, binding (e.g., scFv), transmembrane domain, costimulatory domain, signaling domain, or any combination thereof.

### VII. Indications

In some aspects, the compositions disclosed herein (e.g., polynucleotides encoding CARs of the present disclosure, vectors comprising polynucleotides encoding CARs of the present disclosure, CARs of the present disclosure, or cells expressing CARs of the present disclosure, .eg., CART cells) can be used to treat a disease or condition, e.g., a proliferative disease such as a cancer or malignancy or a precancerous condition such as a myelodysplasia, a myelodysplastic syndrome or a preleukemia.

A “cancer” refers to a broad group of various proliferative diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. As used herein the term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth. In some aspects, the cancer is a tumor. “Tumor,” as used herein, refers to all neoplastic cell growth and proliferation, whether malignant or benign, and all pre-cancerous and cancerous cells and tissues.

In some aspects, the disease is a solid or a liquid tumor. In some aspects, the cancer is a pancreatic cancer. In some aspects, the disease is a hematologic cancer. In some aspects, the hematologic cancer is a leukemia. In some aspects, the cancer is selected from the group consisting of one or more acute leukemias including but not limited to B-cell acute lymphoid leukemia (BALL), T-cell acute lymphoid leukemia (TALL), small lymphocytic leukemia (SLL), acute lymphoid leukemia (ALL) (e.g., relapsing and refractory ALL); one or more chronic leukemias including but not limited to chronic myelogenous leukemia (CML), and chronic lymphocytic leukemia (CLL). Additional hematologic cancers or conditions include, but are not limited to mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and preleukemia. Preleukemia encompasses a diverse collection of hematological conditions united by ineffective production (or dysplasia) of myeloid blood cells. In some aspects, the indication is an atypical and/or non-classical cancer, malignancy, precancerous condition or proliferative disease; and any combination thereof.

In some aspects, the disease is a lymphoma, e.g., MCL or Hodgkin lymphoma. In some aspects, the disease is leukemia, e.g., SLL, CLL and/or ALL. In some aspects, the disease associated with a tumor antigen, e.g., a tumor antigen described herein, is selected from a proliferative disease such as a cancer or malignancy or a precancerous condition such as a myelodysplasia, a myelodysplastic syndrome or a preleukemia, or is a non-cancer related indication associated with expression of a tumor antigen described herein. In some aspects, the disease associated with a tumor antigen described herein is a solid tumor, e.g., a solid tumor described herein, e.g., prostatic, colorectal, pancreatic, cervical, gastric, ovarian, head, or lung cancer.

In some aspects, the cancer is chosen from AML, ALL, B-ALL, T-ALL, B-cell prolymphocytic leukemia, chronic lymphocytic leukemia, CML, hairy cell leukemia, Hodgkin lymphoma, mast cell disorder, myelodysplastic syndrome, myeloproliferative neoplasm, plasma cell myeloma, plasmacytoid dendritic cell neoplasm, or a combination thereof.

In some aspects, the compositions disclosed herein (e.g., polynucleotides encoding CARs of the present disclosure, vectors comprising polynucleotides encoding CARs of the present disclosure, CARs of the present disclosure, or cells expressing CARs of the present disclosure, e.g., CART cells) are used to reduce or decrease a size of a tumor or inhibit a tumor growth in a subject in need thereof. In some aspects, the tumor is a carcinoma (i.e., a cancer of epithelial origin). In some aspects, the tumor is, e.g., selected from the group consisting of gastric cancer, gastroesophageal junction cancer (GEJ), esophageal cancer, colorectal cancer, liver cancer (hepatocellular carcinoma, HCC), ovarian cancer, breast cancer, NSCLC, bladder cancer, lung cancer, pancreatic cancer, head and neck cancer, lymphoma, uterine cancer, renal or kidney cancer, biliary cancer, prostate cancer, testicular cancer, urethral cancer, penile cancer, thoracic cancer, rectal cancer, brain cancer (glioma and glioblastoma), cervical cancer, parotid cancer, larynx cancer, thyroid cancer, adenocarcinomas, neuroblastomas, melanoma, and Merkel Cell carcinoma.

A “cancer” or “cancer tissue” can include a tumor at various stages. In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In other aspects, the cancer or tumor is stage II or stage III, such that, e.g., the cancer or tumor is larger than in stage 0 or stage I, and it has grown into neighboring tissues but it has not metastasized, except potentially to the lymph nodes. In other aspects, the cancer or tumor is stage IV, such that, e.g., the cancer or tumor has metastasized. Stage IV can also be referred to as advanced or metastatic cancer.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the tumor is a solid tumor. A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated with the compositions disclosed herein (e.g., polynucleotides encoding CARs of the present disclosure, vectors comprising polynucleotides encoding CARs of the present disclosure, CARs of the present disclosure, or cells expressing CARs of the present disclosure, e.g., CART cells) include, e.g., Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

### Methods

The present disclosure also provide methods for using of the CARs and CAR-expressing cells of the present disclosure for adoptive therapy. In some aspects, the present disclosure provides a method of stimulating a T cell-mediated immune response to a target cell population or tissue in a subject, comprising administering an effective amount of a cell expressing a CAR of the present disclosure to the subject. Also provided is a method of providing an anti-tumor immunity in a subject in need thereof, the method comprising administering to the subject an effective amount of a cell expressing a CAR of the present disclosure to the subject.

The disclosure also provides a method of treating cancer in a subject in need thereof comprising administering to the subject an effective amount of a cell expressing a CAR of the present disclosure. The disclosure also provides a method of preparing a population of cells, e.g. CART cells, for a therapy comprising transducing a population of cells isolated from a subject with the a polynucleotide or vector of the present disclosure. In some aspects, the transduction comprises culturing the cell under suitable condition.

The disclosure also provides a method of generating a persisting population of genetically engineered cells in a subject diagnosed with cancer, the method comprising administering to the subject a cell genetically engineered to express a CAR of the present disclosure.

The disclosure also provides a method of expanding a population of genetically engineered cells (e.g., T cells) in a subject diagnosed with cancer, the method comprising administering to the subject a cell (e.g., a T cell) genetically engineered to express a CAR of the present disclosure. In some aspects, the cell is a T cell, e.g., an autologous T cell. In other aspects, the T cell is a heterologous T cell. In some aspects of the methods disclosed herein, the subject is a human subject.

The present disclosure also provides a method to improve one or more properties of a CAR therapy comprising inserting an CAR spacer of the present disclosure between an antigen-binding domain and a transmembrane domain of a CAR, wherein the spacer is located between the ligand-binding domain and the transmembrane domain.

In some aspects, the one or more improved properties of the CAR therapy is increased secretion of one or more cytokines. In some aspects, the cytokine secretion induced by the CAR of the present disclosure, i.e., a CAR comprising a CAR spacer of the present disclosure, is increased with respect to the secretion observed after administration of a corresponding CAR comprising a reference spacer, e.g., an IgG1 hinge spacer. In some aspects, the cytokine is an interleukin, e.g., interleukin-2. In some aspect, the cytokine is an interferon, e.g., interferon-gamma.

In some aspects, administration of a CAR of the present disclosure results in an increase in interleukin (e.g., interleukin-2) secretion by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the interleukin secretion observed after administration of a corresponding CAR comprising a reference spacer, e.g., an IgG1 hinge CAR spacer or a “short” reference spacer (SEQ ID NO:4911), instead of a CAR spacer of the present disclosure.

In some aspects, administration of a CAR of the present disclosure results in an increase in interferon (e.g., interferon-gamma) secretion by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the interferon (e.g., interferon-gamma) secretion observed after administration of a corresponding CAR comprising a reference spacer, e.g., an IgG1 hinge CAR spacer or a “short” reference spacer (SEQ ID NO:4911), instead of a CAR spacer of the present disclosure.

In some aspects, administration of a CAR of the present disclosure results in an increase in TNFα secretion by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the TNFα secretion observed after administration of a corresponding CAR comprising a reference spacer, e.g., an IgG1 hinge CAR spacer or a “short” reference spacer (SEQ ID NO:4911), instead of a CAR spacer of the present disclosure.

In some aspects, the present disclosure provides a method to select an optimal CAR spacer comprising comparing killing kinetics (Area Under Curve) versus cytokine (e.g., IL-2 and/or IFNg) in a library of CARs differing in the spacer used (e.g., the spacer is a spacer from the library provided in FIG. 9 or any spacer disclosed herein), and selecting the CAR with the lowest AUC and/or highest cytokine. Also provided is a method to optimize or improve a CAR comprising replacing the CAR's original spacer with a spacer disclosed herein (e.g., a spacer from the library provided in FIG. 9 or any spacer disclosed herein), comparing killing kinetics (Area Under Curve) and cytokine production (e.g., IL-2 and/or IFNg), and selecting the CAR with the lowest AUC and/or highest cytokine production.

The present disclosure also provides a method to select an optimal spacer comprising generating a library of CARs differing in the spacer used (e.g., the spacer is a spacer from the library provided in FIG. 9 or any spacer disclosed herein) and performing sequential kill experiments in which CAR T cells are continually passaged into new target cells until the CAR T cells stop proliferating and killing. The best CAR spacers would be those present in the CAR T cells that would have a more prolonged proliferation and killing activity.

In some aspects, the present disclosure provides a polynucleotide, vector, CAR, composition, kit, cell, or the pharmaceutical composition of the present disclosure for use as a medicament. In some aspects, the present disclosure provides a polynucleotide, vector, CAR, composition, kit, cell, or the pharmaceutical composition of the present disclosure for use as a medicament for the treatment of cancer in a subject in need thereof. In some aspects, the present disclosure provides a polynucleotide, vector, CAR, composition, kit, cell, or the pharmaceutical composition of the present disclosure for the treatment of cancer in a subject in need thereof. In some aspects, the present disclosure provides the use of a polynucleotide, vector, CAR, composition, kit, cell, or the pharmaceutical composition of the present disclosure for the manufacture of a medicament. In some aspects, the present disclosure provides the use of a polynucleotide, vector, CAR, composition, kit, cell, or the pharmaceutical composition of the present disclosure for the manufacture of a medicament for treating cancer in a subject in need thereof.

The present disclosure also provides a composition comprising a polynucleotide encoding a CAR, a vector comprising a polynucleotide encoding a CAR, or a genetically modified cell comprising a polynucleotide or a vector encoding a CAR for treating a subject in need of a CAR therapy. The present disclosure also provides a composition comprising a polynucleotide encoding a CAR, a vector comprising a polynucleotide encoding a CAR, or a genetically modified cell comprising a polynucleotide or a vector encoding a CAR for use as a medicament. Also provided is a composition comprising a polynucleotide encoding a CAR, a vector comprising a polynucleotide encoding a CAR, or a genetically modified cell comprising a polynucleotide or a vector encoding a CAR for used as treatment for cancer in a subject in need of a CAR therapy. Also provided is a composition comprising a polynucleotide encoding a CAR, a vector comprising a polynucleotide encoding a CAR, or a genetically modified cell comprising a polynucleotide or a vector encoding a CAR for the manufacture of a medicament for the treatment for cancer in a subject in need of a CAR therapy.

### Kits

The present disclosure also provides kits, or products of manufacture comprising (i) a CAR of the present disclosure (i.e., CAR comprising a CAR spacer of the present disclosure, i.e., a hinge derived CAR spacer, a loop derived CAR spacer, or a combination thereof), one or more polynucleotides encoding a CAR of the present disclosure, one or more vectors encoding a CAR of the present disclosure, or a composition comprising the polynucleotide(s) or vector(s), and optionally (ii) instructions for use, e.g., instructions for use according to the methods disclosed herein.

The disclosure also provides a kits comprising (i) a cell genetically modified to express a CAR of the present disclosure, i.e., a cell one or more polynucleotides encoding a CAR of the present disclosure, or one or more vectors encoding a CAR of the present disclosure (e.g., a T cell, a natural killer (NK) cell, an natural killer T (NKT) cell, or an ILC cell), or a pharmaceutical composition comprising the cell, and optionally (ii) instructions for use.

In some aspects, the kit or product of manufacture comprises at least a polynucleotide or vector encoding a CAR of the present disclosure, a cell genetically modified to express a CAR of the present disclosure, or a composition (e.g., a pharmaceutical composition) comprising a polynucleotide, vector, or cell disclosed herein, in one or more containers.

In some aspects, the kit or product of manufacture comprises at least a polynucleotide or vector encoding a CAR of the present disclosure, a cell genetically modified to express a CAR of the present disclosure, or a composition (e.g., a pharmaceutical composition) comprising a polynucleotide, vector, or cell disclosed herein, and optionally a brochure.

One skilled in the art will readily recognize that the polynucleotides, vectors, cells, and compositions of the present disclosure, pharmaceutical composition comprising the polynucleotides, vectors, or cells of the present disclosure, or combinations thereof can be readily incorporated into one of the established kit formats which are well known in the art.

In some aspects, the kit or product of manufacture comprises, e.g., a polynucleotide or vector encoding a CAR of the present disclosure, or a composition (e.g., a pharmaceutical composition) comprising a polynucleotide, vector, in dry form in a container (e.g., a glass vial), and optionally a vial with a solvent.

In some aspects, the kit or product of manufacture comprises, e.g., a polynucleotide or vector encoding a CAR of the present disclosure, or a composition (e.g., a pharmaceutical composition) comprising a polynucleotide, vector, in at least one container, and another or more containers with transfection reagents.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); ); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Materials and Methods

To measure CAR expression and Transduction Efficiency, approximately 0.5×10⁶ cells were pelleted after a 6-day culturing post-transduction for flow cytometry analysis. Cells were resuspended in Fixable Viability Dye eFluor 780 (Invitrogen, Cat# 65-0865-14) in PBS for 10 minutes, then washed with Cell Staining Buffer (BioLegend, Cat# 420201).

FMC63 CARs scFv surface expression was detected using an anti-ID-AF647 (Lyell) diluted 1:2000 in Cell Staining Buffer. Transduction Efficiency was determined by surface staining of transduction marker EGFRt using anti-hEGFR-BV421 (BioLegend, Cat# 352911) diluted 1:40 in Cell Staining Buffer. Cells were pelleted after a 20 minutes incubation in the dark, followed by 2× wash with Cell Staining Buffer. All flow cytometric analysis was done on a ZE5 Cell Analyzer (Biorad) and analyzed with FlowJo (Tree Star).

Her CARs scFv surface expression was detected using Protein L-Biotin (Thermo Scientific, Cat# 29997) diluted 1:6000 in Cell Staining Buffer, followed by Strepavidin-BV421 (BioLegend, Cat# 405226) diluted 1:400 in Cell Staining Buffer. Transduction Efficiency was determined by surface staining of transduction marker EGFRt using anti-hEGFR-PE (BioLegend, Cat# 252903), diluted 1:40 in Cell Staining Buffer.

R11, R12 and 2A2 CAR scFv surface expressions were detected using hROR1-Fc-AF647 (Lyell) diluted 1:50 in Cell Staining Buffer. Transduction Efficiency was determined by surface staining of transduction marker EGFRt using anti-hEGFR-PE (BioLegend, Cat# 252903), diluted 1:40 in Cell Staining Buffer.

### Example 1

### Spacer-Dependent Activity by ROR1 CAR-T Cells

This study describes the effects of various spacers on inducing autoactivation as well as ROR1-directed cytokine release and potency by T cells expressing R12 CAR. The reference CAR sequence used to test different spacer lengths and its constituent elements is shown in TABLE 20 below.

Short, intermediate, and long reference spacers were used in the experiments disclosed herein. The “short” reference spacer is an IgG4 hinge-derived spacer of SEQ ID NO: 4911. The “intermediate” reference spacer is an IgG4 hinge-derived spacer of SEQ ID NO:4912. The “long” reference spacer is an IgG4 hinge-derived spacer of SEQ ID NO: 4913.

TABLE 20
R12 Reference CAR sequences

| | |
|---|---|
| SEQ ID NO | Description |

R12 CAR with Short reference spacer CAR against ROR1
SEQ ID NO: 5070

| | |
|---|---|
| 5070 | Full sequence/840aa |
| 4874 | Signal peptide/20aa/aa 1-20 |
| 4875 | scFv/248aa/aa 21-268 |
| 4867 | Spacer/12aa/aa 269-280 |
| 4868 | Transmembrane domain/28aa/aa 281-308 |
| 4869 | 4-1BB costimulatory domain/42aa/aa 309-350 |
| 4870 | CD3z/112aa/aa 351-462 |
| 4871 | P2A/21aa/aa 463-483 |
| 4872 | EGFRt/357aa/aa 484-840 (includes signal peptide set |
| | forth in SEQ ID NO:4865; 22aa/ aa484-505) |

R12 CAR with Short reference spacer CAR against ROR1 with optional
linker SEQ ID NO: 5071

| | |
|---|---|
| 5071 | Full sequence/845aa |
| 4874 | Signal peptide/20aa/aa 1-20 |
| 4875 | scFv/248aa/aa 21-268 |
| 4818 | Optional GGGSG linker/aa 269-273 |
| 4867 | Spacer/12aa/aa 274-285 |
| 4868 | Transmembrane domain/28aa/aa 286-313 |
| 4869 | 4-1BB costimulatory domain/42aa/aa 314-355 |
| 4870 | CD3z/112aa/aa 356-467 |
| 4871 | P2A/21aa/aa 468-488 |
| 4872 | EGFRt/357aa/aa 489-845 |

Methods: All assays were set up in cytokine-free RPMI1640 media supplemented with 10o FBS (cell-assay media). H1975, Jeko1, Nalm6, Raji, A549, and T47D cell lines were purchased from ATCC. To facilitate target cell imaging via the IncuCyte Live Cell Analysis Imaging System, these cell lines were stably labelled with nuclear mKate2 by lentiviral transduction with IncuCyte NucLight Red Lentivirus Reagent (Sartorius). Area under the curve (AUC) values were calculated by integrating the area under normalized target cell killing curves.

Expression Constructs: Lentiviral constructs were generated with bi-cistronic expression cassettes. The coding sequences for (i) a ROR1-specific R12 CAR, (ii) a P2A self-cleaving peptide, and (iii) EGFRt (a truncated EGFR having only Domains III and IV and the transmembrane domain; SEQ ID NO:4868) were linked in frame and placed under the control of an MND promoter. The R12 CAR was derived from the R12 anti-ROR1 antibody (Yang et al., PLoS One. (2011) 6:e21018) and contained one of the indicated spacers, a CD28-derived transmembrane domain, a 4-1BB costimulatory domain, and a CD3 zeta signaling domain.

Estimation of spacer length: When possible, spacer lengths were measured from crystal structures available on the RCSB PDB (Protein Data Bank; http://www.rcsb.org)) using the measurement tool in a molecular visualization software (e.g. PyMOL). Residues with unresolved crystallographic coordinates, consecutive segments of missing density, or spacers for which there are no publicly available crystal structures were each modeled as extended structures with each residue spanning 3.6 Å, the Nᵢ—Nᵢ₊₁ distance in the R conformation.

Cell Culture and Lentiviral Transduction: Pre-selected, cryopreserved primary human CD4+ and CD8+ T cells from normal donors were obtained from Bloodworks (Seattle WA). Human T cells were cultured in OpTmizer medium (Thermo Fisher) supplemented with Immune Cell Serum Replacement (Thermo Fisher), 2 mM L-glutamine (Gibco), 2 mM Glutamax (Gibco), 200 IU/ml IL-2 (R&D systems), 120 IU/ml IL-7 (R&D systems), and 20 IU/ml IL-15 (R&D systems). For lentiviral transduction, the T cells were stimulated with a 1:100 dilution of T cell TransAct (Miltenyi) for 30 hours. Virus was then added to the T cells for 24 hours. Stimulation and viral infection were then terminated by addition of 7 volumes of fresh media without TransAct, and cells were cultured for 7 additional days in Grex-24 plate (Wilson Wolf) prior to cryopreservation in CryoStor CS10 (STEMCELL Technologies) at 3×10⁷ cells/ml. All freshly thawed T cells were normalized for % CAR+ and total T cells by adding Mock (untransduced) T cells to appropriate samples prior to assay set-up.

CAR expression measurement via flow cytometry: To measure CAR expression levels and transduction efficiencies, roughly 0.5×10⁶ cells were pelleted after a 6-day production period following lentiviral transduction for flow cytometry analysis. Cells were resuspended in Fixable Viability Dye eFluor 780 (Invitrogen, Cat# 65-0865-14) in PBS for 10 minutes, then washed with Cell Staining Buffer (BioLegend, Cat# 420201).

R12 CARs scFv surface expression was detected using recombinant human ROR1-Fc chimera protein (R&D, Cat# 9490-RO-050) prelabeled with DyLight™ 650 Microscale Antibody Labeling Kit (ThermoFisher, Cat# 84536) diluted 1:500 in Cell Staining Buffer. Transduction efficiencies were determined by surface staining of transduction marker EGFRt using anti-hEGFR-BV421 (BioLegend, Cat# 352911) diluted 1:40 in Cell Staining Buffer. Cells were pelleted after a 20 minutes incubation in the dark, followed by 2× wash with Cell Staining Buffer. All flow cytometric analysis was done on a ZE5 Cell Analyzer (Biorad) and analyzed with FlowJo (Tree Star).

Assay set-up—target-independent cytokine secretion: Cryopreserved, transduced primary T cells expressing R12 CAR with indicated spacers were each thawed, counted, and resuspended to a cell density equal to 0.5×10⁶ CAR+ cells/ml in cell-assay media. A 100 μl volume, or 50,000 CAR+ cells, were added to a flat-bottom 96 well plate and incubated in a 37° C. incubator. After 24 hours, supernatant samples from each well was collected for IFNγ measurement according to manufacturer's protocol (Meso Scale Discovery).

Assay set-up—target-dependent cytokine secretion: Cryopreserved, transduced primary T cells expressing R12 CAR with indicated spacers were each thawed, counted, resuspended to a cell density equal to 0.5×10⁶ CAR+ cells/ml in cell-assay media, and a 100 μl volume, or 50,000 CAR+ cells, were added to a flat-bottom 96 well plate containing 50,000 H1975 cells and incubated at 37° C. in an IncuCyte Live Cell Analysis System (Sartorius). Each well was imaged every 4 hours for quantifying the number of H1975 cells to assess the kinetics of T cell cytotoxicity. After 24 hours, supernatant from each well was collected for IL-2 and IFNγ measurement according to manufacturer's protocol (Meso Scale Discovery). Target cell lysis was tracked over 4 days.

Assay set-up—serial killing: Cryopreserved, transduced primary T cells expressing R12 CAR with indicated spacers were each thawed, counted, resuspended to a cell density equal to 2×10⁶ CAR+ cells/ml in cell-assay media, and a 1 ml volume, or 0.2×10⁶ CAR+ cells, were added to a 24 well plate containing 0.2×10⁶ Jeko1 cells in 1 ml cell-assay media and incubated at 37C in an IncuCyte Live Cell Analysis System (Sartorius). Each well was imaged every 4 hours for quantifying the number of Jeko1 cells to assess the kinetics of T cell cytotoxicity. At 68 hr and 148 hr time points, 0.5 ml was transferred from the previous co-culture plate to a new 24 well plate containing 0.2×10⁶ fresh Jeko1 cells in 1.5 ml cell-assay media. The numbers of remaining Jeko1 cells over the 3 rounds of co-culture with R12 CART cells were tracked and normalized to the first time point of each round (i.e. 0 hr, 68 hr and 148 hr).

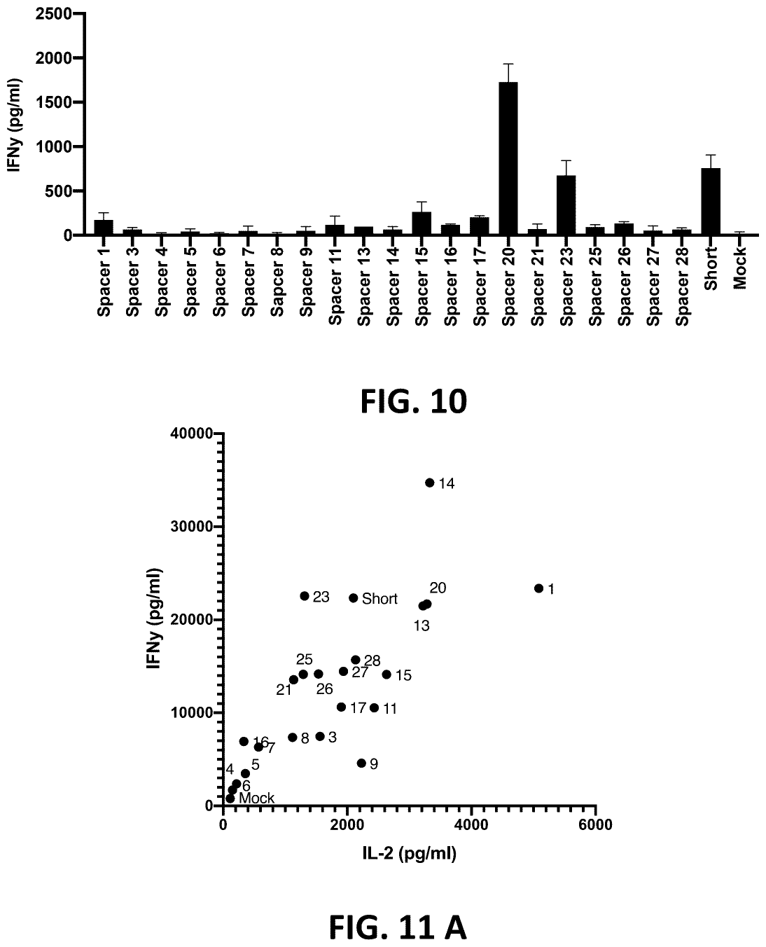

Results: The results show varying levels of target-independent IFN-γ secretion by the T cells expressing different spacers as a component of the CAR design (FIG. 10). Spacers 20 and 23 in particular induced R12 CART cells to behave in an overtly autoactivating manner, leading to increased levels of IFN-γ detection. The benchmark spacer, ‘Short’, also secreted elevated levels of IFN-γ in the absence of the target antigen, human ROR1. The rest of the indicated spacers exhibited low levels of target-independent activation in R12 CART cells.

The results also indica

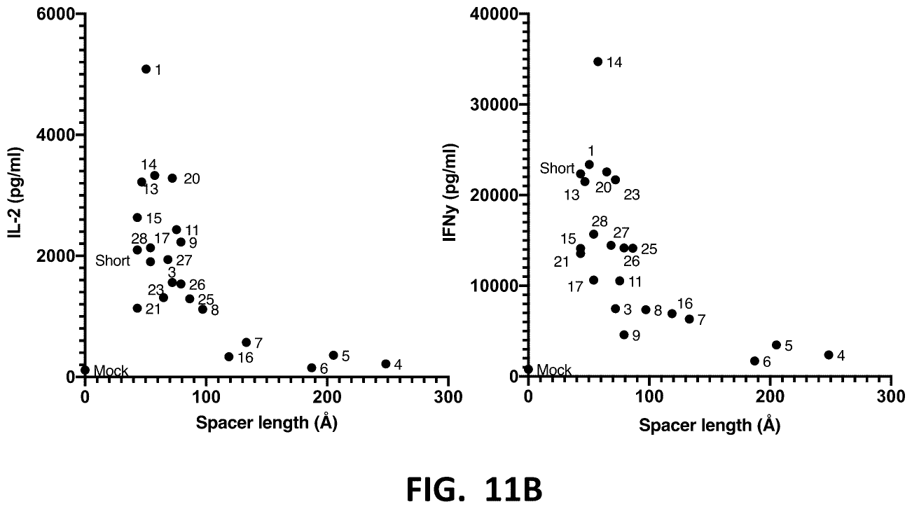

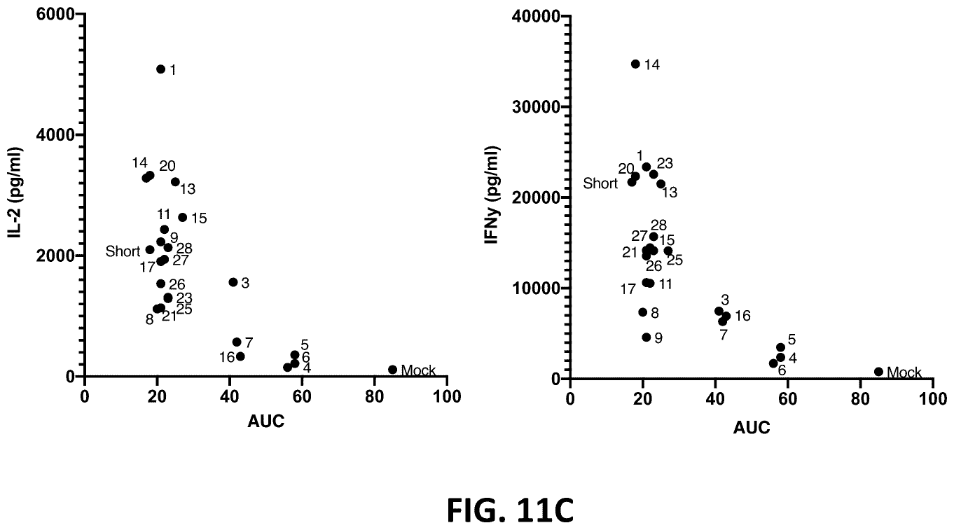

te varying levels of target-dependent IFN-γ and IL-2 secretion by the T cells expressing different spacers as a component of the CAR design (FIG. 11A). Spacer 1 in particular elicited R12 CART cells to secrete the highest level of IL-2 while spacer 14 in particular caused them to secrete the highest level of IFN-7. In the context of spacer lengths, both IFN-γ and IL-2 output by the T cells were highest when CAR spacer lengths were between 43.2 Å and 75.6 Å (FIG. 11B). The full range of spacer lengths tested using R12 CAR was 43.2 Å ˜248.4 Å.

The R12 CART with the benchmark spacer, ‘Short’, that also secreted high levels of IFN-γ and IL-2 following exposure to H1975 cells contains a spacer that is 43.2 Å long. The cytokine output by the R12 CART cells correlated with their target lytic capabilities, as measured by their associated AUC values (FIG. 11C).

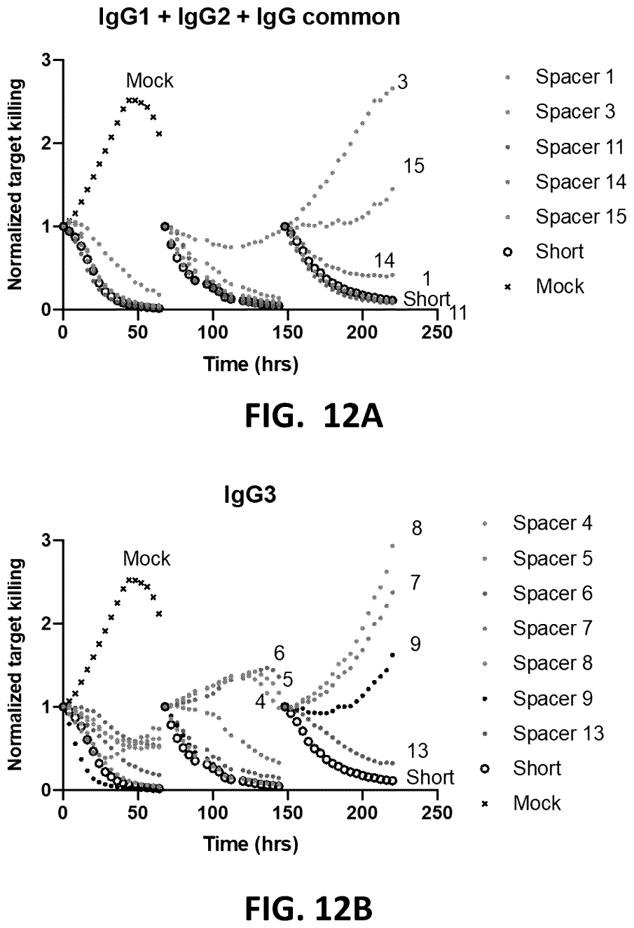

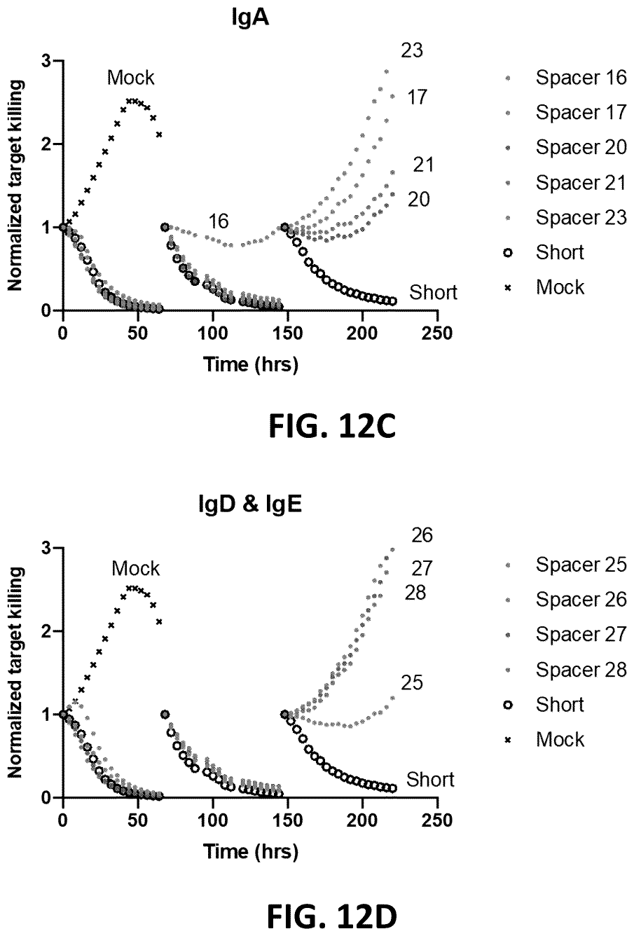

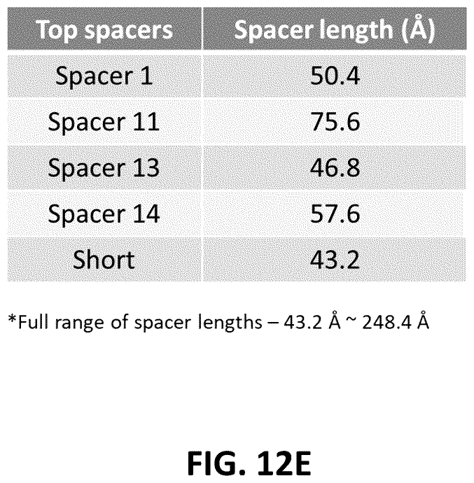

The cytokine secretion profile correlated with the prolonged cytotoxic capabilities by the R12 CART cells as five of the spacers (Spacers 1, 11, 13, 14, and Short) that were between 43.2 Å and 75.6 Å in length were able to sustain potent target lytic activities over the 3 rounds of target exposure (FIGS. 12A, 12B, 12C, 12D, and 12E). The full range of spacer lengths tested using R12 CAR was 43.2 Å ˜248.4 Å. The R12 CART with the benchmark spacer, ‘Short’, that also demonstrated sustained cytotoxic activity against Jeko1 cells contained a spacer that was 43.2 Å long.

TABLE 21
Complete ROR1 CAR sequences. The spacer used in each
ROR1 CAR is indicated in
the first column. Spacer numbering follows FIG. 9 numbering.

| | Complete CAR SEQ ID NO | Complete CAR SEQ ID NO |
|---|---|---|
| Spacer | without optional linker | with optional linker |
| 1 | 5072 | 5049 |
| 11 | 5073 | 5065 |
| 13 | 5074 | 5066 |
| 14 | 5075 | 5067 |

TABLE 21A
Components of R12 CAR sequences w/ Spacer 1

| | |
|---|---|
| SEQ ID NO | Description |

R12 CAR with spacer 1 CAR against ROR1

| | |
|---|---|
| 4874 | Signal peptide/20aa |
| 4875 | scFv/248aa |
| 4830 | Spacer 1 |
| 4868 | Transmembrane domain/28aa |
| 4869 | 4-1BB costimulatory domain/42aa |
| 4870 | CD3z/112aa |
| 4871 | P2A/21aa |
| 4872 | EGFRt/357aa (includes 22aa signal peptide set |
| | forth in SEQ ID NO:4865) |

R12 CAR comprising spacer 1 targeting ROR1 (with optional
linker)

| | |
|---|---|
| 4874 | Signal peptide/20aa |
| 4875 | scFv/245aa |
| 4818 | Optional GGGSG linker |
| 4830 | Spacer 1 |
| 4868 | Transmembrane domain/28aa |
| 4869 | 4-1BB costimulatory domain/42aa |
| 4870 | CD3z/112aa |
| 4871 | P2A/21aa |
| 4865 | Signal peptide/22aa |
| 5064 | EGFRt/335 aa |

TABLE 21B
Components of R12 CAR sequences w/ Spacer 2

| | |
|---|---|
| SEQ ID NO | Description |

R12 CAR comprising spacer 2 targeting ROR1

| | |
|---|---|
| 4874 | Signal peptide/20aa |
| 4875 | scFv/248aa |
| 4831 | Spacer 2 |
| 4868 | Transmembrane domain/28aa |
| 4869 | 4-1BB costimulatory domain/42aa |
| 4870 | CD3z/112aa |
| 4871 | P2A/21aa |
| 4865 | Signal peptide/22aa |
| 5064 | EGFRt/335aa |

R12 CAR comprising spacer 2 targeting ROR1 (with optional linker)

| | |
|---|---|
| 4874 | Signal peptide/20aa |
| 4875 | scFv/248aa |
| 4818 | Optional GGGSG linker |
| 4831 | Spacer 2 |
| 4868 | Transmembrane domain/28aa |
| 4869 | 4-1BB costimulatory domain/42aa |
| 4870 | CD3z/112aa |
| 4871 | P2A/ 21aa |
| 4865 | Signal peptide/22aa |
| 5064 | EGFRt/335aa |

TABLE 21C
Components of R12 CAR sequences w/ Spacer 3

| | |
|---|---|
| SEQ ID NO | Description |

R12 CAR comprising spacer 3 targeting ROR1

| | |
|---|---|
| 4874 | Signal peptide/20aa |
| 4875 | scFv/248aa |
| 4832 | Spacer 3 |
| 4868 | Transmembrane domain/28aa |
| 4869 | 4-1BB costimulatory domain/42aa |
| 4870 | CD3z/112aa |
| 4871 | P2A/21aa |
| 4865 | Signal peptide/22aa |
| 5064 | EGFRt/335aa |

R12 CAR comprising spacer 3 targeting ROR1 (with optional linker)

| | |
|---|---|
| 4874 | Signal peptide/20aa |
| 4875 | scFv/248aa |
| 4818 | Optional GGGSG linker |
| 4832 | Spacer 3 |
| 4868 | Transmembrane domain/28aa |
| 4869 | 4-1BB costimulatory domain/42aa |
| 4870 | CD3z/112aa |
| 4871 | P2A/21aa |
| 4865 | Signal peptide/22aa |
| 5064 | EGFRt/335aa |

### Example 2

### Spacer Dependent Activity by CD19 CAR-T Cells

This study describes the effects of various Ig-derived spacers on inducing CAR-T auto-activation, as well as human CD 19 directed cytokine release and potency. The reference CAR sequence used to test different spacer lengths and its constituent elements is shown in TABLE 22 below. The same experimental methods used in example 1 were used in the present example.

TABLE 22
FMC63 Reference CAR sequences

| | |
|---|---|
| SEQ ID NO | Description |

FMC63 CAR comprising short reference spacer targeting CD19
SEQ ID NO:5076

| | |
|---|---|
| 5076 | Full sequence/837aa |
| 4874 | Signal peptide/20aa/aa 1-20 |
| 4866 | scFv/245aa/aa 21-268 |
| 4876 | Spacer/12aa/aa 266-277 |
| 4877 | Transmembrane domain/28aa/aa 278-305 |
| 4878 | 4-1BB costimulatory domain/42aa/aa 306-347 |
| 4879 | CD3z/112aa/aa 348-459 |
| 4880 | P2A/21aa/aa 460-480 |
| 4865 | Signal peptide/22aa |
| 5064 | EGFRt/335aa/aa 503-837 |

FMC63 CAR components comprising short reference spacer targeting
CD19 (with optional linker)

| | |
|---|---|
| 4874 | Signal peptide/20aa/aa 1-20 |
| 4866 | scFv/245aa/aa 21-265 |
| 5088 | Optional GGGGSG linker |
| 4876 | Spacer/12aa |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 4881 | EGFRt/335aa |

CD 19-specific FMC63 CARs were delivered using lentiviral constructs coding for bi-cistronic expression cassettes. The coding sequences for (i) a CD19-specific FMC63 CAR (ii) a P2A self-cleaving peptide, and (iii) EGFRt (a truncated EGFR having only Domains III and IV and the transmembrane domain; SEQ TD NO:4877) were linked in frame and placed under the control of an MND promoter. The FMC63 CARs were derived from the FMC63 anti-CD19 antibodies and contained one of the indicated spacers, a CD28-derived transmembrane domain, a 4-1 costimulatory domain, and a CD3 zeta signaling domain. Exemplary CAR constructs, e.g., with Spacer 1, Spacer 2, and Spacer 3, are shown below.

TABLE 23
Complete CD19 CAR sequences. The spacer used in each CD19 CAR is indicated in the
first column. Spacer numbering follows FIG. 9 numbering.

| | Complete CAR SEQ ID NO | Complete CAR SEQ ID NO |
|---|---|---|
| Spacer | without optional linker | with optional linker |
| 7 | SEQ ID NO:5077 without optional GGGGSG | 5077 |
| | linker of SEQ ID NO: 5088 before Spacer 7 | |
| 8 | SEQ ID NO:5078 without optional GGGGSG | 5078 |
| | linker of SEQ ID NO: 5088 before Spacer 8 | |
| 9 | SEQ ID NO:5079 without optional GGGGSG | 5079 |
| | linker of SEQ ID NO: 5088 before Spacer 9 | |
| 10 | SEQ ID NO:5080 without optional GGGGSG | 5080 |
| | linker of SEQ ID NO: 5088 before Spacer 10 | |
| 11 | SEQ ID NO:5081 without optional GGGGSG | 5081 |
| | linker of SEQ ID NO: 5088 before Spacer 11 | |
| 14 | SEQ ID NO:5082 without optional GGGGSG | 5082 |
| | linker of SEQ ID NO: 5088 before Spacer 14 | |
| 16 | SEQ ID NO:5083 without optional GGGGSG | 5083 |
| | linker of SEQ ID NO: 5088 before Spacer 16 | |
| 28 | SEQ ID NO:5084 without optional GGGGSG | 5084 |
| | linker of SEQ ID NO: 5088 before Spacer 24 | |

TABLE 23A
Components of FMC63 CAR sequences with Spacer 1

| | |
|---|---|
| SEQ ID NO | Description |

FMC63 CAR comprising spacer 1 targeting CD19

| | |
|---|---|
| 4874 | Signal peptide/20aa |
| 4866 | scFv/245aa |
| 4830 | Spacer 1 |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 5064 | EGFRt/335aa |

FMC63 CAR comprising spacer 1 CAR targeting CD19 with optional
linker

| | |
|---|---|
| 4874 | Signal peptide/20aa |
| 4866 | scFv/245aa |
| 5088 | Optional GGGGSG linker |
| 4830 | Spacer 1 |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 5064 | EGFRt/335aa |

TABLE 23B
Components of FMC63 CAR sequences with Spacer 2

| | |
|---|---|
| SEQ ID NO | Description |

FMC63 CAR comprising spacer 2 targeting CD19

| | |
|---|---|
| 4874 | Signal peptide/20aa |
| 4866 | scFv/245aa |
| 4831 | Spacer 2 |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 5064 | EGFRt/335aa |

FMC63 CAR comprising spacer 2 targeting CD19 with optional
linker

| | |
|---|---|
| 4874 | Signal peptide/20aa |
| 4866 | scFv/245aa |
| 5088 | Optional GGGGSG linker |
| 4831 | Spacer 2 |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 5064 | EGFRt/335aa |

TABLE 23C
Components of FMC63 CAR sequences with Spacer 3

| | |
|---|---|
| SEQ ID NO | Description |

FMC63 CAR comprising spacer 3 targeting CD19

| | |
|---|---|
| 4874 | Signal peptide/20aa |
| 4866 | scFv/245aa |
| 4832 | Spacer 3 |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 5064 | EGFRt/335aa |

FMC63 CAR comprising spacer 3 targeting CD19 with optional linker

| | |
|---|---|
| 4874 | Signal peptide/20aa |
| 4866 | scFv/245aa |
| 5088 | Optional GGGGSG linker |
| 4832 | Spacer 3 |
| 4877 | Transmembrane domain/28aa |
| 4878 | 4-1BB costimulatory domain/42aa |
| 4879 | CD3z/112aa |
| 4880 | P2A/21aa |
| 4865 | Signal peptide/22aa |
| 5064 | EGFRt/335aa |

FMC63 CARs scFv surface expression was detected using an anti-ID-AF647 (Lyell) diluted 1:2000 in Cell Staining Buffer. Transduction Efficiency was determined by surface staining of transduction marker EGFRt using anti-hEGFR-BV421 (BioLegend, Cat# 352911) diluted 1:40 in Cell Staining Buffer. Cells were pelleted after a 20 minutes incubation in the dark, followed by 2× wash with Cell Staining Buffer. All flow cytometric analysis was done on a ZE5 Cell Analyzer (Biorad) and analyzed with FlowJo (Tree Star).

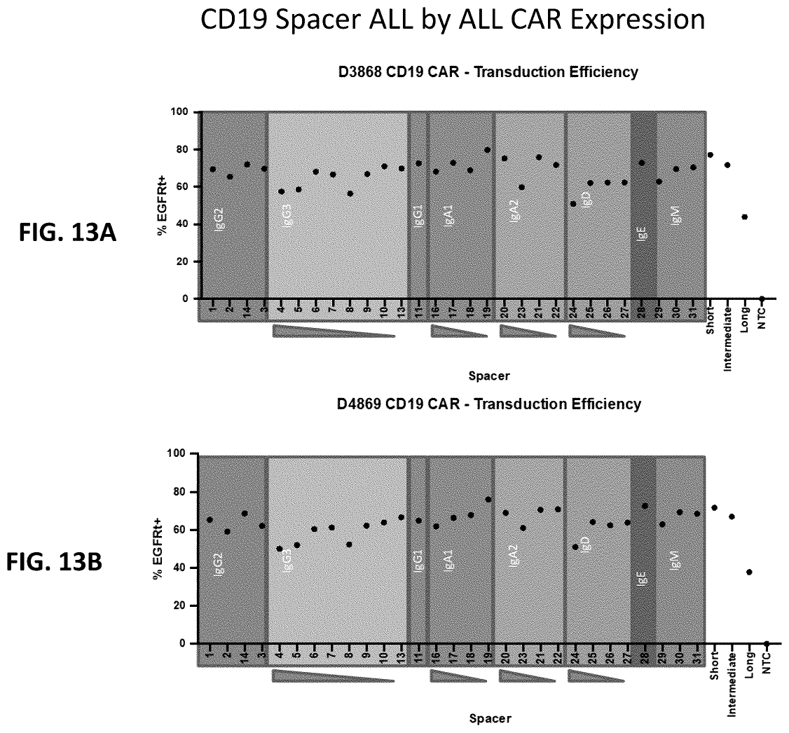

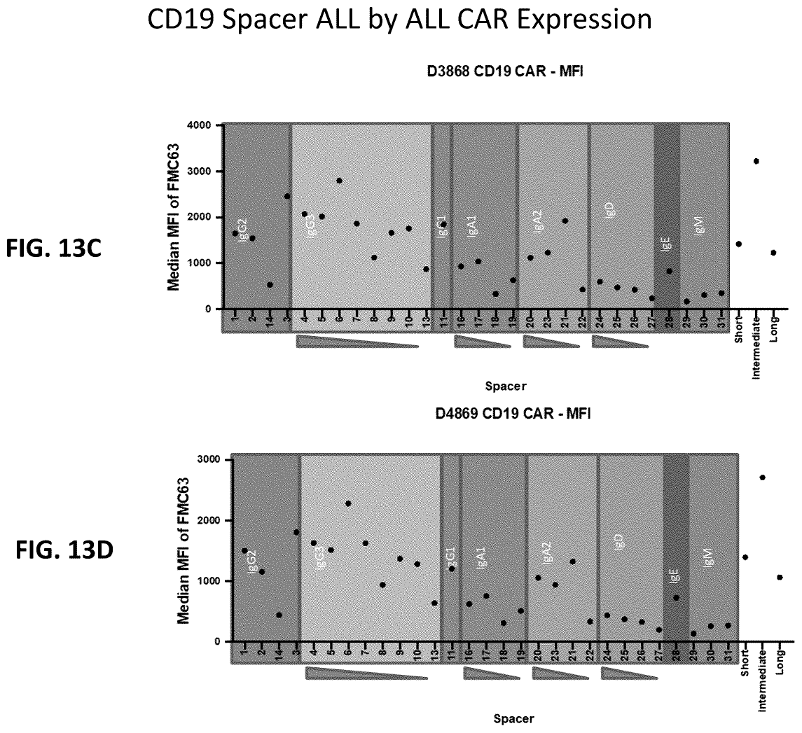

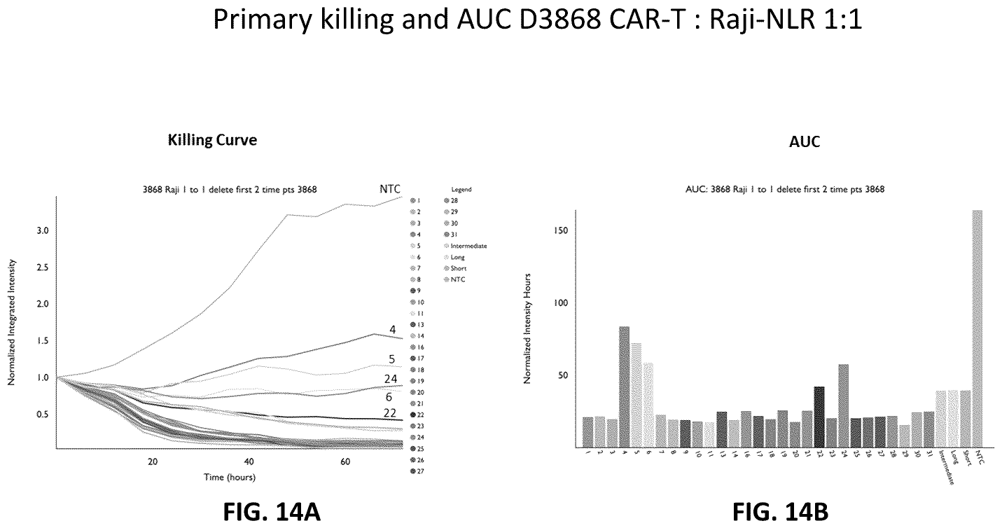

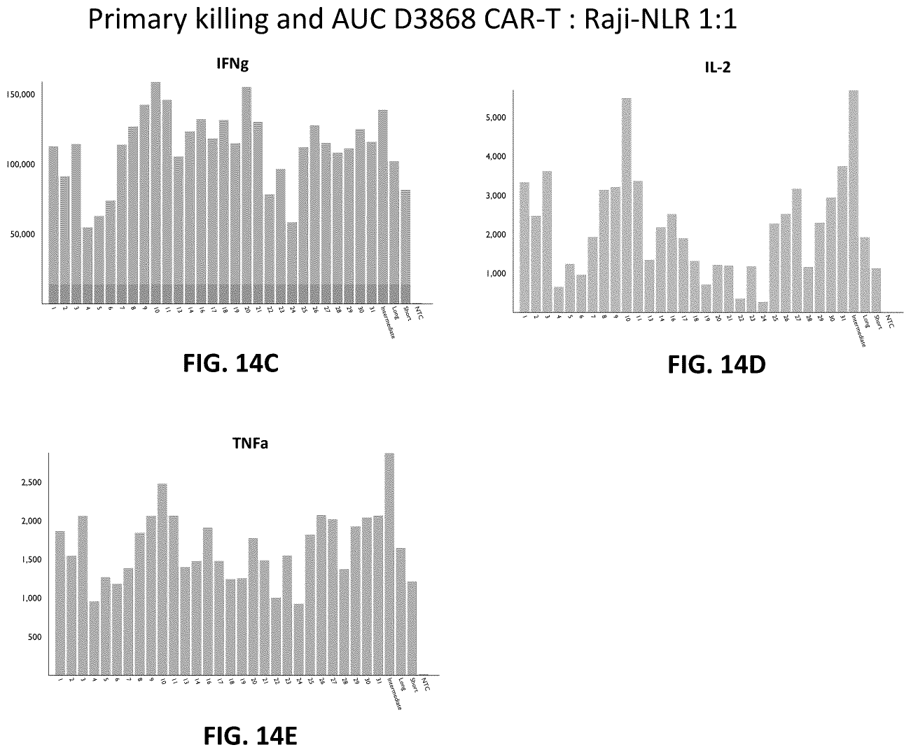

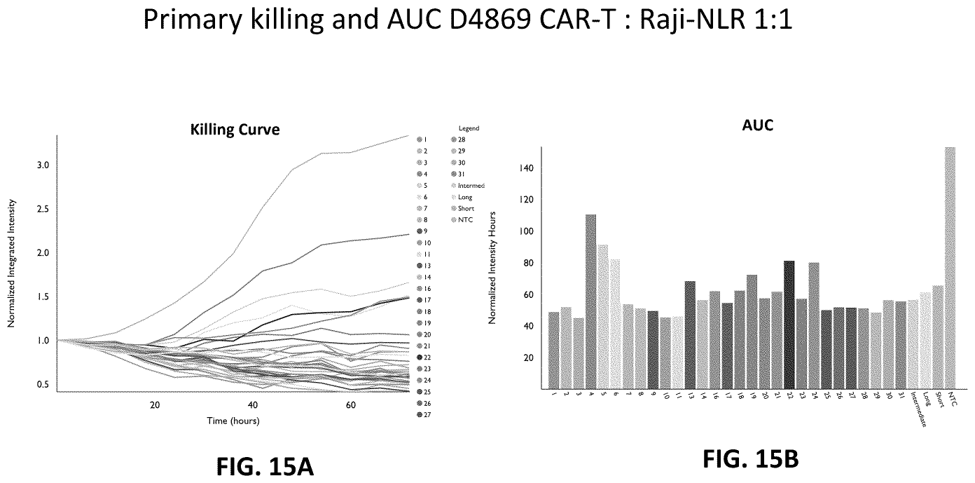

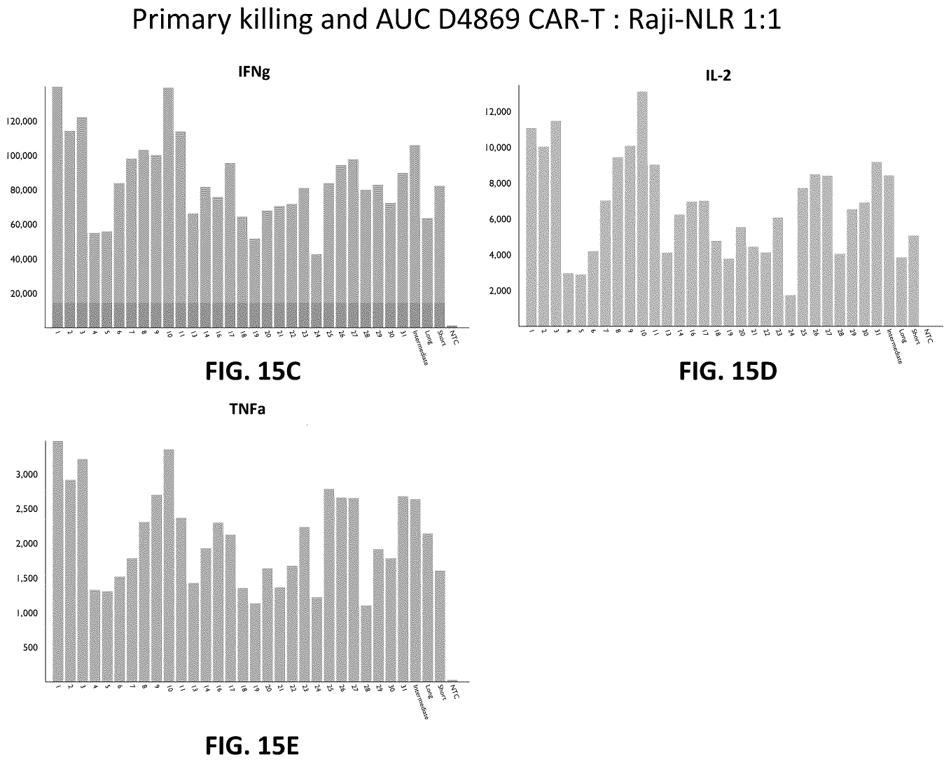

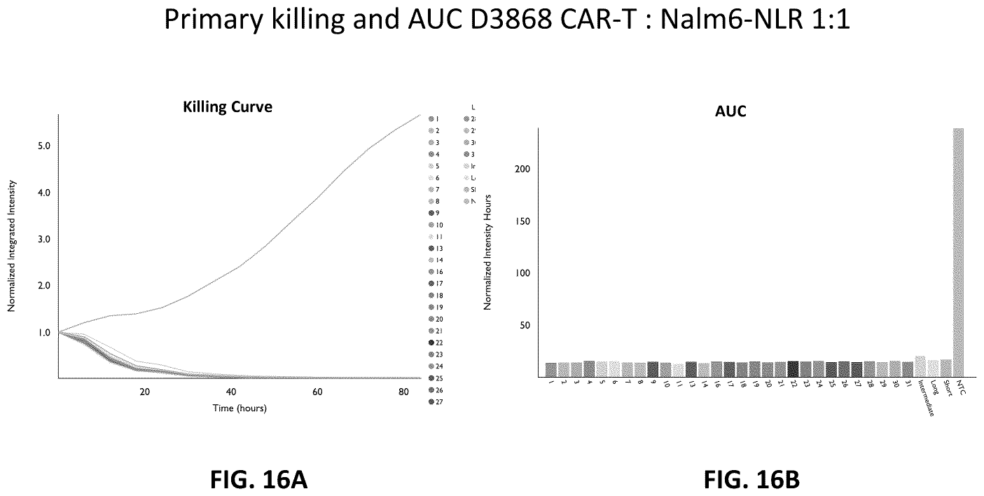

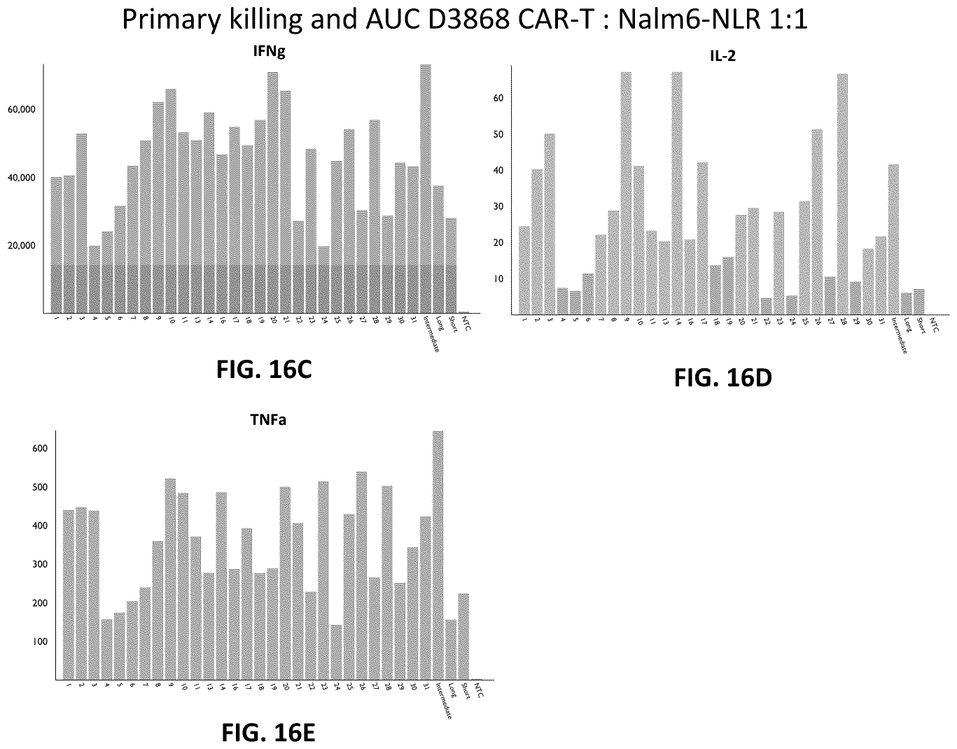

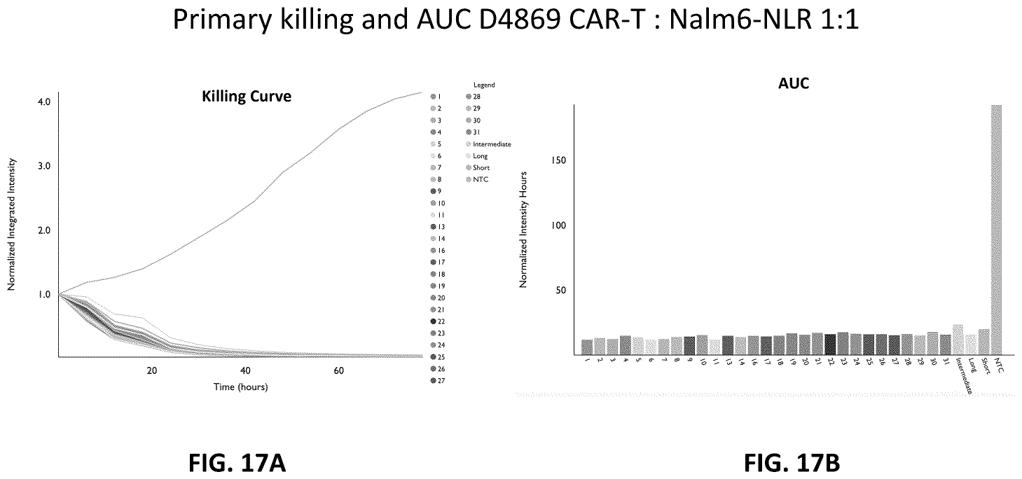

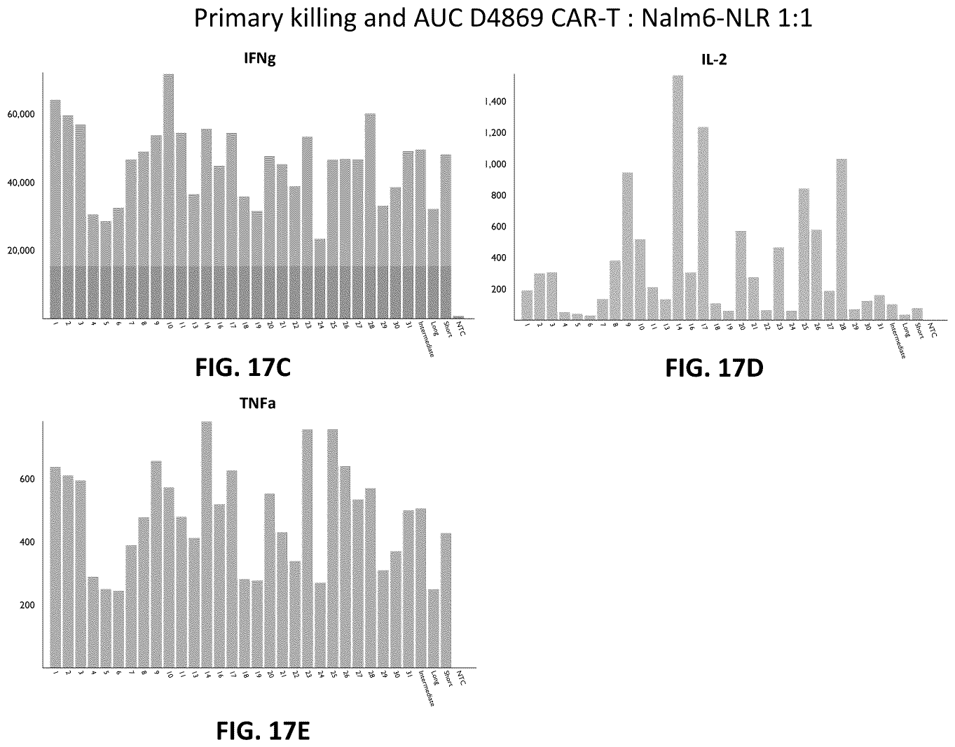

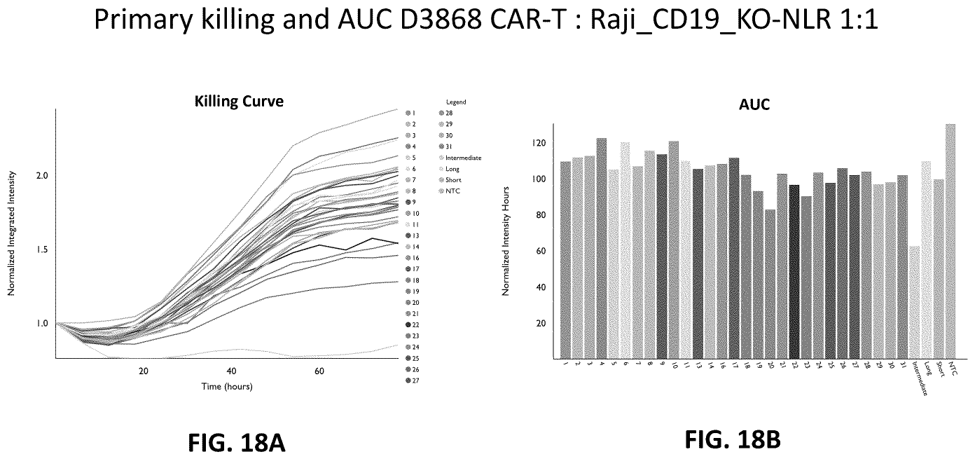

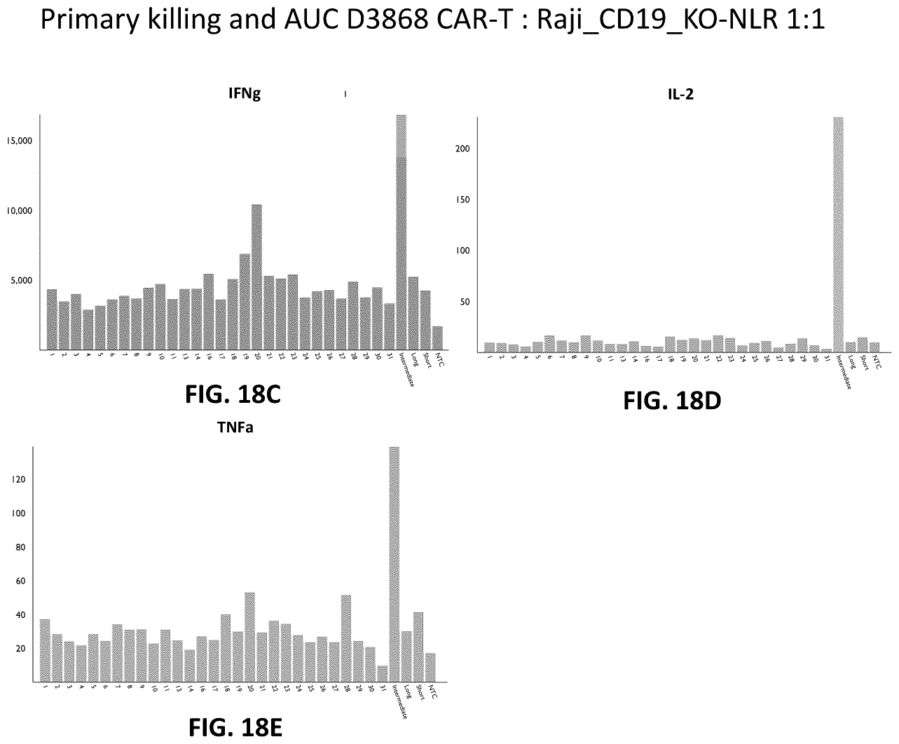

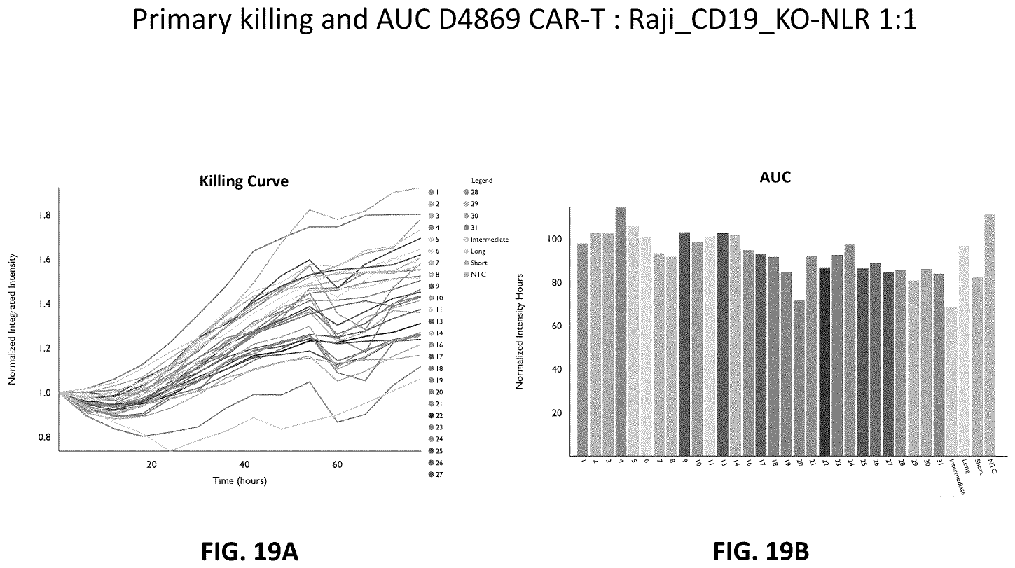

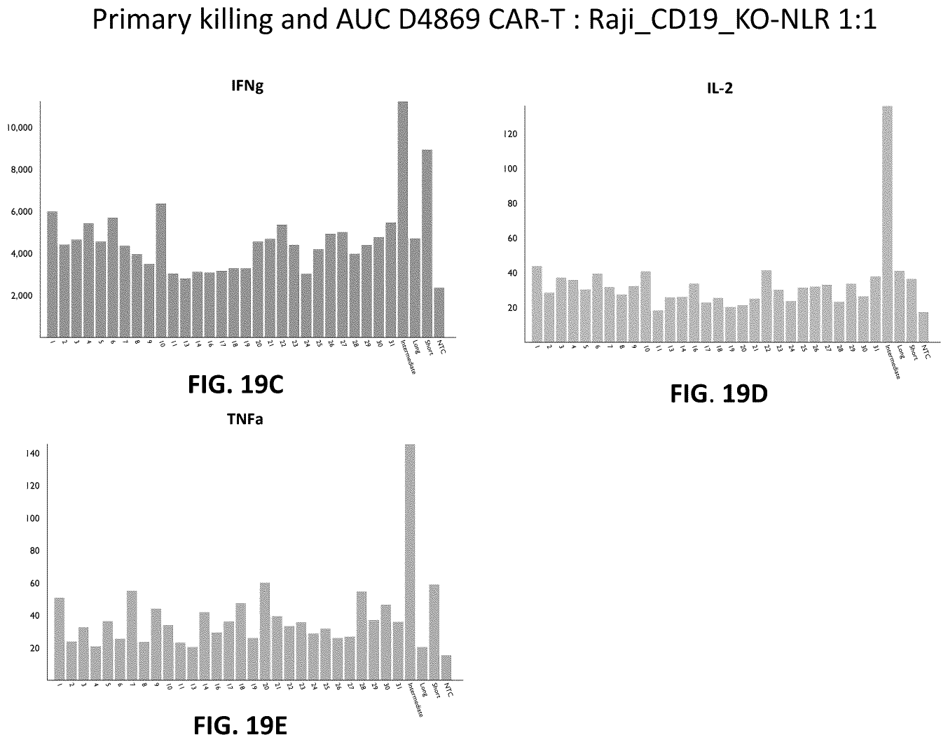

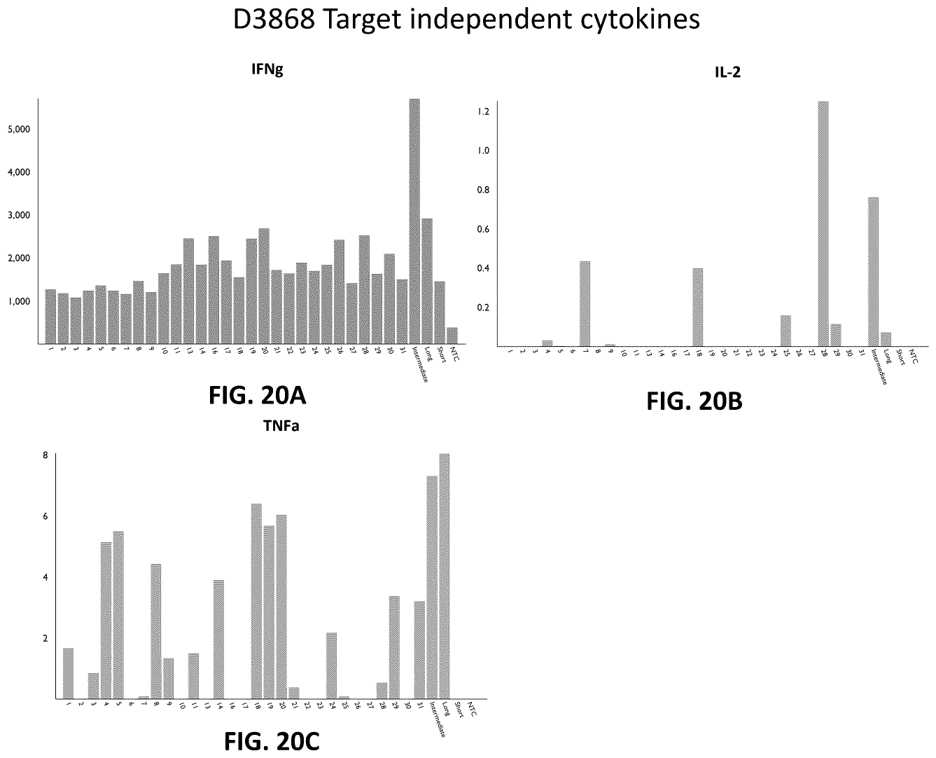

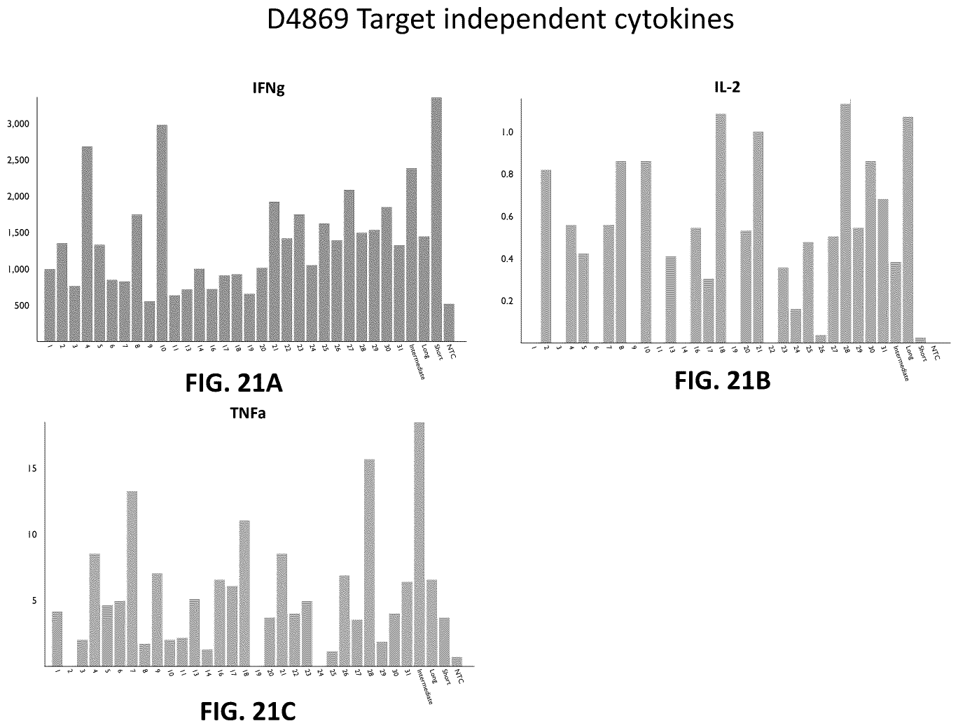

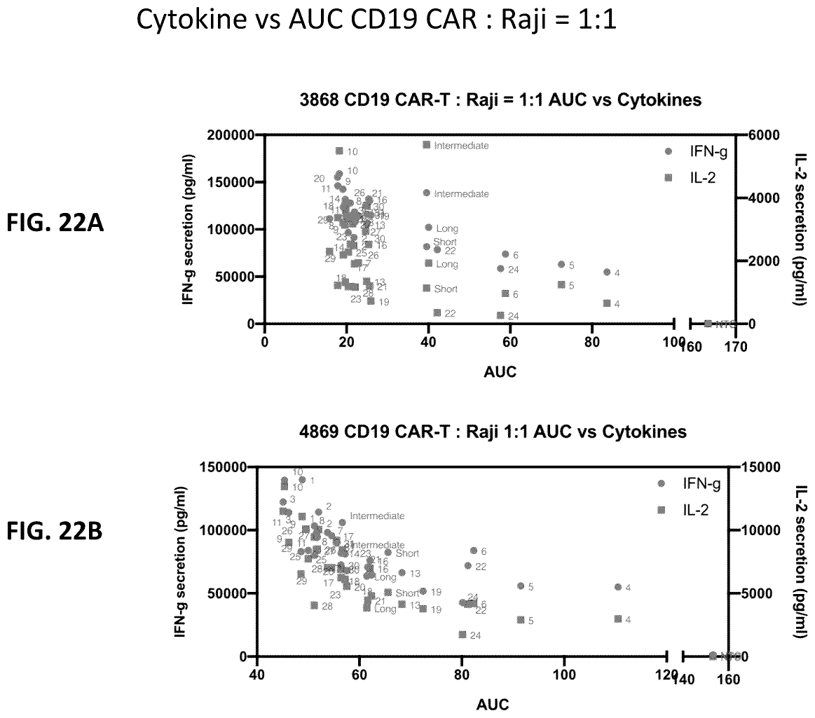

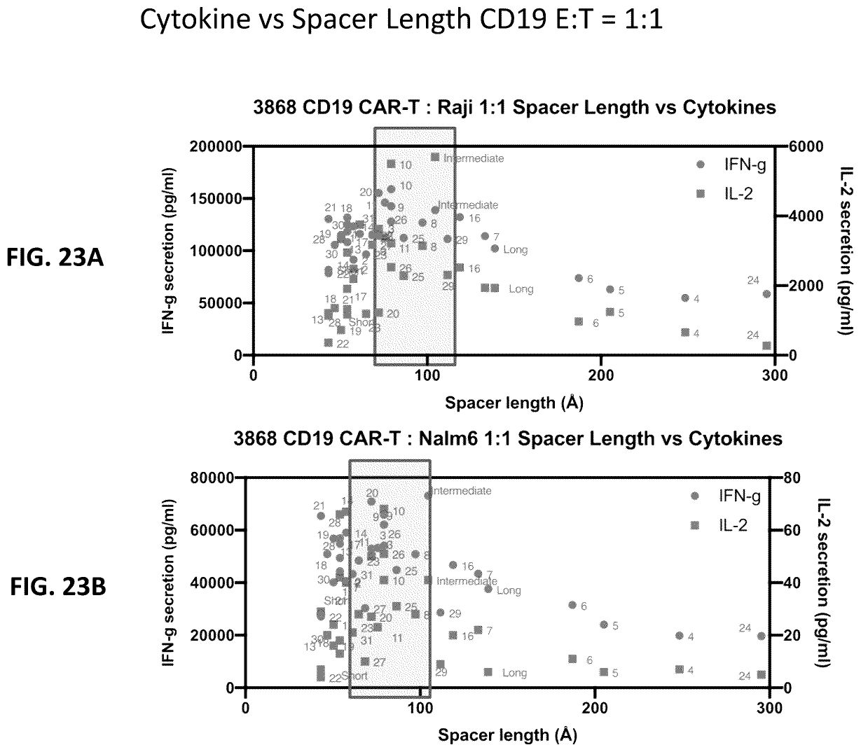

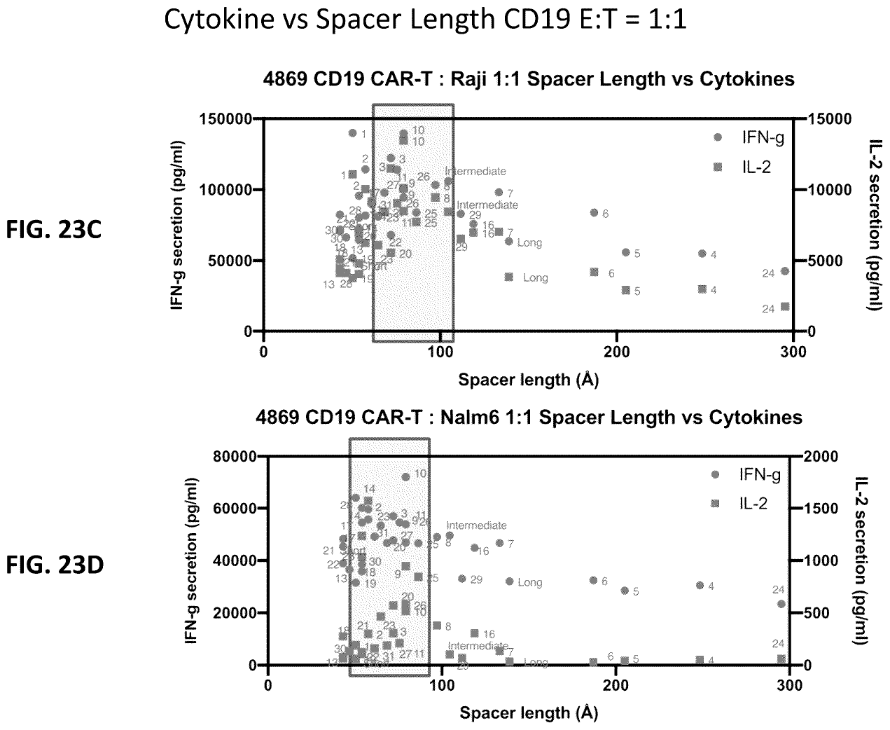

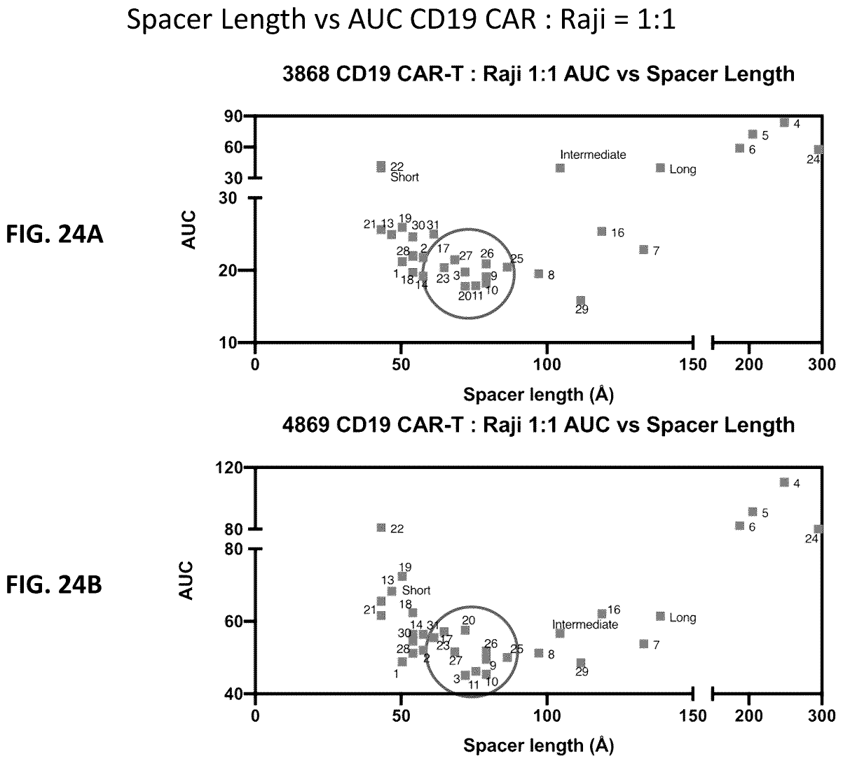

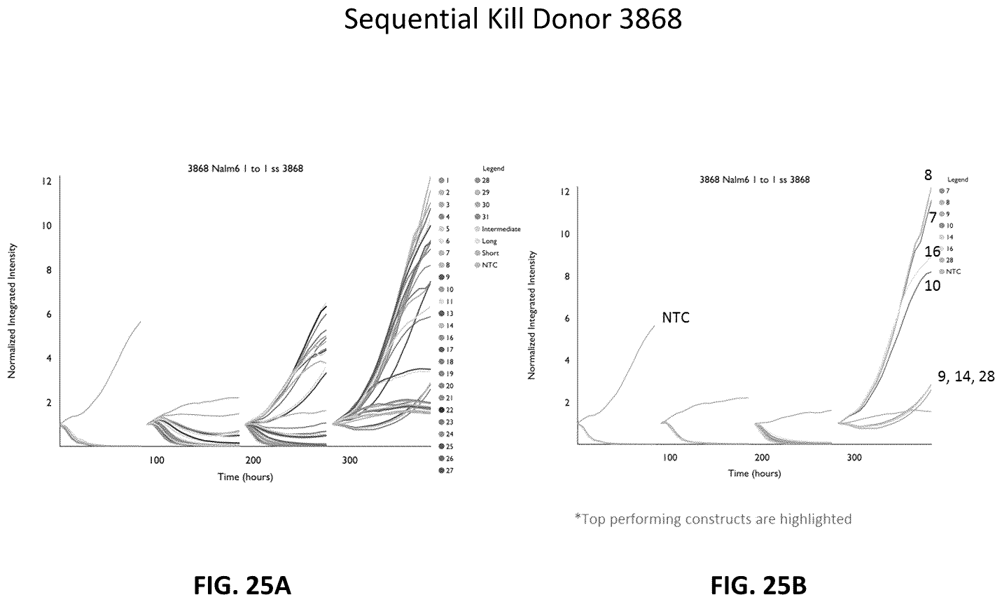

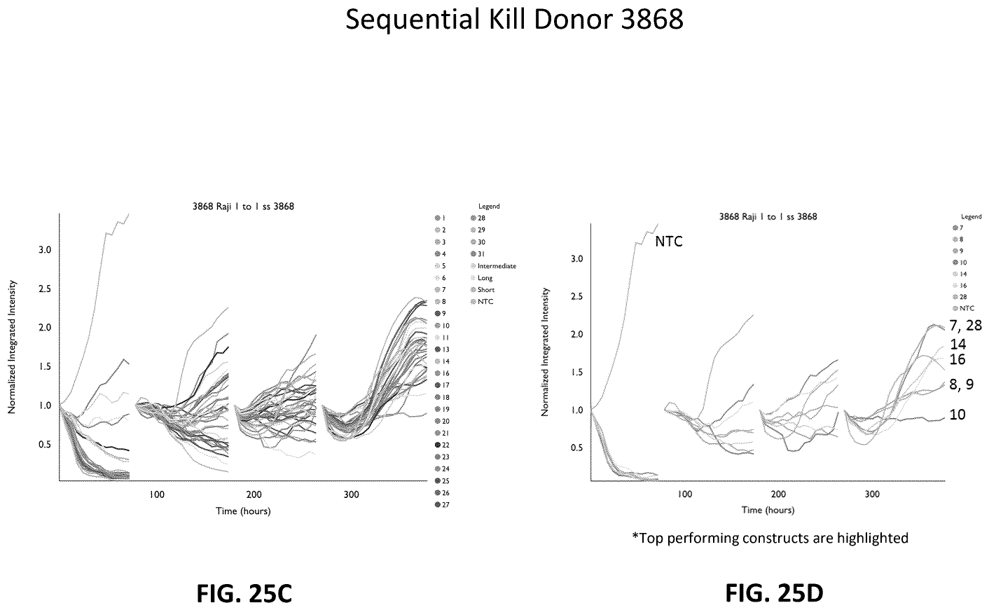

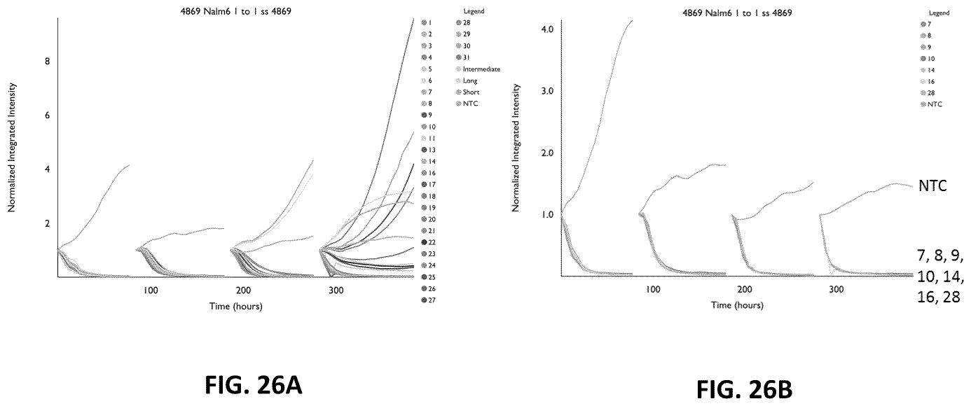

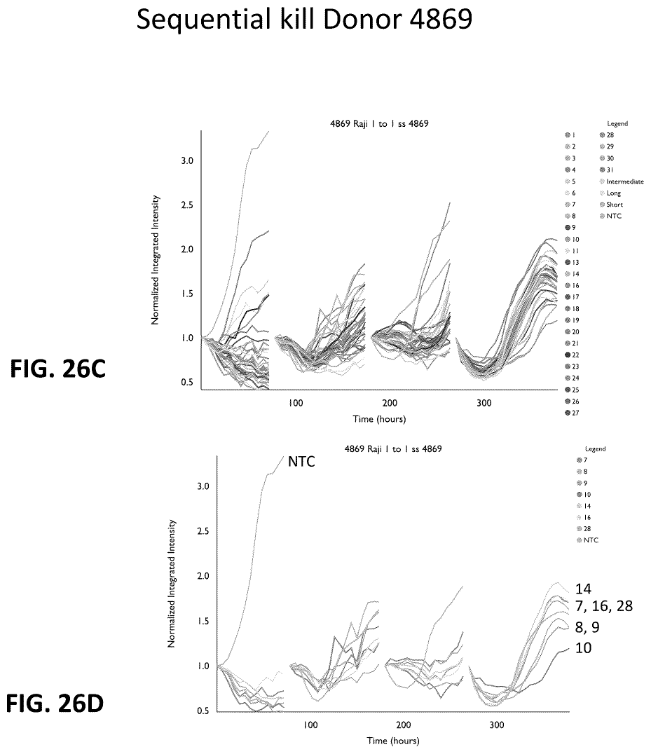

Healthy donor T cells were transduced with lentiviruses encoding FMC63 CARs comprising Ig-derived spacers to a transduction level of between 40% and 80% based on EGFRt staining (FIG. 13A, FIG. 13C), and their CAR expression levels were assessed by staining with an anti-FMC63 antibody conjugated to AlexaFluor647 (FIG. 13B, FIG. 13D). CART cells were challenged with Raji-NLR (FIGS. 13A-D, 14A-E), Nalm6-NLR (FIGS. 15A-E, 16A-E), and Raji_CD19_KO-NLR (FIGS. 18A-E, 19A-E) to determine killing kinetics and target-dependent cytokine production. CART cells were also cultured in the absence of target cells to assess target-independent cytokine production, which is an indicator of autoactivation. Target-independent cytokine secretion was generally low (FIGS. 20A-C, 21A-C). The amount of cytokine production after 24-hr post co-culture correlated with faster killing kinetics, with spacers 7, 8, 9, 10, 11, 14, 16, and 28 performing especially well (FIGS. 22A-B). IFN-γ and IL-2 production were highest when CAR spacer lengths were between 50 Å and 110 Å (FIGS. 23A-D). Similarly, short term cytotoxicity was effective when spacer lengths were between 50 Å and 110 Å (FIGS. 24A-B). We observed that FMC63 CART cells comprising spacers 7, 8, 9, 10, 14, 16, and 28 were able to sustain potent target lytic activities over several rounds of target exposure (FIGS. 25A-D, 26A-D).

### Example 3

### Spacer-Dependent Activity of Her2 CAR-T Cells

This study describes the effects of various Ig-derived spacers on inducing CAR-T auto-activation, as well as Her2 directed cytokine release and potency. The same experimental methods used in example 1 were used in the present example.

Anti-Her2-derived CARs were delivered using lentiviral constructs coding for bi-cistronic expression cassettes. The coding sequences for (i) an anti-Her2-derived CAR (ii) a P2A self-cleaving peptide, and (iii) EGFRt (a truncated EGFR having only Domains III and IV and the transmembrane domain) were linked in frame and placed under the control of an MND promoter. The anti-Her2 CARs comprised scFvs derived from the trastuzumab anti-Her2 antibody and contained one of the indicated spacers, a CD28-derived transmembrane domain, a 4-1BB costimulatory domain, and a CD3 zeta signaling domain.

Anti-Her2 CARs scFv surface expression was detected using Protein L-Biotin (Thermo Scientific, Cat# 29997) diluted 1:6000 in Cell Staining Buffer, followed by Strepavidin-BV421 (BioLegend, Cat# 405226) diluted 1:400 in Cell Staining Buffer. Transduction Efficiency was determined by surface staining of transduction marker EGFRt using anti-hEGFR-PE (BioLegend, Cat# 252903), diluted 1:40 in Cell Staining Buffer.

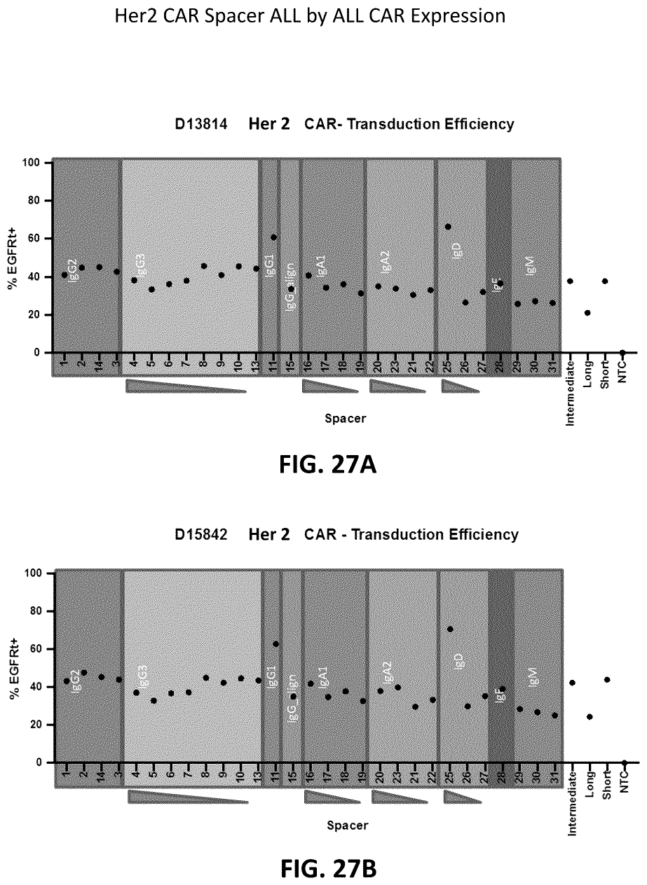

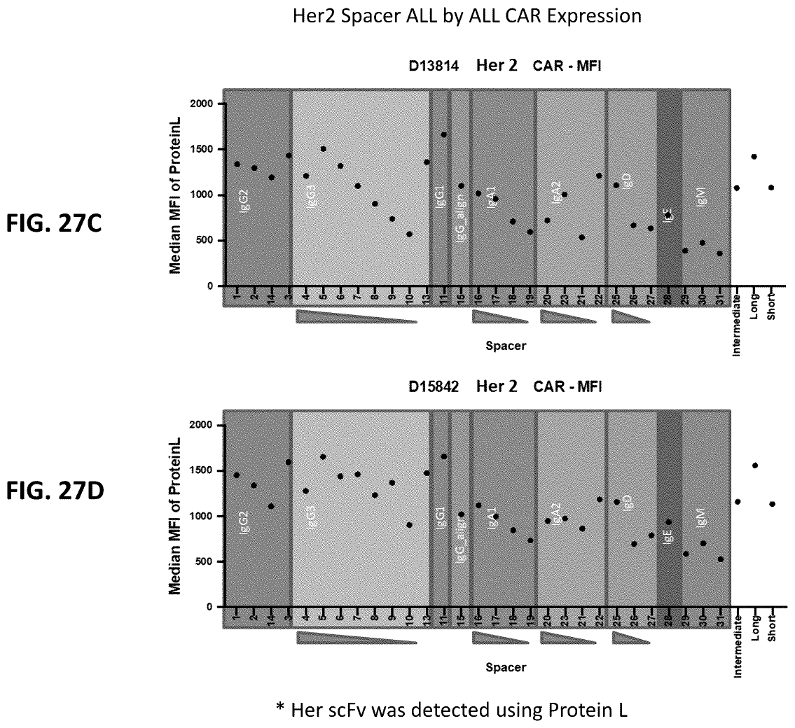

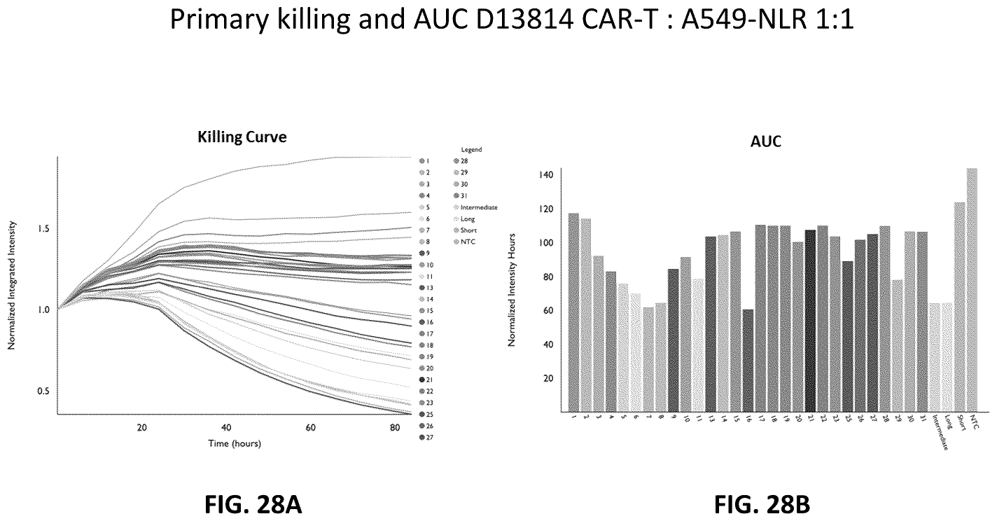

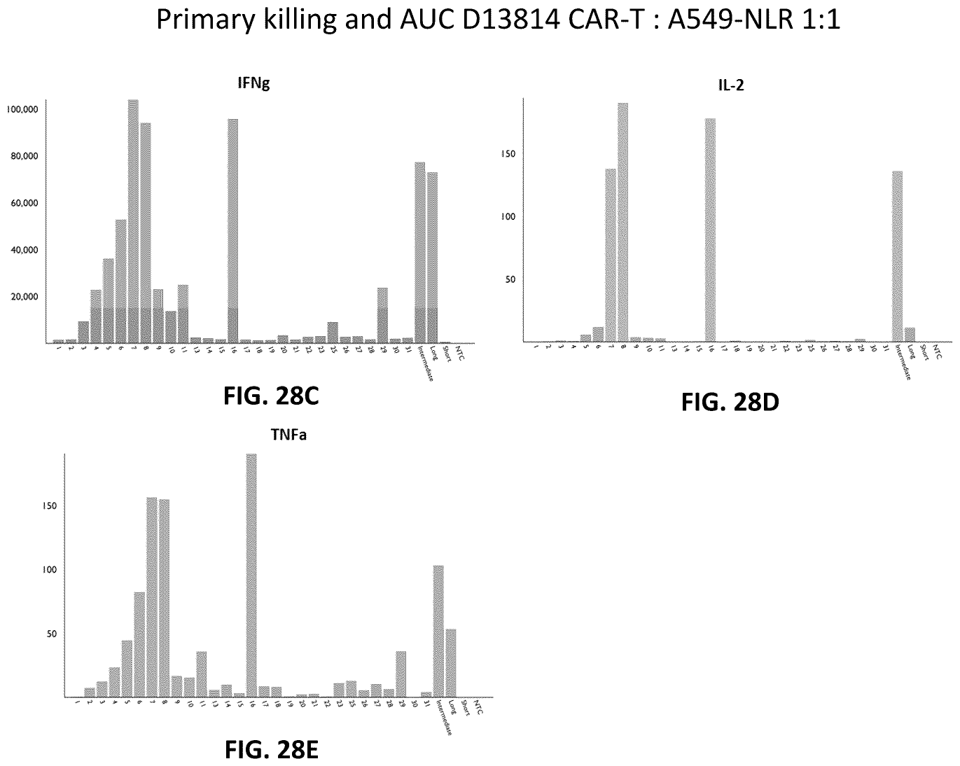

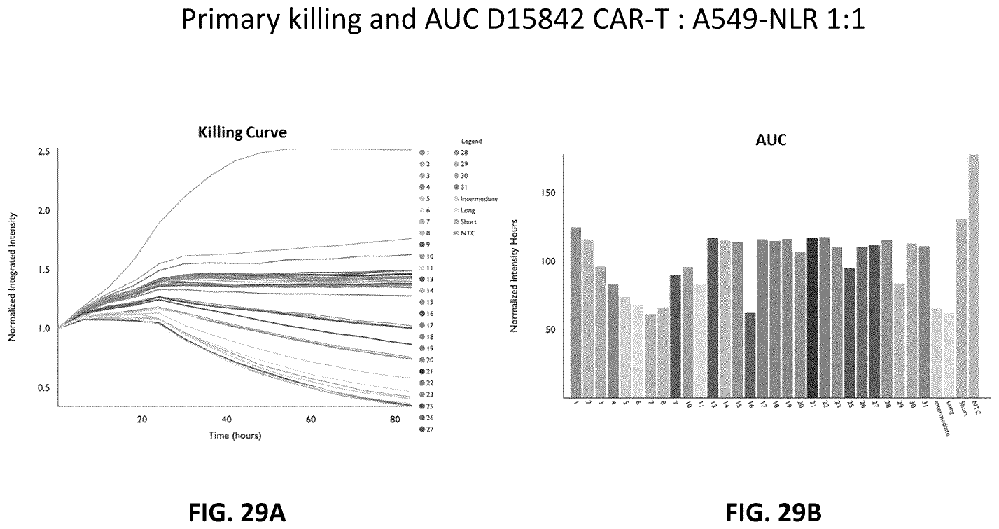

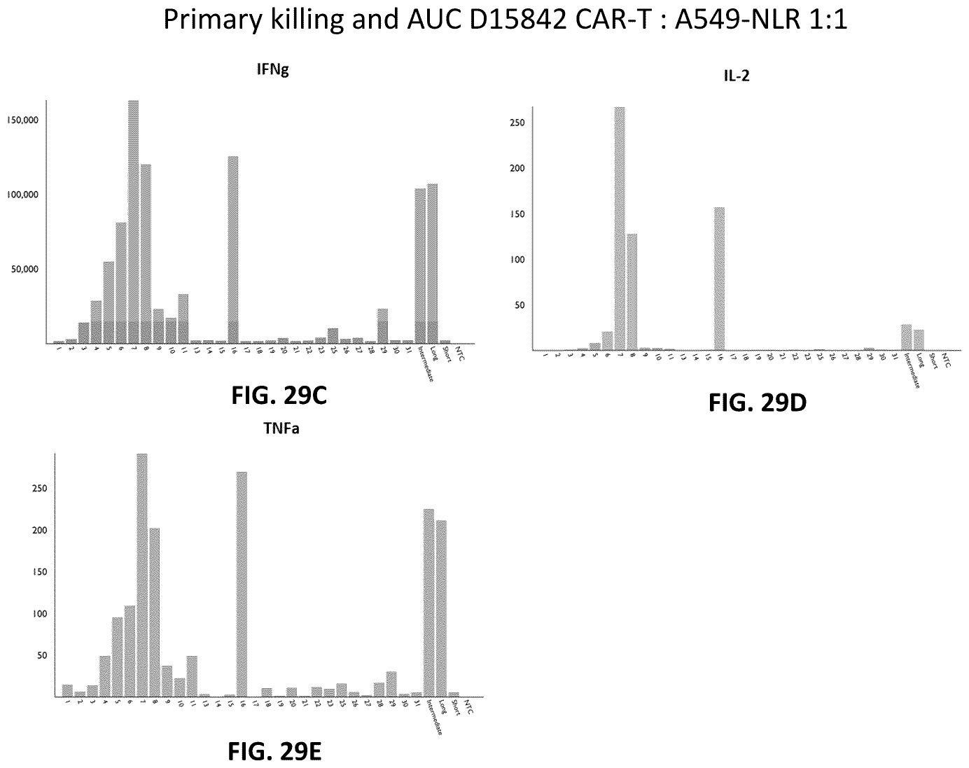

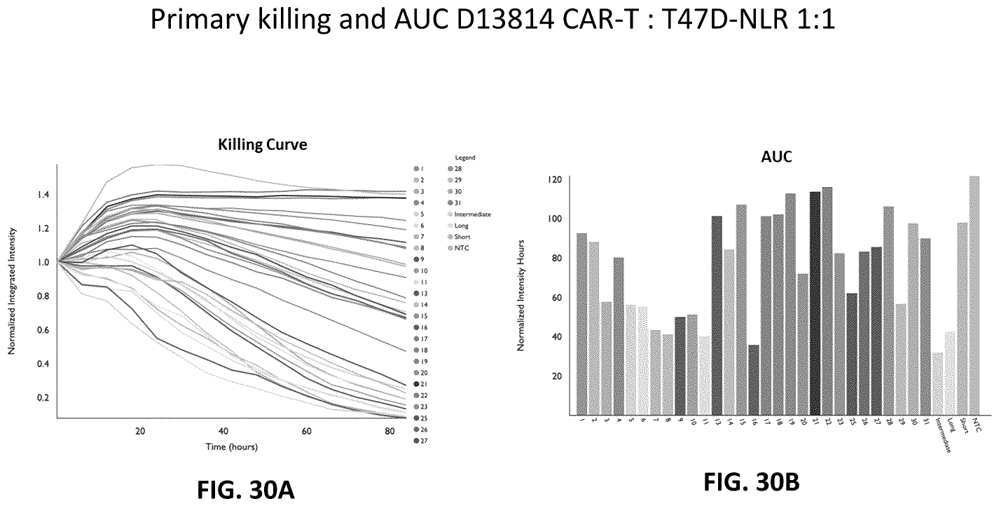

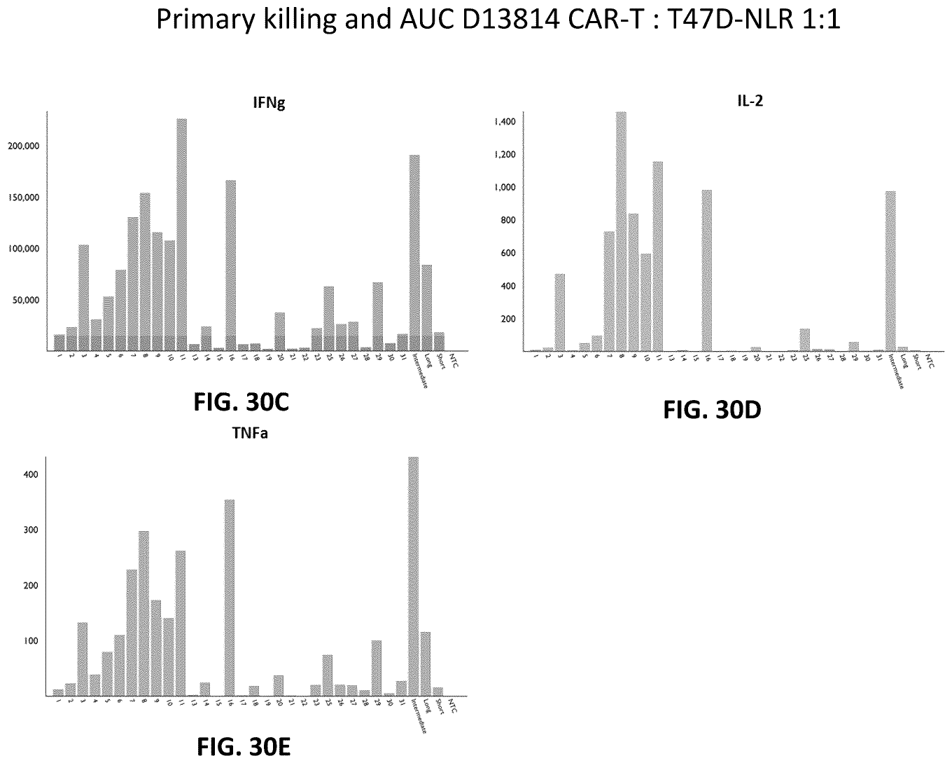

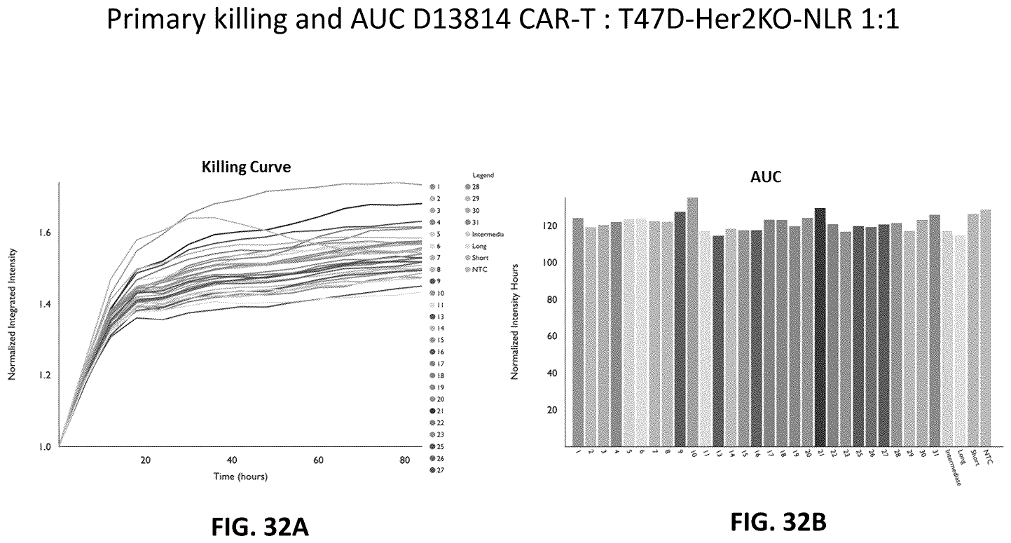

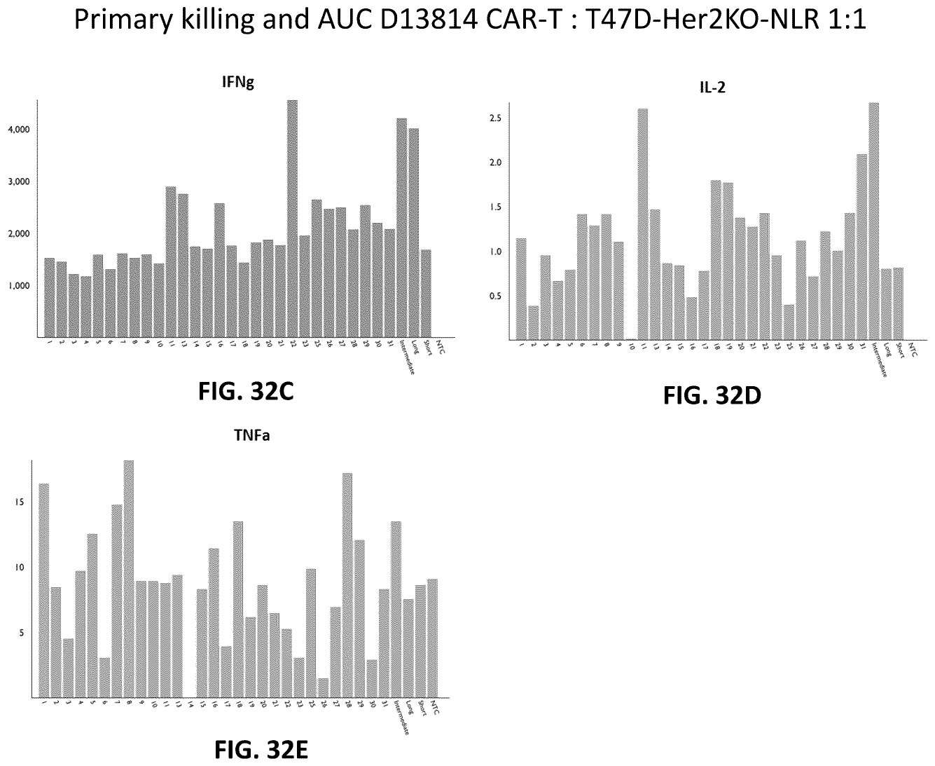

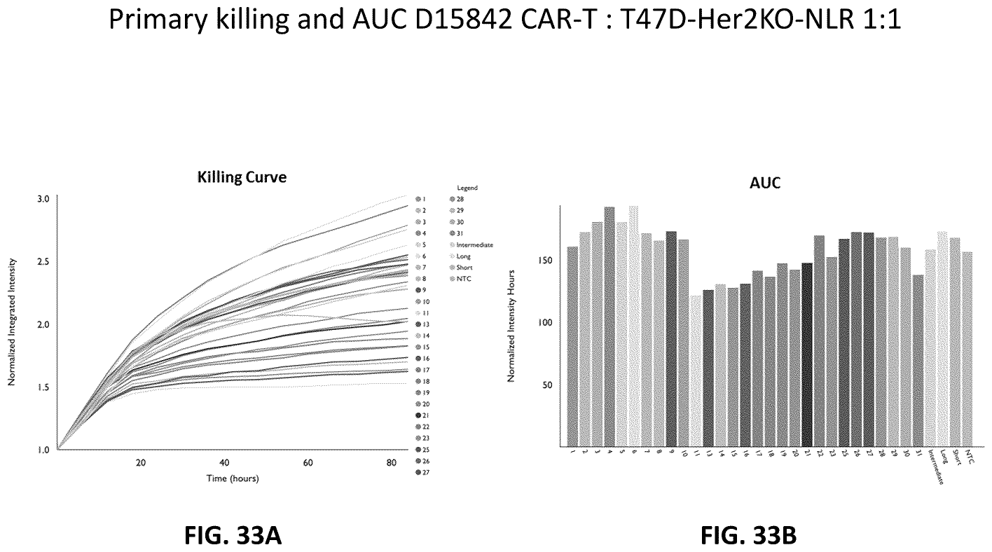

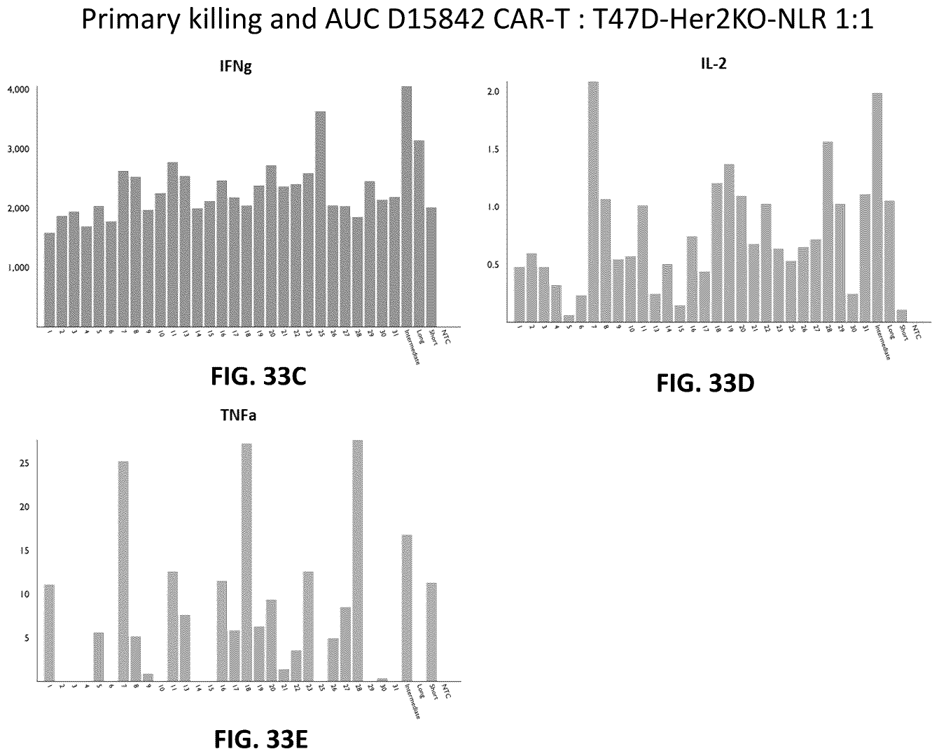

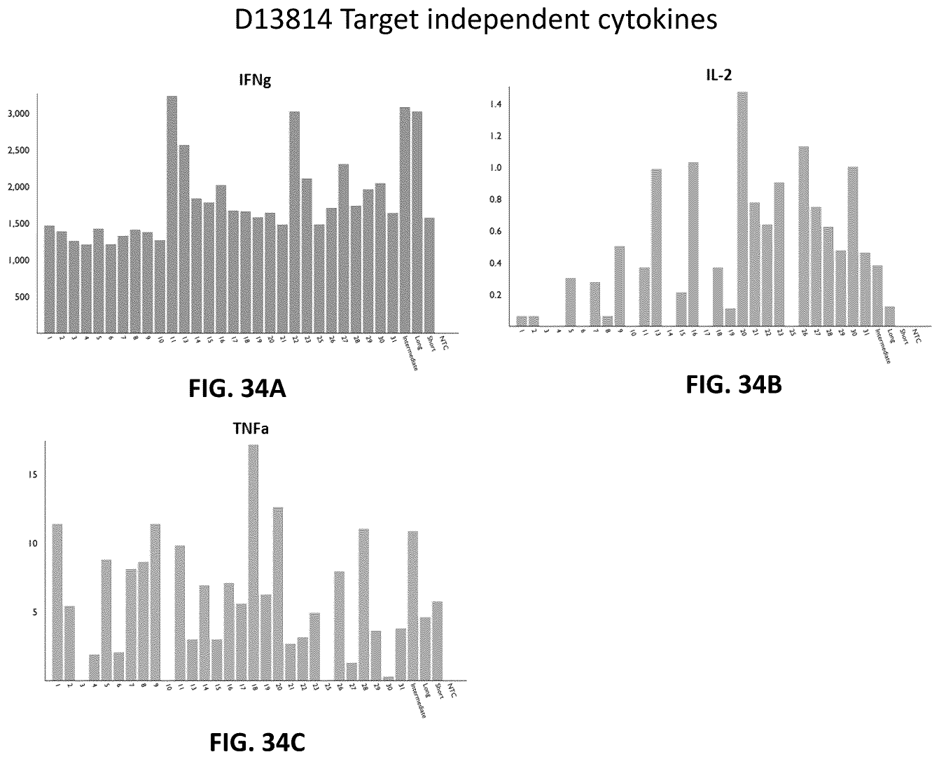

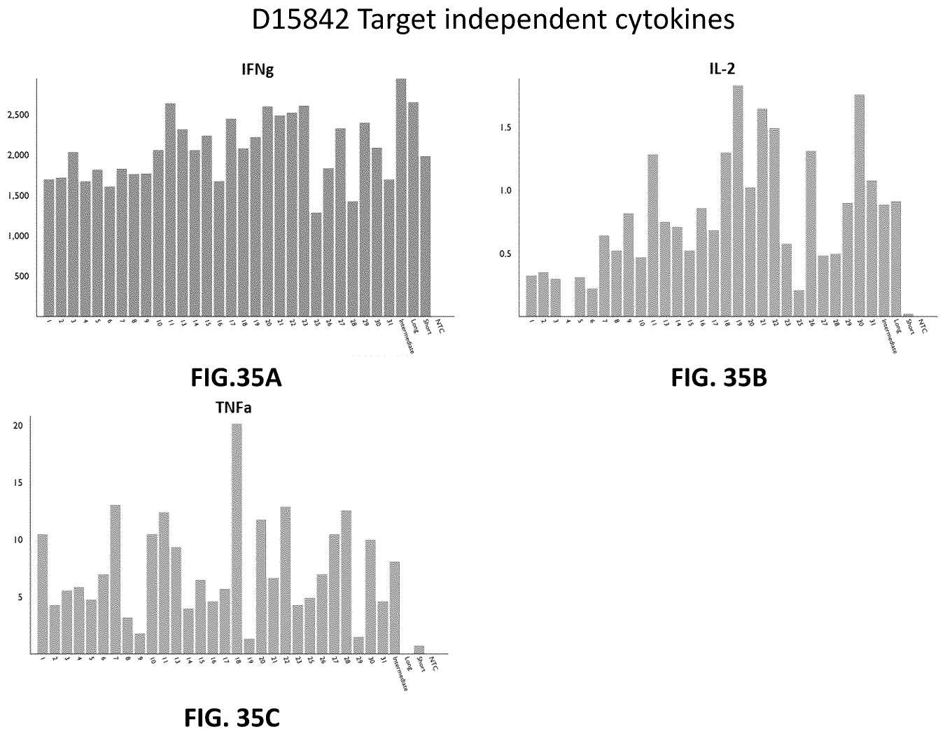

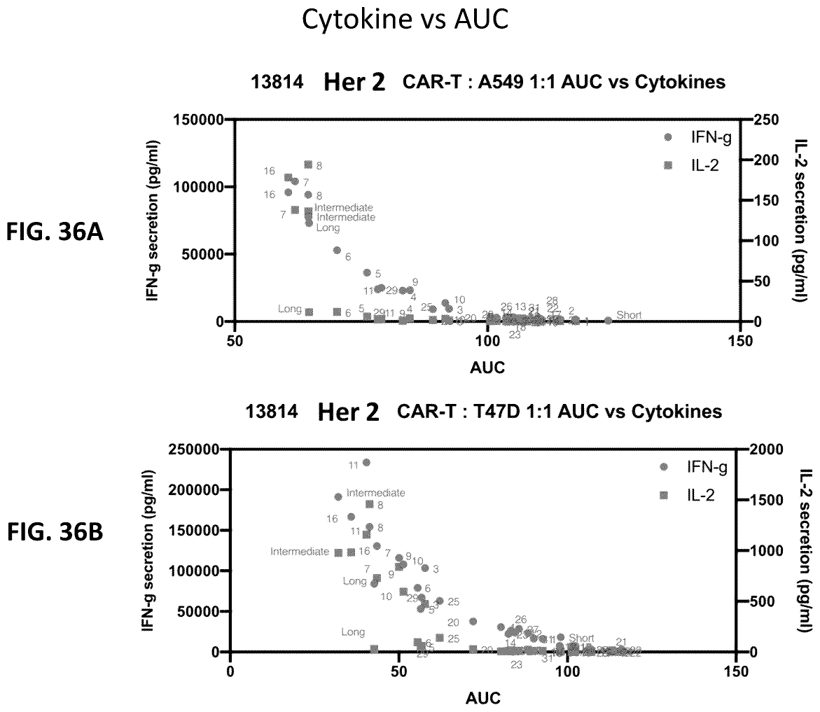

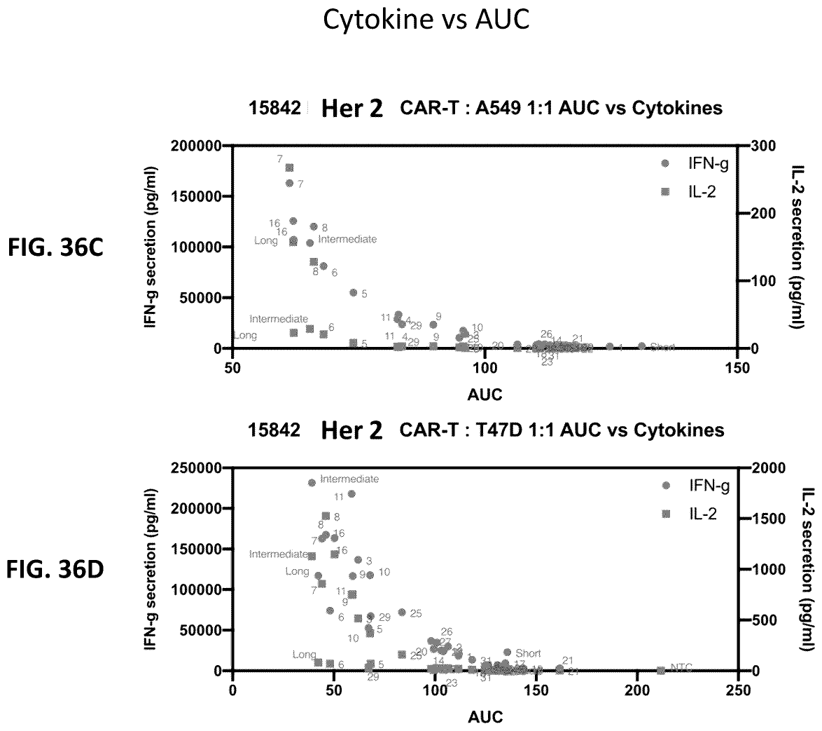

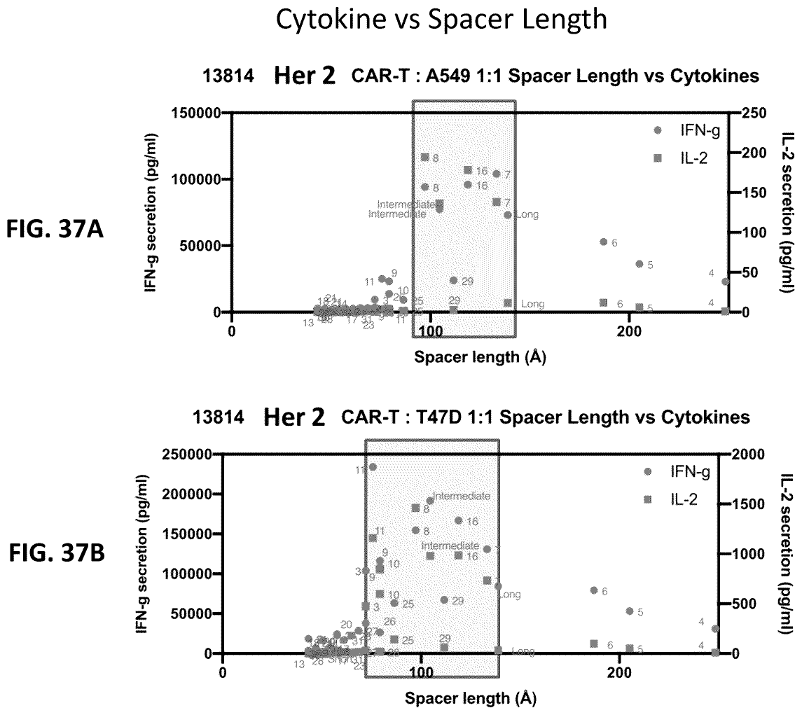

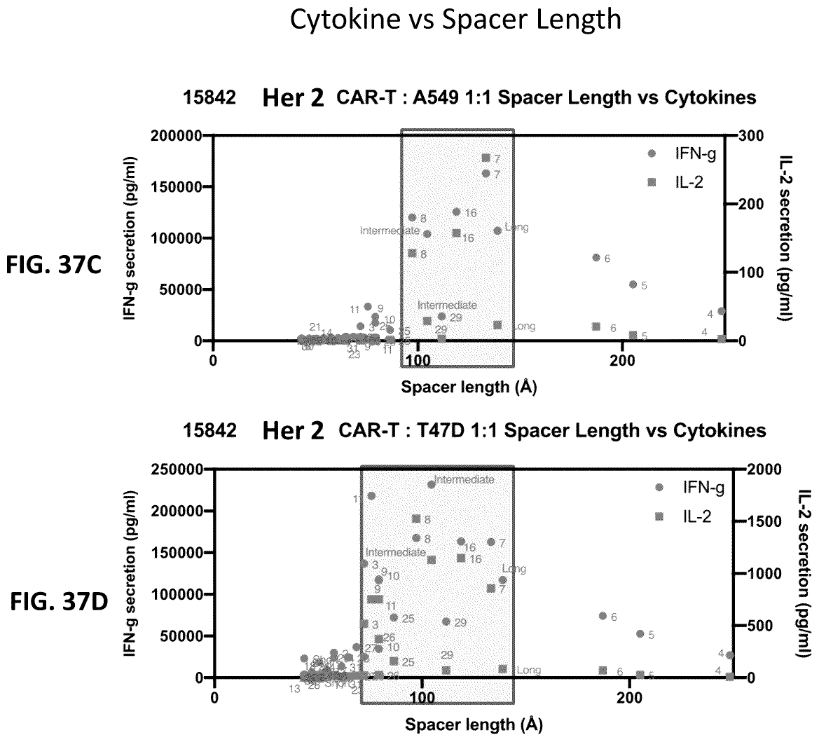

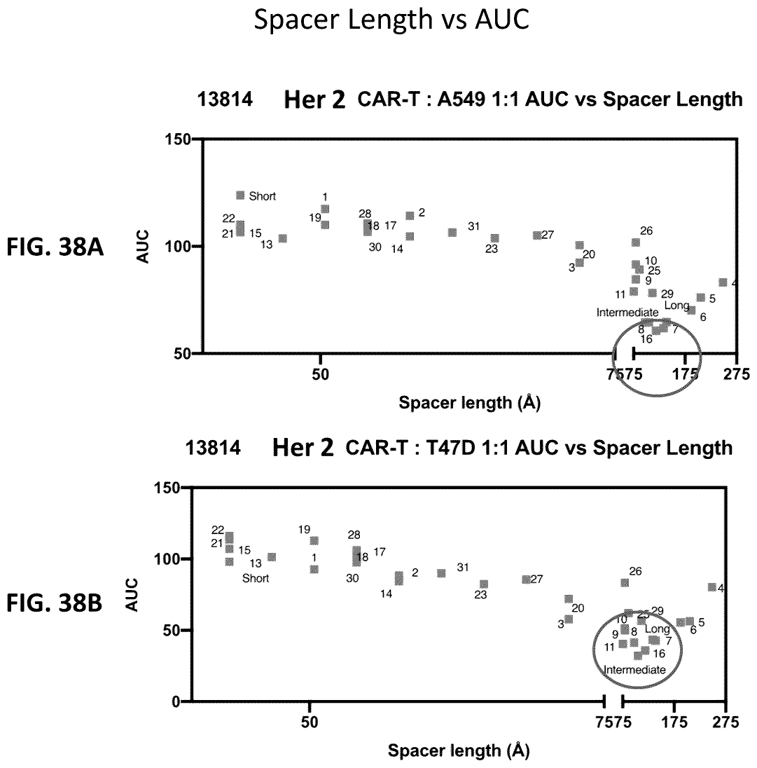

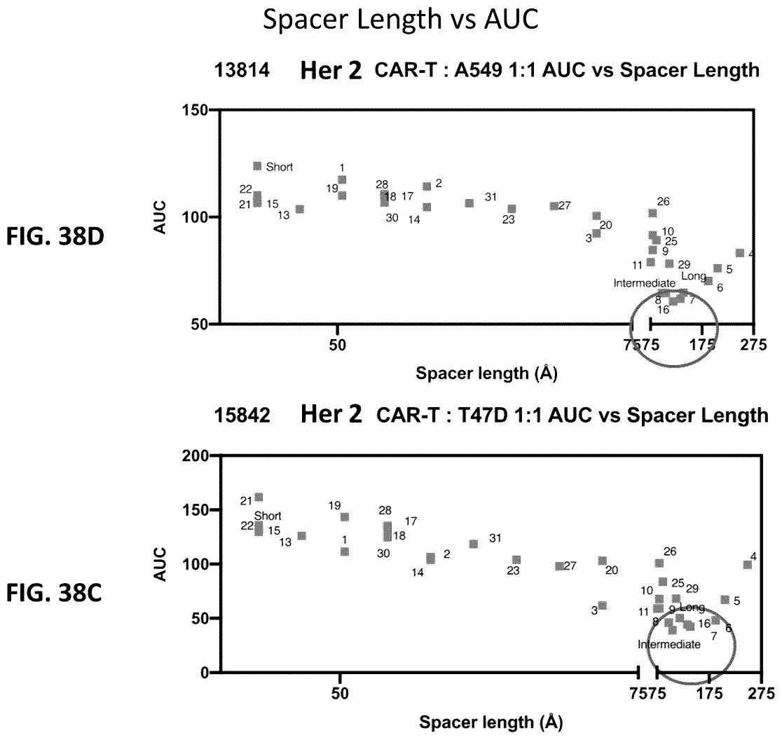

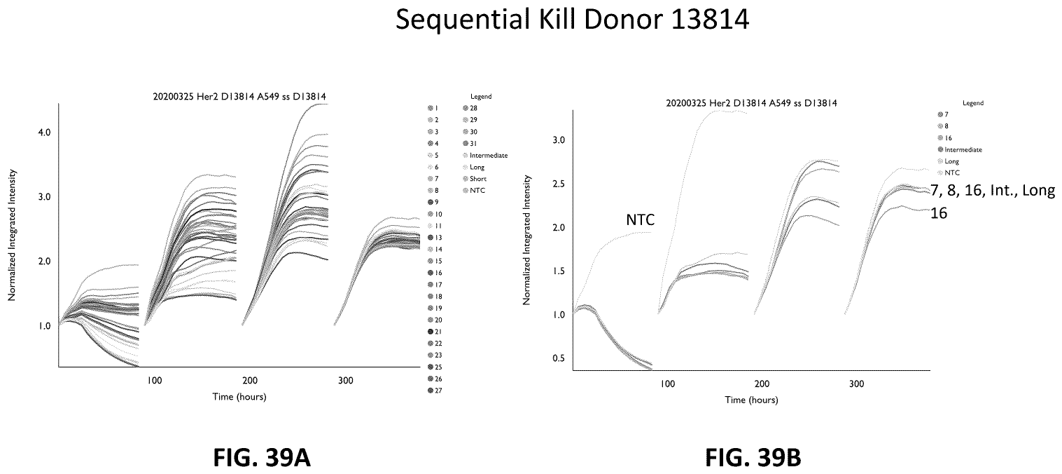

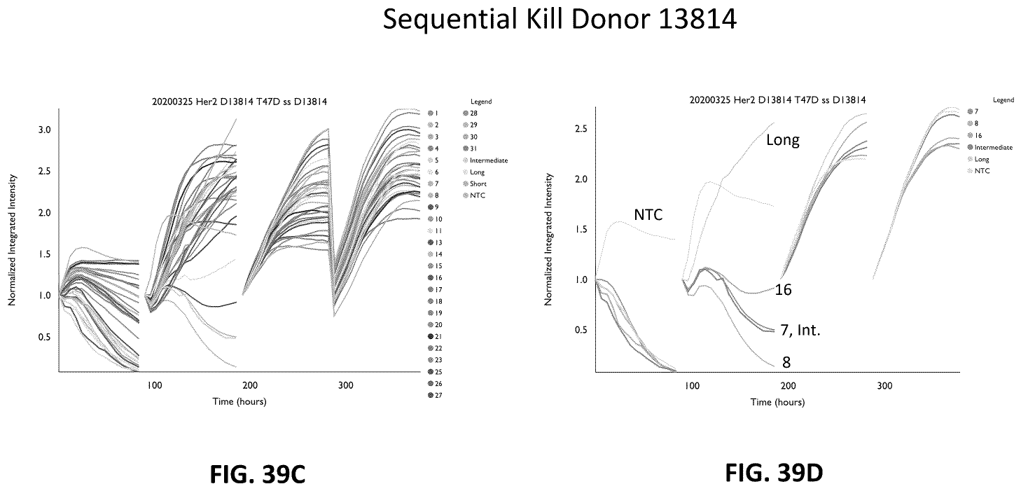

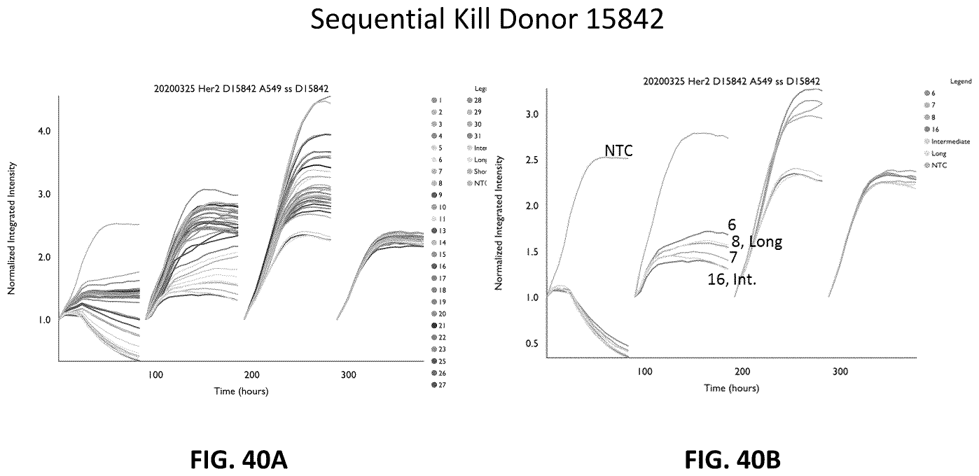

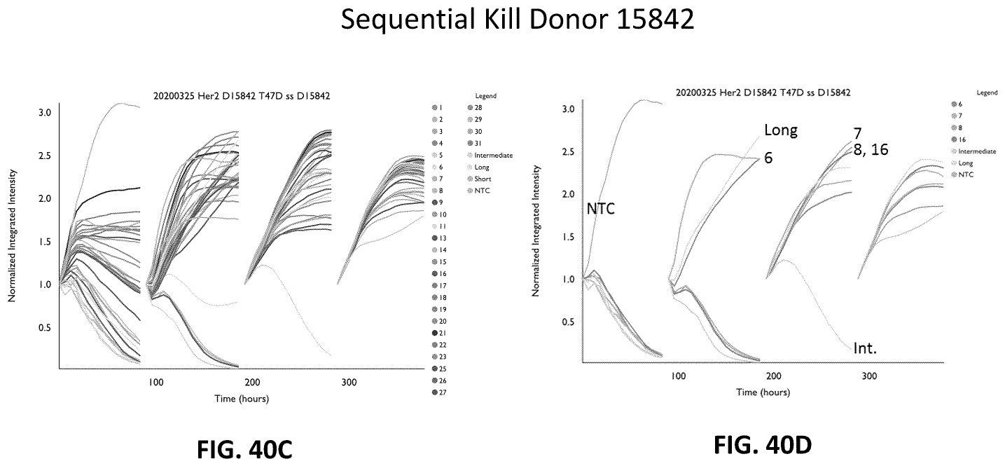

Healthy donor T cells were transduced with lentiviruses encoding Anti-Her2 CARs comprising Ig-derived spacers to a transduction level of between 20% and 70% based on EGFRt staining (FIG. 27A, FIG. 27C), and their CAR expression levels were assessed by staining with Protein L-Biotin, followed by Strepavidin-BV421 (FIG. 27B, FIG. 27D). CART cells were challenged with A549-NLR (FIGS. 28A-E, 29A-E), T47D-NLR (FIGS. 30A-E, 31A-E), and T47D-Her2KO-NLR (FIGS. 32A-E, 33A-E) to determine killing kinetics and target-dependent cytokine production. CAR-T cells were also cultured in the absence of target cells to assess target-independent cytokine production, which is an indicator of autoactivation. Target-independent cytokine secretion was generally low (FIGS. 34A-C, 35A-C). The amount of cytokine production after 24-hr post co-culture correlated with faster killing kinetics, with spacers 6, 7, 8, 11, 16, and Intermediate performing especially well (FIGS. 36A-D). IFN-γ and IL-2 production were highest when CAR spacer lengths were between ˜57 Å and ˜122 Å (FIGS. 37A-D). Similarly, short term cytotoxicity was effective when spacer lengths were between ˜57 Å and ˜122 Å (FIGS. 38A-D). We observed that anti-Her2-derived CART cells comprising spacers 6, 7, 8, 16, and Intermediate were best able to sustain target lytic activities over repeated rounds of target exposure (FIGS. 39A-D, FIGS. 40A-D).

TABLE 24
Complete anti-Her2 CAR sequences. The spacer used in each Her2
CAR is indicated in the first column. Spacer numbering
follows FIG. 9 numbering. The scFv linker of SEQ ID NO: 5003,
can be optionally replaced with SEQ ID NO: 5004.

| | Complete CAR SEQ ID NO | Complete CAR SEQ ID NO |
|---|---|---|
| Spacer | Without optional LINKER | With optional linker |
| 6 | 5005 | 5035 |
| 7 | 5006 | 5036 |
| 8 | 5007 | 5037 |
| 16 | 5008 | 5038 |

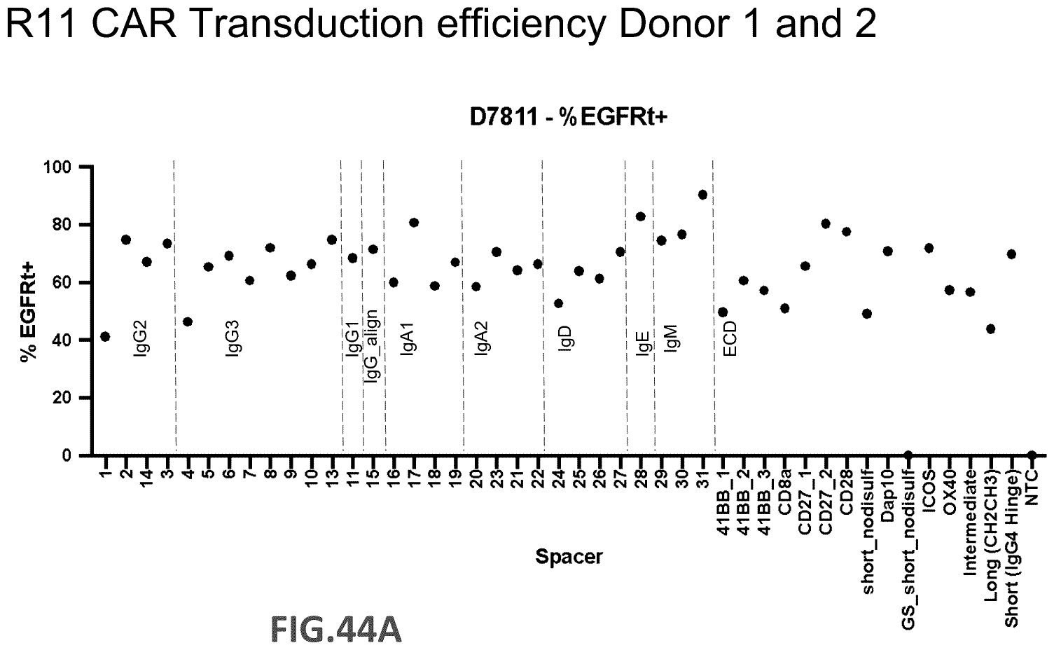

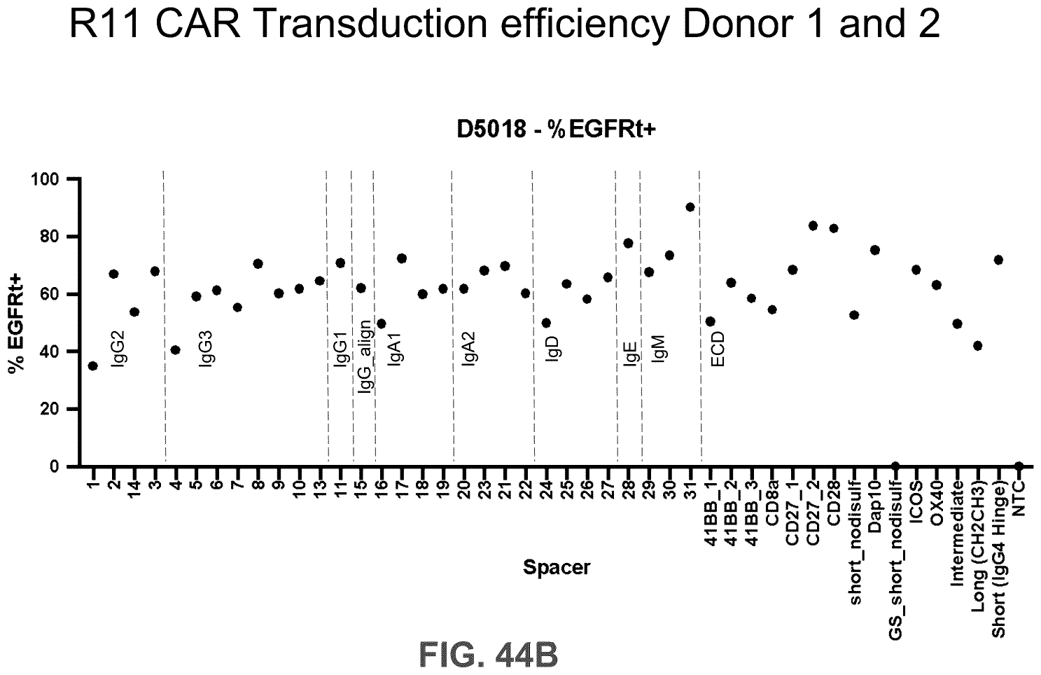

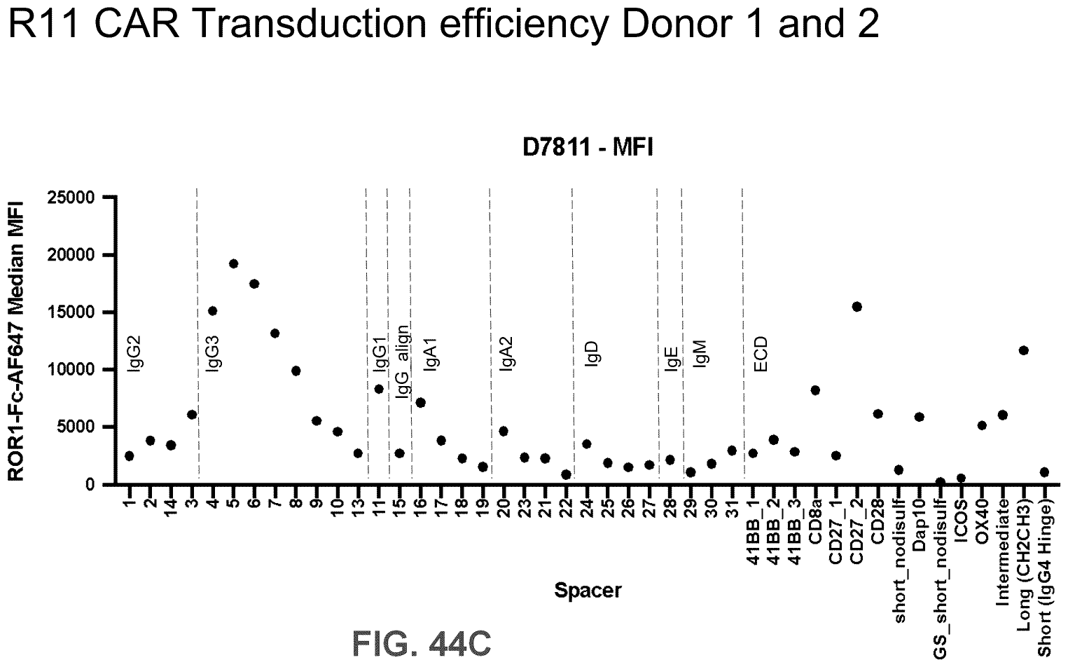

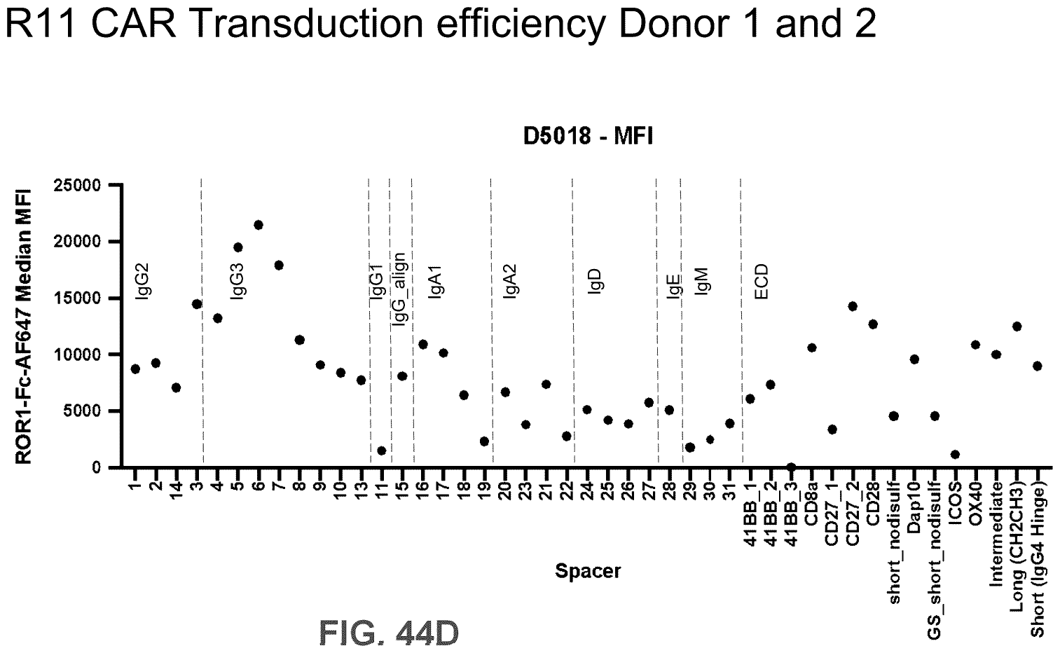

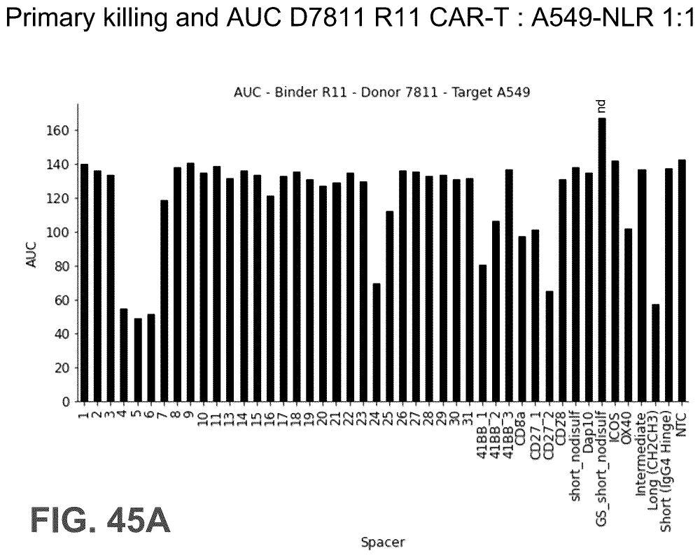

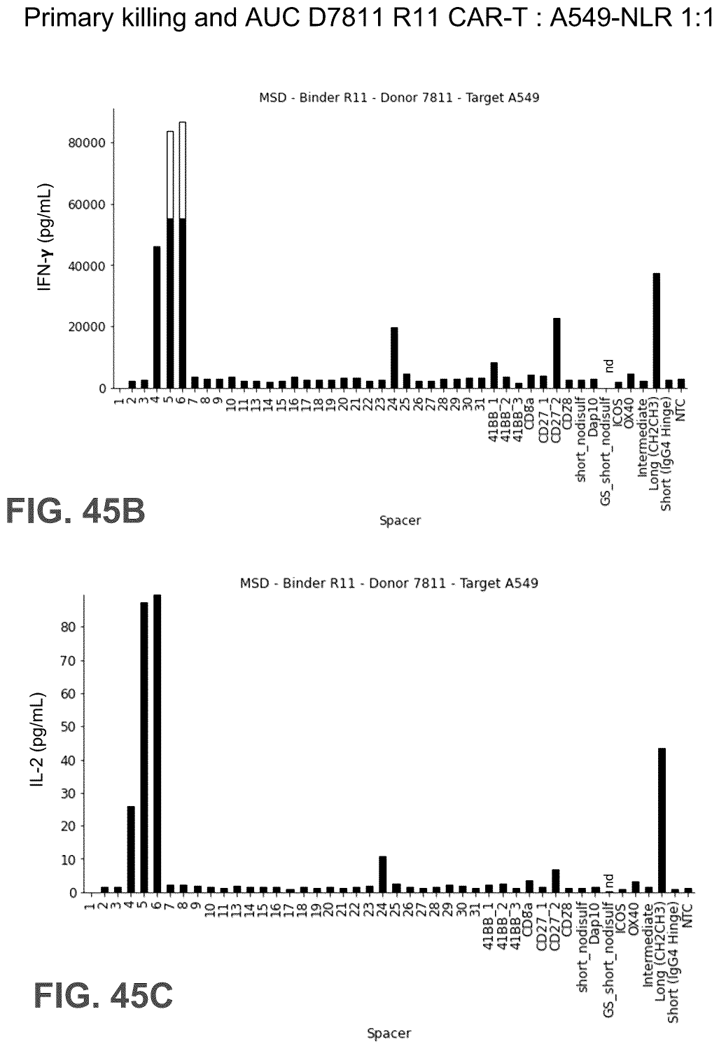

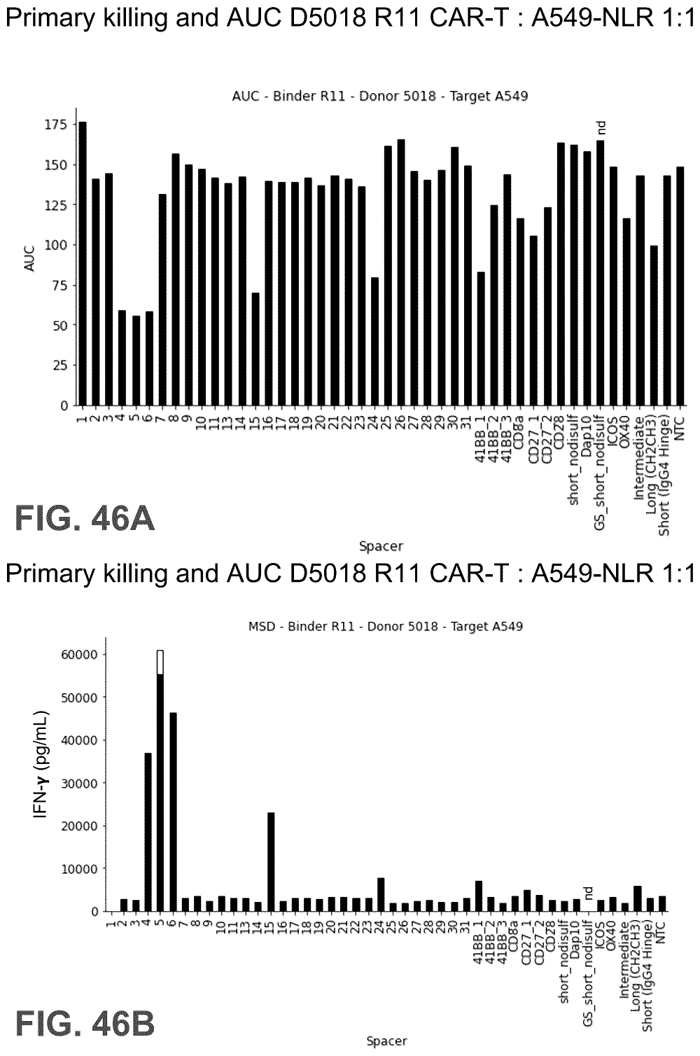

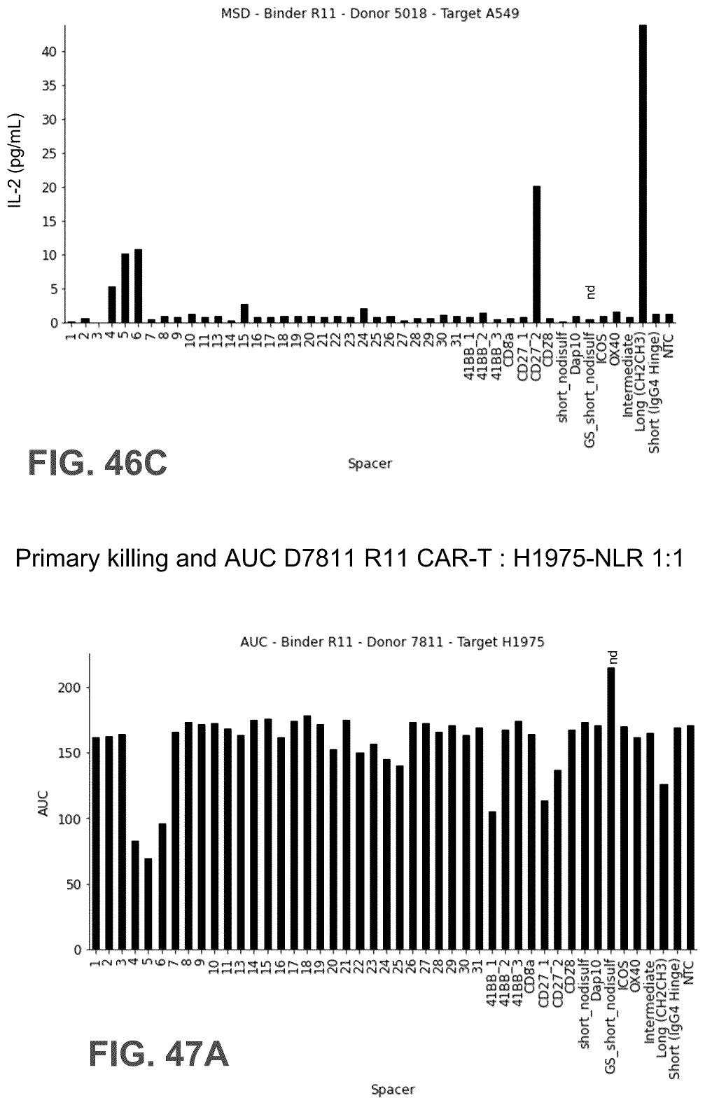

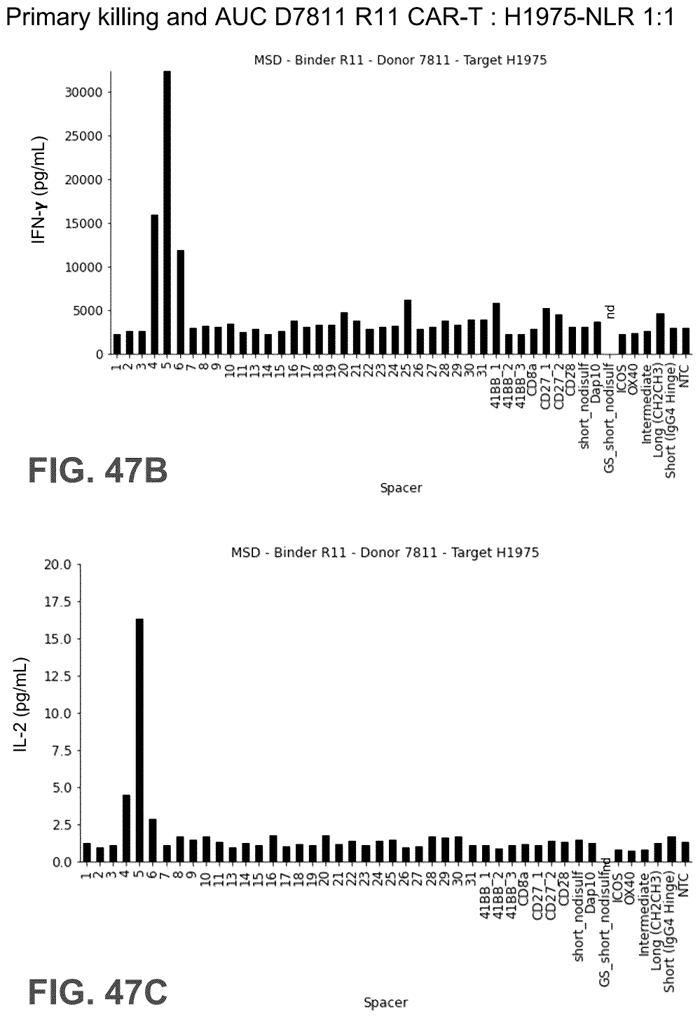

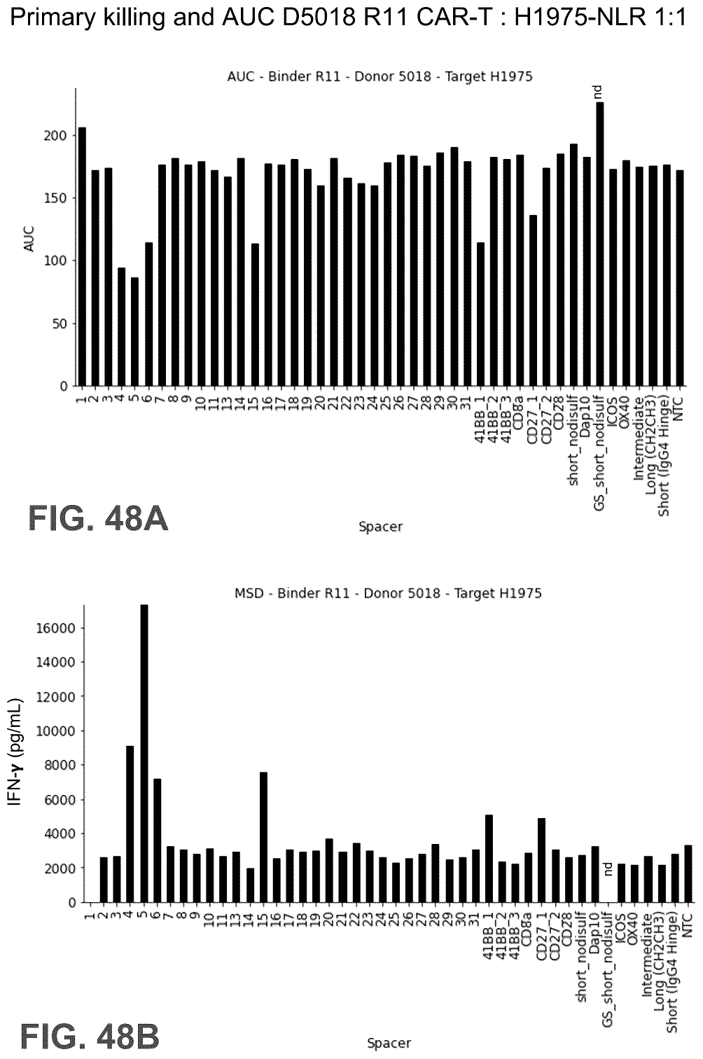

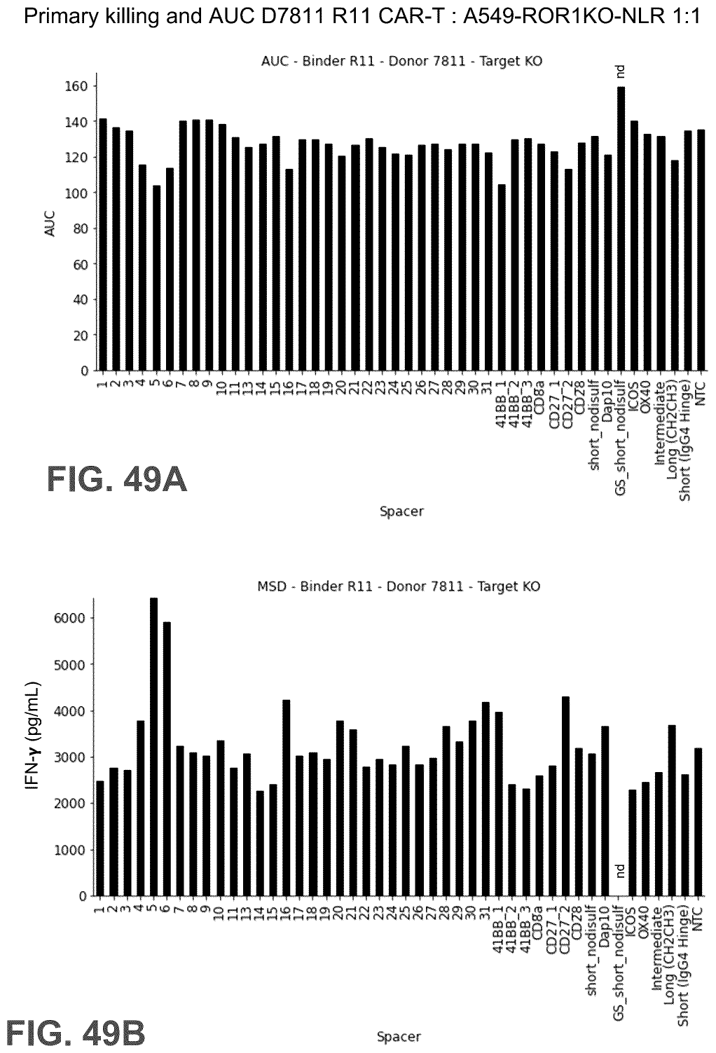

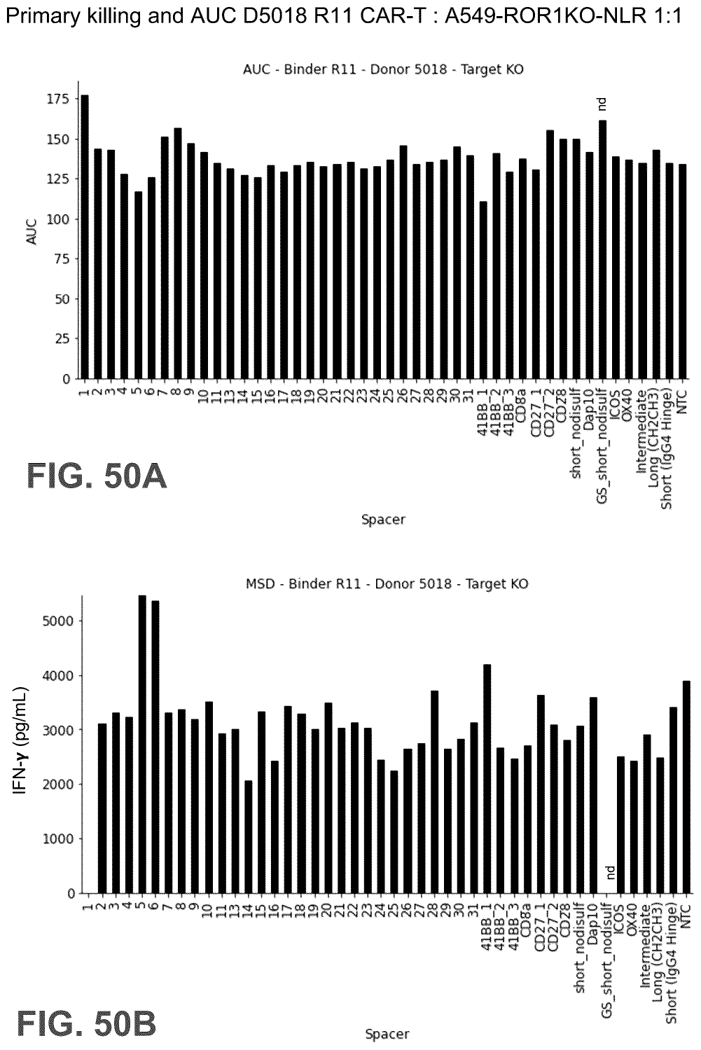

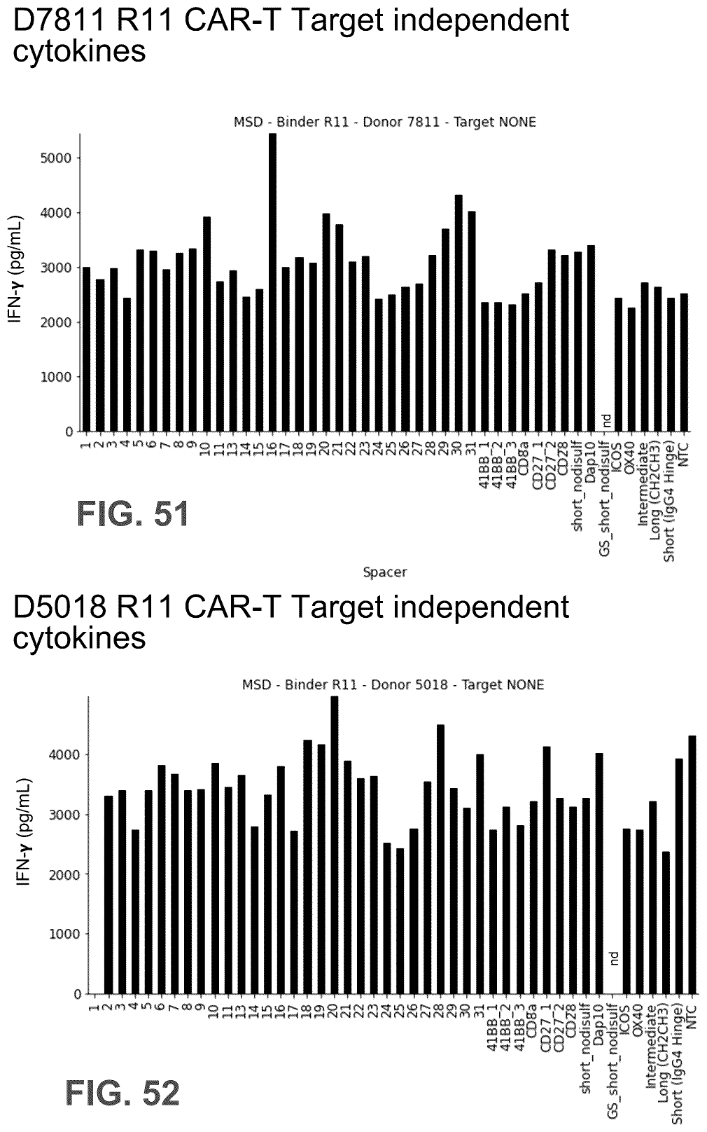

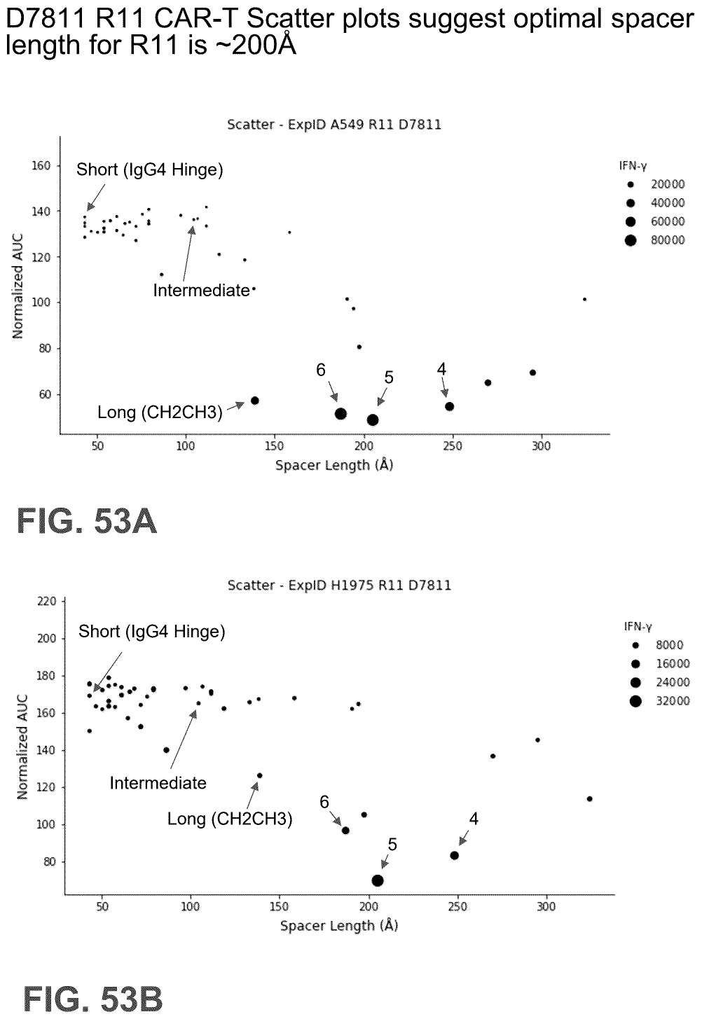

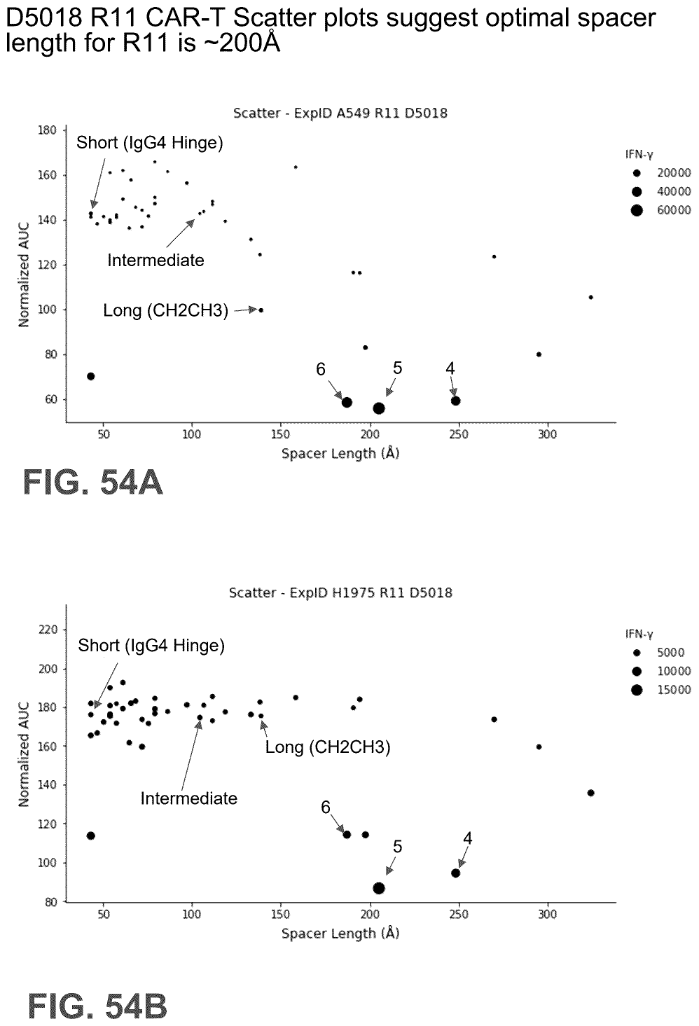

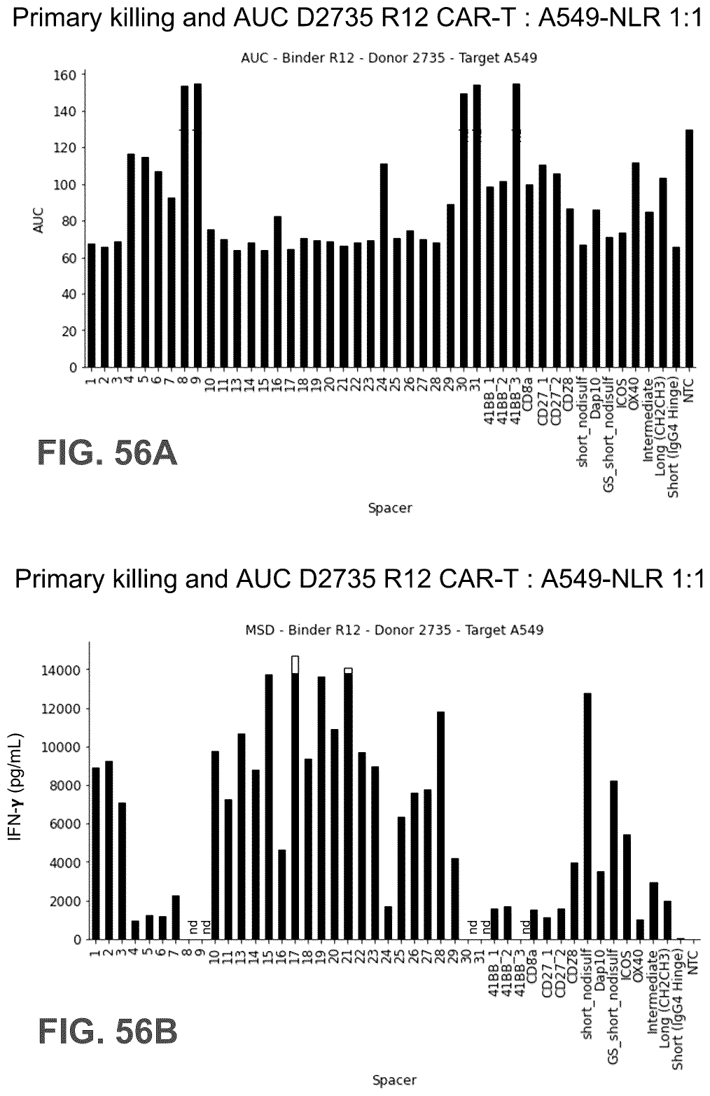

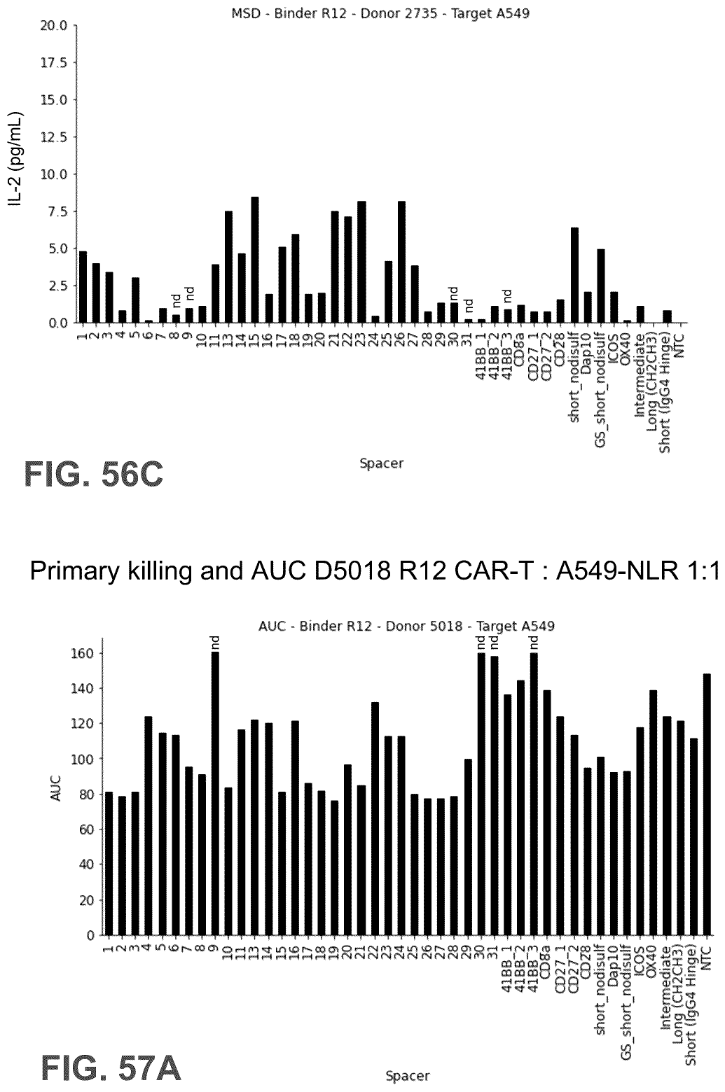

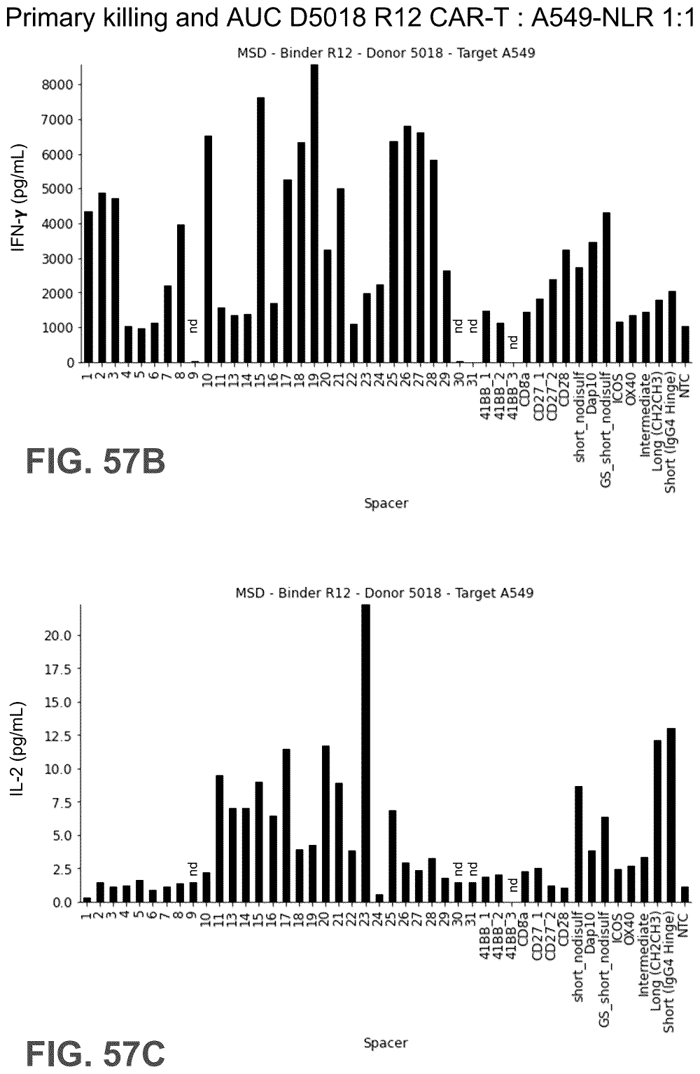

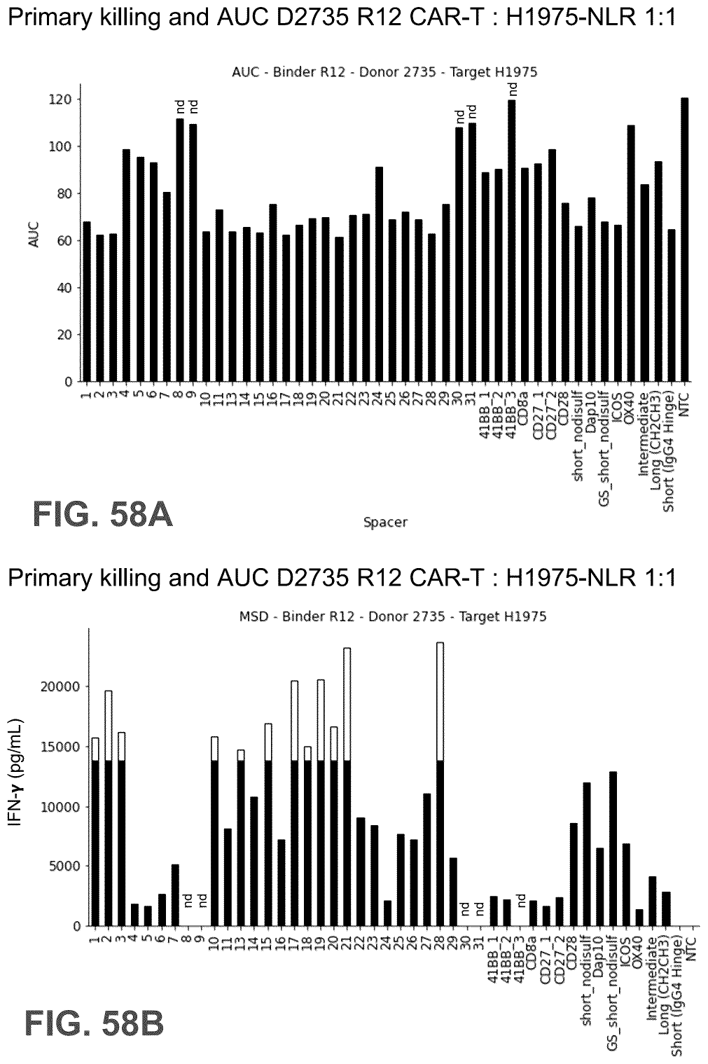

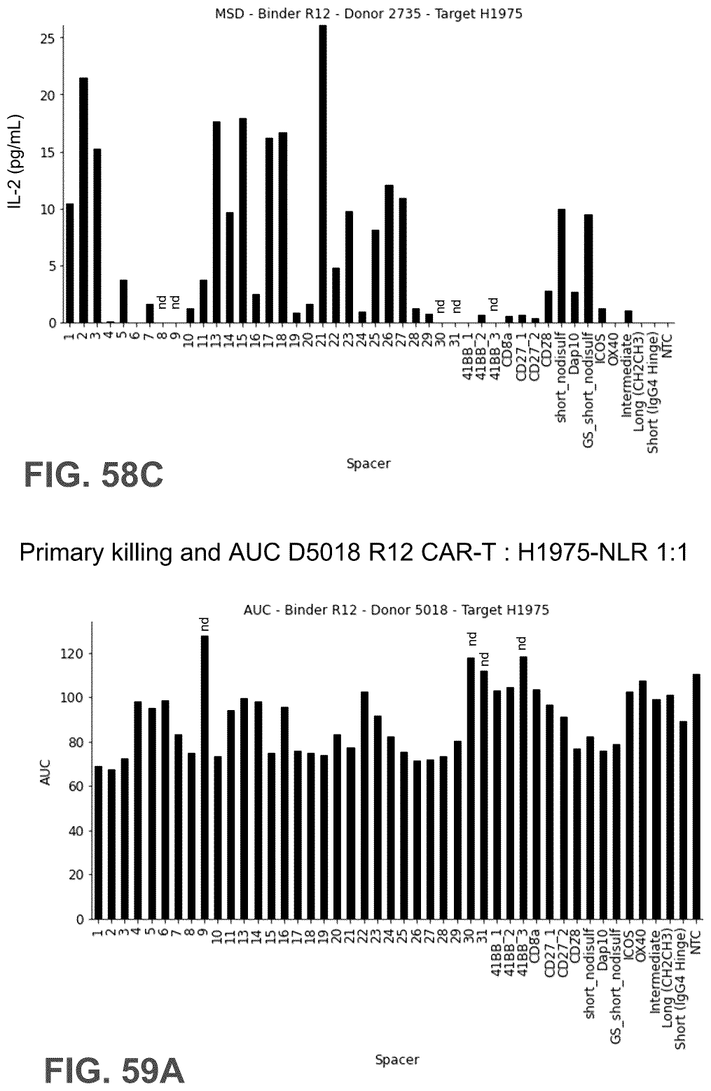

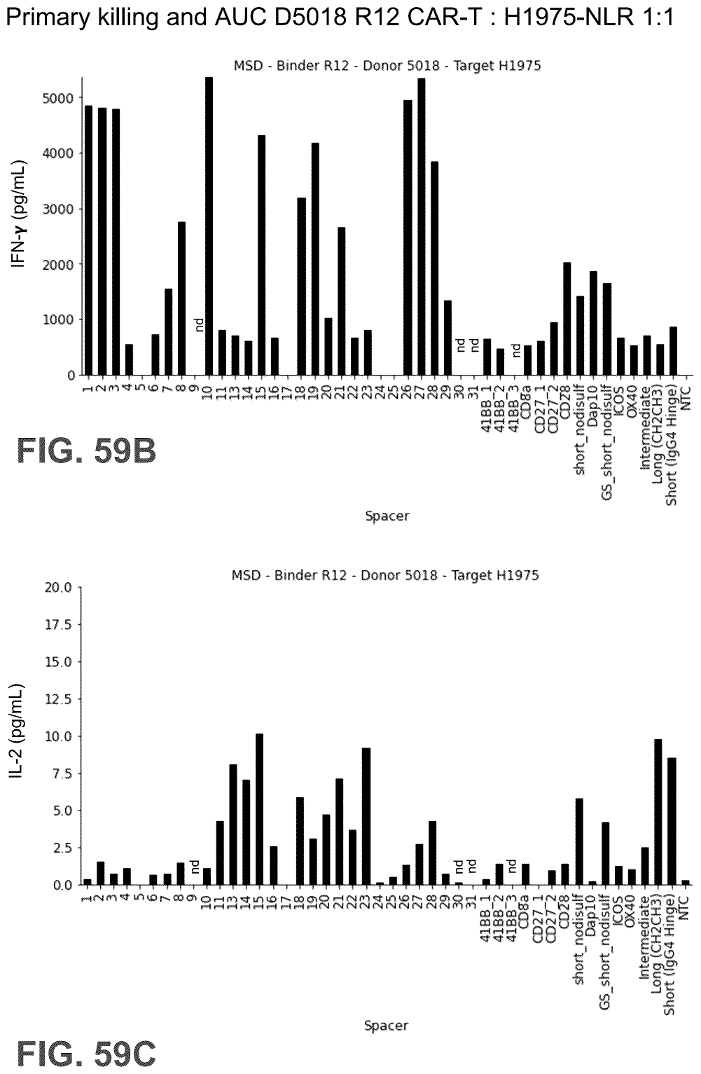

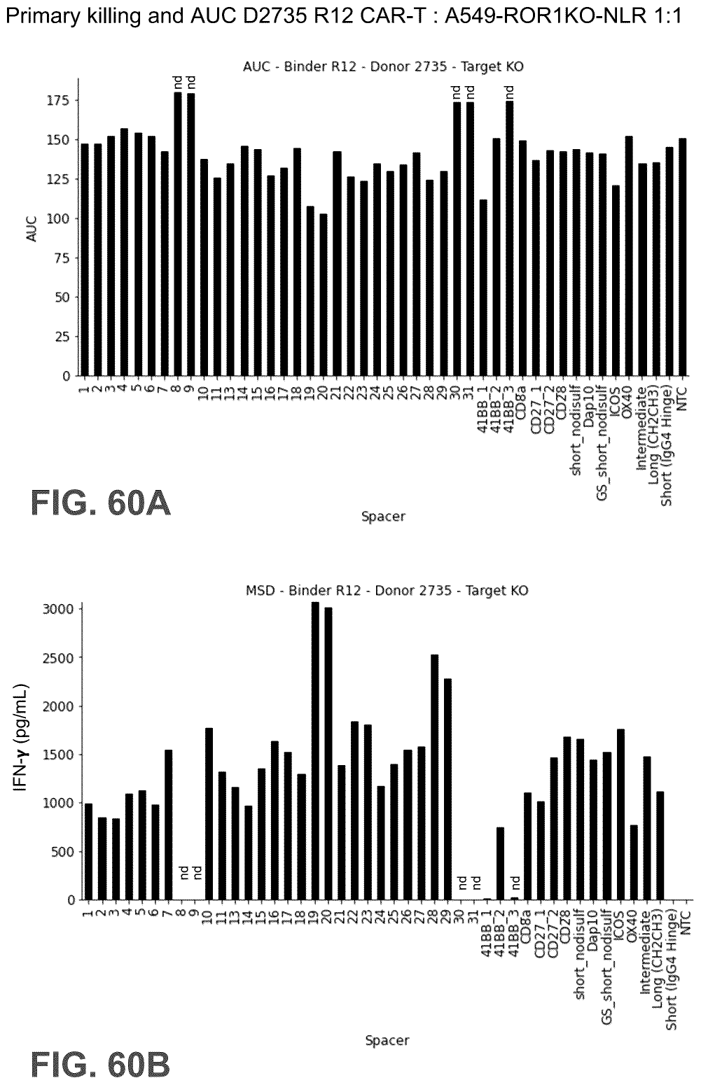

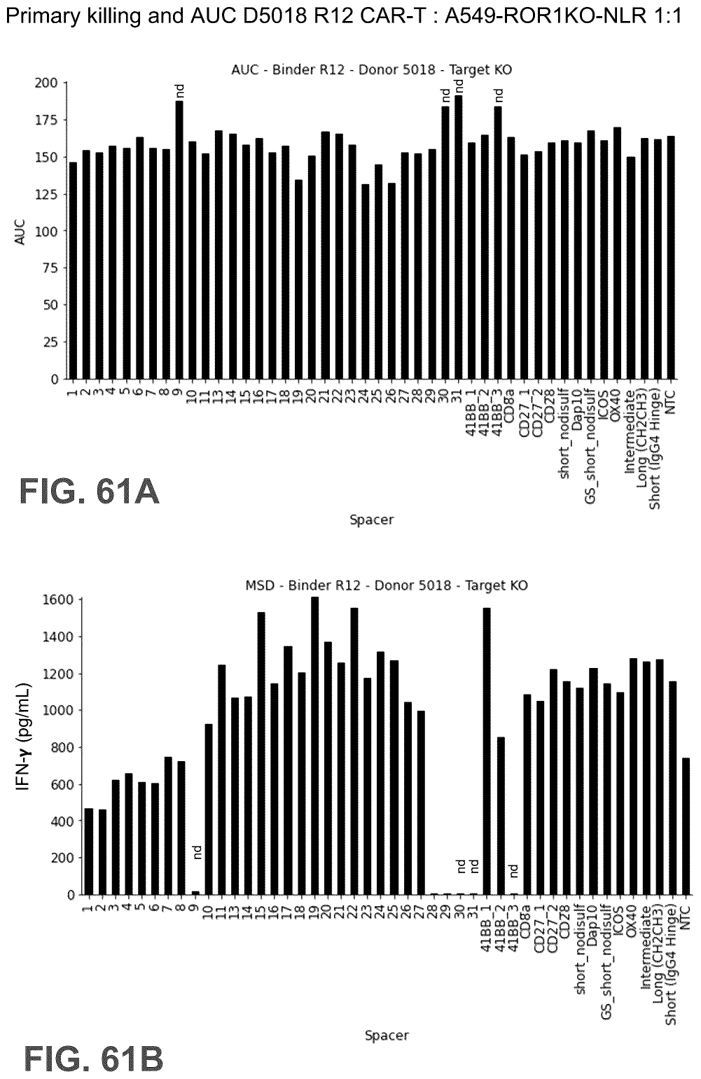

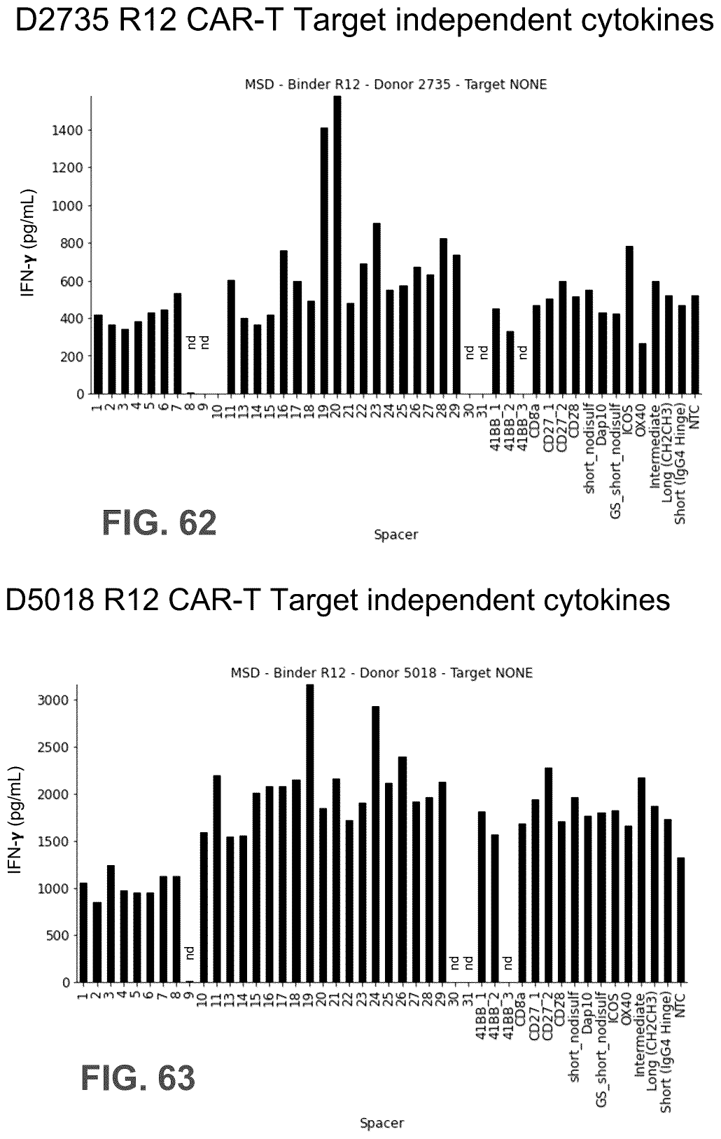

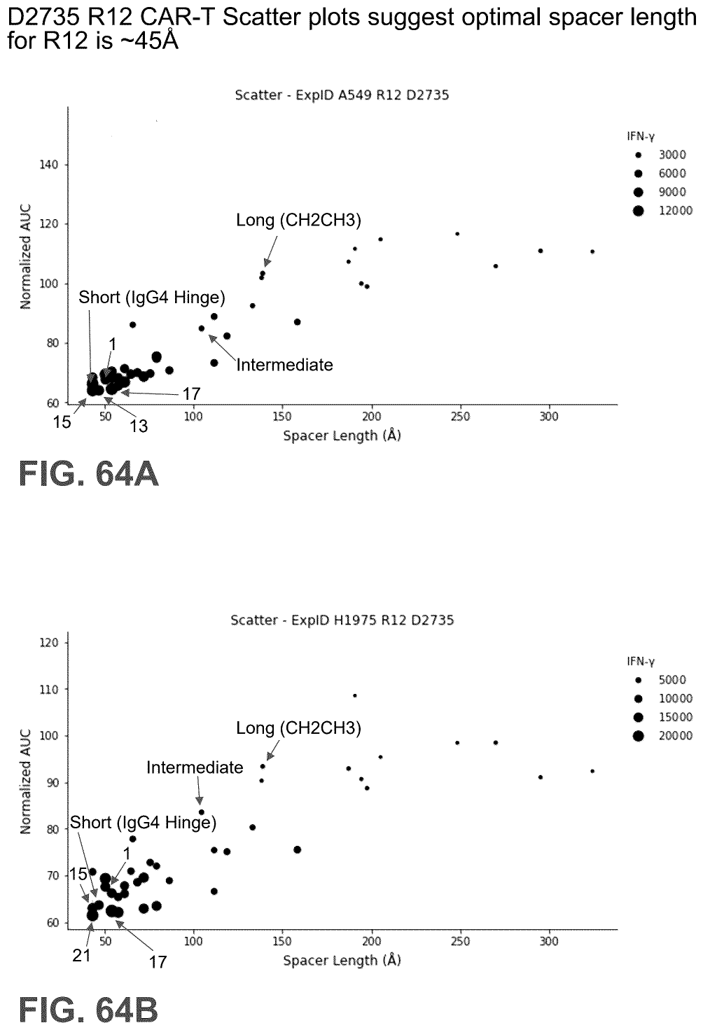

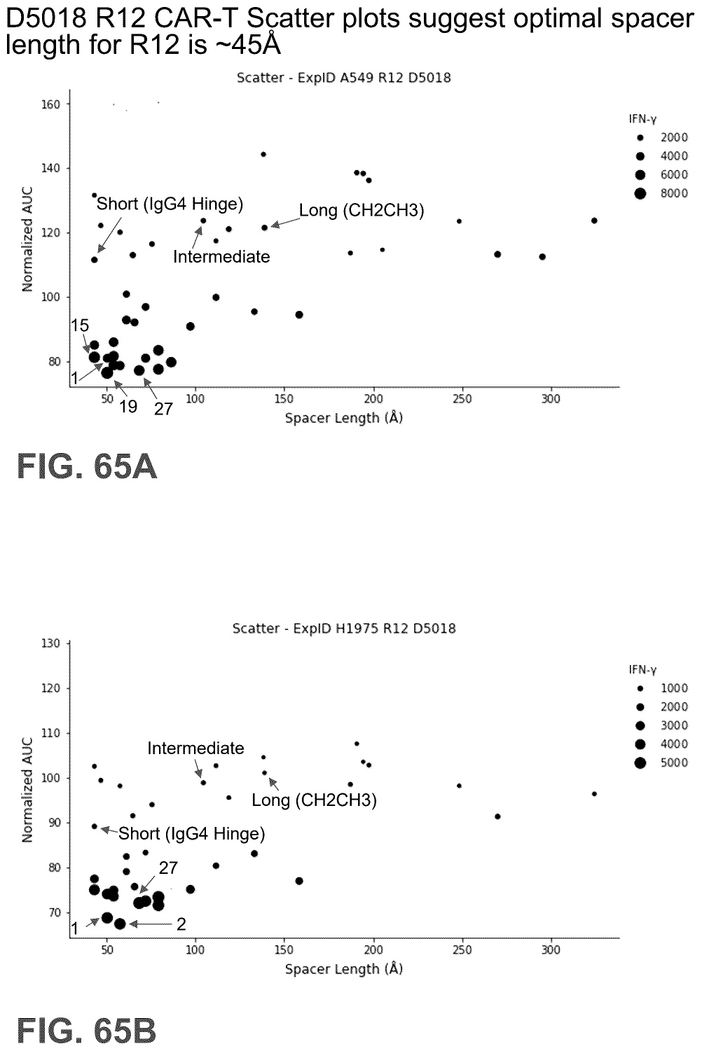

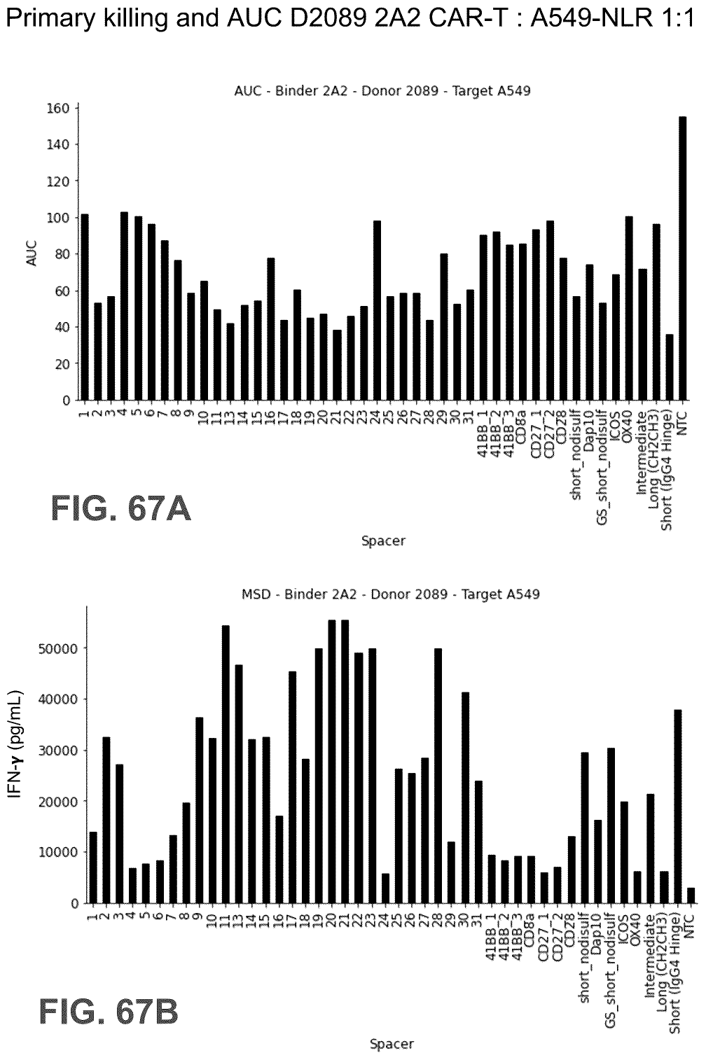

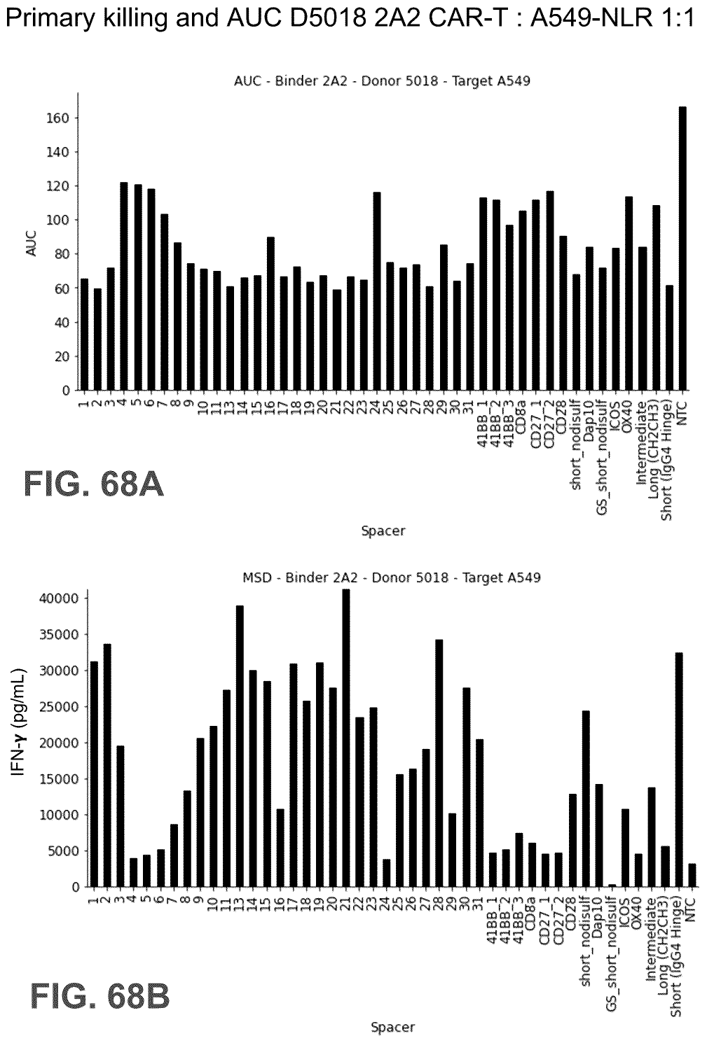

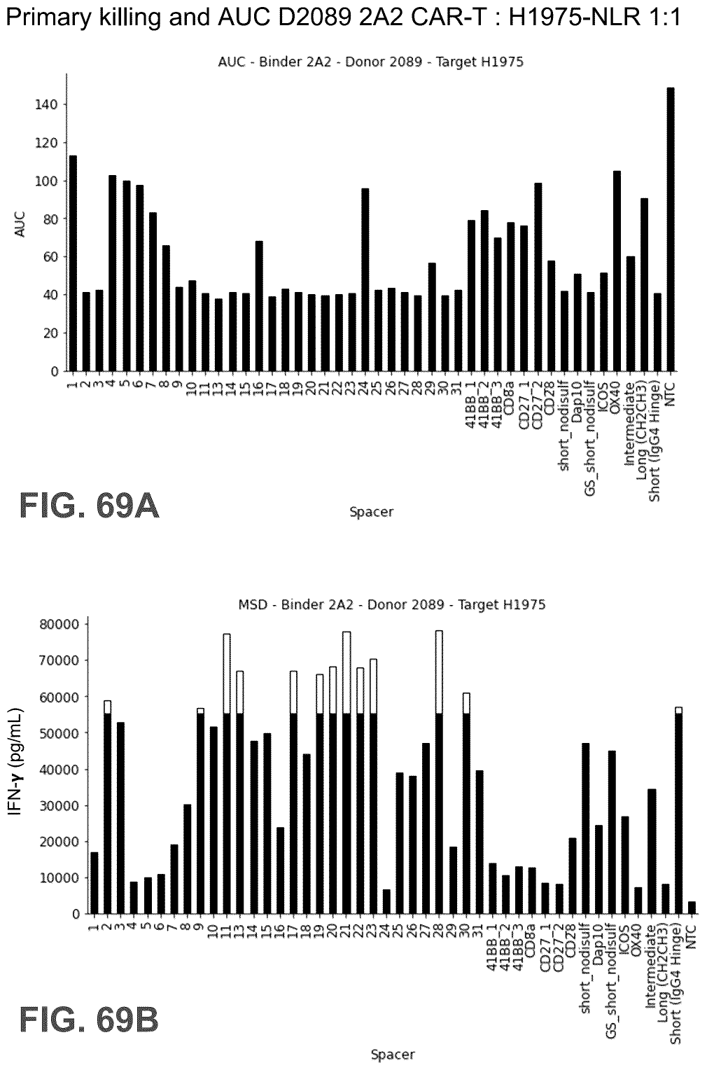

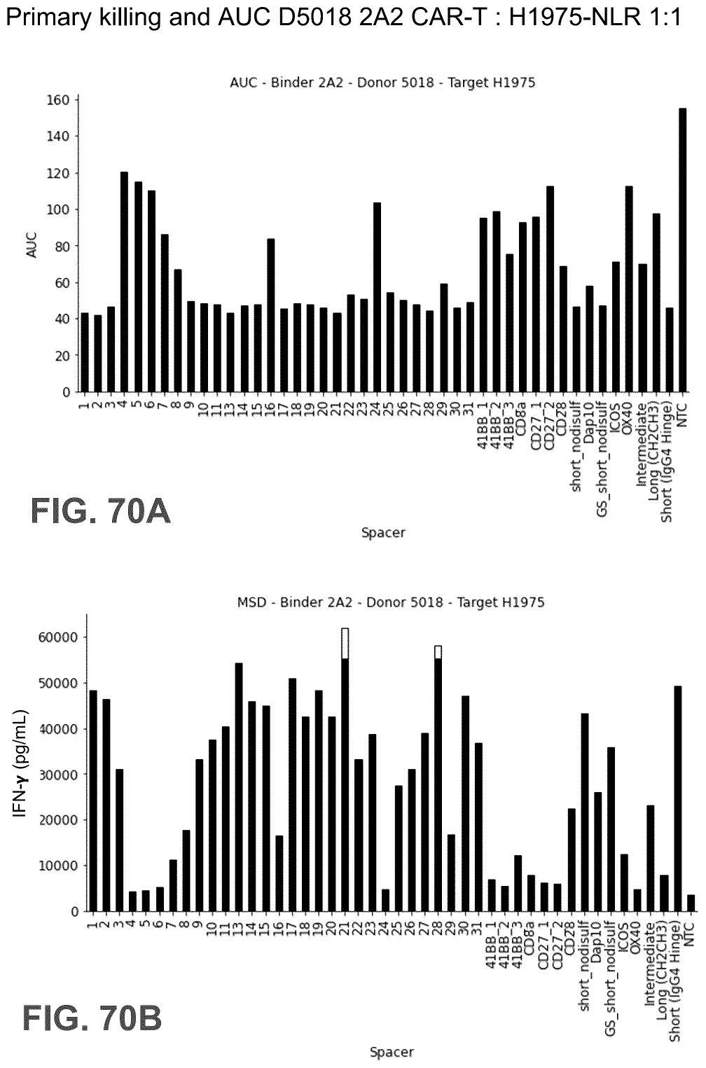

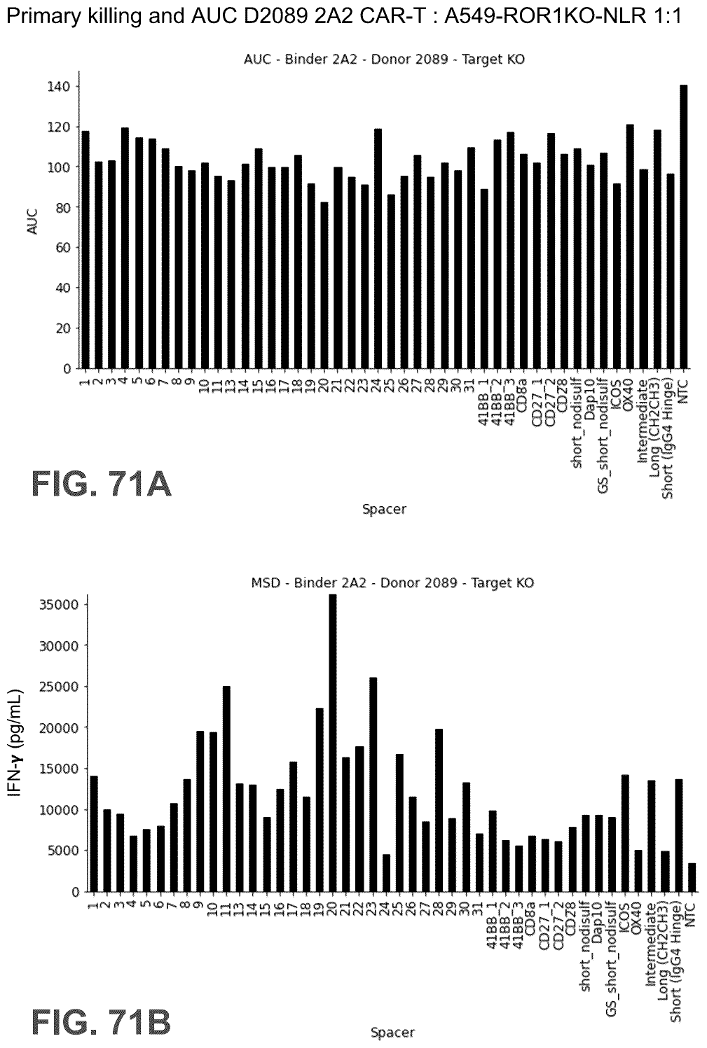

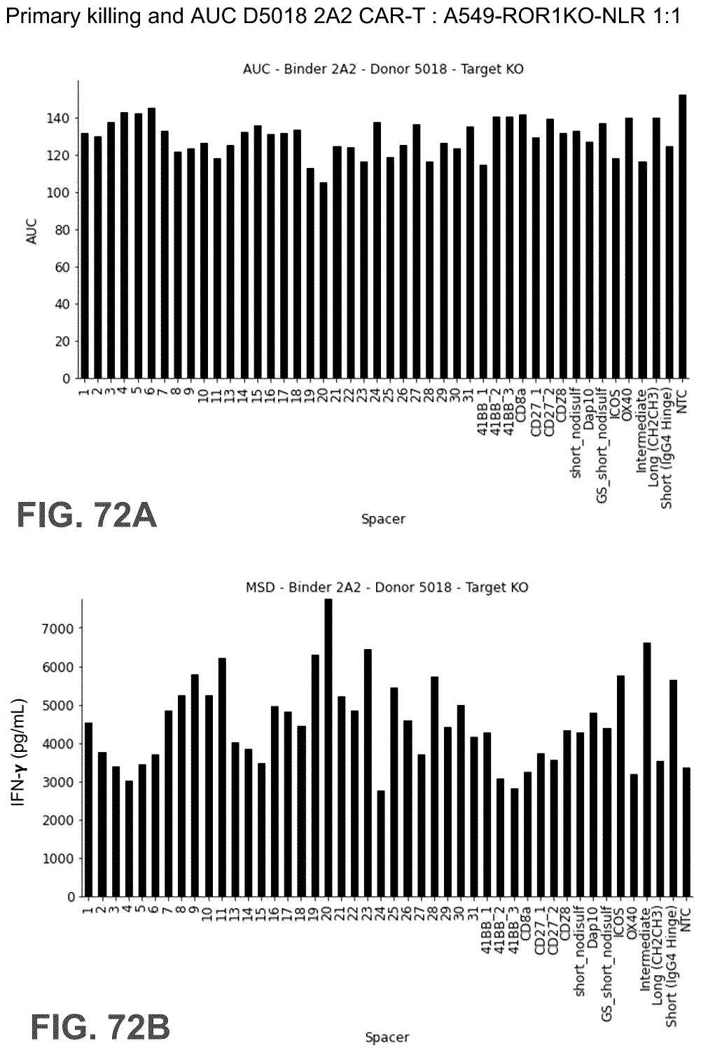

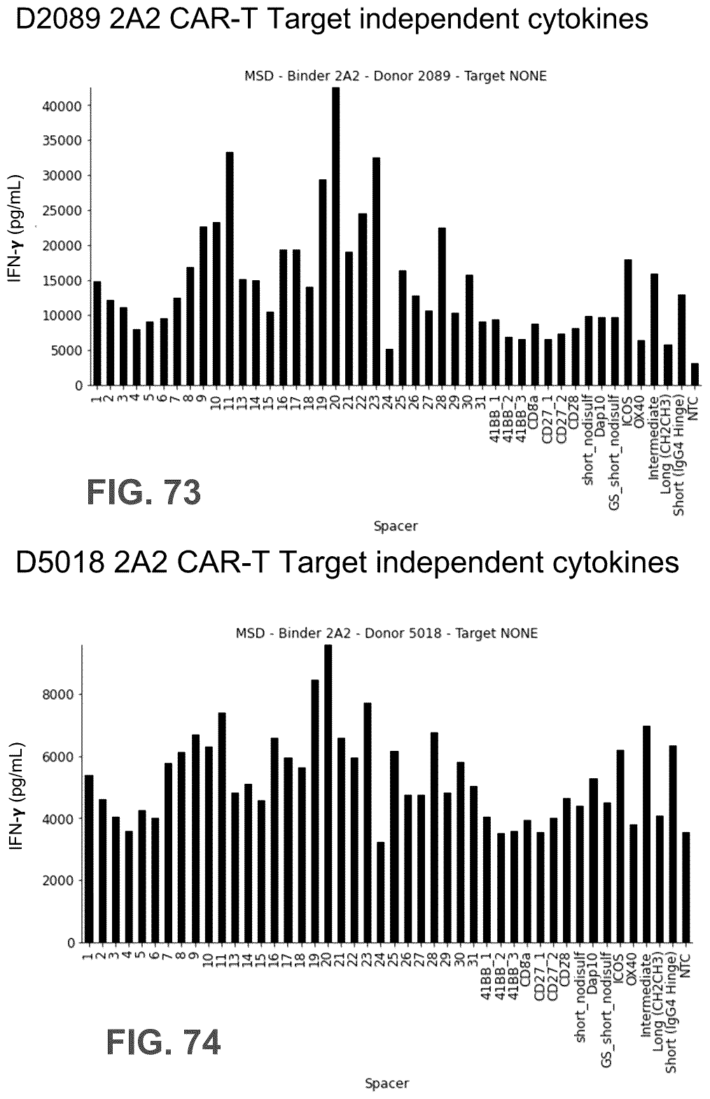

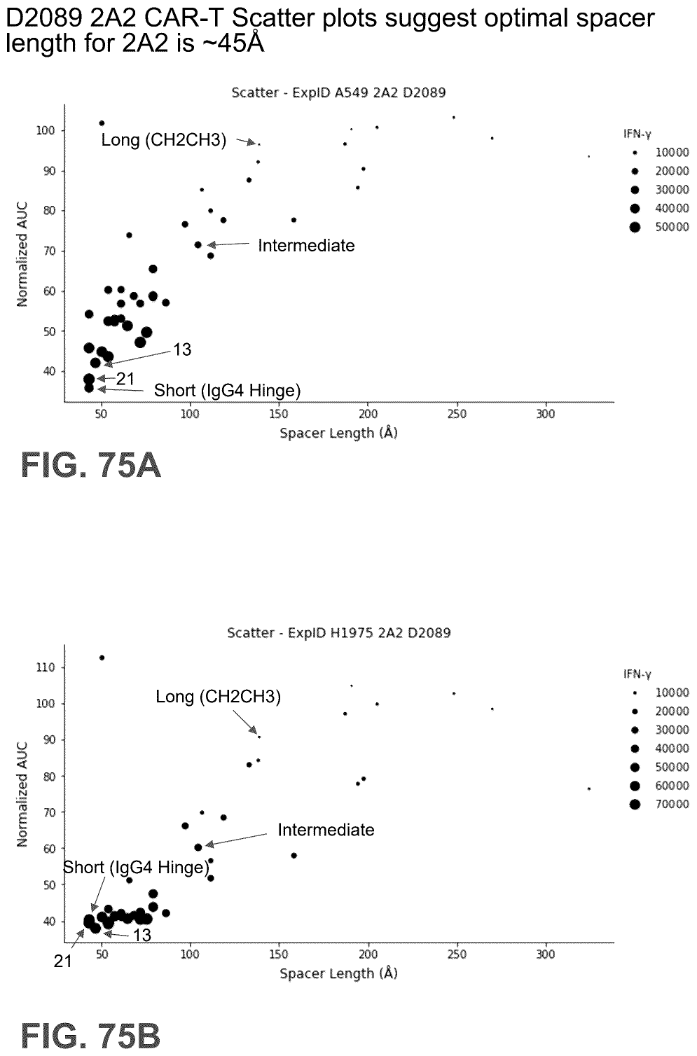

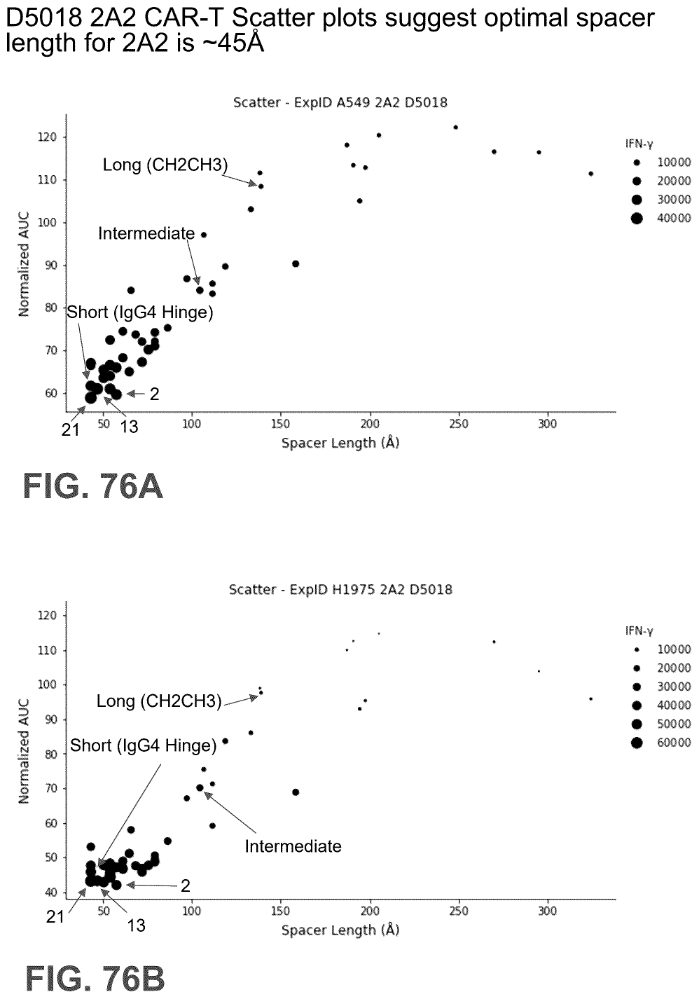

TABLE 25
Spacers used in Example 4 corresponding FIGS. 44A-77E.

| | Linker | Spacer | Length | | Linker | Spacer | Length |
|---|---|---|---|---|---|---|---|
| Spacer | SEQ | SEQ | (including | Spacer | SEQ | SEQ | (including |
| Identifier | ID NO | ID NO | linker) Å | Identifier | ID NO | ID NO | linker) Å |
| 1 | 4818 | 4830 | 50.4 | 25 | 4818 | 1768 | 86.4 |
| 2 | 4818 | 4831 | 57.6 | 26 | 4818 | 1889 | 79.2 |
| 3 | 4818 | 4832 | 72 | 27 | 4818 | 2015 | 68.4 |
| 4 | 4818 | 4833 | 248.4 | 28 | 4818 | 4856 | 54 |
| 5 | 4818 | 4834 | 205.2 | 29 | 4818 | 4857 | 111.6 |
| 6 | 4818 | 4835 | 187.2 | 30 | 4818 | 4858 | 54 |
| 7 | 4818 | 4836 | 133.2 | 31 | 4818 | 4859 | 61.2 |
| 8 | 4818 | 4837 | 97.2 | 41BB_1 | 4818 | 5009 | 197.6 |
| 9 | 4818 | 4838 | 79.2 | 41BB_2 | 4818 | 5010 | 138.3 |
| 10 | 4818 | 4839 | 79.2 | 41BB_3 | 4818 | 5011 | 106.7 |
| 11 | 4818 | 4840 | 75.6 | CD8a | 4818 | 5012 | 194.4 |
| 13 | 4818 | 4841 | 46.8 | CD27_1 | 4818 | 5013 | 324.4 |
| 14 | 4818 | 4842 | 57.6 | CD27_2 | 4818 | 5014 | 270 |
| 15 | 4818 | 4843 | 43.2 | CD28 | 4818 | 5015 | 158.4 |
| 16 | 4818 | 4844 | 118.8 | Short_nodisulf | 4818 | 5016 | 61.2 |
| 17 | 4818 | 4845 | 54 | Dap10 | 4818 | 5017 | 65.8 |
| 18 | 4818 | 4846 | 54 | GS_short_nodisulf | 4818 | 5018 | 61.2 |
| 19 | 4818 | 4847 | 50.4 | ICOS | 4818 | 5019 | 111.6 |
| 20 | 4818 | 4848 | 72 | OX40 | 4818 | 5020 | 190.8 |
| 21 | 4818 | 4849 | 43.2 | Intermediate | 4818 | 4912 | 104.5 |
| 22 | 4818 | 4850 | 43.2 | Long (CH2CH3) | 4818 | 4913 | 138.9 |
| 23 | 4818 | 4851 | 64.8 | Short (IgG4 Hinge) | 4818 | 4911 | 43.2 |
| 24 | 4818 | 4852 | 295.2 | | | | |

*In some aspects, the optional linker of SEQ ID NO: 4818 can be replaced with a linker of SEQ ID NO: 5088

### Example 4

### Spacer-Dependent Activity of ROR1 CAR-T Cells

R11 CAR: This study describes the effects of various Ig-derived or extracellular-domain-derived spacers on inducing CAR-T auto-activation, as well as human ROR1 directed cytokine release and potency. ROR1-specific R11 CARs were delivered using lentiviral constructs coding for bi-cistronic expression cassettes. The coding sequences for (i) a ROR1-specific R11 CAR, (ii) a P2A self-cleaving peptide, and (iii) EGFRt (a truncated EGFR having only Domains III and IV and the transmembrane domain; SEQ ID NO:26) were linked in frame and placed under the control of an MND promoter. The R11 CARs were derived from the R11 anti-ROR1 antibody and each comprises one of the indicated spacers, a CD28-derived transmembrane domain, a 4-1BB costimulatory domain, and a CD3 zeta signaling domain.

The results indicate varying levels of target-dependent IFN-7 and IL-2 secretion by the CAR-T cells expressing R11 CARs having different spacers as a component of the CAR design. Spacers 4 (ELKTPLGDTTHTCPRCPEPKSCDTPPPCPRCPEPKSCDTPPPCPRCPEPKSCDTPPPCPRCPAP; SEQ ID NO: 4833, or GGGGSGELKTPLGDTTHTCPRCPEPKSCDTPPPCPRCPEPKSCDTPPPCPRCPEPKSCDTPPPC PRCPAP; SEQ ID NO: 5039 when including the linker of SEQ ID NO:5088), 5 (CPRCPEPKSCDTPPPCPRCPEPKSCDTPPPCPRCPEPKSCDTPPPCPRCPAP; SEQ ID NO: 4834; or GGGGSGCPRCPEPKSCDTPPPCPRCPEPKSCDTPPPCPRCPEPKSCDTPPPCPRCPAP; SEQ ID NO: 5040 when including the link of SEQ ID NO:5088) and 6 (EPKSCDTPPPCPRCPEPKSCDTPPPCPRCPEPKSCDTPPPCPRCPAP; SEQ ID NO: 4835; or GGGGSGEPKSCDTPPPCPRCPEPKSCDTPPPCPRCPEPKSCDTPPPCPRCPAP; SEQ ID NO: 5041 when including the linker of SEQ ID NO:5088) in particular elicited R11 CAR-T cells to secrete the highest level of cytokines and the highest killing (lowest AUC in IncuCyte kinetic killing curves) in both donors. No tonic signaling was observed in any of the CAR-T cells In the context of spacer lengths, both IFN-7 and IL-2 output by the T cells were highest when CAR spacer lengths ranged from 187-248 Å. See FIGS. 44A-44D, 45A-45C, 46A-46C, 47A-47C, 48A-48B, 49A-49B, 50A-50B, 51, 52, 53A-53B, and 54A-54B.

R12 CAR: This study describes the effects of various Ig-derived or extracellular-domain-derived spacers on inducing CAR-T auto-activation, as well as human ROR1 directed cytokine release and potency. ROR1-specific R12 CARs were delivered using lentiviral constructs coding for bi-cistronic expression cassettes. The coding sequences for (i) a ROR1-specific R12 CAR, (ii) a P2A self-cleaving peptide, and (iii) EGFRt (a truncated EGFR having only Domains III and IV and the transmembrane domain; SEQ ID NO:26) were linked in frame and placed under the control of an MND promoter. The R12 CARs were derived from the R12 anti-ROR1 antibody and each comprises one of the indicated spacers, a CD28-derived transmembrane domain, a 4-1BB costimulatory domain, and a CD3 zeta signaling domain.

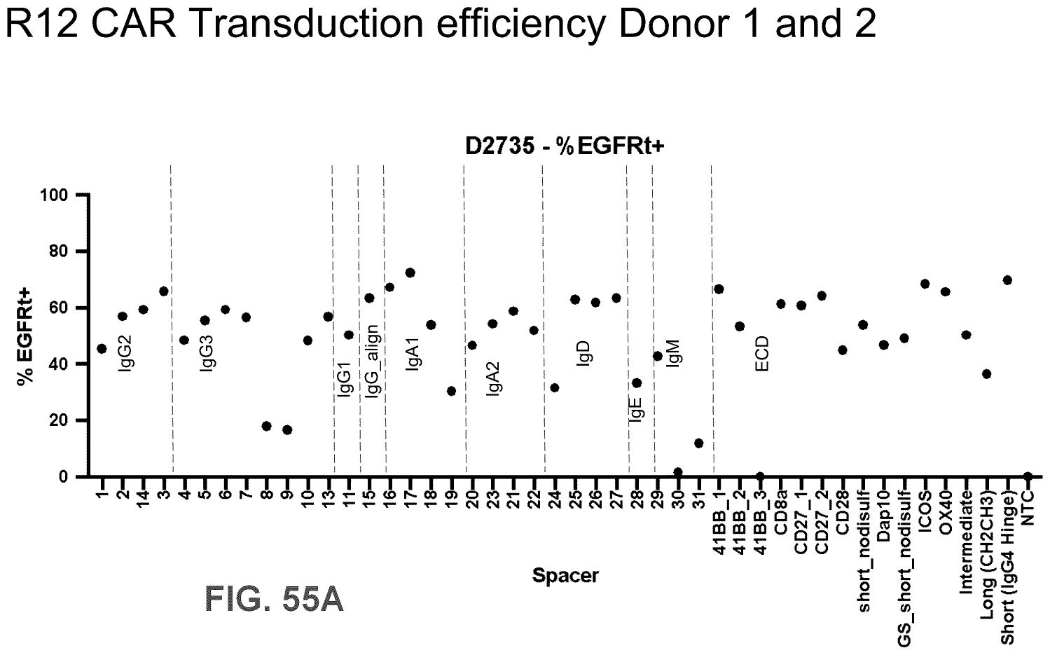

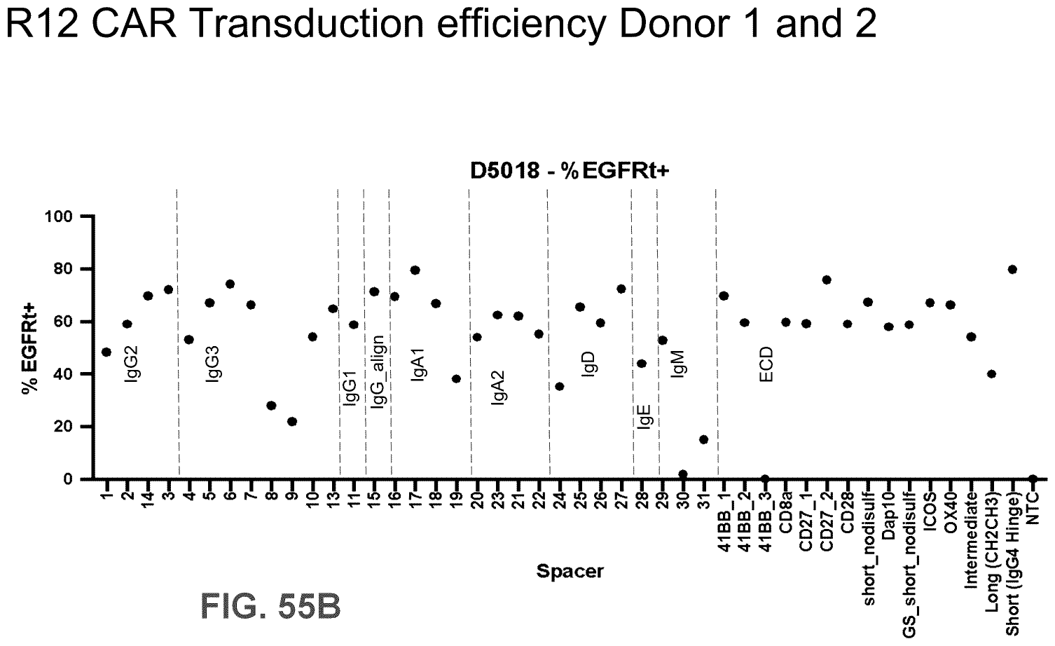

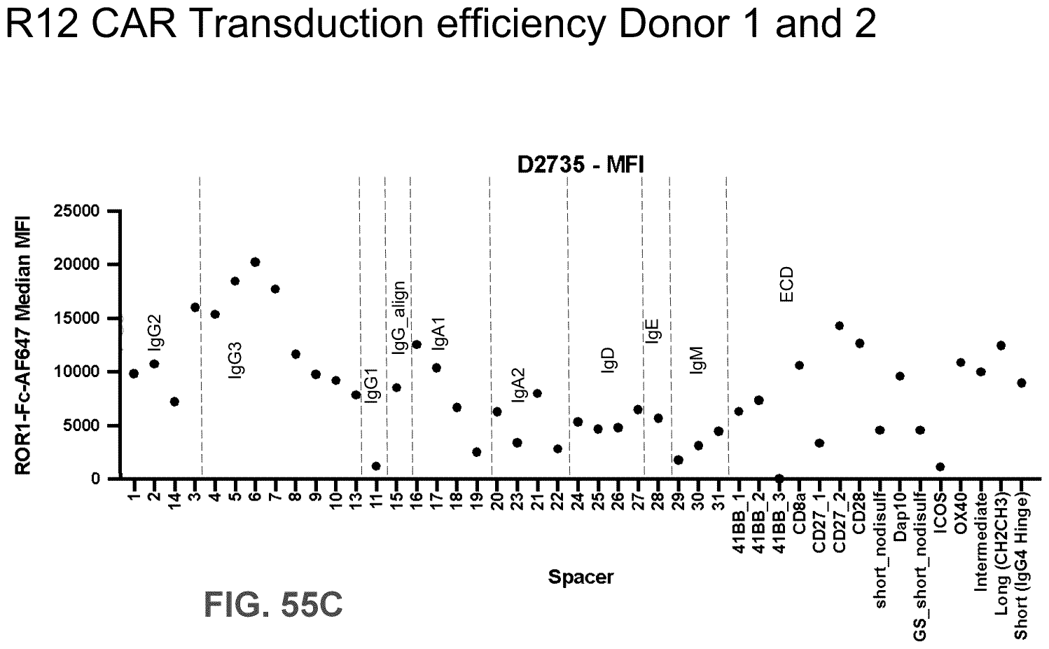

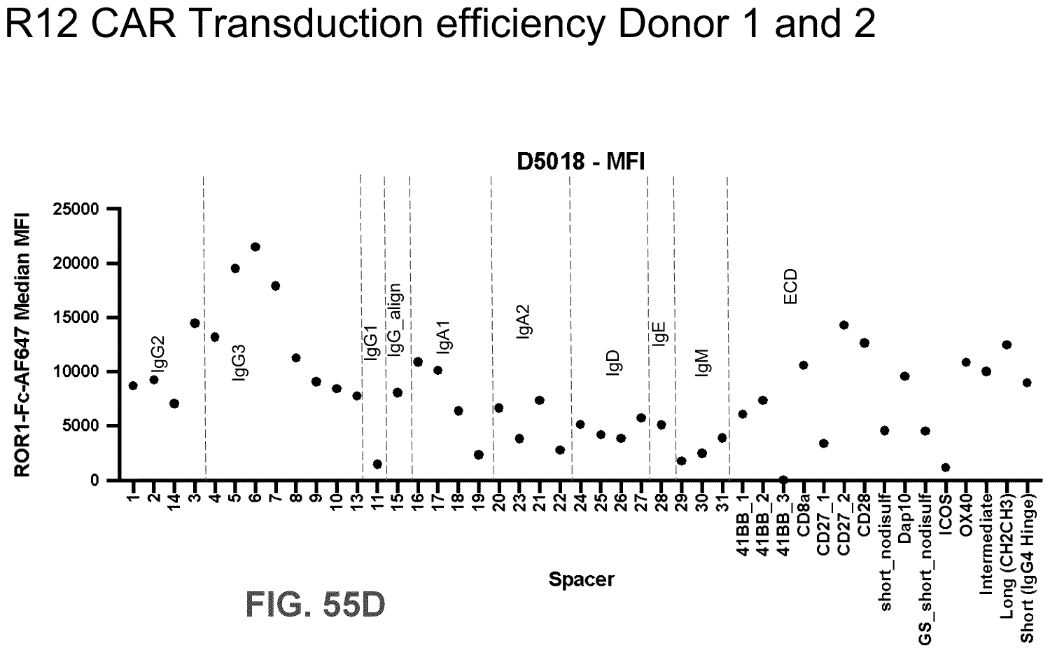

The results indicate varying levels of target-dependent IFN-7 and IL-2 secretion by the CAR-T cells expressing R12 CARs having different spacers as a component of the CAR design. Spacer 1 (KPCPPCKCP; SEQ ID NO: 4830; or GGGSGKPCPPCKCP; SEQ ID NO: 5042 when including the link of SEQ ID NO:4818), 15 (PPKPKDT; SEQ ID NO: 4843; or GGGSGPPKPKDT; SEQ ID NO: 5043 when including the link of SEQ ID NO:4818) and 21 (VPCPVPP; SEQ ID NO: 4849; or GGGSGVPCPVPP; SEQ ID NO: 5044 when including the link of SEQ ID NO:4818) in particular elicited R12 CAR-T cells to secrete the highest level of cytokines and the highest killing (lowest AUC in IncuCyte kinetic killing curves) in both donors.). No tonic signaling was observed in any of the CAR-T cells. In the context of spacer lengths, both IFN-7 and IL-2 output by the T cells were highest when CAR spacer lengths were approximately 45 Å. See FIGS. 55A-55D, 56A-56C, 57A-57C, 58A-58C, 59A-59B, 60A-60B, 61A-61B, 62, 63, 64A-64B, and 65A-65B.

2A2 CAR: This study describes the effects of various Ig-derived or extracellular-domain-derived spacers on inducing CAR-T auto-activation, as well as human ROR1 directed cytokine release and potency. ROR1-specific 2A2 CARs were delivered using lentiviral constructs coding for bi-cistronic expression cassettes. The coding sequences for (i) a ROR1-specific 2A2 CAR, (ii) a P2A self-cleaving peptide, and (iii) EGFRt (a truncated EGFR having only Domains III and IV and the transmembrane domain; SEQ ID NO:26) were linked in frame and placed under the control of an MND promoter. The 2A2 CARs were derived from the 2A2 anti-ROR1 antibody and each comprises one of the indicated spacers, a CD28-derived transmembrane domain, a 4-1BB costimulatory domain, and a CD3 zeta signaling domain.

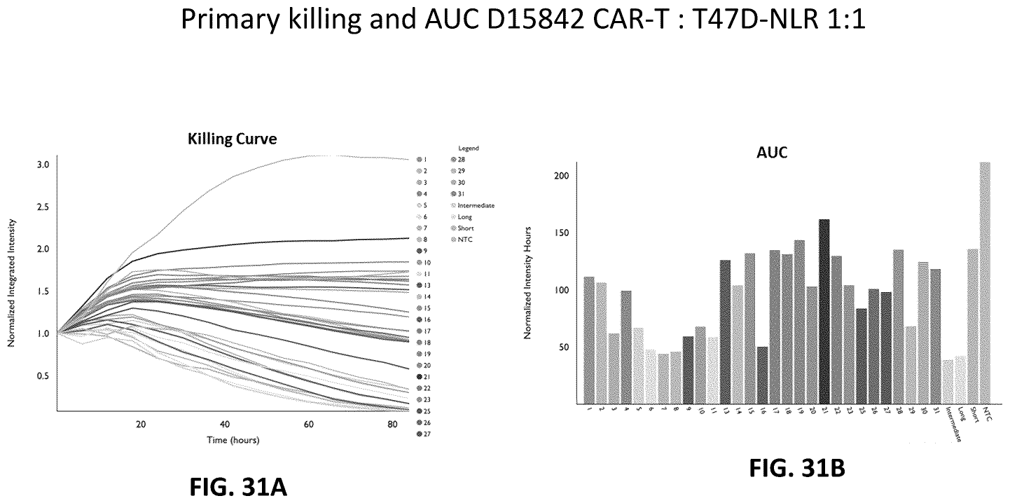

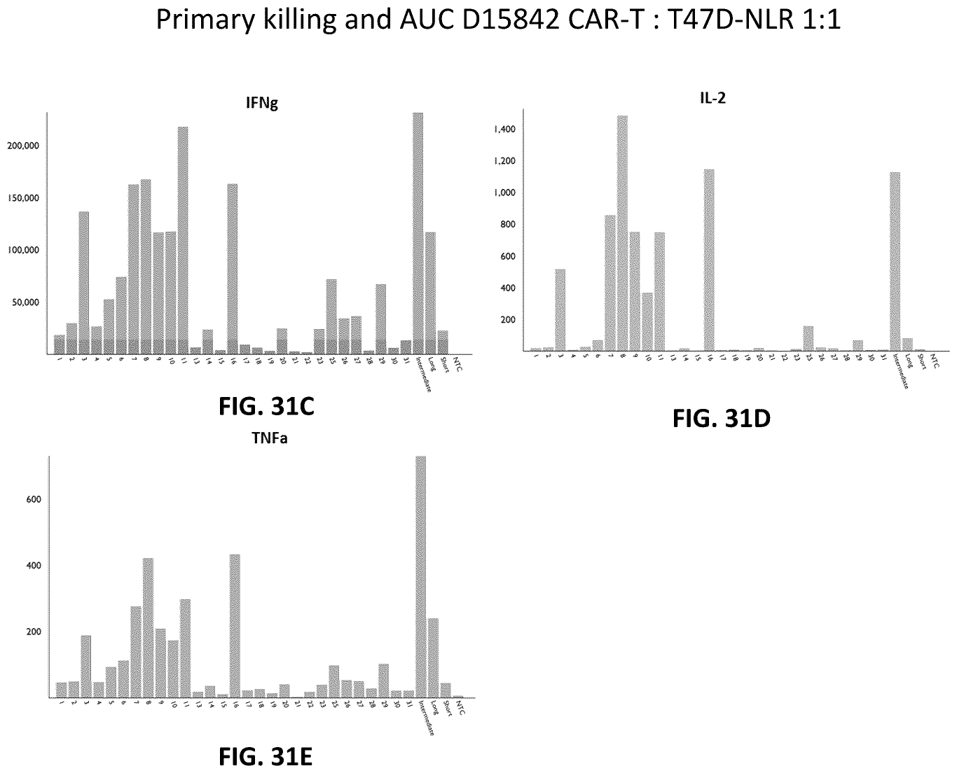

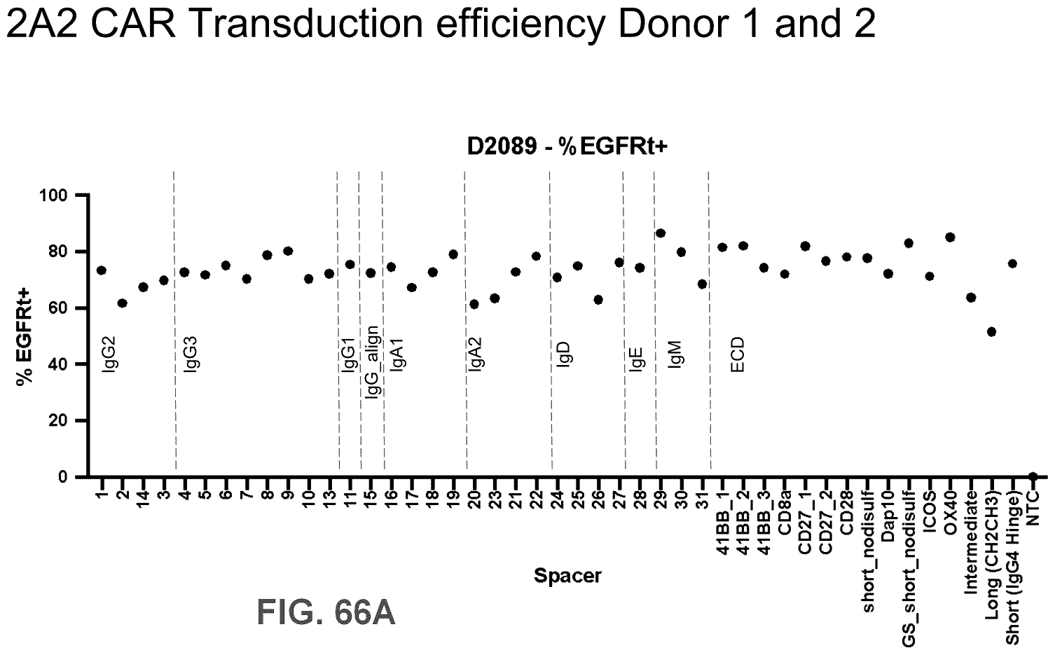

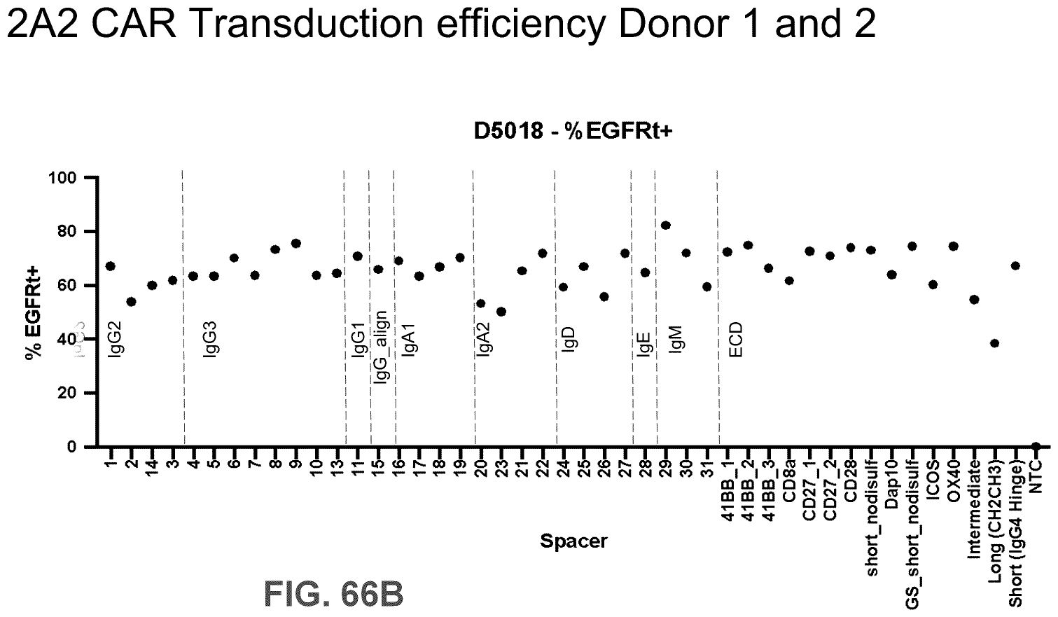

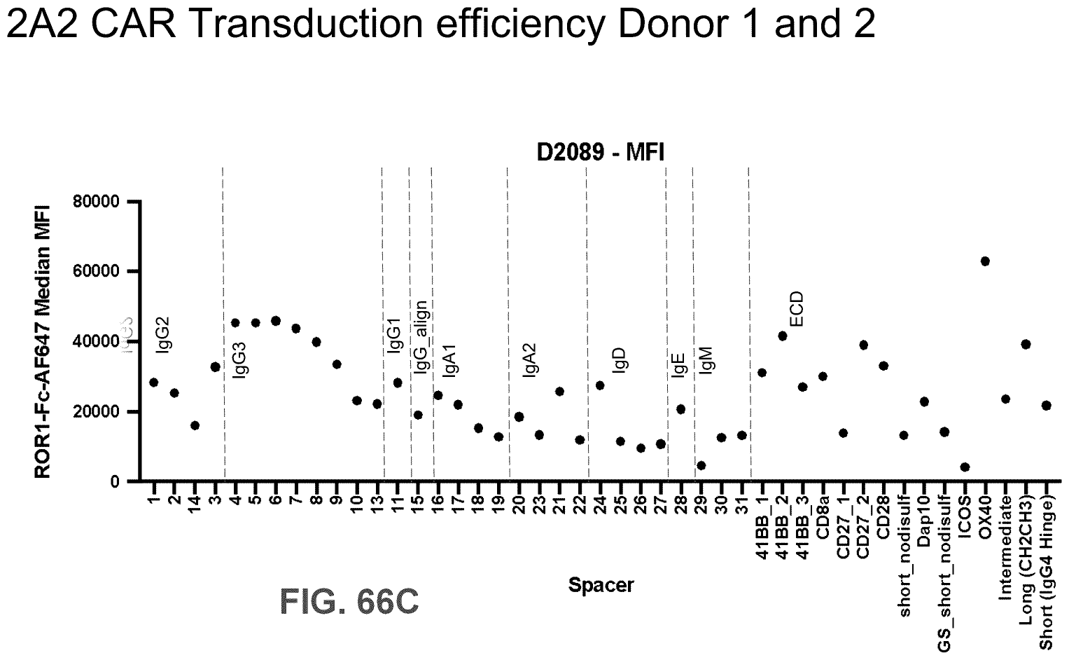

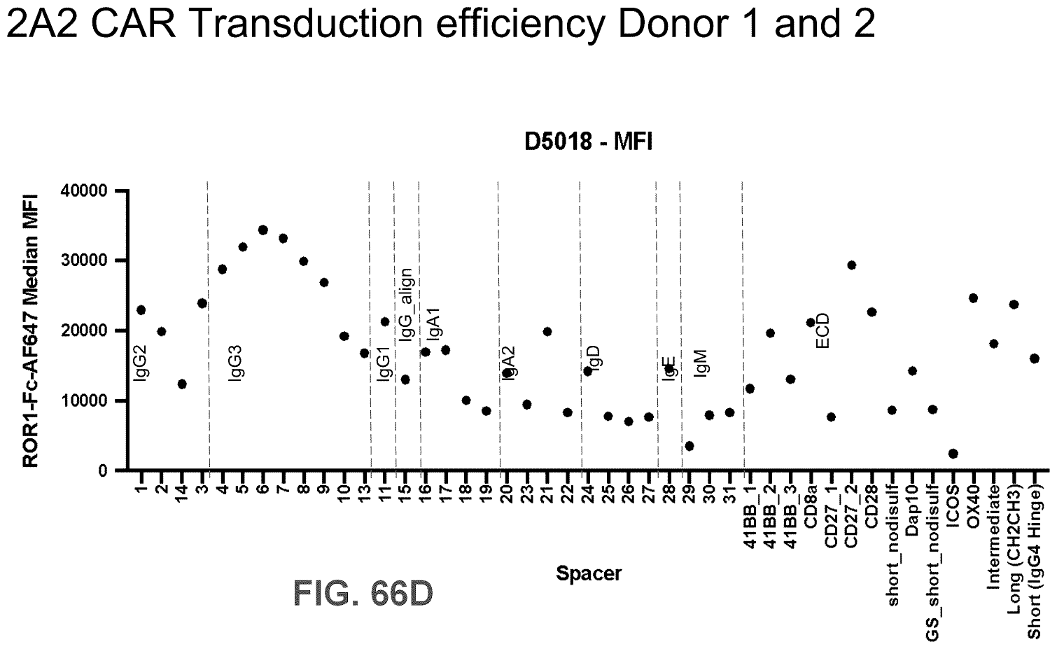

The results indicated varying levels of target-dependent IFN-7 and IL-2 secretion by the CAR-T cells expressing different spacers as a component of the CAR design. Spacer 13 (PCPRCPAP; SEQ ID NO: 4841; or GGGGSGPCPRCPAP; SEQ ID NO: 5045 when including the link of SEQ ID NO:5088), 21 (VPCPVPP; SEQ ID NO: 4849; or GGGGSGVPCPVPP; SEQ ID NO: 5044 when including the link of SEQ ID NO:5088) and 28 (SVCSRDFTPP; SEQ ID NO: 4856; or GGGGSGSVCSRDFTPP; SEQ ID NO: 5046 when including the link of SEQ ID NO:5088) in particular elicited 2A2 CAR-T cells to secrete the highest level of cytokines and the highest killing (lowest AUC in IncuCyte kinetic killing curves) in both donors. No tonic signaling was observed in any of the CAR-T cells as shown in FIG. 30 and FIG. 31. In the context of spacer lengths, both IFN-γ and IL-2 output by the T cells were highest when CAR spacer lengths were approximately 45 Å. See FIGS. 66A-66D, 67A-67C, 68A-68C, 69A-69C, 70A-70B, 71A-71B, 72A-72B, 73, 74, 75A-75B, and 76A-76B.

TABLE 26
Sequences of most active CARs identified

| | ROR1 | | |
|---|---|---|---|
| | R11 | R12 | 2A2 |
| Description | SEQ ID NO | SEQ ID NO | SEQ ID NO |
| Signal peptide | 4874 | 4874 | 4874 |
| scFv | 5048 | 4875 | 5047 |
| Gly/Ser linker | 4818 | 5088 | 4818 |
| | Spacer 4833 | 4830 (Spacer 1) | 4841 (Spacer 13) |
| (Spacer 4) | 4834 (Spacer 5) | 4843 (Spacer 15) | 4849 (Spacer 21) |
| | 4835 (Spacer 6) | 4849 (Spacer 21) | 4856 (Spacer 28) |
| Transmembrane | 4868 | 4868 | 4868 |
| domain | | | |
| 4-1BB | 4869 | 4869 | 4869 |
| costimulatory | | | |
| domain | | | |
| CD3z | 4870 | 4870 | 4870 |
| P2A | 4871 | 4871 | 4871 |
| EGFRt including | 4872 | 4872 | 4872 |
| GMCSF signal | | | |
| peptide of SEQ | | | |
| ID NO: 4865 | | | |

### Summary of Examples 1-4

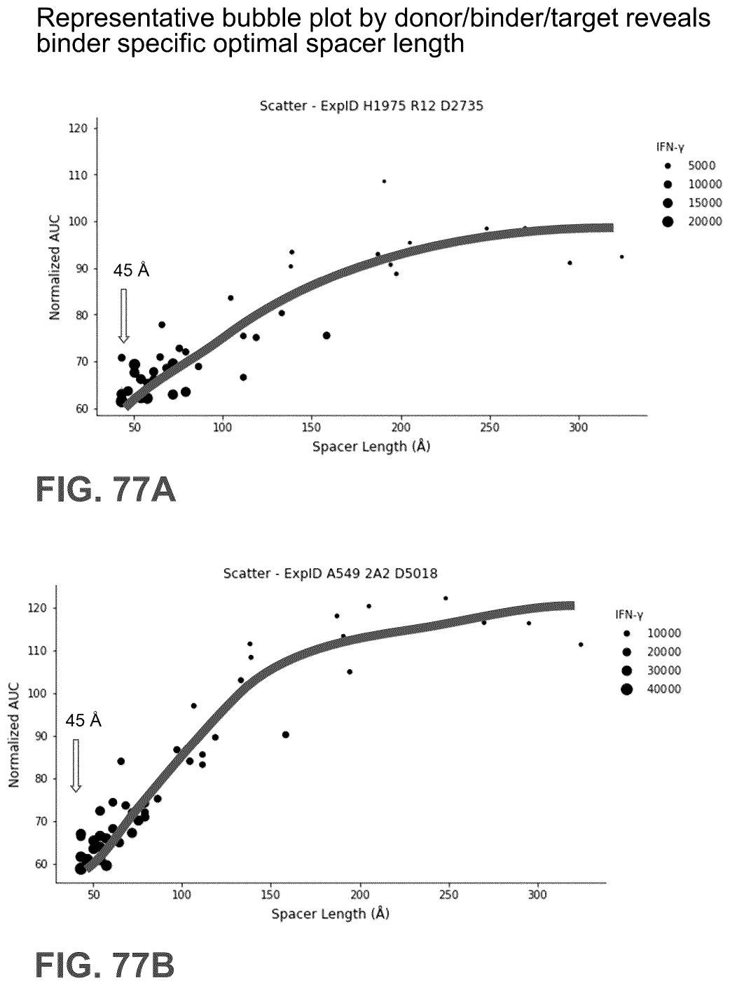

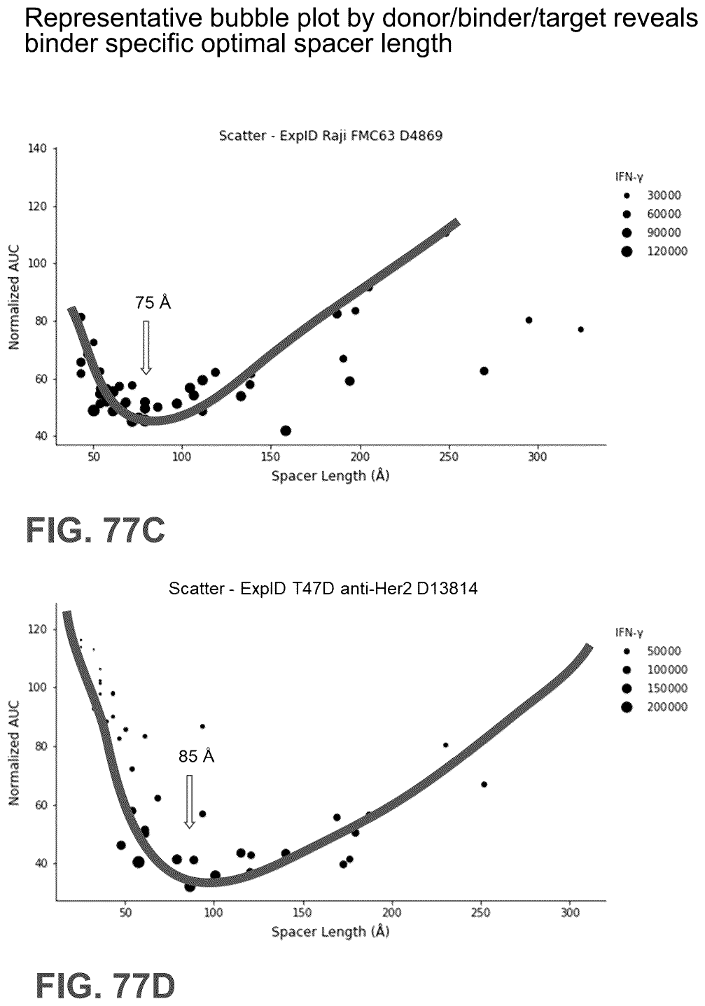

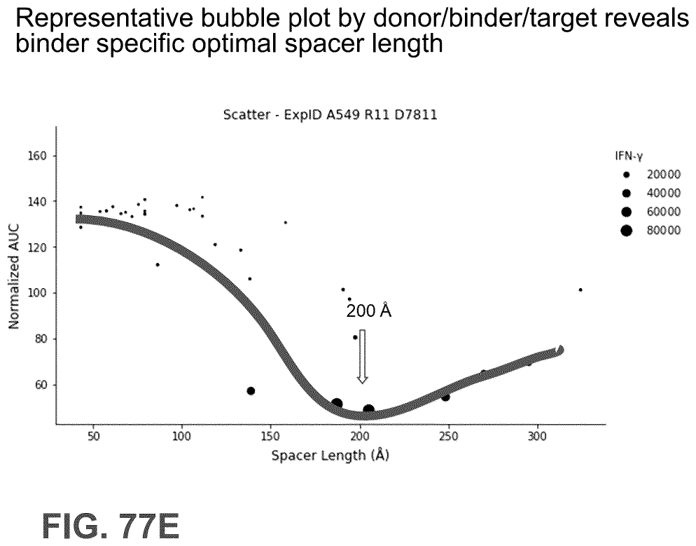

The results indicated that the optimal spacer differs depending on the binder. Generally, CARs targeted against membrane-proximal epitopes prefer long spacers, and CARs targeted against membrane-distal epitopes prefer short spacers. The optimal spacer lengths for R12, 2 Å2, FMC63, Her and R11 CARs are shown in FIGS. 77A-77E, respectively. Full sequences of the CARs with optimal CAR spacers identified according to the methods disclosed herein are provided in TABLE 27, below. The results have demonstrated that the disclosed collection of spacers have the proper biophysical properties (e.g., shape, flexibility/rigidity) to produce highly active CARs, and the entire collection is useful for empirically determining the optimal spacer for a given CAR/target antigen pair, either my using the disclosed spacers as a screening library, or for rational design based on the dimensions of spacers, binding groups, and antigen position with respect to the signaling synapse (see FIG. 77A-77E).

TABLE 27
Full CAR sequences for CARs targeting ROR1 (CARs comprising R11, R12 and 2A2 binders),
CD19 (CARs comprising FMC63 binder), and Her2 (CARs comprising 4D5 binder).
binders bolded
linkers italicized
spacers underlined
Constituent elements of the CAR can independently be absent or replaced with an
homologous element disclosed herein, known in the art, or a variant or derivative
thereof. For example, a transmembrane domain disclosed herein can be replaced with a
functionally equivalent transmembrane domain. Likewise, one or more of the 4-1BB
costimulatory domain, CD3z domain, P2A domain, or
EGFRt domain can be replaced with a functionally equivalent domain.

| | | |
|---|---|---|
| | Complete | |
| | CAR | |
| | SEQ ID | |
| Spacer | NO | Sequence |

R12 CARs against ROR1

| | | |
|---|---|---|
| 1 | 5049 | MVLQTQVFISLLLWISGAYGQEQLVESGGRLVTPGGSLTLSCKASGEDFSAYYMSWVRQAPGKG |
| | | LEWIATIYPSSGKTYYATWVNGRFTISSDNAQNTVDLQMNSLTAADRATYFCARDSYADDGALF |
| | | NIWGPGTLVTISSGGGGSGGGGSGGGGSELVLTQSPSVSAALGSPAKITCTLSSAHKTDTIDWY |
| | | QQLQGEAPRYLMQVQSDGSYTKRPGVPDRFSGSSSGADRYLIIPSVQADDEADYYCGADYIGGY |
| | | VFGGGTQLTVTGGGGSGKPCPPCKCPMFWVLVVVGGVLACYSLLVTVAFIIFWVKRGRKKLLYI |
| | | FKQPFMRPVQTTQEEDGCSCRFPEEEEGGCELRVKFSRSADAPAYQQGQNQLYNELNLGRREEY |
| | | DVLDKRRGRDPEMGGKPRRKNPQEGLYNELQKDKMAEAYSEIGMKGERRRGKGHDGLYQGLSTA |
| | | TKDTYDALHMQALPPRSGATNFSLLKQAGDVEENPGPMLLLVTSLLLCELPHPAFLLIPRKVCN |
| | | GIGIGEFKDSLSINATNIKHFKNCTSISGDLHILPVAFRGDSFTHTPPLDPQELDILKTVKEIT |
| | | GFLLIQAWPENRTDLHAFENLEIIRGRTKQHGQFSLAVVSLNITSLGLRSLKEISDGDVIISGN |
| | | KNLCYANTINWKKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCAHYIDGPHCVKT |
| | | CPAGVMGENNTLVWKYADAGHVCHLCHPNCTYGCTGPGLEGCPTNGPKIPSIATGMVGALLLLL |
| | | VVALGIGLFM |
| 15 | 5050 | MVLQTQVFISLLLWISGAYGQEQLVESGGRLVTPGGSLTLSCKASGEDFSAYYMSWVRQAPGKG |
| | | LEWIATIYPSSGKTYYATWVNGRFTISSDNAQNTVDLQMNSLTAADRATYFCARDSYADDGALF |
| | | NIWGPGTLVTISSGGGGSGGGGSGGGGSELVLTQSPSVSAALGSPAKITCTLSSAHKTDTIDWY |
| | | QQLQGEAPRYLMQVQSDGSYTKRPGVPDRFSGSSSGADRYLIIPSVQADDEADYYCGADYIGGY |
| | | VFGGGTQLTVTGGGGSGPPKPKDTMFWVLVVVGGVLACYSLLVTVAFIIFWVKRGRKKLLYIFK |
| | | QPFMRPVQTTQEEDGCSCRFPEEEEGGCELRVKFSRSADAPAYQQGQNQLYNELNLGRREEYDV |
| | | LDKRRGRDPEMGGKPRRKNPQEGLYNELQKDKMAEAYSEIGMKGERRRGKGHDGLYQGLSTATK |
| | | DTYDALHMQALPPRSGATNFSLLKQAGDVEENPGPMLLLVTSLLLCELPHPAFLLIPRKVCNGI |
| | | GIGEFKDSLSINATNIKHFKNCTSISGDLHILPVAFRGDSFTHTPPLDPQELDILKTVKEITGF |
| | | LLIQAWPENRTDLHAFENLEIIRGRTKQHGQFSLAVVSLNITSLGLRSLKEISDGDVIISGNKN |
| | | LCYANTINWKKLFGTSGQKTKIISNRGENSCKATGQVCHALCSPEGCWGPEPRDCVSCRNVSRG |
| | | RECVDKCNLLEGEPREFVENSECIQCHPECLPQAMNITCTGRGPDNCIQCAHYIDGPHCVKTCP |
| | | AGVMGENNTLVWKYADAGHVCHLCHPNCTYGCTGPGLEGCPTNGPKIPSIATGMVGALLLLLVV |
| | | ALGIGLFM |
| 21 | 5051 | MVLQTQVFISLLLWISGAYGQEQLVESGGRLVTPGGSLTLSCKASGEDFSAYYMSWVRQAPGKG |
| | | LEWIATIYPSSGKTYYATWVNGRFTISSDNAQNTVDLQMNSLTAADRATYFCARDSYADDGALF |
| | | NIWGPGTLVTISSGGGGSGGGGSGGGGSELVLTQSPSVSAALGSPAKITCTLSSAHKTDTIDWY |
| | | QQLQGEAPRYLMQVQSDGSYTKRPGVPDRFSGSSSGADRYLIIPSVQADDEADYYCGADYIGGY |
| | | VFGGGTQLTVTGGGGSGVPCPVPPMFWVLVVVGGVLACYSLLVTVAFTIFWVKRGRKKLLYIFK |
| | | QPFMRPVQTTQEEDGCSCREPEEEEGGCELRVKFSRSADAPAYQQGQNQLYNELNLGRREEYDV |
| | | LDKRRGRDPEMGGKPRRKNPQEGLYNELQKDKMAEAYSEIGMKGERRRGKGHDGLYQGLSTATK |
| | | DTYDALHMQALPPRSGATNFSLLKQAGDVEENPGPMLLLVTSLLLCELPHPAFLLIPRKVCNGI |
| | | GIGEFKDSLSINATNIKHFKNCTSISGDLHILPVAFRGDSFTHTPPLDPQELDILKTVKEITGF |
| | | LLIQAWPENRTDLHAFENLEIIRGRTKQHGQFSLAVVSLNITSLGLRSLKEISDGDVIISGNKN |
| | | LCYANTINWKKLEGTSGQKTKIISNRGENSCKATGQVCHALCSPEGCWGPEPRDCVSCRNVSRG |
| | | RECVDKCNLLEGEPREFVENSECIQCHPECLPQAMNITCTGRGPDNCIQCAHYIDGPHCVKTCP |
| | | AGVMGENNTLVWKYADAGHVCHLCHPNCTYGCTGPGLEGCPTNGPKIPSIATGMVGALLLLLVV |
| | | ALGIGLFM |

FMC63 CARs against CD19

| | | |
|---|---|---|
| 9 | 5052 | MVLQTQVFISLLLWISGAYGDIQMTQTTSSLSASLGDRVTISCRASQDISKYLNWYQQKPDGTV |
| | | KLLIYHTSRLHSGVPSRFSGSGSGTDYSLTISNLEQEDIATYFCQQGNTLPYTFGGGTKLEITG |
| | | STSGSGKPGSGEGSTKGEVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYYCAKHYYYGGSYAMDYWG |
| | | QGTSVTVSSGGGGSGEPKSCDTPPPCPRCPAPMFWVLVVVGGVLACYSLLVTVAFTIFWVKRGR |
| | | KKLLYIFKQPFMRPVQTTQEEDGCSCRFPEEEEGGCELRVKFSRSADAPAYQQGQNQLYNELNL |
| | | GRREEYDVLDKRRGRDPEMGGKPRRKNPQEGLYNELQKDKMAEAYSEIGMKGERRRGKGHDGLY |
| | | QGLSTATKDTYDALHMQALPPRSGATNFSLLKQAGDVEENPGPMLLLVTSLLLCELPHPAFLLI |
| | | PRKVCNGIGIGEFKDSLSINATNIKHFKNCTSISGDLHILPVAFRGDSFTHTPPLDPQELDILK |
| | | TVKEITGFLLIQAWPENRTDLHAFENLEIIRGRTKQHGQFSLAVVSLNITSLGLRSLKEISDGD |
| | | VIISGNKNLCYANTINWKKLEGTSGQKTKIISNRGENSCKATGQVCHALCSPEGCWGPEPRDCV |
| | | SCRNVSRGRECVDKCNLLEGEPREFVENSECIQCHPECLPQAMNITCTGRGPDNCIQCAHYIDG |
| | | PHCVKTCPAGVMGENNTLVWKYADAGHVCHLCHPNCTYGCTGPGLEGCPTNGPKIPSIATGMVG |
| | | ALLLLLVVALGIGLFM |
| 10 | 5053 | MVLQTQVFISLLLWISGAYGDIQMTQTTSSLSASLGDRVTISCRASQDISKYLNWYQQKPDGTV |
| | | KLLIYHTSRLHSGVPSRFSGSGSGTDYSLTISNLEQEDIATYFCQQGNTLPYTFGGGTKLEITG |
| | | STSGSGKPGSGEGSTKGEVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYYCAKHYYYGGSYAMDYWG |
| | | QGTSVTVSSGGGGSGEPKSCDKTHTCPPCPAPMFWVLVVVGGVLACYSLLVTVAFTIFWVKRGR |
| | | KKLLYIFKQPFMRPVQTTQEEDGCSCRFPEEEEGGCELRVKFSRSADAPAYQQGQNQLYNELNL |
| | | GRREEYDVLDKRRGRDPEMGGKPRRKNPQEGLYNELQKDKMAEAYSEIGMKGERRRGKGHDGLY |
| | | QGLSTATKDTYDALHMQALPPRSGATNFSLLKQAGDVEENPGPMLLLVTSLLLCELPHPAFLLI |
| | | PRKVCNGIGIGEFKDSLSINATNIKHFKNCTSISGDLHILPVAFRGDSFTHTPPLDPQELDILK |
| | | TVKEITGFLLIQAWPENRTDLHAFENLEIIRGRTKQHGQFSLAVVSLNITSLGLRSLKEISDGD |
| | | VIISGNKNLCYANTINWKKLEGTSGQKTKIISNRGENSCKATGQVCHALCSPEGCWGPEPRDCV |
| | | SCRNVSRGRECVDKCNLLEGEPREFVENSECIQCHPECLPQAMNITCTGRGPDNCIQCAHYIDG |
| | | PHCVKTCPAGVMGENNTLVWKYADAGHVCHLCHPNCTYGCTGPGLEGCPTNGPKIPSIATGMVG |
| | | ALLLLLVVALGIGLFM |
| 11 | 5054 | MVLQTQVFISLLLWISGAYGDIQMTQTTSSLSASLGDRVTISCRASQDISKYLNWYQQKPDGTV |
| | | KLLIYHTSRLHSGVPSRFSGSGSGTDYSLTISNLEQEDIATYFCQQGNTLPYTFGGGTKLEITG |
| | | STSGSGKPGSGEGSTKGEVKLQESGPGLVAPSQSLSVTCTVSGVSLPDYGVSWIRQPPRKGLEW |
| | | LGVINGSETTYYNSALKSRLTIIKDNSKSQVFLKMNSLQTDDTAIYYCAKHYYYGGSYAMDYWG |
| | | QGTSVTVSSGGGGSGPKSCDKTHTCPPCPAPMFWVLVVVGGVLACYSLLVTVAFTIFWVKRGRK |
| | | KLLYIFKQPFMRPVQTTQEEDGCSCRFPEEEEGGCELRVKFSRSADAPAYQQGQNQLYNELNLG |
| | | RREEYDVLDKRRGRDPEMGGKPRRKNPQEGLYNELQKDKMAEAYSEIGMKGERRRGKGHDGLYQ |
| | | GLSTATKDTYDALHMQALPPRSGATNFSLLKQAGDVEENPGPMLLLVTSLLLCELPHPAFLLIP |
| | | RKVCNGIGIGEFKDSLSINATNIKHFKNCTSISGDLHILPVAFRGDSFTHTPPLDPQELDILKT |
| | | VKEITGFLLIQAWPENRTDLHAFENLEIIRGRTKQHGQFSLAVVSLNITSLGLRSLKEISDGDV |
| | | IISGNKNLCYANTINWKKLEGTSGQKTKIISNRGENSCKATGQVCHALCSPEGCWGPEPRDCVS |
| | | CRNVSRGRECVDKCNLLEGEPREFVENSECIQCHPECLPQAMNITCTGRGPDNCIQCAHYIDGP |
| | | HCVKTCPAGVMGENNTLVWKYADAGHVCHLCHPNCTYGCTGPGLEGCPTNGPKIPSIATGMVGA |
| | | LLLLLVVALGIGLFM |

4D5 CARs against Her2

| | | |
|---|---|---|
| 7 | 5055 | MVLQTQVFISLLLWISGAYGDIQMTQSPSSLSASVGDRVTITCRASQDVNTAVAWYQQKPGKAP |
| | | KLLIYSASFLYSGVPSRFSGSRSGTDFTLTISSLQPEDFATYYCQQHYTTPPTFGQGTKVEIKG |
| | | STSGSGKPGSGEGSGEVQLVESGGGLVQPGGSLRLSCAASGENIKDTYIHWVRQAPGKGLEWVA |
| | | RIYPTNGYTRYADSVKGRFTISADTSKNTAYLQMNSLRAEDTAVYYCSRWGGDGFYAMDYWGQG |
| | | TLVTVSSEPKSCDTPPPCPRCPEPKSCDTPPPCPRCPAPMFWVLVVVGGVLACYSLLVTVAFII |
| | | FWVKRGRKKLLYIFKQPFMRPVQTTQEEDGCSCRFPEEEEGGCELRVKFSRSADAPAYQQGQNQ |
| | | LYNELNLGRREEYDVLDKRRGRDPEMGGKPRRKNPQEGLYNELQKDKMAEAYSEIGMKGERRRG |
| | | KGHDGLYQGLSTATKDTYDALHMQALPPRSGATNESLLKQAGDVEENPGPMLLLVTSLLLCELP |
| | | HPAFLLIPRKVCNGIGIGEFKDSLSINATNIKHFKNCTSISGDLHILPVAFRGDSFTHTPPLDP |
| | | QELDILKTVKEITGFLLIQAWPENRTDLHAFENLEIIRGRTKQHGQFSLAVVSLNITSLGLRSL |
| | | KEISDGDVIISGNKNLCYANTINWKKLEGTSGQKTKIISNRGENSCKATGQVCHALCSPEGCWG |
| | | PEPRDCVSCRNVSRGRECVDKCNLLEGEPREFVENSECIQCHPECLPQAMNITCTGRGPDNCIQ |
| | | CAHYIDGPHCVKTCPAGVMGENNTLVWKYADAGHVCHLCHPNCTYGCTGPGLEGCPTNGPKIPS |
| | | IATGMVGALLLLLVVALGIGLFM |
| 8 | 5056 | MVLQTQVFISLLLWISGAYGDIQMTQSPSSLSASVGDRVTITCRASQDVNTAVAWYQQKPGKAP |
| | | KLLIYSASFLYSGVPSRFSGSRSGTDFTLTISSLQPEDFATYYCQQHYTTPPTFGQGTKVEIKG |
| | | STSGSGKPGSGEGSGEVQLVESGGGLVQPGGSLRLSCAASGENIKDTYIHWVRQAPGKGLEWVA |
| | | RIYPTNGYTRYADSVKGRFTISADTSKNTAYLQMNSLRAEDTAVYYCSRWGGDGFYAMDYWGQG |
| | | TLVTVSSCPRCPEPKSCDTPPPCPRCPAPMFWVLVVVGGVLACYSLLVTVAFTIFWVKRGRKKL |
| | | LYIFKQPFMRPVQTTQEEDGCSCRFPEEEEGGCELRVKFSRSADAPAYQQGQNQLYNELNLGRR |
| | | EEYDVLDKRRGRDPEMGGKPRRKNPQEGLYNELQKDKMAEAYSEIGMKGERRRGKGHDGLYQGL |
| | | STATKDTYDALHMQALPPRSGATNFSLLKQAGDVEENPGPMLLLVTSLLLCELPHPAFLLIPRK |
| | | VCNGIGIGEFKDSLSINATNIKHFKNCTSISGDLHILPVAFRGDSFTHTPPLDPQELDILKTVK |
| | | EITGFLLIQAWPENRTDLHAFENLEIIRGRTKQHGQFSLAVVSLNITSLGLRSLKEISDGDVII |
| | | SGNKNLCYANTINWKKLEGTSGQKTKIISNRGENSCKATGQVCHALCSPEGCWGPEPRDCVSCR |
| | | NVSRGRECVDKCNLLEGEPREFVENSECIQCHPECLPQAMNITCTGRGPDNCIQCAHYIDGPHC |
| | | VKTCPAGVMGENNTLVWKYADAGHVCHLCHPNCTYGCTGPGLEGCPTNGPKIPSIATGMVGALL |
| | | LLLVVALGIGLFM |
| 16 | 5057 | MVLQTQVFISLLLWISGAYGDIQMTQSPSSLSASVGDRVTITCRASQDVNTAVAWYQQKPGKAP |
| | | KLLIYSASFLYSGVPSRFSGSRSGTDFTLTISSLQPEDFATYYCQQHYTTPPTFGQGTKVEIKG |
| | | STSGSGKPGSGEGSGEVQLVESGGGLVQPGGSLRLSCAASGFNIKDTYIHWVRQAPGKGLEWVA |
| | | RIYPTNGYTRYADSVKGRFTISADTSKNTAYLQMNSLRAEDTAVYYCSRWGGDGFYAMDYWGQG |
| | | TLVTVSSTVPCPVPSTPPTPSPSTPPTPSPSCCHPMFWVLVVVGGVLACYSLLVTVAFTIFWVK |
| | | RGRKKLLYIFKQPFMRPVQTTQEEDGCSCRFPEEEEGGCELRVKFSRSADAPAYQQGQNQLYNE |
| | | LNLGRREEYDVLDKRRGRDPEMGGKPRRKNPQEGLYNELQKDKMAEAYSEIGMKGERRRGKGHD |
| | | GLYQGLSTATKDTYDALHMQALPPRSGATNFSLLKQAGDVEENPGPMLLLVTSLLLCELPHPAF |
| | | LLIPRKVCNGIGIGEFKDSLSINATNIKHFKNCTSISGDLHILPVAFRGDSFTHTPPLDPQELD |
| | | ILKTVKEITGFLLIQAWPENRTDLHAFENLEIIRGRTKQHGQFSLAVVSLNITSLGLRSLKEIS |
| | | DGDVIISGNKNLCYANTINWKKLEGTSGQKTKIISNRGENSCKATGQVCHALCSPEGCWGPEPR |
| | | DCVSCRNVSRGRECVDKCNLLEGEPREFVENSECIQCHPECLPQAMNITCTGRGPDNCIQCAHY |
| | | IDGPHCVKTCPAGVMGENNTLVWKYADAGHVCHLCHPNCTYGCTGPGLEGCPTNGPKIPSIATG |
| | | MVGALLLLLVVALGIGLFM |

2A2 CARs against ROR1

| | | |
|---|---|---|
| 13 | 5058 | MVLQTQVFISLLLWISGAYGQVQLQQSGAELVRPGASVTLSCKASGYTFSDYEMHWVIQTPVHG |
| | | LEWIGAIDPETGGTAYNQKFKGKAILTADKSSSTAYMELRSLTSEDSAVYYCTGYYDYDSFTYW |
| | | GQGTLVTVSAGGGGSGGGGSGGGGSDIVMTQSQKIMSTTVGDRVSITCKASQNVDAAVAWYQQK |
| | | PGQSPKLLIYSASNRYTGVPDRFTGSGSGTDFTLTISNMQSEDLADYFCQQYDIYPYTFGGGTK |
| | | LEIKTGGGGSGPCPRCPAPMFWVLVVVGGVLACYSLLVTVAFIIFWVKRGRKKLLYIFKQPFMR |
| | | PVQTTQEEDGCSCRFPEEEEGGCELRVKFSRSADAPAYQQGQNQLYNELNLGRREEYDVLDKRR |
| | | GRDPEMGGKPRRKNPQEGLYNELQKDKMAEAYSEIGMKGERRRGKGHDGLYQGLSTATKDTYDA |
| | | LHMQALPPRSGATNFSLLKQAGDVEENPGPMLLLVTSLLLCELPHPAFLLIPRKVCNGIGIGEF |
| | | KDSLSINATNIKHFKNCTSISGDLHILPVAFRGDSFTHTPPLDPQELDILKTVKEITGFLLIQA |
| | | WPENRTDLHAFENLEIIRGRTKQHGQFSLAVVSLNITSLGLRSLKEISDGDVIISGNKNLCYAN |
| | | TINWKKLFGTSGQKTKIISNRGENSCKATGQVCHALCSPEGCWGPEPRDCVSCRNVSRGRECVD |
| | | KCNLLEGEPREFVENSECIQCHPECLPQAMNITCTGRGPDNCIQCAHYIDGPHCVKTCPAGVMG |
| | | ENNTLVWKYADAGHVCHLCHPNCTYGCTGPGLEGCPTNGPKIPSIATGMVGALLLLLVVALGIG |
| | | LFM |
| 21 | 5059 | MVLQTQVFISLLLWISGAYGQVQLQQSGAELVRPGASVTLSCKASGYTFSDYEMHWVIQTPVHG |
| | | LEWIGAIDPETGGTAYNQKFKGKAILTADKSSSTAYMELRSLTSEDSAVYYCTGYYDYDSFTYW |
| | | GQGTLVTVSAGGGGSGGGGSGGGGSDIVMTQSQKIMSTTVGDRVSITCKASQNVDAAVAWYQQK |
| | | PGQSPKLLIYSASNRYTGVPDRFTGSGSGTDFTLTISNMQSEDLADYFCQQYDIYPYTFGGGTK |
| | | LEIKTGGGGSGVPCPVPPMFWVLVVVGGVLACYSLLVTVAFIIFWVKRGRKKLLYIFKQPFMRP |
| | | VQTTQEEDGCSCRFPEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQGLSTATKDTYDAL |
| | | HMQALPPRSGATNFSLLKQAGDVEENPGPMLLLVTSLLLCELPHPAFLLIPRKVCNGIGIGEFK |
| | | DSLSINATNIKHFKNCTSISGDLHILPVAFRGDSFTHTPPLDPQELDILKTVKEITGFLLIQAW |
| | | PENRTDLHAFENLEIIRGRTKQHGQFSLAVVSLNITSLGLRSLKEISDGDVIISGNKNLCYANT |
| | | INWKKLFGTSGQKTKIISNRGENSCKATGQVCHALCSPEGCWGPEPRDCVSCRNVSRGRECVDK |
| | | CNLLEGEPREFVENSECIQCHPECLPQAMNITCTGRGPDNCIQCAHYIDGPHCVKTCPAGVMGE |
| | | NNTLVWKYADAGHVCHLCHPNCTYGCTGPGLEGCPTNGPKIPSIATGMVGALLLLLVVALGIGL |
| | | FM |
| 28 | 5960 | MVLQTQVFISLLLWISGAYGQVQLQQSGAELVRPGASVTLSCKASGYTFSDYEMHWVIQTPVHG |
| | | LEWIGAIDPETGGTAYNQKFKGKAILTADKSSSTAYMELRSLTSEDSAVYYCTGYYDYDSFTYW |
| | | GQGTLVTVSAGGGGSGGGGSGGGGSDIVMTQSQKIMSTTVGDRVSITCKASQNVDAAVAWYQQK |
| | | PGQSPKLLIYSASNRYTGVPDRFTGSGSGTDFTLTISNMQSEDLADYFCQQYDIYPYTFGGGTK |
| | | LEIKTGGGGSGSVCSRDFTPPMFWVLVVVGGVLACYSLLVTVAFIIFWVKRGRKKLLYIFKQPF |
| | | MRPVQTTQEEDGCSCRFPEEEEGGCELRVKFSRSADAPAYQQGQNQLYNELNLGRREEYDVLDK |
| | | RRGRDPEMGGKPRRKNPQEGLYNELQKDKMAEAYSEIGMKGERRRGKGHDGLYQGLSTATKDTY |
| | | DALHMQALPPRSGATNFSLLKQAGDVEENPGPMLLLVTSLLLCELPHPAFLLIPRKVCNGIGIG |
| | | EFKDSLSINATNIKHFKNCTSISGDLHILPVAFRGDSFTHTPPLDPQELDILKTVKEITGFLLI |
| | | QAWPENRTDLHAFENLEIIRGRTKQHGQFSLAVVSLNITSLGLRSLKEISDGDVIISGNKNLCY |
| | | ANTINWKKLFGTSGQKTKIISNRGENSCKATGQVCHALCSPEGCWGPEPRDCVSCRNVSRGREC |
| | | VDKCNLLEGEPREFVENSECIQCHPECLPQAMNITCTGRGPDNCIQCAHYIDGPHCVKTCPAGV |
| | | MGENNTLVWKYADAGHVCHLCHPNCTYGCTGPGLEGCPTNGPKIPSIATGMVGALLLLLVVALG |
| | | IGLFM |

R11 CARs against ROR1

| | | |
|---|---|---|
| 4 | 5061 | MVLQTQVFISLLLWISGAYGQSVKESEGDLVTPAGNLTLTCTASGSDINDYPISWVRQAPGKGL |
| | | EWIGFINSGGSTWYASWVKGRFTISRTSTTVDLKMTSLTTDDTATYFCARGYSTYYGDFNIWGP |
| | | GTLVTISSGGGGSGGGGSGGGGSELVMTQTPSSTSGAVGGTVTINCQASQSIDSNLAWFQQKPG |
| | | QPPTLLIYRASNLASGVPSRFSGSRSGTEYTLTISGVQREDAATYYCLGGVGNVSYRTSFGGGT |
| | | EVVVKTGGGGSGELKTPLGDTTHTCPRCPEPKSCDTPPPCPRCPEPKSCDTPPPCPRCPEPKSC |
| | | DTPPPCPRCPAPMFWVLVVVGGVLACYSLLVTVAFTIFWVKRGRKKLLYIFKQPFMRPVQTTQE |
| | | EDGCSCRFPEEEEGGCELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDTYDALHMQALP |
| | | PRSGATNFSLLKQAGDVEENPGPMLLLVTSLLLCELPHPAFLLIPRKVCNGIGIGEFKDSLSIN |
| | | ATNIKHFKNCTSISGDLHILPVAFRGDSFTHTPPLDPQELDILKTVKEITGFLLIQAWPENRTD |
| | | LHAFENLEIIRGRTKQHGQFSLAVVSLNITSLGLRSLKEISDGDVIISGNKNLCYANTINWKKL |
| | | FGTSGQKTKIISNRGENSCKATGQVCHALCSPEGCWGPEPRDCVSCRNVSRGRECVDKCNLLEG |
| | | EPREFVENSECIQCHPECLPQAMNITCTGRGPDNCIQCAHYIDGPHCVKTCPAGVMGENNTLVW |
| | | KYADAGHVCHLCHPNCTYGCTGPGLEGCPTNGPKIPSIATGMVGALLLLLVVALGIGLFM |
| 5 | 5062 | MVLQTQVFISLLLWISGAYGQSVKESEGDLVTPAGNLTLTCTASGSDINDYPISWVRQAPGKGL |
| | | EWIGFINSGGSTWYASWVKGRFTISRTSTTVDLKMTSLTTDDTATYFCARGYSTYYGDFNIWGP |
| | | GTLVTISSGGGGSGGGGSGGGGSELVMTQTPSSTSGAVGGTVTINCQASQSIDSNLAWFQQKPG |
| | | QPPTLLIYRASNLASGVPSRFSGSRSGTEYTLTISGVQREDAATYYCLGGVGNVSYRTSFGGGT |
| | | EVVVKTGGGGSGCPRCPEPKSCDTPPPCPRCPEPKSCDTPPPCPRCPEPKSCDTPPPCPRCPAP |
| | | MFWVLVVVGGVLACYSLLVTVAFTIFWVKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHMQALPPRSGATNFSLLK |
| | | QAGDVEENPGPMLLLVTSLLLCELPHPAFLLIPRKVCNGIGIGEFKDSLSINATNIKHFKNCTS |
| | | ISGDLHILPVAFRGDSFTHTPPLDPQELDILKTVKEITGFLLIQAWPENRTDLHAFENLEIIRG |
| | | RTKQHGQFSLAVVSLNITSLGLRSLKEISDGDVIISGNKNLCYANTINWKKLFGTSGQKTKIIS |
| | | NRGENSCKATGQVCHALCSPEGCWGPEPRDCVSCRNVSRGRECVDKCNLLEGEPREFVENSECI |
| | | QCHPECLPQAMNITCTGRGPDNCIQCAHYIDGPHCVKTCPAGVMGENNTLVWKYADAGHVCHLC |
| | | HPNCTYGCTGPGLEGCPTNGPKIPSIATGMVGALLLLLVVALGIGLFM |
| 6 | 5063 | MVLQTQVFISLLLWISGAYGQSVKESEGDLVTPAGNLTLTCTASGSDINDYPISWVRQAPGKGL |
| | | EWIGFINSGGSTWYASWVKGRFTISRTSTTVDLKMTSLTTDDTATYFCARGYSTYYGDFNIWGP |
| | | GTLVTISSGGGGSGGGGSGGGGSELVMTQTPSSTSGAVGGTVTINCQASQSIDSNLAWFQQKPG |
| | | QPPTLLIYRASNLASGVPSRFSGSRSGTEYTLTISGVQREDAATYYCLGGVGNVSYRTSFGGGT |
| | | EVVVKTGGGGSGEPKSCDTPPPCPRCPEPKSCDTPPPCPRCPEPKSCDTPPPCPRCPAPMFWVL |
| | | VVVGGVLACYSLLVTVAFIIFWVKRGRKKLLYIFKQPFMRPVQTTQEEDGCSCRFPEEEEGGCE |
| | | LRVKFSRSADAPAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPRSGATNESLLKQAGDV |
| | | EENPGPMLLLVTSLLLCELPHPAELLIPRKVCNGIGIGEFKDSLSINATNIKHEKNCTSISGDL |
| | | HILPVAFRGDSFTHTPPLDPQELDILKTVKEITGFLLIQAWPENRTDLHAFENLETIRGRTKQH |
| | | GQFSLAVVSLNITSLGLRSLKEISDGDVIISGNKNLCYANTINWKKLFGTSGQKTKIISNRGEN |
| | | SCKATGQVCHALCSPEGCWGPEPRDCVSCRNVSRGRECVDKCNLLEGEPREFVENSECTQCHPE |
| | | CLPQAMNITCTGRGPDNCIQCAHYIDGPHCVKTCPAGVMGENNTLVWKYADAGHVCHLCHPNCT |
| | | YGCTGPGLEGCPTNGPKIPSIATGMVGALLLLLVVALGIGLFM |

TABLE 28
Summary of properties of selected CARs

| | | | Spacer | Nalm6 | Raji | | | |
|---|---|---|---|---|---|---|---|---|
| | | | Length with | normalized | normalized | Nalm6 | Nalm6 | Raji |
| Binder | Donor | Spacer | Linker (Å) | AUC | AUC | IFNg | IL-2 | IFNg |
| FMC63 | 3868 | 9 | 79.2 | 14.9 | 19.1 | 62067 | 68 | 142559 |
| FMC63 | 3868 | 10 | 79.2 | 14.2 | 18.3 | 65929 | 41 | 158893 |
| FMC63 | 3868 | 11 | 75.6 | 13.2 | 17.9 | 53252 | 23 | 146062 |
| FMC63 | 4869 | 9 | 79.2 | 14.3 | 49.5 | 53816 | 945 | 100333 |
| FMC63 | 4869 | 10 | 79.2 | 15.5 | 45.4 | 72105 | 517 | 139572 |
| FMC63 | 4869 | 11 | 75.6 | 12.2 | 46.2 | 54543 | 211 | 114046 |
| | | | | A549 | T47D | | | |
| | | | Spacer | normalized | normalized | A549 | A549 | T47D |
| Binder | Donor | Spacer | Length (Å) | AUC | AUC | IFNg | IL-2 | IFNg |
| Herceptin* | 15842 | 7 | 115.2 | 61.3 | 44.2 | 163107 | 267 | 162769 |
| Herceptin* | 15842 | 8 | 79.2 | 66.1 | 46.1 | 120256 | 128 | 167636 |
| Herceptin* | 15842 | 16 | 100.8 | 62.0 | 50.3 | 125690 | 157 | 163484 |
| Herceptin* | 13814 | 7 | 115.2 | 61.9 | 43.5 | 104083 | 138 | 130715 |
| Herceptin* | 13814 | 8 | 79.2 | 64.5 | 41.3 | 94188 | 194 | 154415 |
| Herceptin* | 13814 | 16 | 100.8 | 60.6 | 35.8 | 95916 | 178 | 166721 |
| | | | Spacer | A549 | H1975 | | | |
| | | | Length with | normalized | normalized | A549 | A549 | H1975 |
| Binder | Donor | Spacer | Linker (Å) | AUC | AUC | IFNg | IL-2 | IFNg |
| R12 | 2735 | 1 | 50.4 | 67.6 | 67.6 | 8935 | 4 | 15716 |
| R12 | 2735 | 15 | 43.2 | 64.0 | 63.0 | 13752 | 8 | 16921 |
| R12 | 2735 | 21 | 43.2 | 66.3 | 61.5 | 14082 | 7 | 23225 |
| R12 | 5018 | 1 | 50.4 | 80.9 | 68.8 | 4349 | 0 | 4846 |
| R12 | 5018 | 15 | 43.2 | 81.2 | 75.0 | 7613 | 9 | 4315 |
| | | | Spacer | Nalm6 | Raji | | | |
| | | | Length with | normalized | normalized | Nalm6 | Nalm6 | Raji |
| Binder | Donor | Spacer | Linker (Å) | AUC | AUC | IFNg | IL-2 | IFNg |
| R12 | 5018 | 21 | 43.2 | 85.0 | 77.4 | 5001 | 8 | 2651 |
| R11 | 7811 | 4 | 248.4 | 54.6 | 83.1 | 46161 | 26 | 15901 |
| R11 | 7811 | 5 | 205.2 | 48.8 | 69.6 | 83558 | 87 | 32411 |
| R11 | 7811 | 6 | 187.2 | 51.4 | 96.5 | 86709 | 89 | 11873 |
| R11 | 5018 | 4 | 248.4 | 59.1 | 94.5 | 37008 | 5 | 9128 |
| R11 | 5018 | 5 | 205.2 | 55.8 | 86.7 | 60935 | 10 | 17320 |
| R11 | 5018 | 6 | 187.2 | 58.4 | 114.2 | 46294 | 10 | 7174 |
| 2A2 | 2089 | 13 | 46.8 | 42.0 | 37.9 | 46579 | 1 | 67057 |
| 2A2 | 2089 | 21 | 43.2 | 38.0 | 39.4 | 55445 | 1 | 78043 |
| 2A2 | 2089 | 28 | 54 | 43.6 | 39.5 | 49905 | 1 | 78267 |
| 2A2 | 5018 | 13 | 46.8 | 61.0 | 43.2 | 38935 | 3 | 54335 |
| 2A2 | 5018 | 21 | 43.2 | 58.9 | 43.2 | 41252 | 3 | 61898 |
| 2A2 | 5018 | 28 | 54 | 61.0 | 44.3 | 34242 | 2 | 58088 |
| | | | | | Raji CD19 | Raji | | |
| | | | Spacer | | KO | CD19 | Raji | No |
| | | | Length with | | normalized | KO | CD19 | Target |
| Binder | Donor | Spacer | Linker (Å) | Raji IL-2 | AUC | IFNg | KO IL-2 | IFNg |
| FMC63 | 3868 | 9 | 79.2 | 3216 | 238307 | 4480 | 17 | 1211 |
| FMC63 | 3868 | 10 | 79.2 | 5497 | 247388 | 4745 | 12 | 1649 |
| FMC63 | 3868 | 11 | 75.6 | 3372 | 245963 | 3667 | 9 | 1853 |
| FMC63 | 4869 | 9 | 79.2 | 10092 | 223873 | 3501 | 32 | 556 |
| FMC63 | 4869 | 10 | 79.2 | 13465 | 199757 | 6382 | 41 | 2984 |
| FMC63 | 4869 | 11 | 75.6 | 9042 | 240626 | 3048 | 18 | 638 |
| | | | | | T47D Her2 | T47D | | |
| | | | | | KO | Her2 | T47D | No |
| | | | Spacer | | normalized | KO | Her2 KO | Target |
| Binder | Donor | Spacer | Length (Å) | T47D IL-2 | AUC | IFNg | IL-2 | IFNg |
| Herceptin* | 15842 | 7 | 115.2 | 857 | 509961 | 2632 | 2 | 1830 |
| Herceptin* | 15842 | 8 | 79.2 | 1525 | 563903 | 2532 | 1 | 1764 |
| Herceptin* | 15842 | 16 | 100.8 | 1147 | 713877 | 2470 | 1 | 1674 |
| Herceptin* | 13814 | 7 | 115.2 | 731 | 827793 | 1619 | 1 | 1331 |
| Herceptin* | 13814 | 8 | 79.2 | 1459 | 823493 | 1533 | 1 | 1415 |
| Herceptin* | 13814 | 16 | 100.8 | 984 | 804135 | 2581 | 0 | 2024 |
| | | | | | Raji CD19 | Raji | | |
| | | | Spacer | | KO | CD19 | Raji | No |
| | | | Length with | | normalized | KO | CD19 | Target |
| Binder | Donor | Spacer | Linker (Å) | Raji IL-2 | AUC | IFNg | KO IL-2 | IFNg |
| | | | | | A549 | A549 | | |
| | | | Spacer | | ROR1 KO | ROR1 | A549 | No |
| | | | Length with | | normalized | KO | ROR1 | Target |
| Binder | Donor | Spacer | Linker (Å) | H1975 IL-2 | AUC | IFNg | KO IL-2 | IFNg |
| R12 | 2735 | 1 | 50.4 | 10 | 283488 | 995 | 0 | 419 |
| R12 | 2735 | 15 | 43.2 | 17 | 262223 | 1353 | 3 | 415 |
| R12 | 2735 | 21 | 43.2 | 26 | 264326 | 1391 | 0 | 478 |
| R12 | 5018 | 1 | 50.4 | 0 | 275978 | 465 | 1 | 1051 |
| R12 | 5018 | 15 | 43.2 | 10 | 299606 | 1528 | 1 | 2004 |
| R12 | 5018 | 21 | 43.2 | 7 | 301079 | 1257 | 1 | 2157 |
| R11 | 7811 | 4 | 248.4 | 4 | 275413 | 3778 | 2 | 2437 |
| R11 | 7811 | 5 | 205.2 | 16 | 238046 | 6430 | 3 | 3317 |
| R11 | 7811 | 6 | 187.2 | 2 | 256889 | 5902 | 2 | 3292 |
| R11 | 5018 | 4 | 248.4 | 1 | 301271 | 3221 | 1 | 2744 |
| R11 | 5018 | 5 | 205.2 | 2 | 279139 | 5458 | 1 | 3391 |
| R11 | 5018 | 6 | 187.2 | 1 | 297120 | 5353 | 1 | 3814 |
| 2A2 | 2089 | 13 | 46.8 | 1 | 161899 | 13199 | 1 | 15196 |
| 2A2 | 2089 | 21 | 43.2 | 2 | 175504 | 16360 | 1 | 19127 |
| 2A2 | 2089 | 28 | 54 | 1 | 168503 | 19835 | 1 | 22465 |
| 2A2 | 5018 | 13 | 46.8 | 14 | 225124 | 4030 | 0 | 4824 |
| 2A2 | 5018 | 21 | 43.2 | 13 | 233789 | 5220 | 0 | 6602 |
| 2A2 | 5018 | 28 | 54 | 8 | 215509 | 5721 | 1 | 6763 |

*Her2 spacer length calculations do not include the GGGSG linker of SEQ ID NO: 4818

### Example 5

### In Vivo Anti-Tumor Activity of R12 CARs

Methods: NCI-H1975 tumor cells were implanted into mice with a take rate of 100%. Animals were randomized into treatment groups of 5 animals 11 days post tumor implantation based on tumor volume with a mean tumor size of 101.91 mm3+/−1.24 mm3. All groups received appropriate intravenous T cell treatment on day 12. Tumor measurements were collected by caliper and body weights collected by digital scale 2 times per week. Blood was collected for PK analysis via the submental region 24 hours post T cell dose, and weekly thereafter for 5 weeks total. Animals were euthanized when tumors reached 2000 mm3 in size, body weight loss exceeded 20% or were moribund.

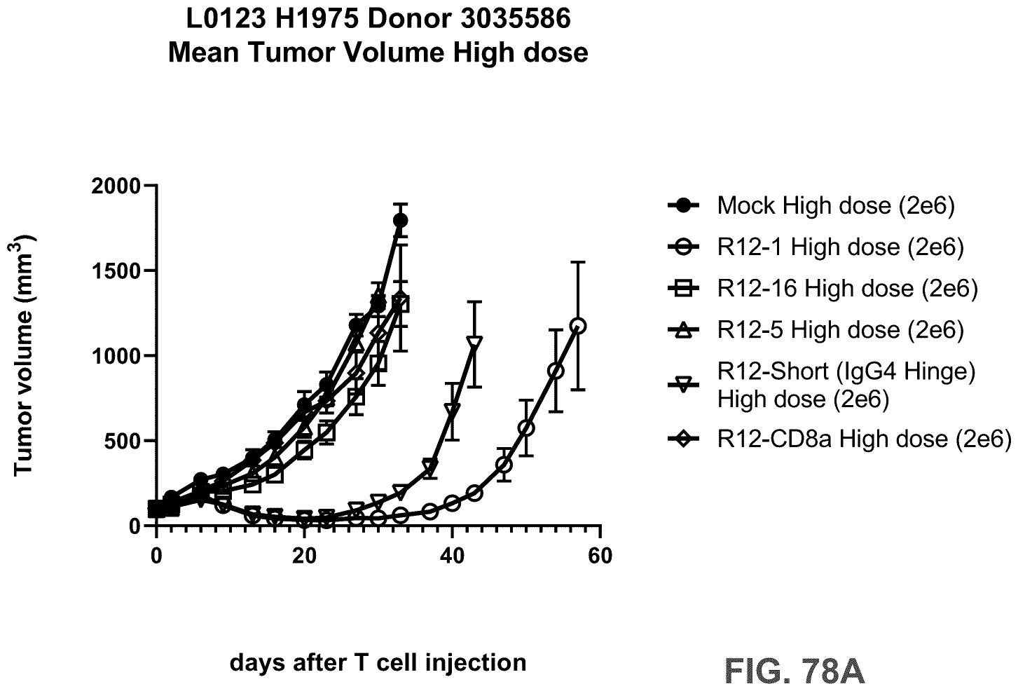

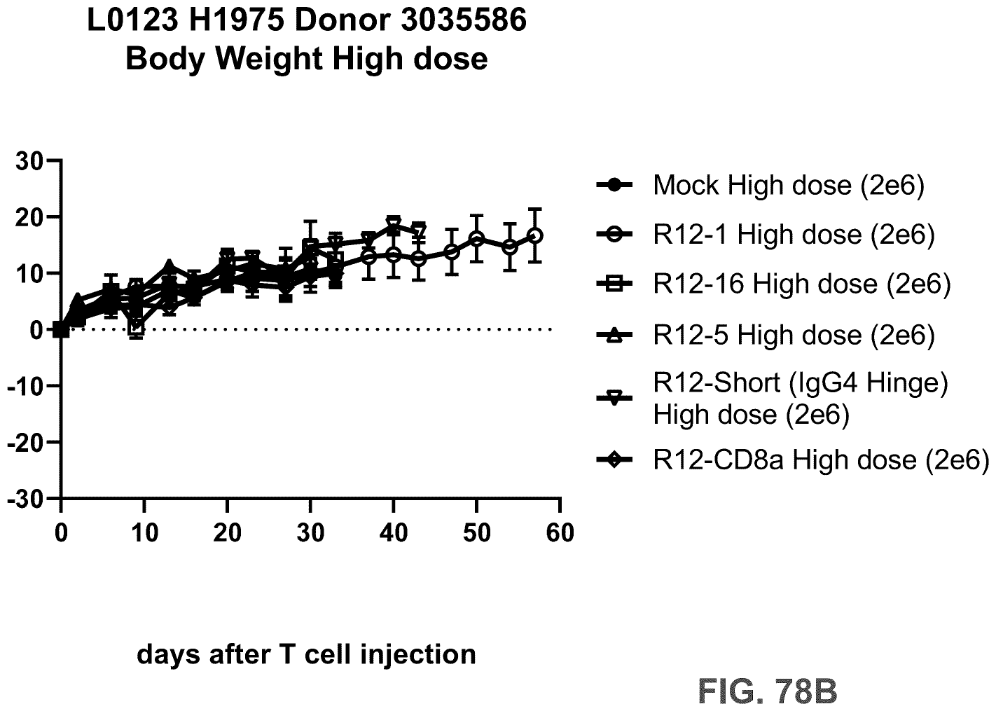

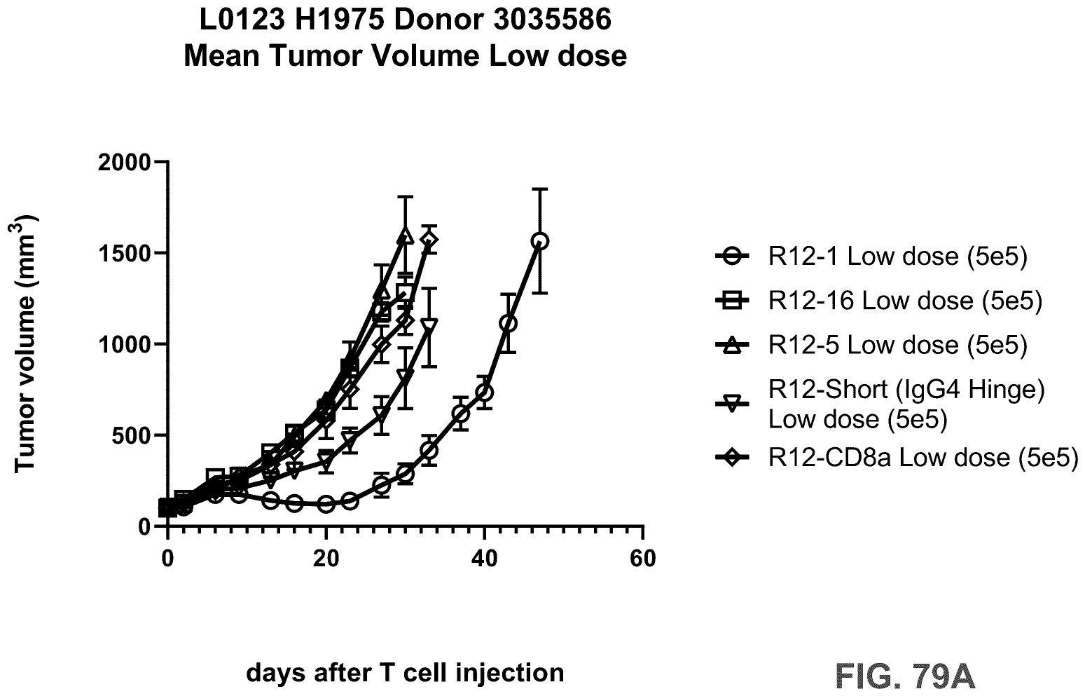

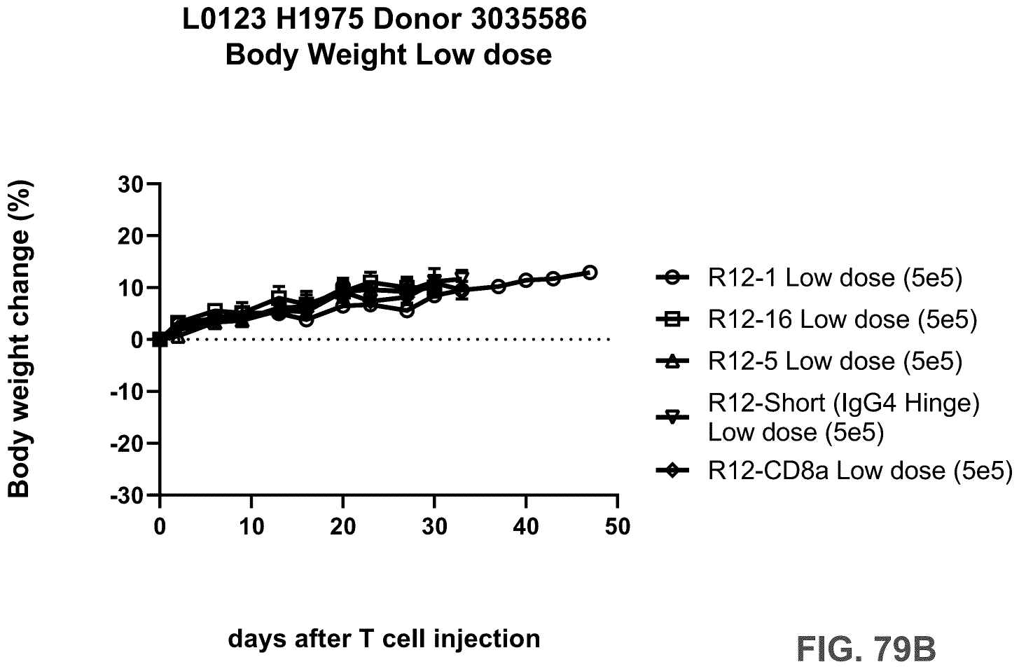

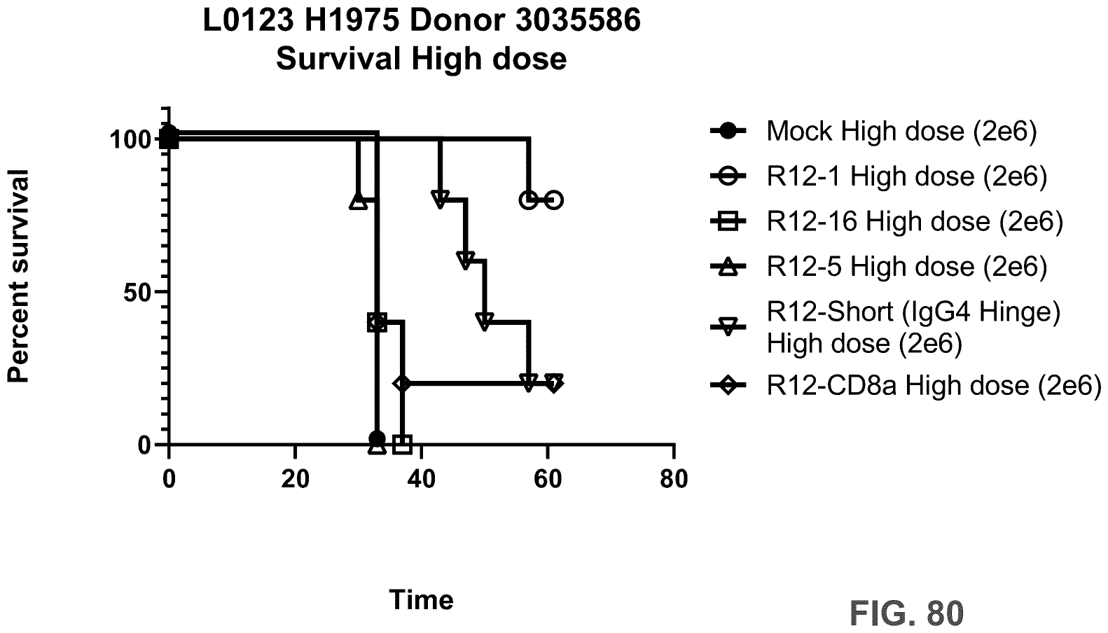

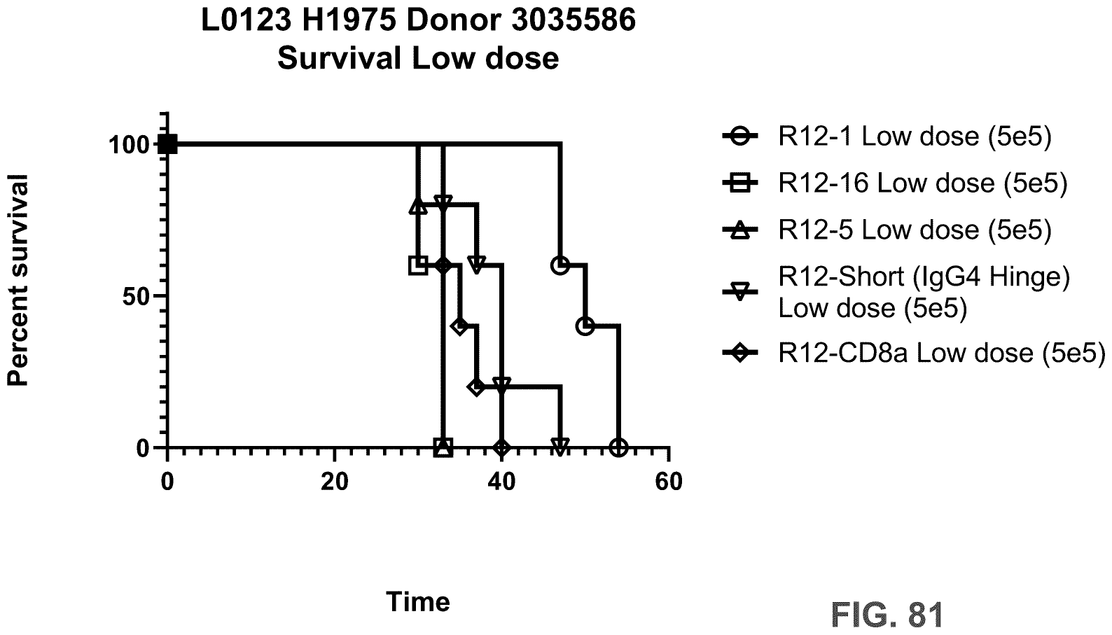

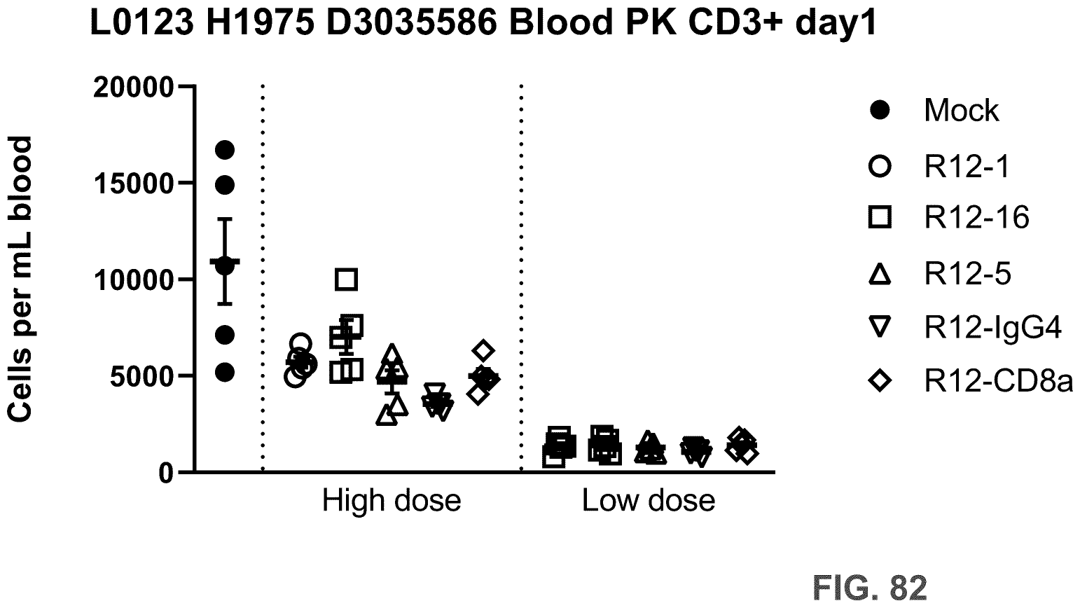

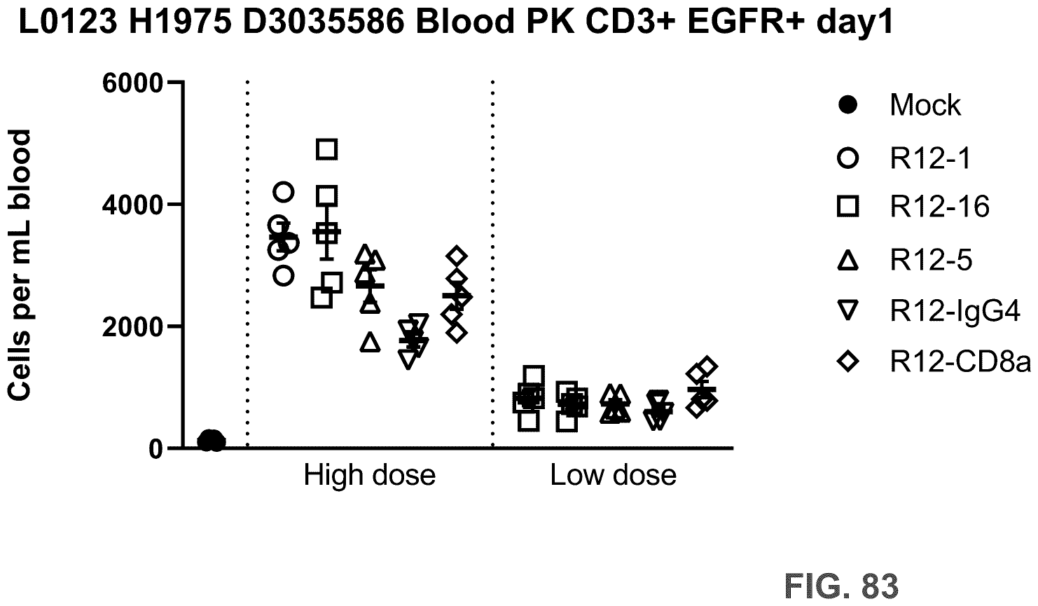

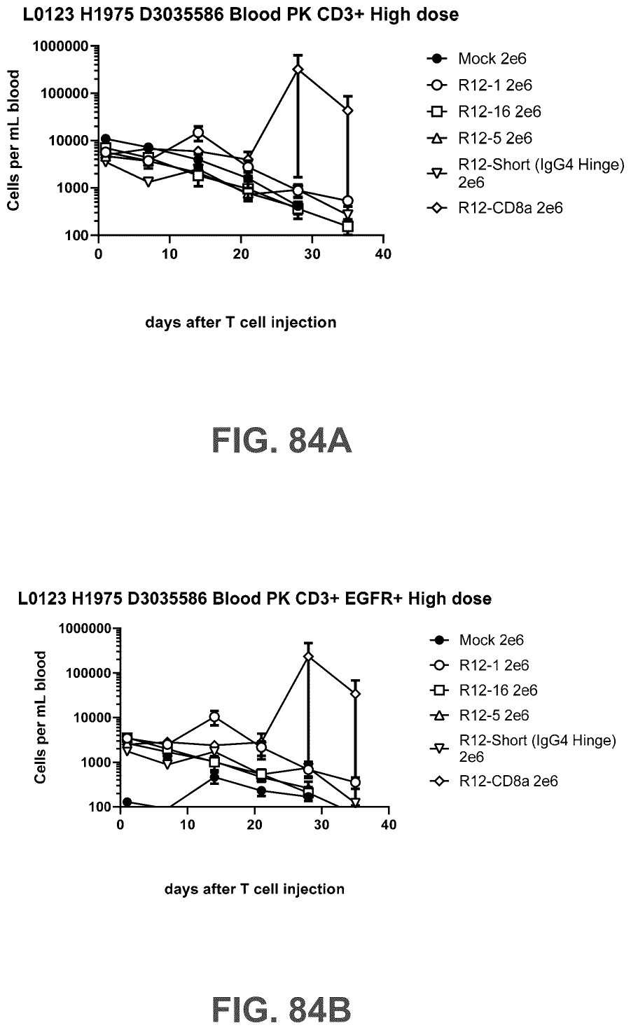

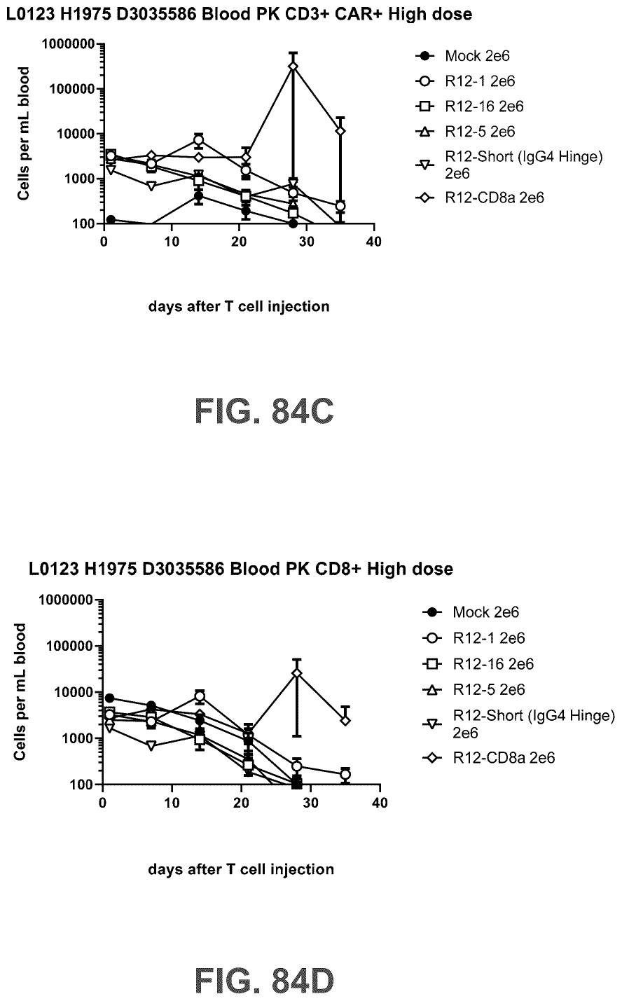

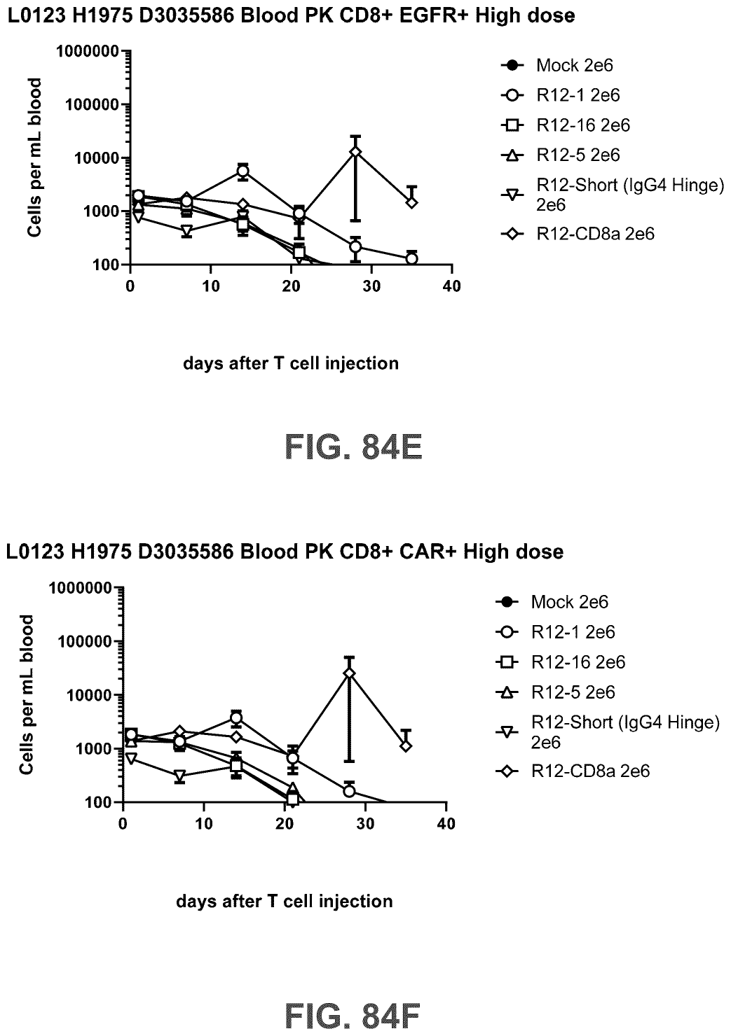

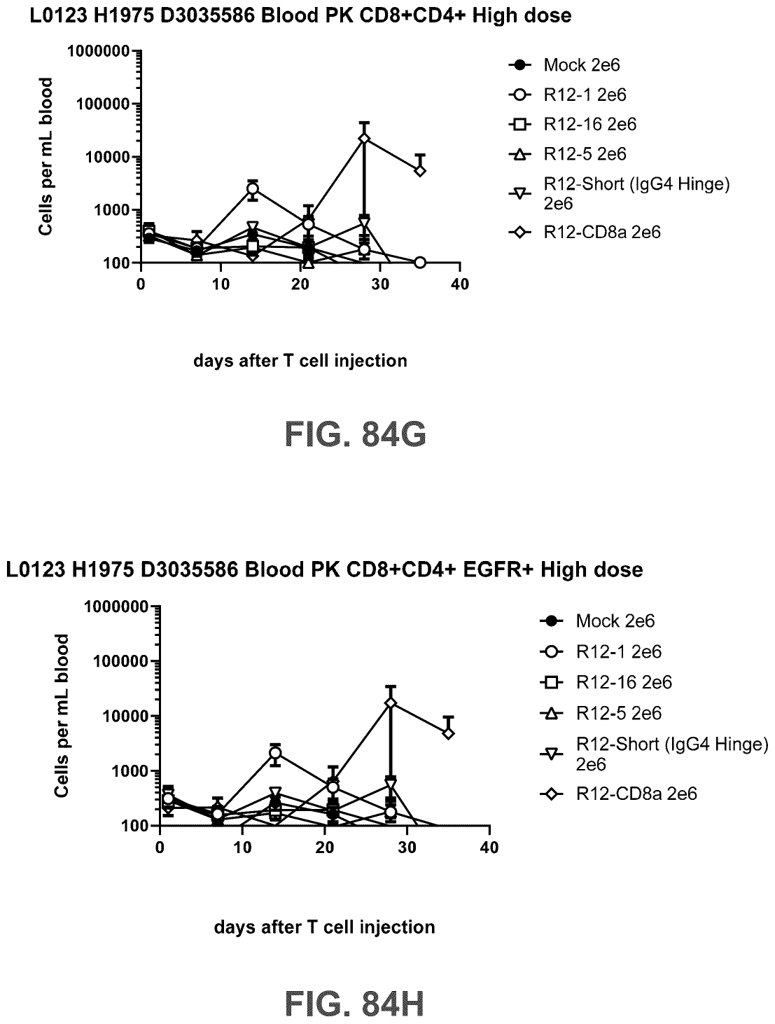

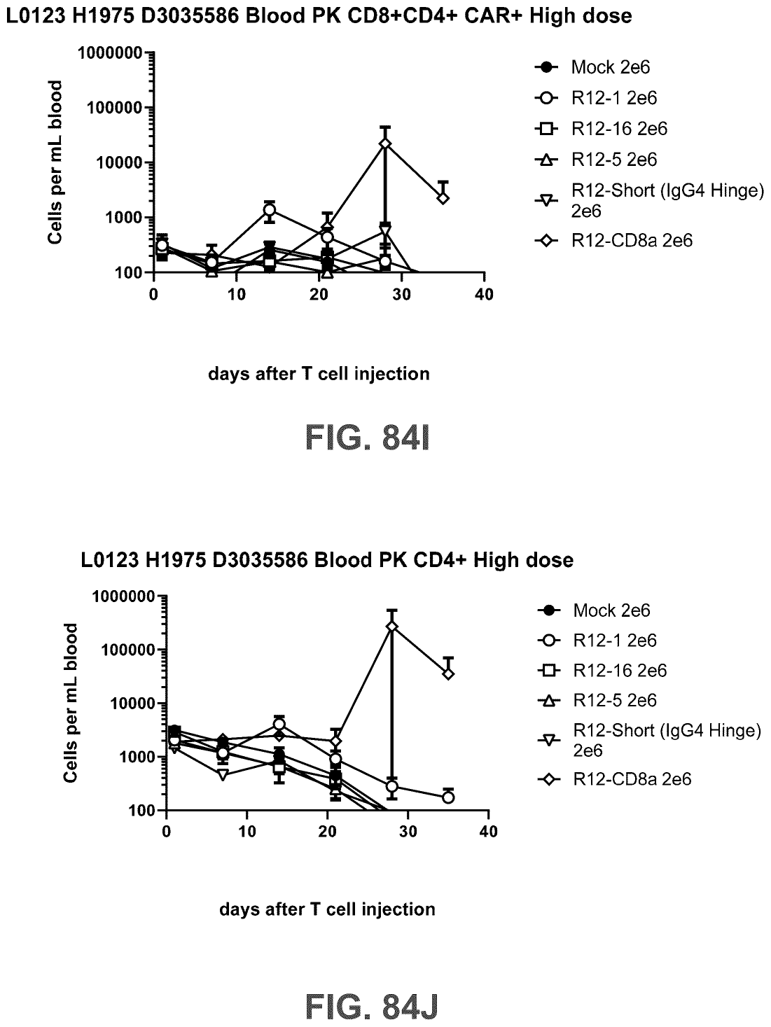

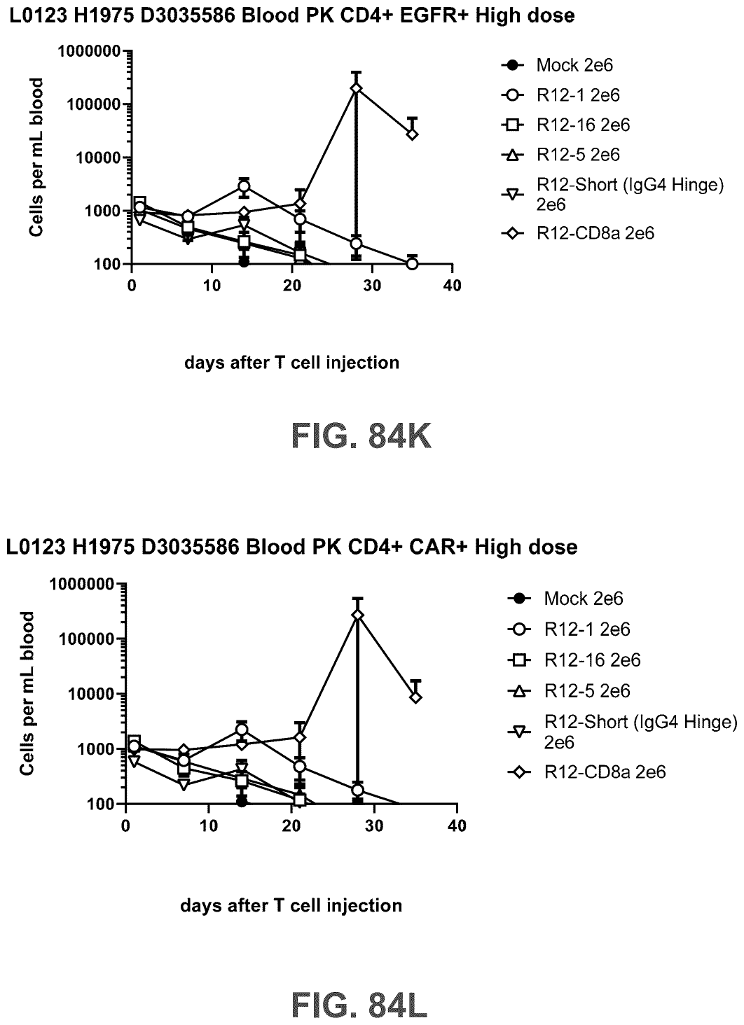

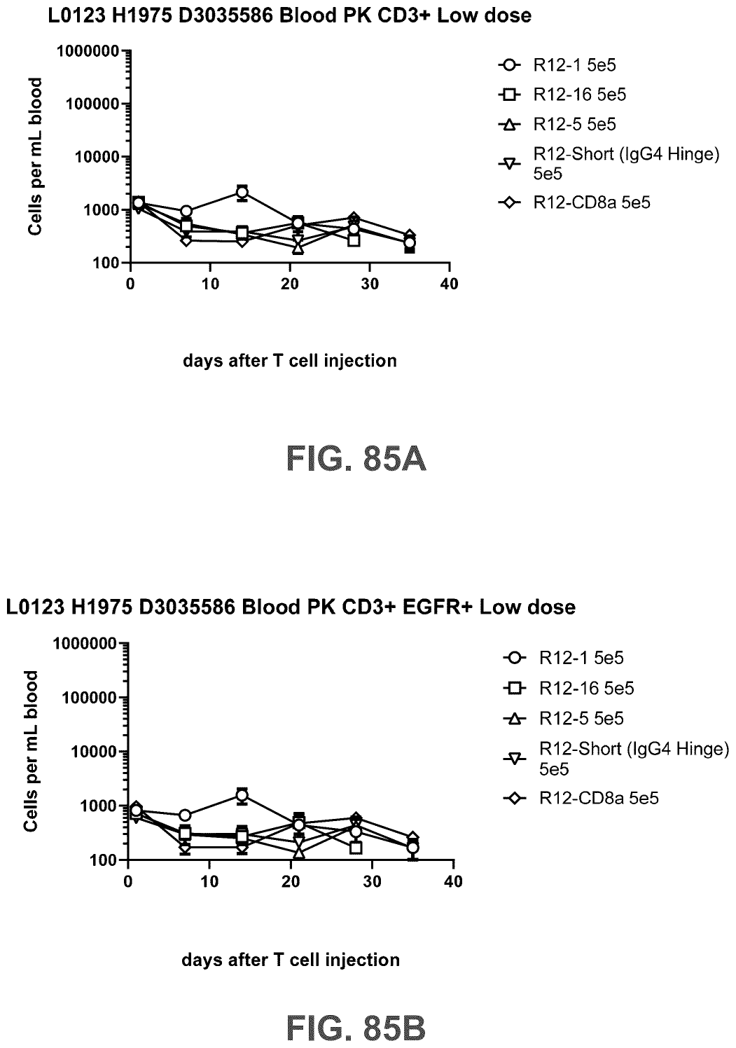

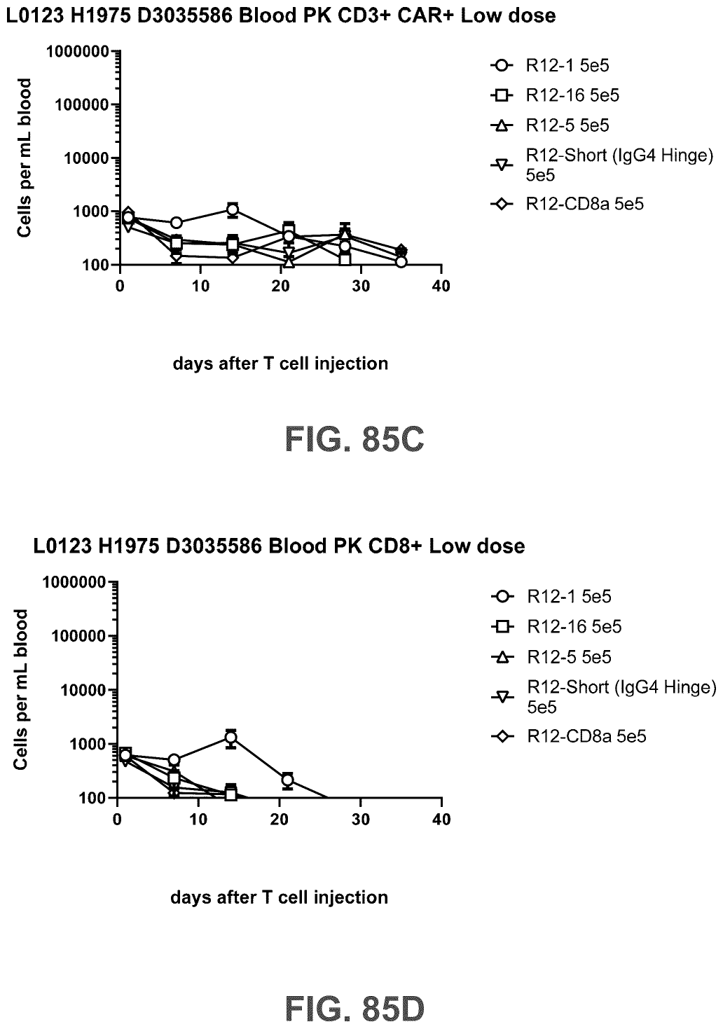

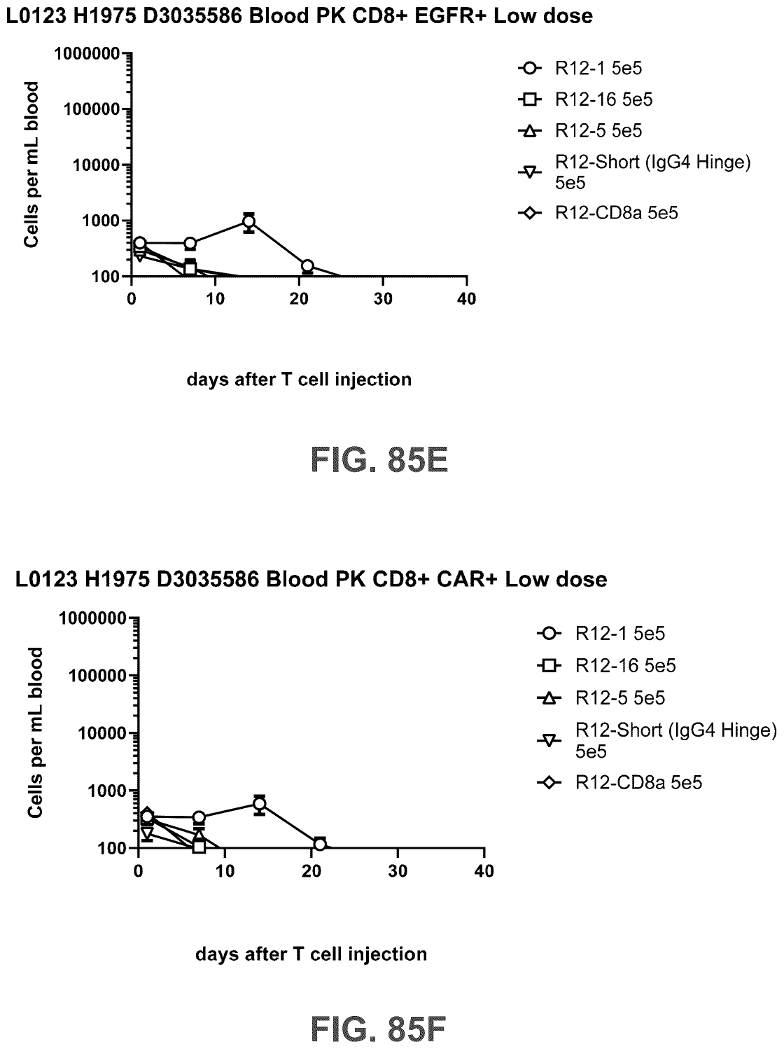

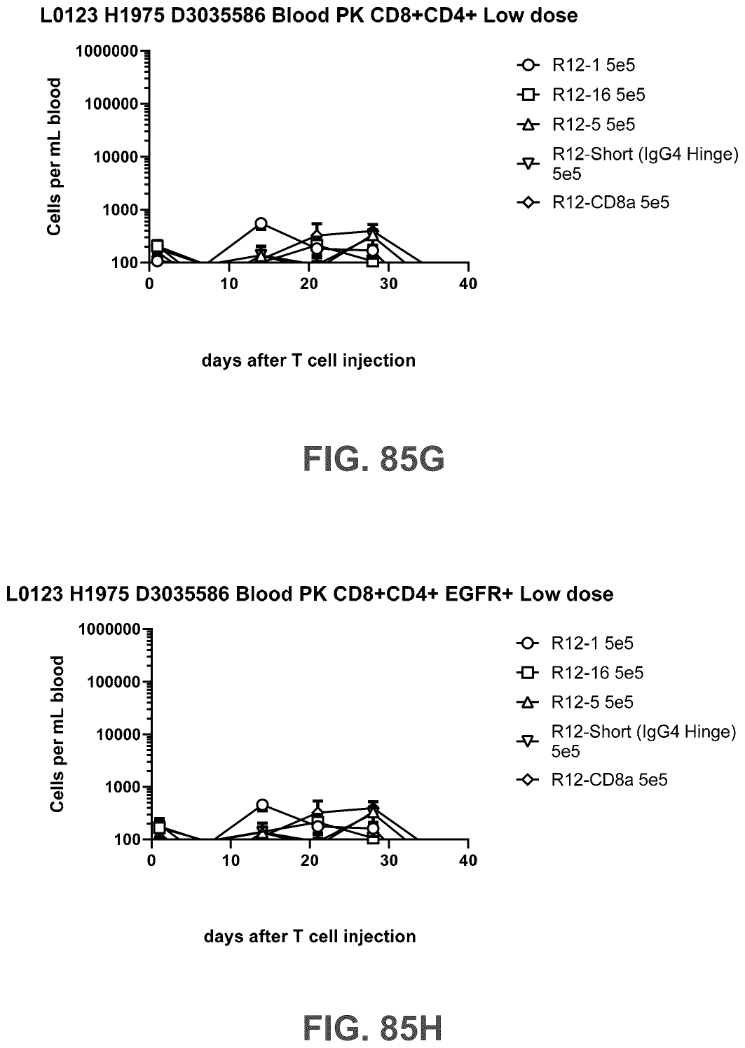

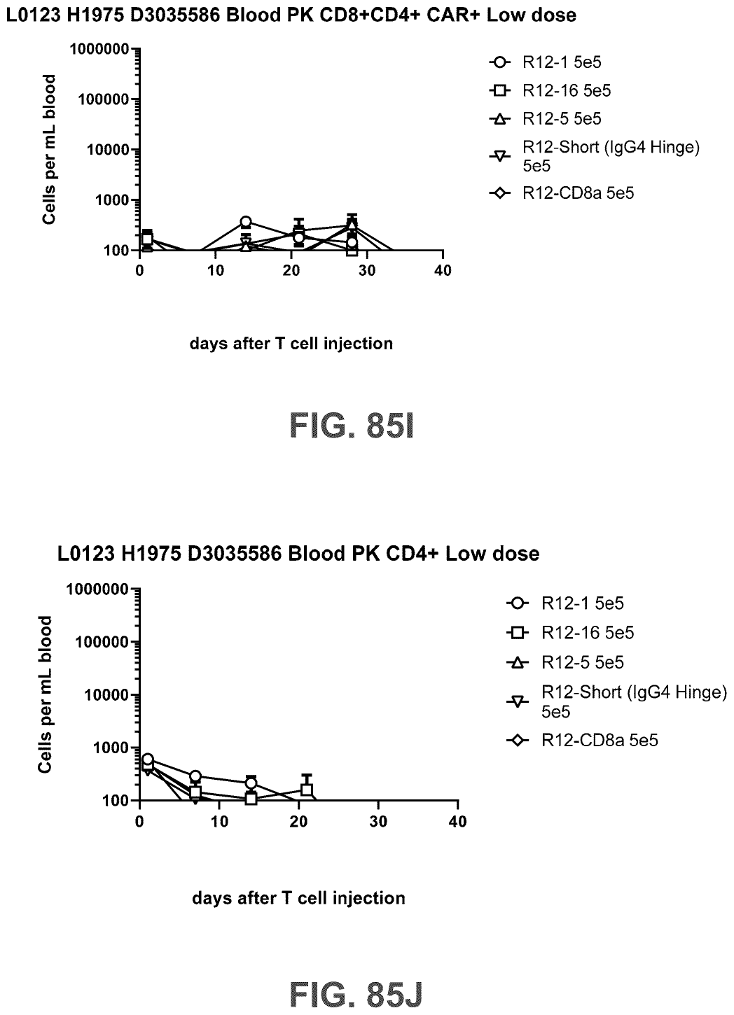

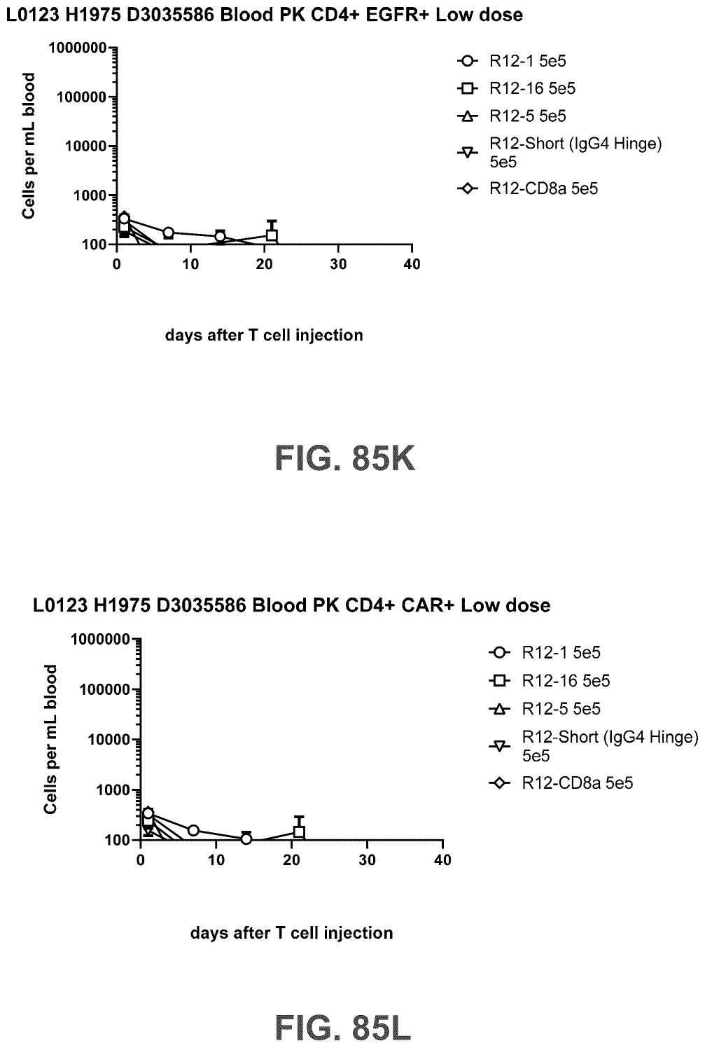

Results: The study was successfully conducted and demonstrated that R12-1 (CAR comprising Spacer 1; SEQ ID NO: 5049) had the highest anti-tumor activity of all products tested, shown by lowest tumor volumes, extended survival, and a marked increase in peripheral CAR+ T cell counts (for both CD4+ and CD8+ T cells) by day 14 at both dose levels (see FIGS. 82A to 85L; PK graphs show mean +/−standard error of the mean of all animals alive at the time of sampling). R12-IgG4 (CAR with “Short” reference spacer of SEQ ID NO: 4911) was the second strongest CAR T cell product, also extending survival but without expansion of T cells in the blood. A single animal in the R12-CD8a (CAR with CD8a spacer) group had a delayed but strong anti-tumor response which coincided with strong expansion of CAR T cells into the blood at day 28. No other animal in that group had an anti-tumor response and thus the R12-CD8a group is considered not efficacious. R12-5 (CAR comprising Spacer 5) and R12-16 (CAR comprising Spacer 16) products did not show significant anti-tumor activity at any dose level tested. See FIG. 78A to FIG. 85L.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

1-154. (canceled)

155. A polynucleotide comprising a nucleic acid sequence encoding a chimeric antigen receptor (CAR) comprising
(i) an antigen-binding domain that binds to an epitope on a tumor antigen expressed on a target cell;
(ii) a transmembrane domain;
(iii) an intracellular domain; and,
(iv) a CAR spacer located between the antigen-binding domain and the transmembrane domain comprising an amino acid sequence derived from a human immunoglobulin hinge and/or constant region or a functional fragment thereof,
wherein
(a) the CAR spacer is between about 150 amino acids and about 125 amino acids in length; and, the distance between the epitope and the surface of the target cell membrane is less than about 10 Å;
(b) the CAR spacer is between about 125 amino acids and about 100 amino acids in length; and, the distance between the epitope and the surface of the target cell membrane is less than about 10 Å;
(c) the CAR spacer is between about 100 amino acids and about 75 amino acids in length; and, the distance between the epitope and the surface of the target cell membrane is less than about 10 Å;
(d) the CAR spacer is between about 75 amino acids and about 36 amino acids in length; and, the distance between the epitope and the surface of the target cell membrane is less than about 15 Å;
(e) the CAR spacer is between about 35 amino acids and about 21 amino acids in length; and, the distance between the epitope and the surface of the target cell membrane is between about 15 Å and about 25 Å;
(f) the CAR spacer is between about 20 amino acids and about 16 amino acids in length; and, the distance between the epitope and the surface of the target cell membrane is between about 25 Å and about 35 Å;
(g) the CAR spacer is between about 15 amino acids and about 11 amino acids in length; and, the distance between the epitope and the surface of the target cell membrane is between about 35 Å and about 45 Å; or,
(h) the CAR spacer is between about 10 amino acids and about 5 amino acids in length; and, the distance between the epitope and the surface of the target cell membrane is more than about 45 Å.

156. The polynucleotide of claim 155, wherein
(a) the distance between the epitope and the surface of the target cell membrane is less than about 10 Å and the CAR spacer is 125, 126, 127, 128, 129, 130, 131, 132, 133, 134, 135, 136, 137, 138, 139, 140, 141, 142, 143, 144, 145, 146, 147, 148, 149 or 150 amino acids in length;
(b) the distance between the epitope and the surface of the target cell membrane is less than about 10 Å and the CAR spacer is 100, 101, 102, 103, 104, 105, 106, 107, 108, 109, 110, 111, 112, 113, 114, 115, 116, 117, 118, 119, 120, 121, 122, 123, 124 or 125 amino acids amino acids in length;
(c) the distance between the epitope and the surface of the target cell membrane is less than about 10 Å and the CAR spacer is 75, 76, 77, 78, 79, 80, 81, 82, 83, 84, 85, 86, 87, 88, 89, 90, 91, 92, 93, 94, 95, 96, 97, 98, 99 or 100 amino acids in length;
(d) the distance between the epitope and the surface of the target cell membrane is less than about 15 Å and the CAR spacer is 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 amino acids in length;
(e) the distance between the epitope and the surface of the target cell membrane is between about 15 Å and about 25 Å and the CAR spacer is 21, 22, 23, 24, 25, 26, 27, 28, 29, 30, 31, 32, 33, 34 or 35 amino acids in length;
(f) the distance between the epitope and the surface of the target cell membrane is between about 25 Å and about 35 Å and the CAR spacer is 16, 17, 18, 19 or 20 amino acids in length;
(g) the distance between the epitope and the surface of the target cell membrane is between about 35 Å and about 45 Å and the CAR spacer is 11, 12, 13, 14, or 15 amino acids in length; or,
(h) the distance between the epitope and the surface of the target cell membrane is more than about 45 Å and the CAR spacer is 5, 6, 7, 8, 9 or 10 amino acids in length.

157. The polynucleotide of claim 155, wherein the tumor antigen comprises ROR1, HER2, AFP, TRAC, TCRP, BCMA, CLL-1, CS1, CD38, CD19, TSHR, CD123, CD22, CD30, CD70, CD171, CD33, EGFRvIII, GD2, GD3, Tn Ag, PSMA, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, and any combinations thereof.

158. The polynucleotide of claim 155, wherein the CAR spacer located between the antigen-binding domain and the transmembrane domain comprises an amino acid sequence derived from a human or mouse immunoglobulin hinge and/or constant region or a functional fragment thereof, wherein the immunoglobulin is selected from the group consisting of mouse IgG2, human IgG1, human IgG2, human IgG3, human IgA1, human IgA2, human IgD, human IgE, and human IgM.

159. The polynucleotide of claim 155, wherein the CAR spacer comprises an amino acid sequence having at least 70%, 75%, 80%, 85%, 90%, 95%, 97%, 99%, or 100% sequence identity to
(i) a mouse IgG2 sequence selected from the group consisting of SEQ ID NOS: 4830, 4831, and 4832;
(ii) a human IgG3 sequence selected from the group consisting of SEQ ID NOS: 4833, 4834, 4835, 4836, 4837, 4838, and 4841;
(iii) a human IgG1 sequence selected from the group consisting of SEQ ID NOS: 4839, 4840, and 4843;
(iv) a human IgG2 sequence of SEQ ID NO: 4842;
(v) a human IgG1 sequence selected from the group consisting of SEQ ID NOS: 4839, 4840, and 4843;
(vi) a human IgAQ1 sequence selected from the group consisting of SEQ ID NOS: 4844, 4845, 4846, and 4847;
(vii) a human IgA2 sequence selected from the group consisting of SEQ ID NOS: 4848, 4849, 4850, and 4851;
(viii) a human IgD sequence selected from the group consisting of SEQ ID NOS: 4852, 4853, 4854, and 4855;
(ix) a human IgE sequence of SEQ ID NO: 4856; or
(x) a human IgM sequence selected from the group consisting of SEQ ID NOS: 4857, 4848, and 4859.

160. The polynucleotide of claim 155, wherein the CAR spacer further comprises an N-terminal Glycine/Serine linker selected from the group consisting of the sequence GGGSG (SEQ ID NO: 4818) and the sequence GGGGSG (SEQ ID NO: 5088).

161. The polynucleotide of claim 155, wherein the antigen-binding domain comprises the six complementary determining regions (CDRs) derived from the R11 antibody, R12 antibody, or 2A2 antibody.

162. The polynucleotide of claim 155, wherein the antigen-binding domain comprises:
(i) VH CDR1 of SEQ ID NO: 4888; VH CDR2 of SEQ ID NO: 4889; and VH CDR3 of SEQ ID NO:
4890; and/or VL CDR1 of SEQ ID NO: 4892; VL CDR2 of SEQ ID NO: 4893; and VL CDR3 of SEQ ID NO: 4894;
(ii) VH CDR1 of SEQ ID NO: 4896; VH CDR2 of SEQ ID NO: 4897; and VH CDR23 of SEQ ID NO: 4898; and/or VL CDR1 of SEQ ID NO: 4900; VL CDR2 of SEQ ID NO: 4901; and VL CDR3 of SEQ ID NO: 4902; or,
(iii) VH CDR1 of SEQ ID NO: 4904; VH CDR2 of SEQ ID NO: 4905; and VH CDR23 of SEQ ID NO: 4906; and/or VL CDR1 of SEQ ID NO: 4908; VL CDR2 of SEQ ID NO: 4909; and VL CDR3 of SEQ ID NO: 4910.

163. The polynucleotide of claim 155, wherein the antigen-binding domain comprises a VH and a VL, and wherein:
(i) the VH comprises SEQ ID NO: 4887; or the VL comprises SEQ ID NO: 4891;
(ii) the VH comprises SEQ ID NO: 4895; or the VL comprises SEQ ID NO: 4899; or,
(iii) the VH comprises SEQ ID NO: 4903; or the VL comprises SEQ ID NO: 4907.

164. The polynucleotide of claim 155, wherein the CAR comprises an amino acid sequence set forth is SEQ ID NO: 5061, 5062, 5063, 5058, 5059, 5060, 5049, 5050, or 5051.

165. A vector comprising the polynucleotide of claim 155.

166. A composition comprising the polynucleotide of claim 155.

167. A CAR encoded by the polynucleotide of claim 155.

168. A cell genetically modified to express a CAR, comprising the polynucleotide of claim 155.

169. The cell of claim 168, which comprises a T cell, a natural killer (NK) cell, an natural killer T (NKT) cell, an ILC cell, a macrophage, or an antigen presenting cell.

170. A pharmaceutical composition comprising the cell of claim 168.

171. A culture medium comprising the cell of claim 168.

172. A method of treating cancer or providing an anti-tumor immunity in a subject in need thereof comprising administering to the subject an effective amount of a vector, composition, cell, or pharmaceutical composition comprising the polynucleotide of claim 155.

173. A method of preparing a population of cells for a therapy comprising transducing a population of cells isolated from a subject with a polynucleotide or vector comprising the polynucleotide of claim 155.

174. A method of generating a persisting population of genetically engineered cells in a subject with cancer, the method comprising administering to the subject a cell genetically engineered to express a CAR encoded by the polypeptide of claim 155.
